(19) United States
(12) Patent Application Publication
(10) Pub. No.: US 2014/0161729 A1
(43) Pub. Date: Jun. 12, 2014

(54) COFLUORONS AND METHODS OF MAKING AND USING THEM

(71) Applicants: Francis Barany, New York, NY (US); Maneesh Pingle, New York, NY (US); Donald E. Bergstrom, West Lafayette, IN (US); Sarah F. Giardina, New York, NY (US); Lee Daniel Arnold, Mt. Sinai, NY (US)

(72) Inventors: Francis Barany, New York, NY (US); Maneesh Pingle, New York, NY (US); Donald E. Bergstrom, West Lafayette, IN (US); Sarah F. Giardina, New York, NY (US); Lee Daniel Arnold, Mt. Sinai, NY (US)

(73) Assignee: CORNELL UNIVERSITY, Ithaca, NY (US)

(21) Appl. No.: 14/110,019

(22) PCT Filed: Apr. 9, 2012
(86) PCT No.: PCT/US12/00198

§ 371 Date: Feb. 28, 2014

Related U.S. Application Data
(60) Provisional application No. 61/457,481, filed on Apr. 7, 2011.

(51) Int. Cl. G01N 33/573; C07D 405/06; C07D 491/107; C07D 211/26; C07D 263/06; C07F 5/02; C07D 401/06

(52) U.S. Cl. CPC G01N 33/573; C07F 5/025; C07D 405/06; C07D 401/06; C07D 211/26; C07D 263/06; C07D 491/107
USPC 424/9.6; 546/13; 546/196; 546/199; 546/245; 544/137; 546/17; 435/7.8

(57) ABSTRACT

The present invention is directed to method of using a collection of monomers capable of forming multimers as a fluorescence reporter in different applications such as ligand detection/screening, disease diagnosis, drug discovery or screening, fluorescent labeling and imaging, or other fluorescent methodologies. Each monomer in the collection includes one or more ligand elements useful for binding to a target molecule with a dissociation constant of less than 300 μM and a linker element connected to the ligand elements directly or indirectly through a connector. Association of linker elements of different combinations of monomers, with their ligand elements bound to the target molecule to form a multimer, will generate a unique fluorescent signature different from that produced by those monomers either alone or in association with each other in the absence of the target molecule, when subjected to electromagnetic excitement.

This application claims the benefit of U.S. Provisional Patent Application Ser. No. 61/457,481, filed Apr. 7, 2011, which is hereby incorporated by reference in its entirety.

This invention was made with government support under Public Health Service grant 5U01AI075470-04 from the National Institute of Allergy and Infectious Diseases. The government has certain rights in this invention.

### FIELD OF THE INVENTION

The present invention is directed to cofluorons and methods of making and using them.

### BACKGROUND OF THE INVENTION

Fluorescence has been used broadly in biological systems for tracking, identifying, sorting, or analyzing biological molecules. Often, reporter molecules are excited at a given wavelength followed by fluorescent emission at a specific frequency where there is minimal. or no background from the excitation light, as relatively few cellular components are naturally fluorescent.

Methodologies for detection and visualization of target ligands have employed fluorescent dyes or tags as binding reporters for the target ligands. Fluorescent dyes or tags are widely used in various applications as detection reagents, for instance, in labeling a component of a sample and determining the presence, quantity or location of that component.

For target-specific detection or visualization, target-associative fluorescent tags have typically been used. For example, an antibody-associative fluorescent tag can be associated with an antibody to confer the property of fluorescence upon the antibody, producing a fluorescent antibody used as target-associative tag that can be directed against structures to which the antibody has an affinity. A target-specific fluorescence-tagged antibody can achieve a tight binding to the target and good specificity through the diversity generated in its complementarity-determining regions.

Assays employing fluorescent antibody to target the selective macromolecules or interactions based on the specific antibody affinities have gained popularity in many areas, ranging from drug discovery, medical diagnostics and imaging, and environmental monitoring to quality control of food. For example, an approach to cancer diagnosis and imaging involves directing the fluorescent antibodies or fluorescent antibody fragments to disease tissues, where the antibody or antibody fragment can target a diagnostic agent to the disease site.

Currently, antibody/antigen interaction-based immunoassays, particularly heterogeneous immunoassays (e.g., enzyme-linked immunosorbant immunoassay), are the most commonly used biological assaying techniques for drug screening and medical diagnostics. Fluorescence tagged-antibodies can be employed in the immunoassays for signal generating and reporting. In these heterogeneous immunoassays, antibodies (or antigens) are often immobilized on solid surfaces and detection antigens (or detection antibodies) are captured on the modified surfaces through either direct or competitive binding. The detection antibody (or antigens) can be covalently linked to an enzyme, or can itself be detected by a secondary antibody that is linked to an enzyme through bioconjugation (e.g., in a sandwiched enzyme immunoassay). Between each of the above steps, the plate is typically washed with a mild detergent solution to remove any proteins or antibodies that are not specifically bound. Additionally, chromogenic or fluorescent marker labeling typically occurs in a separate step and is followed by another washing step to remove dyes or fluorescent labels so that they do not interfere with scoring. Fluorescent signal generating and reporting step follows the final wash step. On one hand, these heterogeneous immunoassay methods are usually quite general and selective; on the other hand, they are expensive, labor-intensive, and time-consuming. For example, for many applications such as high throughput drug screening where a large number of assays are carried out daily, these additional washing steps can complicate the procedures and results, and add significant cost to the methods.

Moreover, techniques employing fluorescent antibodies are limited to targeting ligand interactions or activities that are on the surface of tumors or circulating targets. Because antibodies are too large to permeate cells, fluorescent antibodies are not able to use their specificity to detect or monitor intracellular interactions or activities.

Traditionally, to visualize proteins in living cells or to detect and/or image intracellular interactions, recombinant proteins with fluorescent tags have been used and introduced into cells to stain the target of interest. For example, green fluorescent protein (GFP) derived from Aequorea victoria, a jellyfish, and various GFP mutants, such as yellow fluorescent protein (YFP) or cyan fluorescent protein (CFP), and red fluorescent protein (RFP) which has been isolated from sea anemone (Discoma sp.) have been developed to cover an expanded range of the fluorescent spectrum. These fluorescent proteins can be fused with other proteins by gene recombinant technique, and subsequent detecting and monitoring of the expression and transportation of these fusion proteins can be carried out.

These techniques, however, involve expensive and time consuming procedures. Further, they utilize invasive reconstruction of proteins and/or genes, and hence may interfere with the interactions or activities to be detected. Additionally, the requirement to re-engineer the cells being studied prevents this methodology from being used directly on living disease tissues, such as freshly excised tumor tissue and living tumor cells from biopsies.

Thus, the current fluorescent reporting methodologies, particularly when applied to biological systems, do not address the urgent need to find the appropriate reporting agents. Commercially available small fluorescent tags typically do not have the required specificity. Fluorescent antibodies have the required specificity to distinguish among different target macromolecules or interactions based on the specific antibody affinities; however, they are too large to enter cells. Recombinant techniques using fusion proteins containing fluorescent tags have been designed to be introduced to cells; however, they involve expensive and time consuming procedures, and they are invasive techniques that may interfere with the interactions or activities to be probed.

Thus, there is a need for easy and convenient reporter agents or imaging probes that can detect the presence of biological macromolecules, or interactions and activities of macromolecules, particularly in vivo, in a non-invasive way.

The present invention is directed to answering these needs in the art.

### SUMMARY OF THE INVENTION

One aspect of the present invention relates to a method of detecting the presence or absence of a target molecule in a sample. The method includes providing a sample potentially containing one or more target molecules. Also provided is a set of one to six monomers. Each monomer comprises one or more ligand elements, which are useful for binding to a target molecule with a dissociation constant less than 300 μM, and a linker element being connected directly or indirectly through a connector to the one or more ligand elements. The linker element is capable of forming a bond with one or more linker elements of either the same or a different monomer of the set of monomers. Association of the linker elements, with their ligand elements bound to the target molecule to form a multimer, will generate a unique fluorescent signature different from that produced by those monomers either alone or in association with each other in the absence of target, when subjected to electromagnetic excitation. The sample is contacted with the set of monomers under conditions effective to allow the ligand elements to bind to the target molecules, if the target molecules are present in the sample. The monomers are subjected to reaction conditions effective for the linker elements of either the same or different monomers to undergo bond forming to form multimers, if the target molecules are present in the sample. The presence or absence of target molecule in the sample is then detected based on the fluorescent signature of the sample subjected to the contacting and the subjecting.

Another embodiment of the present invention is directed to a method of detecting the presence or absence of a virus, bacterium or fungus in a sample. The method includes providing a sample potentially containing one or more virus, bacterium or fungus. Also provided is a set of one to six monomers. Each monomer comprises one or more ligand elements, which are useful for binding to one or more target molecules on the surface of, or internally within the virus, bacterium or fungus, with a dissociation constant less than 300 μM, and a linker element being connected directly or indirectly through a connector to the one or more ligand elements. The linker element is capable of forming a bond with one or more linker elements of either the same or a different monomer of the set of monomers. Association of the linker elements, with their ligand elements bound to the one or more target molecules on the surface of, or internally within the virus, bacterium or fungus to form a multimer, will generate a unique fluorescent signature different from that produced by those monomers either alone or in association with each other in the absence of the virus, bacterium or fungus target, when subjected to electromagnetic excitation. The sample is contacted with the set of monomers under conditions effective to allow the ligand elements to bind to the target molecules on the surface of, or internally within the virus, bacterium or fungus, if such target molecules are present in the sample. The monomers are subjected to reaction conditions effective for the linker elements of either the same or different monomers to undergo bond forming to form multimers, if such target molecules are present in the sample. The presence or absence of the virus, bacterium, or fungus in the sample is then detected based on the fluorescent signature of the sample subjected to the contacting and the subjecting.

Yet another embodiment of the present invention is directed to a method of detecting the macromolecular association of one or more target molecules in a sample. The method includes providing a sample potentially containing one or more target molecules capable of undergoing a molecular association. Also provided is a set of one to six monomers. Each monomer comprises one or more ligand elements, which are useful for binding to the one or more target molecules capable of undergoing a molecular association with a dissociation constant less than 300 μM, and a linker element being connected directly or indirectly through a connector to the one or more ligand elements. The linker element is capable of forming a bond with one or more linker elements of either the same or a different monomer of the set of monomers. Association of the linker elements, with their ligand elements bound to the one or more target molecules capable of undergoing a molecular association to form a multimer, will generate a unique fluorescent signature different from that produced by those monomers either alone or in association with each other in the absence of the one or more target molecules capable of undergoing a molecular association, when subjected to electromagnetic excitation. The sample is contacted with the set of monomers under conditions effective to allow the ligand elements to bind to the one or more target molecules capable of undergoing a molecular association, if such target molecules are present in the sample. The monomers are subjected to reaction conditions effective for the linker elements of either the same or different monomers to undergo bond forming to form multimers, if such target molecules are present in the sample. The presence or absence of the one or more target molecules capable of undergoing a molecular association in the sample is then detected based on the fluorescent signature of the sample subjected to the contacting and the subjecting.

Another aspect of the present invention relates to a method of screening for combinations of monomers useful as fluorescent reporters. The method comprises providing a collection of monomers. Each of the monomers comprises one or more ligand elements, which are useful for binding to a target molecule with a dissociation constant less than 300 μM, and a linker element being connected directly or indirectly through a connector to the one or more ligand elements. The linker element is capable of forming a bond with one or more linker elements of either the same or a different monomer of the collection of monomers. Association of the linker elements, with their ligand elements bound to the target molecule to form a multimer, will generate a unique fluorescent signature different from that produced by those monomers either alone or in association with each other in the absence of target, when subjected to electromagnetic excitation. The combinations of the collection of monomers are contacted with the target molecule under conditions effective to allow the ligand elements to bind to the target molecules. The monomers are subjected to reaction conditions effective for the linker elements of either the same or different monomers to undergo bond forming to form multimers. This subjecting step can occur either before, after, or during the contacting step. As a result of the contacting and the subjecting, the combinations of monomers that form multimers and generate a fluorescent signature, which is different from that produced by those monomers either alone or in association with each other in the absence of target, are then identified.

Yet another aspect of the present invention relates to a method of screening for ligands. The method comprises providing a collection of monomers. Each of the monomers comprises one or more ligand elements having a potential to bind to a target molecule and a linker element being connected directly or indirectly through a connector to the one or more ligand elements. The linker element is capable of forming a bond with one or more linker elements of either the same or a different monomer of the collection of monomers. Association of the linker elements, with their ligand elements bound to the target molecule to form a multimer, will generate a unique fluorescent signature different from that produced by those monomers either alone or in association with each other in the absence of target, when subjected to electromagnetic excitation. The combinations of the collection of monomers are contacted with the target molecule under conditions effective to allow the ligand elements to bind to the target molecules. The monomers are subjected to reaction conditions effective for the linker elements of either the same or different monomers to undergo bond forming to form multimers. This subjecting step can occur either before, after, or during the contacting step. As a result of the contacting and the subjecting, the combinations of monomers that form multimers by binding of their ligands to the target molecule and binding of their linker elements, and that generate a fluorescent signature, which is different from that produced by those monomers either alone or in association with each other in the absence of target, are then identified.

An additional aspect of the present invention relates to a collection of monomers capable of forming a multimer useful as a fluorescence reporter. Each monomer comprises one or more ligand elements which are useful for binding to a target molecule with a dissociation constant less than 300 μM and a linker element being connected directly or indirectly through a connector to the one or more ligand elements. The linker element is capable of forming a bond with one or more linker elements of either the same or a different monomer of the collection of monomers. Association of the linker elements, with their ligand elements bound to the target molecule to form a multimer, will generate a unique fluorescent signature different from that produced by those monomers either alone or in association with each other in the absence of target, when subjected to electromagnetic excitation.

Another aspect of the present invention relates to a multimer useful as a fluorescence reporter. The multimer comprises a plurality of covalently or non-covalently linked monomers. Each monomer comprises one or more ligand elements which are useful for binding to a target molecule with a dissociation constant less than 300 μM and a linker element being connected directly or indirectly through a connector to the one or more ligand elements. The linker element is capable of forming a bond with one or more linker elements of either the same or a different monomer of the plurality of monomers. Association of the linker elements, with their ligand elements bound to the target molecule to form a multimer, will generate a unique fluorescent signature different from that produced by those monomers either alone or in association with each other in the absence of target, when subjected to electromagnetic excitation.

The present invention provides a novel class of fluorescent reporting molecules, referred to here as “cofluorons.” Unlike fluorescence-tagged antibodies, cofluorons are stable, synthetic chemicals such that individual cofluoron monomers can self-assemble into tight-binding fluorescent reporter ligands at the site of the target molecule, which serves as a template to promote the oligomerization of cofluoron monomers. Compared to conventional fluorescent methodologies employing fluorescent-tagged antibodies, cofluorons are relative smaller molecules that are advantageous in variety of aspects such as manufacturing or storage.

Additionally, because cofluoron monomers self-assemble to form the tight-binding multimers at the site of the target, where the target promotes the formation of multimers, and because the fluorescent signals generated by cofluoron multimers when bound to the target molecules are different from the fluorescent signatures produced by either the individual cofluoron monomers or the cofluoron multimers when not bound to the target molecules, detection of target molecules can be a one-step detection, that is, direct detection of signals after adding cofluoron in the samples. Hence, unlike the fluorescent-tagged antibodies, which require not only the attachment of antibodies (or the ligand to be detected) to a solid support but also at least one or more additional labeling and washing steps, utilization of cofluorons for fluorescent reporting can be simply, time-saving, and cost-effective.

A cofluoron monomer is composed of one or more ligand elements that bind to the target and a linker element. The linker element of one cofluoron monomer may combine with one or more linker element of either the same or a different cofluoron monomer to form a cofluoron dimer. This process may occur in vivo. In some cases, the linker element binding to each other may be essentially irreversible. In additional cases, the linker elements bind to each other with the aid of a cofactor. In other cases, the linker elements are in a precursor form, and are activated upon entering the body or cells. The linker elements can bind to each other through one or more reversible or irreversible covalent bonds. The linker elements can also bind to each other through non-covalent interaction such as hydrophobic, polar, ionic and hydrogen bonding. In the presence of the target, the combinations of multiple (weak) interactions between the ligand elements of one cofluoron monomer and a target protein, the ligand element of a second cofluoron monomer and the target protein, as well as the two cofluorons with each other combine to produce a tight binding cofluoron dimer with highly specific binding to its target. Upon association to cofluoron dimers and cofluoron dimers binding to the target molecules, the cofluoron dimer generates a unique fluorescent signature different from that produced by individual cofluoron monomers either alone or in association with each other in the absence of target molecules. Hence, cofluorons may be used as fluorescent reporting agents in macromolecular systems.

The concept may be extended to include multimer cofluorons and multimer targets.

The linker elements of the present invention can have a broad range of molecular weight depending on applications. However, it can be designed to be low molecular weight moieties (e.g., with molecular weights less than 2000 daltons) that associate with each other in vivo that may or may not react with cellular components. Each linker element has attachment points for introducing diverse ligands. They are compatible with “click chemistry”. In some cases, the association between the linker elements is reversible, allowing for dynamic combinatorial chemistry selection of the best ligands that have the highest binding affinity and produce the best fluorescent signals. The linker elements allow in vivo assembly of multiple small ligands to produce multimeric structures.

The present invention provides a novel methodology of using cofluorons as fluorescent reporters to detect the presence or absence of target molecules or event or activity associated with the presence or absence of target molecules.

For example, cofluorons can be used to detect and/or monitor the presence or absence of macromolecular targets such as proteins, nucleic acids, carbohydrates; lipid, intracellular proteins, surface proteins, viral proteins, viral structural macromolecules, bacterial proteins, or bacterial macromolecules. Cofluorons may also be designed to target multiple targets or multiple sites on a target. Because many target molecules often associate to an event or activity that is of interest, cofluorons can hence be designed to target event or activity such as association of macromolecular targets, protein interactions, protein localization, protein tracking, protein trafficking, cellular process, metabolism of cells, intracellular and extracellular compartmentalization, cell signaling, disease state, disease progression, disease prognosis, disease remission, or therapeutic molecule binding.

Exemplary cofluoron designs include (a) cofluorons with identical ligand elements, which bind to adjacent identical binding pockets of a target, and combine on their linker-element portions to create a fluorescent signal, (b) cofluorons with different ligand elements, which bind to adjacent targets, and combine on their linker-element portions to create a fluorescent signal, (c) cofluorons where a ligand element has both “donor” and “acceptor” linker elements (whose geometry prevents formation of intramolecular covalent bonds), such that two or more cofluorons bind to the surface of a target (such as a surface of a virus) through two or more target proteins. These designs may be used to cover the surface of a virus or bacteria with a multiple copies of fluorescent molecules, allowing for convenient detection of such pathogens, either in vivo or in the environment.

Cofluorons possess the binding specificity to target molecule reporting due to the specificity of ligand elements in cofluorons to the target molecules. Hence, cofluorons can be used as organelle-, cell- or tissue-specific fluorescent labels to identify diseased or infected tissues and cell, specific tissue and cell types such as neuronal tracers. For instance, cofluorons provided herein can be used as reporters to trace disease-specific genetic anomalies. Moreover, cofluorons can also be used in cell sorting techniques to separate different cell lines. For example, when the target molecule is associated with cell surfaces, the method can further comprise sorting the cells based on the fluorescent signature of the multimer.

Cofluorons can also be used to quantitatively analyze the target molecule or activity or event associated with the target molecule in a sample. For example, the fluorescence generated in the sample containing an unknown amount of the target molecule can be measured using the method described above with the cofluorons. This measurement can be compared with the fluorescence measured from a sample containing a known amount of the target molecule. The amount of the target molecule present in the former sample can then be determined based on the comparing. This quantification method can be found useful in many different application areas such as analyzing environmental samples for the amount of microorganisms, blood samples for the amount of glucose, or other biosensing assays.

Cofluorons can stain proteins in living cells, thus serving as a tool for both research and diagnostic purposes. Unlike the traditional method of visualization of proteins in living cells, which is an expensive and time-consuming procedure using recombinant proteins with fluorescent tags that must be introduced into the cell, cofluorons can be used as individual monomers that, depending on the molecular weight, can be designed to be cell permeable, enter the cell and combine inside the cells to form cofluoron multimers that bind to the intracellular target molecules. This allows cofluorons to trace target molecules such as proteins in cells, organelles or tissues in their natural state, without overly expressing the protein of interest, or attaching a large fluorescent protein to the target molecule. Thus cofluorons can be used as non-invasive fluorescent reporting agents easily used in biological system for many in vivo applications. This would also allow cofluorons for imaging the target molecules or events or activities associated with the binding of target molecules, such as intracellular proteins and macromolecules, protein interactions, pathway analysis, protein tracking and trafficking tissues, living cells, cell types, cellular processes. All these labeling and imaging methodologies can be carried out in a non-invasive manner in vivo. For example, cofluorons can be used in cancer diagnosis for non-invasively detecting/monitoring skin cancers by using confocal microscopy.

Furthermore, screening using cofluorons for such ligands or drugs (e.g., the fusion protein products) would provide a rapid detection protocol, particularly useful in high-throughput screening. Cofluorons, like coferons, provide a unique opportunity for drug screening due to their combinatorial nature. The ligand elements of cofluoron may be screened for targeting specific protein surfaces or protein interaction domains and interfere or modulate activity of the target proteins. Such ligand elements can therefore be considered as a pharmacophore, and the cofluoron in this sense, can be used as fluorescent coferons for drug discovery and screening. The unique benefit of cofluorons lies on the easy detection due to the fluorescent reporting nature of cofluorons. The ability of linker binding pairs to generate an increase in or wavelength shift in fluorescence signal provides an opportunity to rapidly detect coferon pair binding to the target protein or molecule. Therefore, cofluorons can be used to develop rapid high-throughput screening techniques to determine the binding affinities of coferon candidate pairs.

Additionally, cofluorons can be designed to combine both fluorescent reporting and therapeutic functions into one molecular design. The diagnostic application of the cofluorons may not depend on an efficacious application of the cofluorons, i.e., a specific diagnostic read-out may be possible even without an efficacious result of the cofluoron binding. However, an end-point of dual therapeutic efficacy and effective diagnostics for cofluoron designs would be desirable and can be achieved.

### BRIEF DESCRIPTION OF THE DRAWINGS

FIG. 1 is a schematic drawing of the components used in a cofluoron monomer.

FIGS. 2A to 2J show the variations of the components of cofluoron design. FIG. 2A is a schematic drawing of cofluoron monomers attached to encoded beads via connectors. FIG. 2B is a schematic drawing of a cofluoron monomer with connector. FIG. 2C is a schematic drawing of a cofluoron dimer attached to an encoded bead via a connector to one monomer. FIG. 2D is a schematic drawing of a cofluoron heterodimer with connectors. FIG. 2E is a schematic drawing of a cofluoron homodimer with connectors. FIG. 2F is a schematic drawing of cofluoron monomers attached to encoded beads. FIG. 2G is a schematic drawing of a cofluoron monomer. FIG. 2H is a schematic drawing of a cofluoron dimer attached to an encoded bead via one monomer. FIG. 2I is a schematic drawing of a cofluoron heterodimer. FIG. 2J is a schematic drawing of a cofluoron homodimer.

FIG. 3 is a schematic drawing of the exemplary cofluoron heterodimer formed by reversible association of two cofluoron monomers. The linker elements for individual cofluoron monomers are presented by a dot and semi-circle, respectively.

FIG. 4 is a graph showing the results of fluorescent measurements on the monomer 3,4,5-trihydroxybenzamide and the multimers formed by mixing 3,4,5-trihydroxybenzamide with different concentrations of 2-fluorophenylboronic acid. The multimers were formed by mixing 100 μM 3,4,5-trihydroxybenzamide with 2-fluorophenylboronic acid having concentrations as follows: series 1-9=30 mM, 10 mM, 3 mM, 1 mM, 0.3 mM, 0.1 mM, 0.03 mM, 0.01 mM, and blank respectively. Fluorescence signals were measured on samples in 0.1M HEPES buffer at pH 7.9 (in 50% DMSO), when excited at 340 nm.

FIG. 5 is a graph showing the results of fluorescent measurements on the monomer containing a dihydroxy moiety and the multimers formed by mixing the dihydroxy compound with various boronic acid binding partners. The multimers were formed by mixing 100 μM 7,8-dihydroxy-4-methylcoumarin with 300 μM of various boronic acid binding partners as follows: series 1-9=2-(hydroxymethyl)phenylboronic acid, benzofuran-2-boronic acid, 2-methoxypyrimidine-5-boronic acid, 3,5-difluorophenylboronic acid, 5-quinolinylboronic acid, 2-fluoropyridine-3-boronic acid, 2-(N,N-dimethylamino)pyridine-5-boronic acid, and 2-chloroquinoline-3-boronic acid respectively. Fluorescent signals were measured on samples in 0.1M phosphate buffer at pH 7.4 (in 50% DMSO), when excited at 350 nm.

FIGS. 6A-6B illustrate the wavelength shifts in fluorescence emission for linker elements when binding to their binding partners. The initial linker element is a binding partner for boronic acid family, shown as “SL1” (linker element 1, in FIG. 6A) and “SL3” (linker element 3, in FIG. 6B), respectively. Linker element 1 is 2-Hydroxy-3-naphthalenecarboxamide, whose structure is shown in the inset of FIG. 6A; and linker element 3 is gallic acid ethanolamide, whose structure is shown in the inset of FIG. 6B. The four different boronic acid linker elements, labeled 2a through 2d respectively were: 2a=2-chloroquinoline-3-boronic acid; 2b=2-fluoropyridine-3-boronic acid; 2c=3,5-difluorophenylboronic acid; and 2d=benzofuran-2-boronic acid. In each figure, the combination of both linker elements is indicated by the plus sign, for example, SL1+2d is a combination of 2-hydroxy-3-naphthalenecarboxamide with benzofuran-2-boronic acid. The boronic acids were used at a concentration of 300 μM, while their partners were at a concentration of 100 μM. FIG. 6A shows that addition of 3 different boronic acid linker elements (2b, 2c, and 2d) to the linker element SL1 produced a stronger fluorescent signal, as well as a fluorescent emission wavelength shift to a lower wavelength (i.e. blue shift). FIG. 6B shows that addition of 3 different boronic acid linker elements (2b, 2c, and 2d) to the linker element SL3 produced a stronger fluorescent signal, as well as a fluorescent emission wavelength shift to a higher wavelength (red shift).

FIG. 7 is a graph showing the results of fluorescent measurements on the monomer 2-hydroxy-3-naphthalenecarboxamide and on the multimers formed by mixing 2-hydroxy-3-naphthalenecarboxamide with various boronic acid binding partners. The multimers were formed by mixing 100 μM 2-hydroxy-3-naphthalenecarboxamide with 300 μM of various boronic acid binding partners as follows: series 1-9=2-(hydroxymethyl)phenylboronic acid, benzofuran-2-boronic acid, 2-methoxypyrimidine-5-boronic acid, 3,5-difluorophenylboronic acid, 5-quinolinylboronic acid, 2-fluoropyridine-3-boronic acid, 2-(N,N-dimethylamino)pyridine-5-boronic acid, and 2-chloroquinoline-3-boronic acid, respectively. Fluorescent signals were measured on samples in 0.1M phosphate buffer at pH 7.4 (in 50% DMSO), when excited at 350 nm.

FIG. 8 is a graph showing the results of fluorescent measurements on the monomer 2-hydroxy-3-naphthalenecarboxamide and on the multimers formed by mixing 2-hydroxy-3-naphthalenecarboxamide with various boronic acid binding partners. The multimers were formed similarly as in FIG. 7. Fluorescent signals were measured on samples in similar conditions as in FIG. 7, except in the absence of DMSO.

FIG. 9 is a graph showing the results of fluorescent measurements on the monomer methyl 3,4,5-trihydroxybenzoate and on the multimers formed by mixing methyl 3,4,5-trihydroxybenzoate with various boronic acid binding partners. The multimers were formed by mixing 100 μM methyl 3,4,5-trihydroxybenzoate with 300 μM of various boronic acid binding partners as follows: series 1-9=2-(hydroxymethyl)phenylboronic acid, benzofuran-2-boronic acid, 2-methoxypyrimidine-5-boronic acid, 3,5-difluorophenylboronic acid, 5-quinolinylboronic acid, 2-fluoropyridine-3-boronic acid, 2-(N,N-dimethylamino)pyridine-5-boronic acid, and 2-chloroquinoline-3-boronic acid respectively. Fluorescent signals were measured on samples in 0.1M phosphate buffer at pH 7.4 (in 50% DMSO), when excited at 350 nm.

FIG. 10 is a graph showing the results of fluorescent measurements on the monomer 3,4,5-trihydroxybenzamide and on the multimers formed by mixing 3,4,5-trihydroxybenzamide with various boronic acid binding partners. The multimers were formed by mixing 100 μM 3,4,5-trihydroxybenzamide with 300 μM of various boronic acid binding partners as follows: series 1-9=2-(hydroxymethyl)phenylboronic acid, benzofuran-2-boronic acid, 2-methoxypyrimidine-5-boronic acid, 3,5-difluorophenylboronic acid, 5-quinolinylboronic acid, 2-fluoropyridine-3-boronic acid, 2-(N,N-dimethylamino)pyridine-5-boronic acid, and 2-chloroquinoline-3-boronic acid respectively. Fluorescent signals were measured on samples in 0.1M phosphate buffer at pH 7.4 (in 50% DMSO), when excited at 350 nm.

FIG. 11 shows various linker elements and potential cofluoron monomers that contain boronic acid.

FIG. 12 shows various linker elements and potential cofluoron monomers that contain catechol and gallol.

FIG. 13 is a graph showing fluorescent measurement on the cofluoron multimer formed by binding cofluoron monomers T12 and T27 as well as fluorescent measurements on individual cofluoron monomers. The multimer was formed by mixing 100 μM T12 and 100 μM T27. Fluorescent signals were measured on samples excited at 350 nm.

FIG. 14 is a graph showing the results of fluorescent measurement on the cofluoron multimer formed by binding cofluoron monomers T11 and T24 as well as fluorescent measurements on individual cofluoron monomers. The multimer was formed by mixing 100 μM T12 and 100 μM T27. Fluorescent signals were measured on samples in 0.1M sodium phosphate buffer at pH 7.5, when excited at 350 nm.

FIG. 15 is a graph showing the results of fluorescent measurements on the cofluoron monomer T43 and on the cofluoron multimers formed by binding T43 with various boronic acid binding partners and with various cofluoron monomers. The multimers were formed by mixing 100 μM T43 with 100 μM of various binding partners as follows: series 1-8=blank, benzofuran-2-boronic acid, 3,5-difluorophenylboronic acid, 2-fluoropyridine-3-boronic acid, T10, T11, T12, and T13, respectively. Fluorescent signals were measured on samples in 0.1M phosphate buffer at pH 7.4 (in aqueous), when excited at 360 nm.

FIG. 16 is a graph showing the results of fluorescent measurements on the cofluoron monomer T43 and on the cofluoron multimers formed by binding T43 with various boronic acid binding partners and with various cofluoron monomers. The multimer was formed by mixing 100 μM T43 with 100 μM of various binding partners as follows: series 1-8=blank, benzofuran-2-boronic acid, 3,5-difluorophenylboronic acid, 2-fluoropyridine-3-boronic acid, T33, T34, T35, and T37, respectively. Fluorescent signals were measured on samples in 0.1M phosphate buffer at pH 7.4 (in aqueous), when excited at 360 nm.

FIGS. 17A-17D are fluorescent images demonstrating the permeation of cofluoron monomers T11 and T24 into a human mast cell line and the detection of formation of cofluoron dimer T11-T24 inside the cells, by enhanced fluorescent signals. FIG. 17A is an image of untreated cells as a control showing background staining under the excitation of UV wavelength. FIG. 17B shows a faint staining after cells were treated with 100 μM cofluoron monomer T11; and FIG. 17C shows a somewhat brighter staining after cells were individually treated with 100 μM cofluoron monomer T24. FIG. 17D show a remarkable increase in fluorescence signals in the cells after both cofluoron monomers were added (100 μM each).

FIG. 18 is a graph showing the results of fluorescent measurements on the cofluoron multimers formed by mixing 6 μM T43 and 6 μM of its various binding partners, as well as on the monomer T43, in the presence or absence of 5 μM Tryptase. The tested samples were prepared as follows: series 1-5=T43, T43/T34, T43/T11, T43/T35, and T43/T37, each of which mixed with 5 μM Tryptase; series 6-10=T43, T43/T34, T43/T11, T43/T35, and T43/T37, without Tryptase. Fluorescent signals were measured on samples in 50 μM phosphate buffer at pH 7.4 and 200 mM sodium chloride (in aqueous), when excited at 360 nm.

FIG. 19 is a graph showing the results of fluorescent measurements on the cofluoron multimers formed by mixing 100 μM T147 and 100 μM T27F, as well as fluorescent measurements on individual cofluoron monomers. Fluorescent signals were measured on samples in 0.1M phosphate buffer at pH 7.4 containing 100 μM EDTA, when excited at 300 nm.

FIG. 20 is a graph showing the results of fluorescent measurements on the cofluoron multimers formed by mixing 1.5 μM T147 and 1.5 μM T27F, as well as fluorescent measurements on individual cofluoron monomers, in the presence or absence of 3 μM recombinant human tryptase. Fluorescent signals were measured on samples in 0.1M phosphate buffer at pH 7.5, when excited at 300 nm.

FIG. 21 is a graph showing the results of fluorescent measurements on the cofluoron multimers formed by mixing T109-Spiro and T27F, as well as fluorescent measurements on individual cofluoron monomers. Fluorescent signals were measured on samples in 0.1M phosphate buffer at pH 7.4 containing 100 μM EDTA, when excited at 300 nm.

FIG. 22 is a graph showing the results of fluorescent measurements on the cofluoron multimers formed by mixing T109Spiro and T27F, as well as fluorescent measurements on individual cofluoron monomers. Fluorescent signals were measured on samples in 0.1M phosphate buffer at pH 7.4 containing 100 μM EDTA, when excited at 350 nm.

FIG. 23 is a graph showing the results of fluorescent measurements on the cofluoron multimers formed by mixing T27 and T107, as well as fluorescent measurements on individual cofluoron monomers. Fluorescent signals were measured on samples in 0.1M phosphate buffer at pH 7.4 containing 100 μM EDTA, when excited at 300 nm.

FIG. 24 is a graph showing the results of fluorescent measurements on the cofluoron multimers formed by mixing T27F and T107, as well as fluorescent measurements on individual cofluoron monomers. Fluorescent signals were measured on samples in 0.1M phosphate buffer at pH 7.4 containing 100 μM EDTA, when excited at 300 nm.

FIG. 25 is a graph showing the results of fluorescent measurements on the cofluoron multimers formed by mixing T27 and T51, as well as fluorescent measurements on individual cofluoron monomers. Fluorescent signals were measured on samples in 0.1M phosphate buffer at pH 7.4 containing 100 μM EDTA, when excited at 340 nm.

FIG. 26 is a graph showing the results of fluorescent measurements on the cofluoron multimers formed by mixing T27F and T51, as well as fluorescent measurements on individual cofluoron monomers. Fluorescent signals were measured on samples in 0.1M phosphate buffer at pH 7.4 containing 100 μM EDTA, when excited at 340 nm.

FIG. 27 is a graph showing the results of fluorescent measurements on the cofluoron multimers formed by mixing T27F and T54BASpiro, as well as fluorescent measurements on individual cofluoron monomers. Fluorescent signals were measured when excited at 300 nm.

FIG. 28 is a graph showing the results of fluorescent measurements on the cofluoron multimers formed by mixing T27F and T54BA, as well as fluorescent measurements on individual cofluoron monomers. Fluorescent signals were measured when excited at 330 nm.

FIG. 29 is a graph showing the results of fluorescent measurements on the cofluoron multimers formed by mixing T27F and T54BASpiro, as well as fluorescent measurements on individual cofluoron monomers. Fluorescent signals were measured when excited at 330 nm.

FIG. 30 is a graph showing the results of fluorescent measurements on the cofluoron multimers formed by mixing T27 and T133Spiro, as well as fluorescent measurements on individual cofluoron monomers. Fluorescent signals were measured on samples in 0.1M phosphate buffer at pH 7.4 containing 200 μM EDTA, when excited at 300 nm.

FIG. 31 is a graph showing the results of fluorescent measurements on the cofluoron multimers formed by mixing T27F and T133Spiro, as well as fluorescent measurements on individual cofluoron monomers. Fluorescent signals were measured on samples in 0.1M phosphate buffer at pH 7.4 containing 200 μM EDTA, when excited at 300 nm.

FIG. 32 is a graph showing the results of fluorescent measurements on the cofluoron multimers formed by mixing T27 and T133Spiro, as well as fluorescent measurements on individual cofluoron monomers. Fluorescent signals were measured on samples in 0.1M phosphate buffer at pH 7.4 containing 200 μM EDTA, when excited at 350 nm.

FIG. 33 is a graph showing the results of fluorescent measurements on the cofluoron multimers formed by mixing T27F and T133Spiro, as well as fluorescent measurements on individual cofluoron monomers. Fluorescent signals were measured on samples in 0.1M phosphate buffer at pH 7.4 containing 200 μM EDTA, when excited at 350 nm.

FIG. 34 is a graph showing the results of fluorescent measurements on the cofluoron multimers formed by mixing T27F and T64, as well as fluorescent measurements on individual cofluoron monomers. Fluorescent signals were measured on samples in 0.1M phosphate buffer at pH 7.4 containing 100 μM EDTA, when excited at 350 nm.

FIG. 35 is a graph showing the results of fluorescent measurements on the cofluoron monomer 4-(4-methyl-3-oxido-5-phenyl-1H-imidazol-2-yl)-1,2-benzene diol and the multimers formed by mixing 100 μM 4-(4-methyl-3-oxido-5-phenyl-1H-imidazol-2-yl)-1,2-benzene diol with 300 μM various boronic acid binding partners. Fluorescent signals were measured on samples in 0.1M phosphate buffer at pH 7.4, when excited at 350 nm.

### DETAILED DESCRIPTION OF THE INVENTION

“Cofluorons,” as defined herein, refer to individual monomers that can combine with their partner to form a multimer that bind to a target molecule, and upon binding, the multimer generates a unique fluorescent signature different from that produced by individual cofluoron monomers either alone or in association with each other in absence of target. Hence, cofluorons may be used as reporter agents to detect macromolecules such as proteins, nucleic acids, carbohydrates, lipids, bacterial, viral pathogens, fungus, cancer cells, or macromolecular associations. A cofluoron monomer comprises essentially two parts: one or more ligand elements and a linker element. Cofluorons can also generally include cofluoron dimers or cofluoron multimers.

The basic cofluoron design contains the linker element, which is responsible for interacting with its partner linker element, and the ligand element, which is responsible for binding to the target. The linker element and the ligand element may be directly attached to each other, or linked together by a connector moiety. The linker element and/or connector portion may assist in positioning the ligand element in the ideal conformation or orientation for proper binding to the target. In addition, these elements in and of themselves may also interact with the target. When the linker element or connector makes favorable interactions with the target, the portions of the connector or linker element that interact with the target function as secondary binding ligand elements.

The encryption element, if used, may be attached to the linker element or the connector portion of the molecule. Ideally, it should be linked to the linker element or connector portion in a manner allowing for easy release or cleavage to remove the encryption element. The use of encryption element in the cofluorons are mainly for the purpose of screening for combinations of monomers that can be used for cofluoron reporters or screening for ligands that have potential to bind a target molecule, where the use of different encoding elements for each monomer can aid in identifying the candidate cofluoron multimers and their component cofluoron monomers by distinguishing their encoding elements.

In general, cofluoron reporters contain two ligand elements that bind to the target, and are held together through their respective linker element interactions. In order to assure that the cofluoron reporters bind to a given target, the design of cofluoron usually incorporates selecting from a known set of ligand elements and/or synthesizing a wide range of ligand elements for one or both of the cofluoron monomers that form the dimer.

Once the ligand elements for a cofluoron dimer has been selected for, or screened by various assays, it is important to be able to identify the combinations of monomers that form cofluoron dimers that, when bound to the target, can generate a unique fluorescent signature different from any of those produced by individual cofluoron monomers or by cofluoron dimers in absence of target.

Fluorescence arises when a molecule in its ground state absorbs energy in the form of ultraviolet-visible (UV-Vis) radiation and electrons in the molecule are raised to a higher energy singlet excited state. Some of this excess energy is lost in a non-radiative manner due to interaction with the environment and the molecule internally converts to a relaxed singlet excited stage. The remaining excess energy is dissipated by emission of light and the molecule returns to the ground state. The difference in energy between the absorbed light and the emitted light is referred to as the Stokes shift. In fluorescence spectroscopy, the emitted light is typically of lower energy, i.e. higher wavelength, than the absorbed light. Excitation of the molecule typically can be carried out with a short pulse of light (typically on the order of 10⁻¹⁵ seconds); internal conversion of singlet excited state to the relaxed singlet state typically occurs within 10⁻¹² seconds or faster; and the fluorescence lifetime, the time between the light absorption and subsequent fluorescent emission, can generally be observed on the order of 10⁻⁸ seconds.

A fluorophore generally refers to a molecule that is fluorescent. Quantum yield of a fluorescent molecule refers to the amount of light emitted relative to the amount of light absorbed by the molecule. More efficient fluorophores typically have higher quantum yields and emit light with higher intensity.

In general, aromatic and heteroaromatic organic molecules or molecules with extensive conjugation can be fluorescent. The fluorescent properties of a molecule can be tuned by varying the degree of conjugation of the core structure and by varying substituent groups on the core structure. For example, groups that promote the delocalization of electrons typically enhance fluorescence intensity and/or shift both the absorbance and emission to higher wavelengths. Fluorescence can also arise from excited dimers (referred to as “excimer”), i.e., a short-lived dimer formed from two monomers, where at least one of the monomers is in an excited state. For example, certain polyaromatic molecules (e.g. pyrene) can interact with each other to form excimer that can emit light at a higher wavelength, when one of the monomers is in an excited state. Additionally, fluorescence can also arise from charge transfer complexes, where two or more molecules (or two parts of the same molecule in the case of internal charge transfer complexes) associate and transfer a charge between each other.

The initial cofluoron monomers can be either fluorescent or non-fluorescent in nature. However, when the cofluoron monomers oligomerize, they generate unique fluorescent signatures in the uv, visible or infrared spectrum that are different from either of the cofluoron monomers, allowing one to distinguish the formation of the cofluoron multimers from the initial monomers.

For instance, one or more cofluoron monomers may be fluorescent initially, and can oligomerize to form cofluoron multimers which exhibit a shift in the fluorescence emission wavelength either to lower (blue shift) or higher (red shift) wavelengths. Alternatively, one or more of the cofluoron monomers may be fluorescent initially, and can oligomerize to form cofluoron multimers that have higher quantum yield so that the fluorescent emissions retain at the same wavelengths but the emission intensity is higher. The oligomerization of the initially fluorescent cofluoron monomers with one or more initially fluorescent or non-fluorescent cofluoron monomers can also result in cofluoron multimers that have lower quantum yield so that the fluorescent emission intensity becomes lower. For example, one may also detect and monitor the fluorescent quenching event to identify the formation of cofluoron multimers, although in practice detection of enhancement in fluorescent emission signals may be preferred over quenching of the fluorescent signal. The change in fluorescent emissions when cofluoron monomers forming cofluoron dimers can also include the combination of the shift in emission wavelength and the change in emission intensity. Cofluoron monomers may also be non-fluorescent initially, but may oligomerize to form cofluoron multimers having extended conjugation or the ability to form charge transfer complexes that are fluorescent.

The dissociation constant between the linker elements and their binding partners can be tuned by varying the linker element and its binding partners so that oligomerization of cofluoron monomers occurs predominantly in the presence of the target, whereby cofluoron multimer binds to the target. In this scenario, the fluorescent signature changes produced by the cofluoron multimers that are formed by oligomerization of cofluoron monomers in the presence of the target, if detected, can be used to indicate the presence of the target or measure/monitor the amount of the target in presence.

Alternatively, if the dissociation constant between the linker element and its binding partners is small so that they associate to form some multimers in the absence of target, fluorescent signature changes can be produced by cofluoron multimers even in the absence of target. However, the binding event of the cofluoron multimers to the target can still be identified and monitored by detecting the change in fluorescence signatures of cofluoron multimers, e.g., change in fluorescent polarization, in the presence of target compared to those in the absence of target. This is because cofluorons are generally smaller molecules compared to macromolecules that they bind to, and the polarization of the light changes differently for smaller molecules and larger molecules. When excited with plane polarized light, a fluorophore emits light that has a degree of polarization that is inversely proportional to its molecular rotation. Larger molecules remain relatively stationary during the excited state and the polarization of the light remains relatively constant between excitation and emission. Smaller molecules rotate rapidly during the excited state and the polarization of the light changes relatively large between excitation and emission. Therefore, smaller molecules have low polarization values and larger molecules have high polarization values. When cofluorons bind to larger targets, such as proteins, or bacterial and viral pathogens, they rotate more slowly, and this change in fluorescence polarization can be used to identify and monitoring the binding event of cofluoron multimers to the target.

The fluorescent reporting properties of cofluoron can be affected by many factors, such as solvent, ionic strength, ion concentration, pH, temperature, and etc. This is because the interaction of cofluoron and the target molecules may change upon the environmental change. For example, the changes of fluorescent signatures of cofluorons, upon oligomerization and binding to the target, may be a result of the change of interaction between the fluorophore of the cofluoron with the target. For instance, the fluorescent signature changes may be affected by the degree of hydrophobic interaction between the cofluoron fluorophore and the surface of the target (e.g., protein) that directly in contact with the fluorophore. Further, like standard dyes, the cofluorons may change color upon a shift in pH.

### Cofluoron Monomers and Multimers

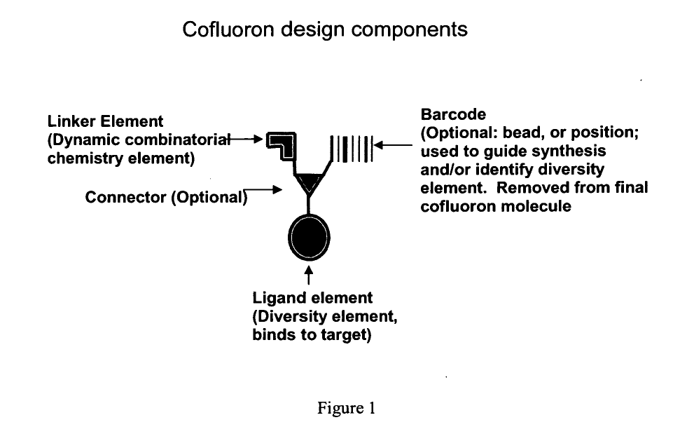

As shown in FIG. 1, the cofluoron monomers may include a linker element, one or more ligand elements, an optional connector, and an optional bar code (i.e. encryption element).

The linker element is a dynamic combinatorial chemistry element that can have a broad range of molecular weight depending on applications. However, it can be designed to be low molecular weight moieties for cell permeability. For example, the linker element may have a molecular weight of less than 2000 daltons, or even lower, for instance, a molecular weight of less than 500 daltons, or from about 45 to about 450 daltons. In one embodiment, the linker element is non-peptidyl. The linker element is responsible for combining with its partner linker element and its attached ligand elements. The linker element of one cofluoron monomer may combine with one or more linker element of either the same or a different cofluoron monomer to form a cofluoron dimer. The linker elements can bind to each other through one or more reversible or irreversible covalent bonds. In some embodiments, the linker element binding to each other may be essentially irreversible. The linker elements can also bind to each other through non-covalent interaction such as hydrophobic, polar, ionic and hydrogen bonding. In some embodiments, the linker elements bind to each other with the aid of a cofactor. In some embodiments, the linker element bonding forms under physiological conditions. The linker element bonding may occur in vivo. In other embodiments, the linker elements are in a precursor form, and are activated upon entering the body or cells. The linker element can reversibly associate with one or more linker elements of either the same or a different monomer with a dissociation constant of less than 300 μM. In some embodiments, the dissociation constant of the linker element pairing ranges from about 100 nM to about 300 μM. The ligand elements are useful for binding to a target molecule with a dissociation constant of less than 300 μM with respect to the target. In some embodiments, the dissociation constant of the ligand element with respect to the target ranges from about 1 nM to 300 μM. In some embodiments, the ligand elements bind to proximate locations of the target molecule such that the distance between the binding locations can be spanned by the cofluorons with their ligand elements bound to the target and the linker elements with or without the connector have associated with each other. The ligand element may have a broad range of molecular weight depending on applications. However, it can be designed to be low molecular weight moieties for cell permeability. The linker element and the one or more ligand elements may be connected directly to each other or linked together by a connector moiety. An optional connector binds the linker element and the one or more ligand elements, assists in synthesis of the cofluoron monomer, and may assist in positioning the ligand elements in the ideal conformation or orientation for proper binding to the target.

The cofluoron monomer may further comprise an encoding element or “bar code” moiety. This encoding element can be coupled with the one or more ligand elements and/or linker element directly, or indirectly through a connector for easy release or cleavage. The encoding element is included to guide synthesis and to identify cofluoron monomers. In some embodiments, the encoding element is a labeled bead or solid support. The encoding element is typically removed from final cofluoron reporters.

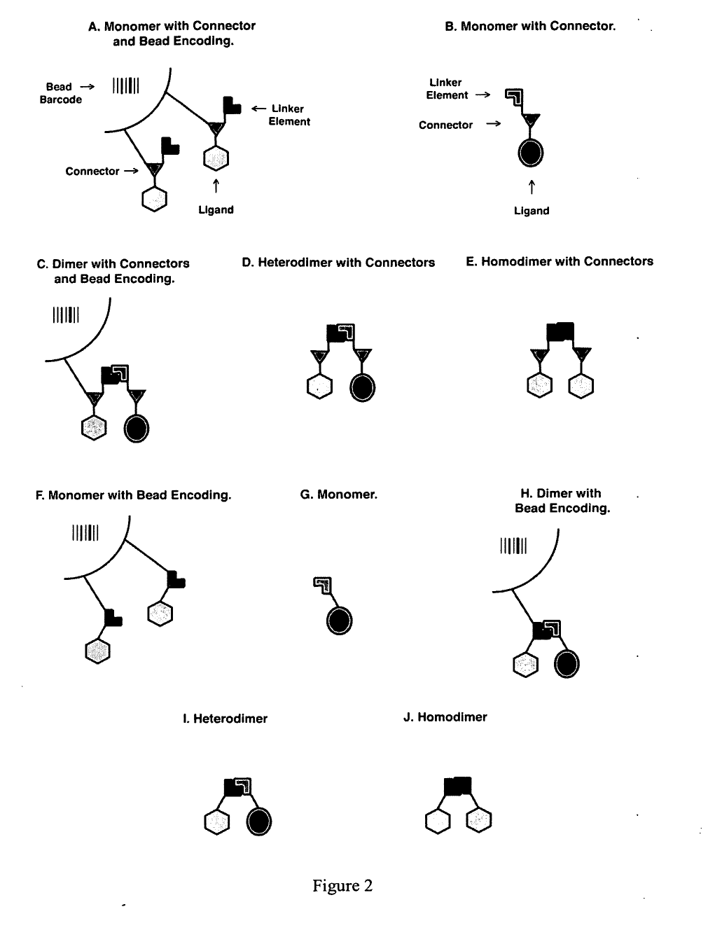

FIGS. 2A to 2J show the variations of the components of cofluoron design. FIG. 2A is a schematic drawing of cofluoron monomers attached to encoded beads via connectors. FIG. 2B is a schematic drawing of a cofluoron monomer with connector. FIG. 2C is a schematic drawing of a cofluoron dimer attached to an encoded bead via a connector to one monomer. FIG. 2D is a schematic drawing of a cofluoron heterodimer with connectors. FIG. 2E is a schematic drawing of a cofluoron homodimer with connectors. FIG. 2F is a schematic drawing of cofluoron monomers attached to encoded beads. FIG. 2G is a schematic drawing of a cofluoron monomer. FIG. 2H is a schematic drawing of a cofluoron dimer attached to an encoded bead via one monomer. FIG. 2I is a schematic drawing of a cofluoron heterodimer. FIG. 2J is a schematic drawing of a cofluoron homodimer.

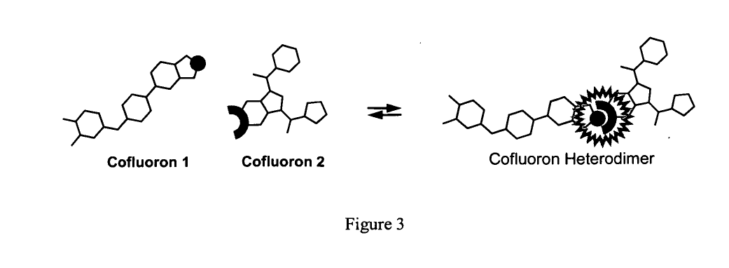

In some embodiments, cofluoron reporters, formed at the target site, contain two ligand elements that bind to the target, and are held together through their respective linker element interactions. The cofluoron dimer can be either a heterodimer or homodimer. FIG. 3 is a schematic drawing of an exemplary cofluoron heterodimer formed by reversible association of cofluoron monomers, in the absence of a target. In the presence of the target, the combinations of multiple (weak) interactions between the ligand elements of one cofluoron monomer and a target, the ligand element of a second cofluoron monomer and the target, as well as the two cofluorons with each other combine to produce a tight binding cofluoron dimer with highly specific binding to its target. Upon association to cofluoron dimers and cofluoron dimers binding to the target molecules, the cofluoron dimer generates a unique fluorescent signature different from that produced by individual cofluoron monomers either alone or in association with each other in the absence of target molecules.

In some embodiments, at least one of the cofluoron monomers that form a cofluoron multimer has a fluorophore and is capable of fluorescence prior to bonding to another cofluoron monomer. The fluorophore of the fluorescent monomer may come from any of the components of the monomer, including linker element, connector, ligand element, or combination thereof. Association of this fluorescent monomer with a second monomer, having a linking element that is a binding partner with that of the fluorescent monomer, can change its fluorescent signature. The second monomer that binds with the fluorescent monomer may or may not be fluorescent alone. The change of the fluorescent signature can include a change in fluorescence emission intensity, including an increase or a decrease or a complete quenching; a change in fluorescent excitation wavelength or fluorescence emission wavelength, including blue shift or red shift; a change in polarization of fluorescence emission; or combinations thereof.

Cofluorons are multimeric assemblies formed by the association of monomers through chemical bonding of appropriate electrophilic and nucleophilic linker elements of the monomers. Exemplary electrophilic linker elements include boronic acids and oxaboroles such as 8-quinolinylboronoc acid, isoquinoline-6-boronic acid and isoquinoline-5-boronic acid. Exemplary nucleophilic linker elements include catechols, ortho-hydroxyaryl carboxamides, ortho-hydroxyaryl hydroxamic acids and ortho-hydroxyaryl O-alkyl hydroxamates such as 3,4,5-trihydroxybenzamide, 6,7-dihydroxycouomarin, 7,8-dihydroxycoumarin, 2-hydroxy-3-napthalene carboxamide and methyl 3,4,5-trihydroxybenzoate. In some embodiments, none of individual cofluoron monomers forming the cofluoron multimer are fluorescent alone, but their association produces a fluorescent signature.

One aspect of the present invention is directed to a collection of monomers capable of forming a multimer useful as a fluorescence reporter. Each monomer comprises one or more ligand elements which are useful for binding to a target molecule with a dissociation constant less than 300 μM and a linker element being connected directly or indirectly through a connector to the one or more ligand elements. The linker element is capable of forming a bond with one or more linker elements of either the same or a different monomer of the collection of monomers. Association of the linker elements, with their ligand elements bound to the target molecule to form a multimer, will generate a unique fluorescent signature different from that produced by those monomers either alone or in association with each other in the absence of target, when subjected to electromagnetic excitation.

At least some of the monomers in the collection can additionally include an encoding element or “bar code”, where the one or more ligand elements, the linker element, and the encoding element are coupled together. The encoding element can be an oligonucleotide, a labeled bead or a solid support.

The collection of monomers for forming cofluoron multimer useful as fluorescent reporters can include unlimited number of monomers as needed. In some instances, such collection includes a set of one to six monomers, or one or two monomers. Many membrane proteins form multimeric structures, composed of 3, 4, 6, 12, or up to 24 subunits. Often times, these membrane super-structures repeat the same family of proteins several times. Likewise, the surfaces of bacteria and viruses contain groups of proteins repeated in geometric patterns. In considering fluorescent reporters, the simplest form would be using the same ligand element on each monomer cofluoron. However, since the cofluoron linker elements can be different, a single ligand may require two monomeric cofluorons to account for the two linker elements. For some cofluorons, especially those with the potential to form extended conjugation across heteroaromatic systems, it may be advantageous to combine three different linker elements, which nonetheless contain the same ligand elements. Further, since multimeric protein complexes often contain at least two subunits, it would be reasonable to develop such linker elements connected to both subunits, bringing the total number of potential monomers to six. This design does not preclude using the same monomer more than once in a given cofluoron multimer.

### Linker Elements

The concept of the linker element is to coax two small molecules to bind to one another, taking advantage of hydrophobic, polar, ionic, hydrogen bonding, and/or reversible or irreversible covalent interactions. The linker element may or may not be fluorescent.

The substituents on the linker elements can be varied to tune the equilibrium of the reversible association of the linker elements in aqueous solution, and to tune the fluorescent properties of the linker element, if present. For reversible covalent bond formation, linker elements may be derived from boronates.

When different ligand elements are to be presented, heterodimeric linker elements may be desirable, while if identical ligand elements are to be presented (e.g. to a multimeric target), homodimeric linkers may be desirable. Nevertheless, a successful linker element design that binds tightly to an identical linker element with a different ligand may also be used. If the ligands do not influence self-binding, then using two different ligands with identical linker elements should generate the A-B heterodimer approximately half of the time in the absence of the target.

The term “physiological conditions” is hereby defined as aqueous conditions inside the body or the cell, comprising a temperature range of about 35-40° C., a pH range of about 5.5-8, a glucose concentration range of about 1-20 mM, and an ionic strength range of about 110 mM to about 260 mM.

An important variation in the linker element design is to have the linker element come together through two covalent bonds. The advantage of such an approach is that even though the individual reaction may be unfavored, once a single bond is made, the local concentration of the other two groups favors formation of the second covalent bond and helps drive the equilibrium towards linker element formation.

A second and related concept is to prevent or minimize side reactions between the individual linker element and active groups on proteins, amino acids, or other molecules in the cell. Such side reactions may be reduced by designing linker element structures that may be sterically hindered when reacting with a large macromolecule, but more amenable to reacting when aligned with a partner linker element especially when bound to the macromolecular target which can serve as a template to position linkers proximally and promote the reaction.

Further, the architecture of the linker element covalent interactions should favor intermolecular bond formation over intramolecular bond formation.

An additional concept is that a linker element in a monomer may react with and form a covalent adduct with the target thus modifying the linker element and allowing it to interact with a different linker element. Further, the dimer or multimer may also form a covalent adduct with the target.

Finally, when the linker elements are in use, they will each have an affinity to their target, and this too will help assemble the dimeric linker element structure. In other words, the intended macromolecular target helps assemble the cofluoron multimer.

Often cofluorons dynamically and reversibly come together to form multimers with new stereocenters or alternative geometries. For example, boronic acid diesters may be planar (sp² hybridized) at the boron, or may have tetrahedral geometry (sp³ hybridized) in which the sp³ boron is chiral due to an additional donor ligand or hydroxyl group. In the absence of a target, cofluoron dimer or multimer stereoisomers may have similar stability or probability of formation. In the presence of target, certain stereoisomers of cofluoron dimers or multimers will be selectively bound by the macromolecular target, which significantly favors their association and potential formation on the target. If cofluorons form less preferred stereoisomers, geometries or conformers, they will not be as avidly bound by the target, and hence will be liberated to isomerize to the more preferred isomer that will bind to the target. While in solution, diastereomers may have similar stabilities and energies, it is anticipated that each stereoisomer will exhibit differential binding to the target, resulting in the target selecting for the highest affinity diastereomer. Less preferred cofluoron isomers can equilibrate through ring opening or epimerization or dissociation to monomers until the more preferred isomer is produced and bound to the target. Such examples illustrate a key advantage of this technology over existing technologies involving the covalent synthesis, separation of stereoisomers, determination of chirality and testing of fragment assemblies.

Association of Linker Elements with a Co-Factor.

Some linker elements may be brought together with the assistance of a cofactor, either naturally present within the cell, or added exogenously. The cofactor may optionally provide additional affinity to the target.

Linker Elements Based on Forming Reversible Boronate Esters.

These compounds may be ideal for screening purposes, as well as may work in vivo. One potential caveat is that many sugars have diols that may react with the boronic acid containing linker element. Boronates can also complex with amino alcohols and may also compex with amino acids, ortho-hydroxy aryl amides, ortho-hydroxy aryl hydroxamic acids and its derivatives.

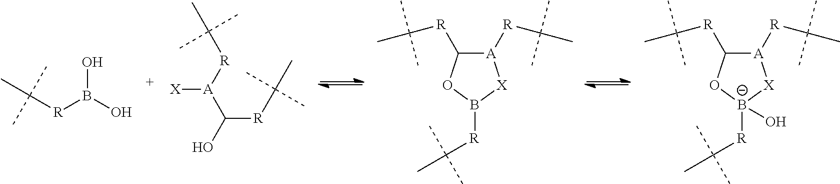

where X, R, R′ and R″ may be varied to tune the equilibrium in aqueous solution, where A=(CH₂)ₙ where n=1, 2, where the equilibrium species with the tetrahedral boron may include one or both stereoisomers and the lines crossed with a dashed line illustrate the one or more bonds formed joining the one or more ligand elements, directly or through a connector, to the molecule.

In some instances, the sp² boronic acid diester is fluorescent, while in other instances, the sp³ boronate is the fluorophore.

Some embodiments for the collection of monomers capable of forming a multimer useful as a fluorescent reporter include a first monomer having a first linker, Z₁ and second monomer having a second linker, Z₂. The second linker Z₂ is a boronic acid or oxaborole moiety capable of binding with Z₁ of the first monomer to form the multimer.

In certain embodiments, the first linker Z₁ of the first monomer is selected from the following groups a)-f):

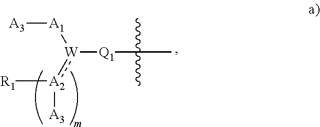

wherein
A₁ is (a) absent; or (b) selected from the group consisting of acyl, substituted or unsubstituted aliphatic, and substituted or unsubstituted heteroaliphatic;
A₂, independently for each occurrence, is (a) absent; or (b) selected from the group consisting of —N—, acyl, substituted or unsubstituted aliphatic, and substituted or unsubstituted heteroaliphatic, provided that at least one of A₁ and A₂ is present; or
A₁ and A₂, together with the atoms to which they are attached, form a substituted or unsubstituted 4-8 membered cycloalkyl or heterocyclic ring;
A₃ is selected from the group consisting of —NHR′, —SH, and —OH;
W is CR′ or N;
R′ is selected from the group consisting of hydrogen, halogen, substituted or unsubstituted aliphatic, substituted or unsubstituted heteroaliphatic, substituted or unsubstituted aryl, substituted or unsubstituted heteroaryl, —NH₂, —NO₂, —SH, and —OH;
m is 1-6;
[symbol] represents a single or double bond; and
R₁ is (a) absent; or (b) selected from the group consisting of hydrogen, halogen, substituted or unsubstituted aliphatic, substituted or unsubstituted heteroaliphatic, substituted or unsubstituted aryl, substituted or unsubstituted heteroaryl, —NH₂, —NO₂, —SH, and —OH;
Q₁ is (a) absent; or (b) selected from the group consisting of substituted or unsubstituted aliphatic and substituted or unsubstituted heteroaliphatic; or
R₁ and Q₁ together with the atoms to which they are attached form a substituted or unsubstituted 4-8 membered cycloalkyl or heterocyclic ring;

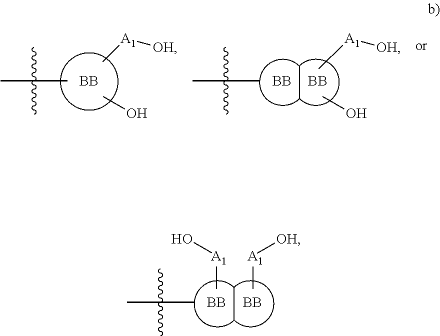

wherein
BB, independently for each occurrence, is a 4-8 membered cycloalkyl, heterocyclic, aryl, or heteroaryl moiety, wherein the cycloalkyl, heterocyclic, aryl, or heteroaryl moiety is optionally substituted with one or more groups represented by R₂, wherein the two substituents comprising —OH have a 1,2 or 1,3 configuration;
each R₂ is independently selected from the group consisting of hydrogen, halogen, oxo, sulfonate, —NO₂, —CN, —OH, —NH₂, —SH, —COOH, —CONHR′, substituted or unsubstituted aliphatic, and substituted or unsubstituted heteroaliphatic, or two R₂ together with the atoms to which they are attached form a fused substituted or unsubstituted 4-6 membered cycloalkyl or heterocyclic bicyclic ring system;
A₁, independently for each occurrence, is (a) absent; or (b) selected from the group consisting of acyl, substituted or unsubstituted aliphatic, and substituted or unsubstituted heteroaliphatic;
R′ is selected from the group consisting of hydrogen, halogen, substituted or unsubstituted aliphatic, substituted or unsubstituted heteroaliphatic, substituted or unsubstituted aryl, substituted or unsubstituted heteroaryl, —NH₂, —NO₂, —SH, and —OH;

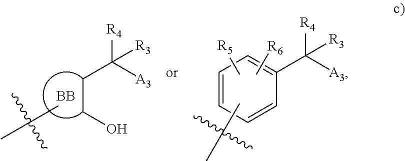

wherein
BB is a substituted or unsubstituted 5- or 6-membered cycloalkyl, heterocyclic, aryl, or heteroaryl moiety;
A₃, independently for each occurrence, is selected from the group consisting of —NHR′ and —OH;
R₃ and R₄ are independently selected from the group consisting of H, C₁₋₄ alkyl, and phenyl, or R₃ and R₄ taken together from a 3-6 membered ring;
R₅ and R₆ are independently selected from the group consisting of H; C₁₋₄ alkyl optionally substituted by hydroxyl, amino, halogen, or thio; C₁₋₄ alkoxy; halogen; —OH; —CN; —COOH; and —CONHR′; or R₅ and R₆ taken together form phenyl or a 4-6 membered heterocycle; and
R′ is selected from the group consisting of hydrogen, halogen, substituted or unsubstituted aliphatic, substituted or unsubstituted heteroaliphatic, substituted or unsubstituted aryl, substituted or unsubstituted heteroaryl, —NH₂, —NO₂, —SH, and —OH;

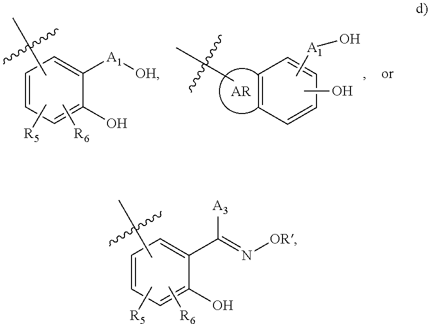

wherein
A₁ is (a) absent; or (b) selected from the group consisting of acyl, substituted or unsubstituted aliphatic, and substituted or unsubstituted heteroaliphatic;
A₃, independently for each occurrence, is selected from the group consisting of —NHR′ and —OH;
AR is a fused phenyl or 4-7 membered aromatic or partially aromatic heterocyclic ring, wherein AR is optionally substituted by oxo; C₁₋₄ alkyl optionally substituted by hydroxyl, amino, halo, or thio; C₁₋₄ alkoxy; —S—C₁₋₄ alkyl; halogen; —OH; —CN; —COOH; or —CONHR′; wherein the two substituents comprising —OH are ortho to each other;
R₅ and R₆ are independently selected from the group consisting of H; C₁₋₄ alkyl optionally substituted by hydroxyl, amino, halo, or thio; C₁₋₄ alkoxy; halogen; —OH; —CN; —COOH; and CONHR′; and
R′ is selected from the group consisting of hydrogen, halogen, substituted or unsubstituted aliphatic, substituted or unsubstituted heteroaliphatic, substituted or unsubstituted aryl, substituted or unsubstituted heteroaryl, —NH₂, —NO₂, —SH, and —OH;

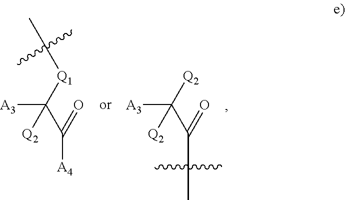

wherein
Q₁ is selected from the group consisting of C₁₋₄ alkyl; alkylene; a bond; C₁₋₆ cycloalkyl; a 5-6 membered heterocyclic ring; and phenyl;
Q₂, independently for each occurrence, is selected from the group consisting of H; C₁₋₄ alkyl; alkylene; a bond; C₁₋₆ cycloalkyl; a 5-6 membered heterocyclic ring; phenyl; substituted or unsubstituted aliphatic; substituted or unsubstituted heteroaliphatic; substituted or unsubstituted aryl; and substituted or unsubstituted heteroaryl;
A₃, independently for each occurrence, is selected from the group consisting of —NH₂ or —OH;
A₄, independently for each occurrence, is selected from the group consisting of —NH—NH₂, —NHOH, —NH—OR″, and —OH;
R″ is selected from the group consisting of H and C₁₋₄ alkyl; and

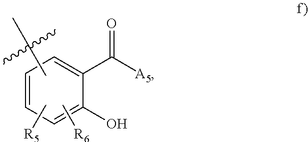

wherein
A₅ is selected from the group consisting of —OH, —NH₂, —SH, and —NHR′″;
R′″ is selected from the group consisting of —NH₂, —OH, and C₁₋₄ alkoxy;
R₅ and R₆ are independently selected from the group consisting of H; C₁₋₄ alkyl optionally substituted by hydroxyl, amino, halo, or thio; C₁₋₄ alkoxy; halogen; —OH; —CN; —COOH; and —CONHR′; or R₅ and R₆ taken together may form a 5-6 membered ring;

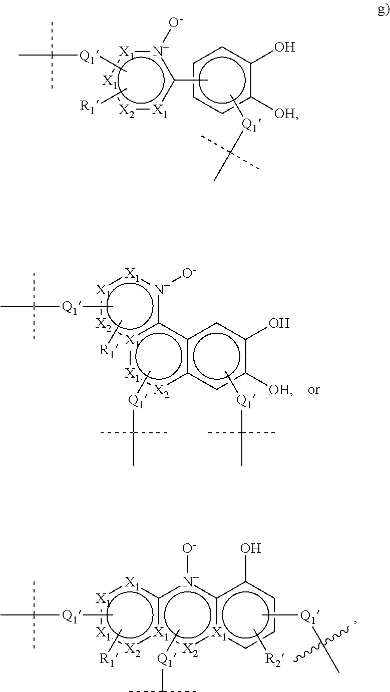

wherein:
------ represents an optional connection points where Z₁ is connected to one or more ligand elements, directly or through a connector;
each X₁ is independently C, N, O or S;
each X₂ is independently absent, C, N, O or S;
each R₁′ and R₂′ are independently be H, substituted or unsubstituted aliphatic, substituted or unsubstituted heteroaliphatic, substituted or unsubstituted aryl, substituted or unsubstituted heteroaryl;
each Q₁′ is independently absent, substituted or unsubstituted aliphatic, substituted or unsubstituted heteroaliphatic, substituted or unsubstituted aryl, substituted or unsubstituted heteroaryl, provided that at least one Q₁′ is present, providing at least one connection point of the formula to the one or more ligand element;
or Q₁′ and R₁′ together with the atoms they attach to form a fused 5- or 6-membered aromatic or heteroaromatic ring when Q₁′ and R₁′ are adjacent;
or Q₁′ and R₂′ together with the atoms they attach to form a fused 5- or 6-membered aromatic or heteroaromatic ring when Q₁′ and R₂′ are adjacent.

In some instances, A₁ may be selected from the group consisting of C₁-C₃ alkylene optionally substituted with one, two, or three halogens, and —C(O)—.

The embodiments below are non-limiting examples of the first linker Z₁.

In one embodiment, Z₁ is

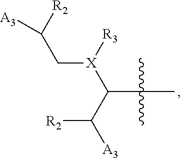

wherein R₂, independently for each occurrence, is selected from the group consisting of H and C₁₋₄ alkyl, or two R₁ moities taken together form a 5- or 6-membered cycloalkyl or heterocyclic ring, wherein R₃ is H, or

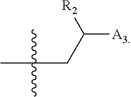

In one embodiment, Z1 is

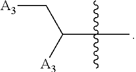

In one embodiment, Z₁ is

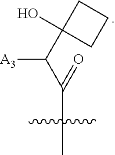

In one embodiment, Z₁ is

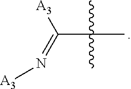

In one embodiment, Z₁ is

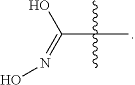 [SMILES: */C(O)=N/O]

In one embodiment, Z₁ is a monosaccharide or a disaccharide.

In one embodiment, Z₁ is selected from the group consisting of

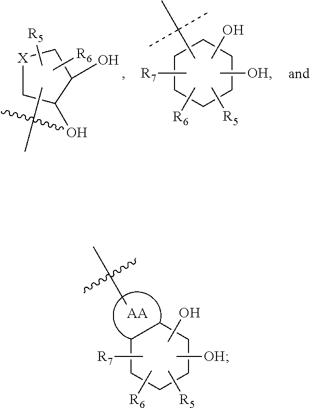

wherein
X is selected from the group consisting of O, S, CH, and NR′, wherein when X is NR′, N may be covalently bonded to the connector;
R′ is selected from the group consisting of H and C₁₋₄alkyl;
R₅, R₆, and R₇ are independently selected from the group consisting of H; C₁₋₄ alkyl optionally substituted by hydroxyl, amino, halo, or thio; C₁₋₄ alkoxy; halogen; —OH; —CN; —COOH; —CONHR′; and a mono- or bicyclic heterocyclic optionally substituted with amino, halo, hydroxyl, oxo, or cyano; and
AA is a 5-6 membered heterocyclic ring optionally substituted by C₁₋₄ alkyl optionally substituted by hydroxyl, amino, halo, or thio; C₁₋₄ alkoxy; halogen; —OH; —CN; —COOH; —CONHR′, or —S—C₁₋₄ alkyl.

In one embodiment, Z₁ is

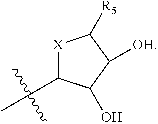

In one embodiment, Z₁ is

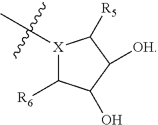

In one embodiment, X is nitrogen.

In one embodiment, Z₁ is

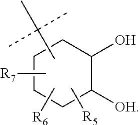

In one embodiment, Z₁ is

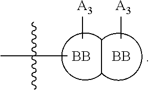

In one embodiment, Z₁ is

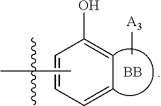

In one embodiment, Z₁ is

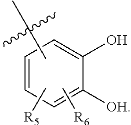

In one embodiment, Z₁ is

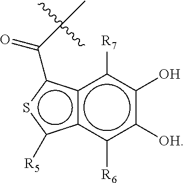

In one embodiment, Z₃ is

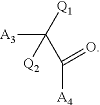

In one embodiment, Z₁ is

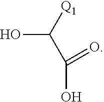

In one embodiment, Z₁ is

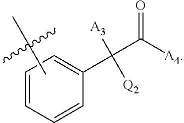

In one embodiment, Z₁ is

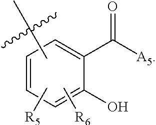

In one embodiment, Z₁ is

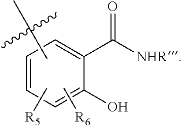

In one embodiment, Z₁ is

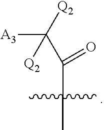

In one embodiment, Z₁ is

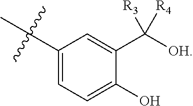

In one embodiment, Z₁ is

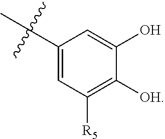

In one embodiment, Z₁ is

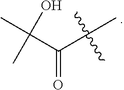 [SMILES: *C(=O)C(C)(C)O]

In one embodiment, Z₁ is selected from the group consisting of

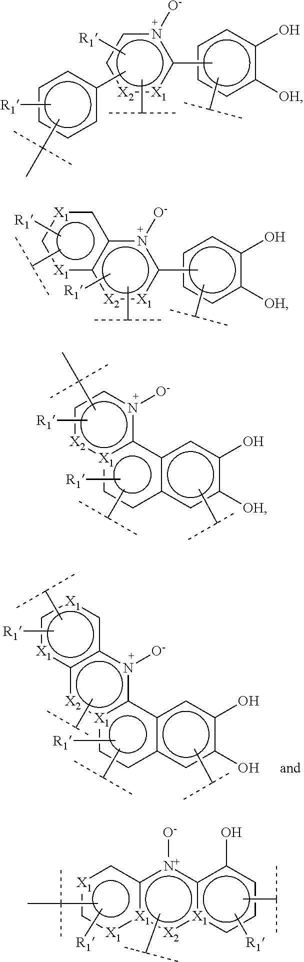

wherein

each X₁ is independently C or N;

each X₂ is independently absent, C or N;

each R₁′ is independently H; —OH; halogen; oxo; C₁₋₄ alkyl or phenyl optionally substituted by hydroxyl, amino, halo or thio; C₂₋₄ alkenyl; C₁₋₄ alkoxy; —S—C₁₋₄ alkyl; —CN; —COOH; —CONHR′; —NO₂ or NHR′, wherein R′ is H or C₁₋₄ alkyl.

In one embodiment, Z₁ is

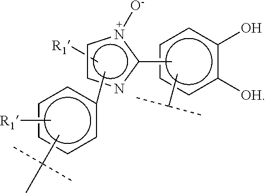

In one embodiment, Z₁ is

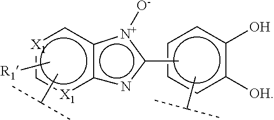

In one embodiment, Z₁ is

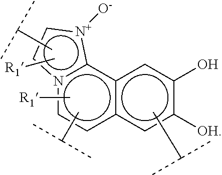

In one embodiment, Z₁ is

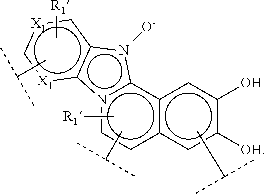

In one embodiment, Z₁ is

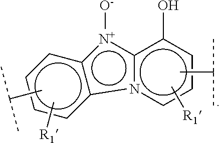

In one embodiment, Z₁ is

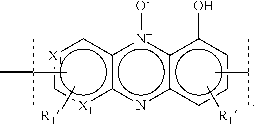

In certain embodiments, the second linker Z₂ from the second monomer is selected from the group consisting of:

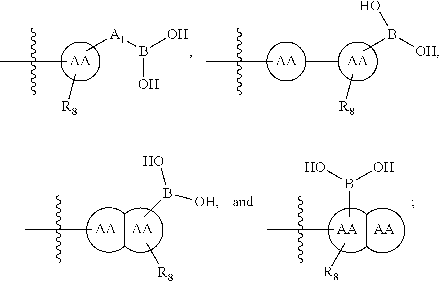

wherein
R₈ is selected from the group consisting of H; halogen; oxo; C₁₋₄ alkyl optionally substituted by hydroxyl, amino, halo or thio; C₂₋₄ alkenyl, C₁₋₄ alkoxy; —S—C₁₋₄ alkyl; —CN; —COOH; and —CONHR′;
A₁ is (a) absent; or (b) selected from the group consisting of acyl, substituted or unsubstituted aliphatic, and substituted or unsubstituted heteroaliphatic;
AA, independently for each occurrence, is phenyl, aryl, or a 5-7 membered heterocyclic or heteroaryl ring having one, two, or three heteroatoms, wherein AA is optionally substituted by one, two, or three substituents selected from the group consisting of halogen; C₁₋₄ alkyl optionally substituted by hydroxyl, amino, halogen, or thio; C₂₋₄ alkenyl, C₁₋₄ alkoxy; —S—C₁₋₄ alkyl; —CN; —COOH; and —CONHR′; or two substituents together with the atoms to which they are attached form a fused 4-6 membered cycloalkyl or heterocyclic bicyclic ring system; and
R′ is H or C₁₋₄ alkyl.

In one embodiment, R₈ and the substituent comprising boronic acid are ortho to each other, and R₈ is —CH₂NH₂.

In one embodiment, Z₂ is selected from the group consisting of:

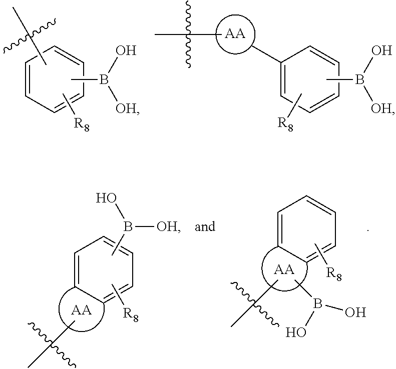

In one embodiment, Z₂ is selected from the group consisting of:

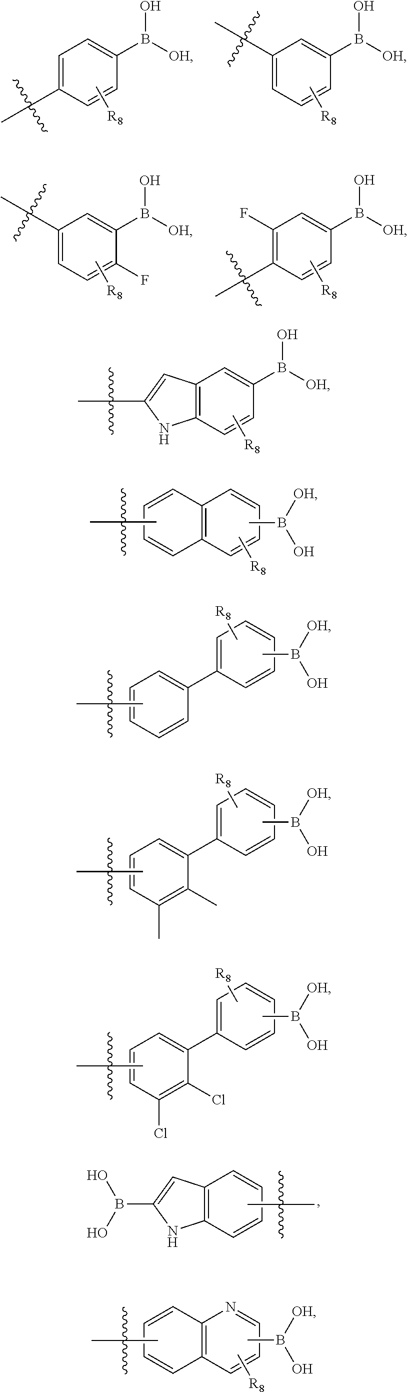

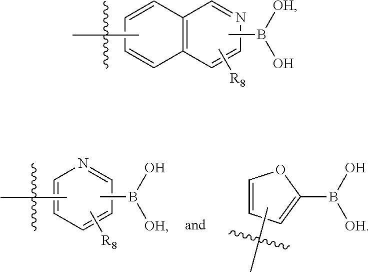

In some embodiments, the second linker Z₂ from the second monomer is selected from the group consisting of:

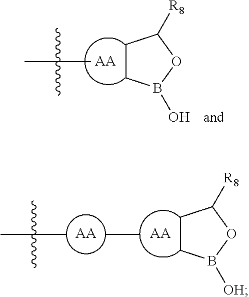

wherein
R₈ is selected from the group consisting of H; halogen; oxo; C₁₋₄ alkyl optionally substituted by hydroxyl, amino, halo or thio; C₂₋₄ alkenyl, C₁₋₄ alkoxy; S—C₁₋₄ alkyl; —CN; —COOH; and —CONHR′;
AA, independently for each occurrence, is a 5-7 membered heterocyclic ring having one, two, or three heteroatoms, or phenyl, wherein AA is optionally substituted by one, two, or three substituents selected from the group consisting of halo; C₁₋₄ alkyl optionally substituted by hydroxyl, amino, halo, or thio; C₂₋₄ alkenyl, C₁₋₄ alkoxy; —S—C₁₋₄ alkyl; —CN; —COOH; and —CONHR′; or two substituents together with the atoms to which they are attached form a fused 4-6 membered cycloalkyl or heterocyclic bicyclic ring system; and
R′ is H or C₁₋₄alkyl.

In some embodiments, the second linker Z₂ from the second monomer is selected from the group consisting of:

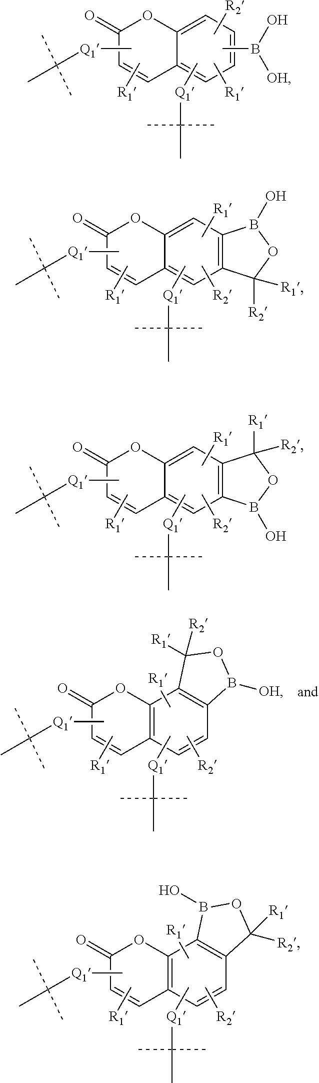

wherein

------ represents an optional connection points where Z₂ is connected to one or more ligand elements, directly or through a connector:

each R₁′ and R₂′ are independently H, substituted or unsubstituted aliphatic, substituted or unsubstituted heteroaliphatic, substituted or unsubstituted aryl, substituted or unsubstituted heteroaryl;

each Q₁′ is independently absent, substituted or unsubstituted aliphatic, substituted or unsubstituted heteroaliphatic, substituted or unsubstituted aryl, substituted or unsubstituted heteroaryl, provided that at least one Q₁′ is present, providing at least one connection point of the formula to the one or more ligand element;

or Q₁′ and R₁′ together with the atoms they attach to form a fused 5- or 6-membered aromatic or heteroaromatic ring when Q₁′ and R₁′ are adjacent;

or Q₁′ and R₂′ together with the atoms they attach to form a fused 5- or 6-membered aromatic or heteroaromatic ring when Q₁′ and R₂′ are adjacent

In one embodiment, Z₂ is selected from the group consisting of:

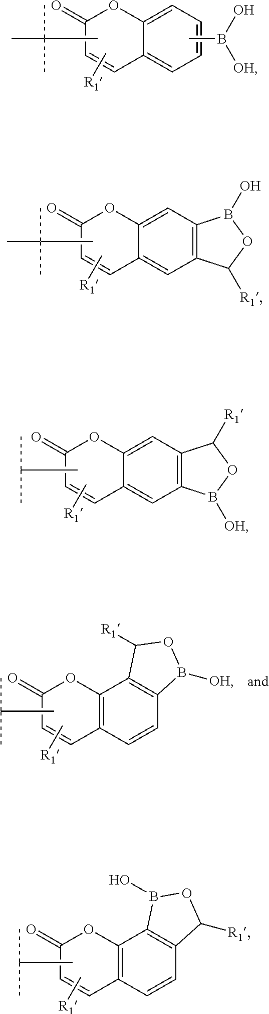

wherein

each R₁′ is independently H; halogen; oxo; C₁₋₄ alkyl or phenyl optionally substituted by hydroxyl, amino, halo or thio; C₂₋₄ alkenyl; C₁₋₄ alkoxy; —S—C₁₋₄ alkyl; —CN; —COOH; —CONHR′; —NO₂ or NHR′, wherein R′ is H or C₁₋₄ alkyl.

In some embodiments, at least one of the linker is an aliphatic, alicyclic or aromatic boronic acid or an oxaborole moiety. The boronic acid or oxaborole moiety is capable of reacting with one or more of its binding partners selected from the group consisting of diols, catechols, ortho-dihydroxycoumarins, amino alcohols, α-hydroxy acids, α-hydroxyamides, ortho-hydroxy-arylcarboxamides, triols, or derivatives thereof, to form boronate esters comprising 5, 6, or 7 membered rings, oxazaborolanes and oxazaborinanes, dioxaborininone or oxazoborininones.

In some embodiments, the generic structure of the boronic acid or oxaborole moiety can be represented by the following chemical moieties, where the lines crossed with a dashed line illustrate the one or more bonds formed joining the one or more ligand elements, directly or through a connector:

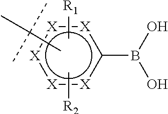

where X=C, N
where R₁, R₂ can be H or an electron withdrawing group such as —F, —Cl, —Br, —I, —CF₃, —CN, —OCH₃, or —NO₂, or when R₁ and R₂ are adjacent, may also include fused 5- or 6-membered aromatic or heteroaromatic ring;

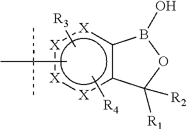

where X=C, N
where R₁, R₂ can be —H, —CH₃, -Ph, or connected to each other through a spiro 3-, 4-, 5- or 6-membered ring
where R₃, R₄ can be H or an electron withdrawing group such as —F, —Cl, —Br, —I, —CF₃, —CN, —OCH₃, or —NO₂, or when R₃ and R₄ are adjacent, may also include fused 5- or 6-membered aromatic or heteroaromatic ring; and

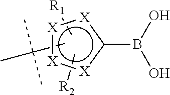

where X=C, N, O, S
where R₁, R₂ can be H or an electron withdrawing group such as —F, —Cl, —Br, —I, —CF₃, —CN, —OCH₃, or —NO₂, or when R₁ and R₂ are adjacent, may also include fused 5- or 6-membered aromatic or heteroaromatic ring.

In some embodiments, the generic structure for the binding partner linker elements of the above boronic acid or oxaborole moiety can be represented by the following chemical moieties, where the lines crossed with a dashed line illustrate the one or more bonds formed joining the one or more ligand elements, directly or through a connector, and where the stereoisomers of in the embodiments shown below are representative of and not limited to the different stereoisomers that used to associate with other linker elements:

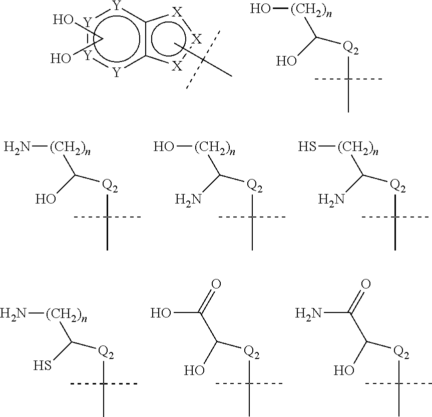

where Q₂ is an aliphatic, alicyclic, or hetero or non-hetero aromatic moiety
where n=1 or 2
where X and Y=C, N, O, or S
where hydroxy groups emanating from the aromatic ring are ortho to each other;

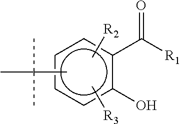

where R₁=—OH, —NH₂—SH, —NHR₄, and R₄=alkyl, —OH, alkoxy, —NH₂
where R₂, R₃ is a H or an electron donating group such as alkyl, alkoxy, aryl, —OH, —COOH, —CONH₂, or when R₂ and R₃ are adjacent, may also include fused 5- or 6-membered aromatic or heteroaromatic ring;

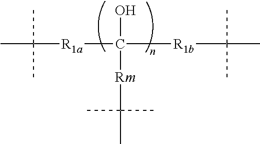

n=2-6
R₁, R_{1b}=—H, —CH₃, —CH₂NH₂, —CH₂NHCH₃, aromatic or heteroaromatic ring, or connected to each other through a 4, 5, 6, 7 or 8-membered ring
Rm=—H, —CH₃, —CH₂NH₂, —CH₂OH, —CH₂CH₂OH, and m=2-6;

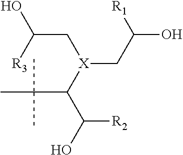

X=C, N
R₁, R₂, R₃=—H, —CH₃, or two R groups connected to each other through a 5 or 6 membered alicyclic ring

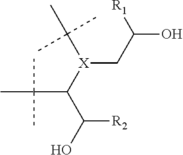

where X=C, N
where R₁, R₂=—H, —CH₃, or two groups connected to each other through a 5- or 6-membered alicyclic ring;

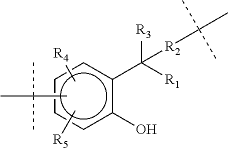

where R₁=—OH, —NH₂, —SH
where R₂ and R₃ can be —H, —CH₃, -Ph, —NOH, or connected to each other through a spiro 3-, 4-, 5- or 6-membered ring
where R₄ and R₅ can be H an electron donating group such as alkyl, alkoxy, aryl, —OH, —COON, —CONH₂, —C(R₂,R₃)OH or when R₄ and R₅ are adjacent, may also include fused 5- or 6-membered aromatic or heteroaromatic ring;

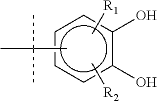

where R₁, R₂ can be H, an electron donating group such as alkyl, alkoxy, aryl, —OH, —COOH, —CONH₂, an electron withdrawing group such as —F, —Cl, —Br, —I, —CF₃, —CN, —OCH₃, or —NO₂, or when R₁ and R₂ are adjacent, may also include fused 5- or 6-membered aromatic or heteroaromatic ring;

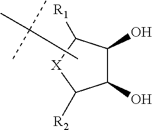

X=C, N, O, S
R₁, R₂=—H, —CH₃, —OH, —CH₂OH, -Adenyl;

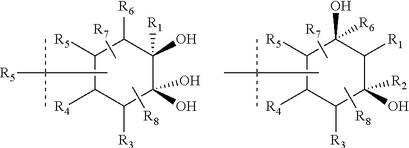

R₁, R₂, R₃, R₄, R₅, R₆=—H, —CH₃
R₇, R₈ are connected to each other to form 3.1.1, 2.2.1 and 2.2.2 bicyclic ring systems such that the hydroxyls are cis to each other; and

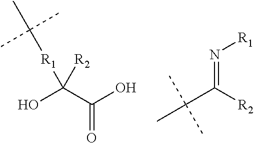

R₁, R₂=—H, —CH₃, -Ph, —C₆H₁₁, —C₅H₉, R₁, R₂=—OH, —NH₂ aromatic or heteroaromatic ring, C₁-C₆-alkyl or C₃-C₈ cycloalkyl.

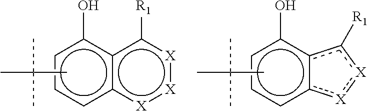

X=C, N X=C, N, O, S
R₁=—OH, —NH₂, —NHR₂, —NHC(═O)R₂, —NHSO₂R₂R₁, R₂=—NH₂, ═O, —OH
   Derivatives Based on Boronic Acid or Oxaborole that Form Covalent Interactions with Diols, Catechols, Ortho-Hydroxycoumarins, Amino Alcohols, Amino Thiols, α-Hydroxy Acids, α-Hydroxyamides, and Ortho-Hydroxy-Aryl Carboxamides, Triols, or Derivatives Thereof.

A typical reaction scheme of aliphatic, alicyclic, and aromatic boronic acids reacting with 1,2-, 1,3-, 1,4-diols to form boronate esters comprising 5, 6, or 7 membered rings are shown as below, e.g., for the reaction of a boronic acid with a 1,2-diol.

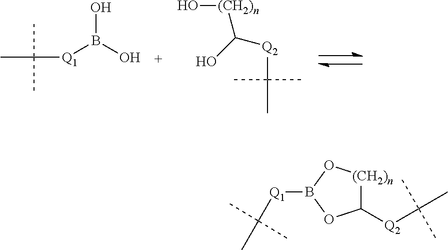

where Q₁ and Q₂ are aliphatic, alicyclic, or hetero or non-hetero aromatic moieties
where n=1, 2 or 3
where the lines crossed with a dashed line illustrate the one or more bonds formed joining the one or more ligand elements, directly or through a connector.

An example of a dimer formed from a boronic acid and an aromatic 1,2-diol is shown below:

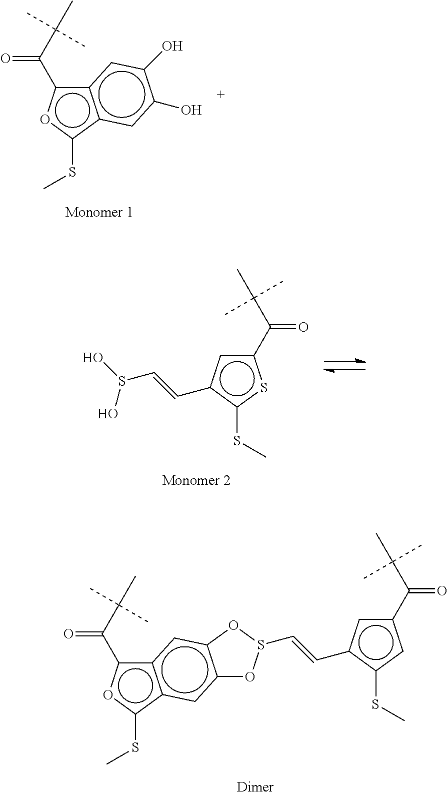

Although only a boronic acid diester with an sp² hybridized boron is shown, boronic acids may also form enantiomeric tetrahedral sp³ boronate ester complexes.

Examples of boronic acid linker element monomers are:

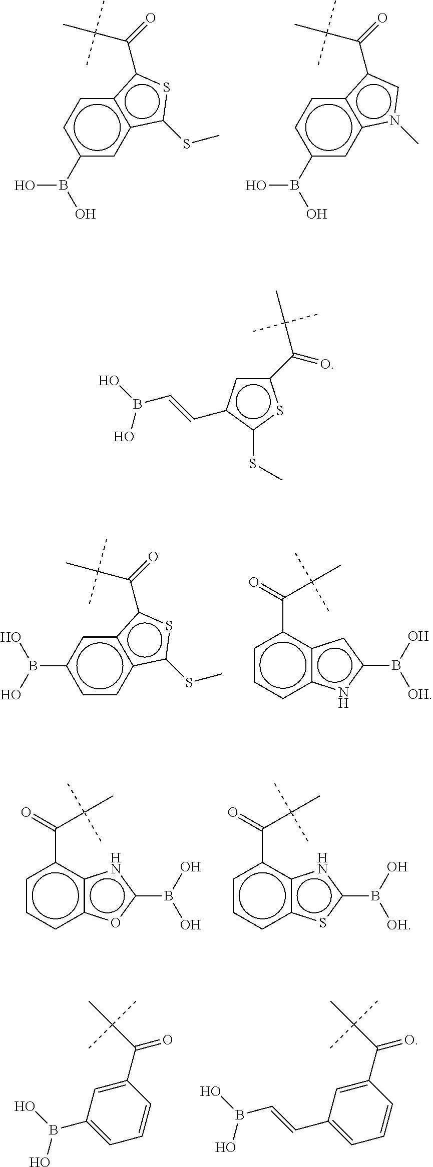

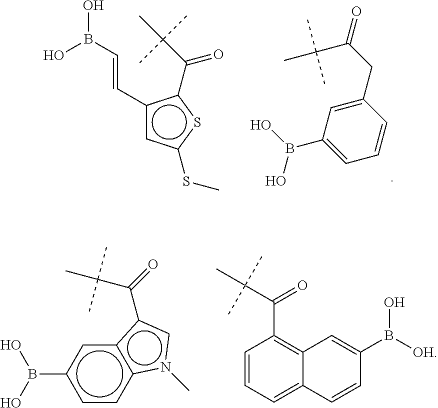

Additional examples of boronic acid linker moieties when appropriately bearing ligand elements for a macromolecular target elements include but are not limited to those listed below:

| | |
|---|---|
| (5-Amino-2-hydroxymethylphe- | 2-(Hydroxymethyl)phenylboronic |
| nyl)boronic acid | acid |
| 2-(N,N-dimethylamino)pyridine-5- | 2-(Trifluoromethyl)pyridine-5- |
| boronic acid hydrate | boronic acid |
| 2-Chloroquinoline-3-boronic acid | 2-Fluorophenylboronic acid |
| 2-Fluoropyridine-3-boronic acid | 2-Fluoropyridine-5-boronic acid |
| 2-Methoxypyridine-5-boronic acid | 2-Methoxypyrimidine-5-boronic |
| | acid |
| 2,3-Difluorophenylboronic acid | 2,4-Bis(trifluoromethyl)phe- |
| | nylboronic |
| | acid |
| 2,4-Bis(trifluoromethyl)phe- | 2,4-Difluorophenylboronic acid |
| nylboronic acid | |
| 2,5-Difluorophenylboronic acid | 2,6-Difluorophenylboronic acid |
| 2,6-Difluorophenylboronic acid | 2,6-Difluoropyridine-3-boronic |
| | acid hydrate |
| 3-(Trifluoromethyl)phenylboronic | 3-Fluorophenylboronic acid |
| acid | |
| 3-Nitrophenylboronic acid | 3,4-Difluorophenylboronic acid |
| 3,5-Bis(trifluoromethyl)phe- | 3,5-Difluorophenylboronic acid |
| nylboronic acid | |
| 4-Fluorophenylboronic acid | 4-Nitrophenylboronic acid |
| 5-Quinolinylboronic acid | Benzofuran-2-boronic acid |
| Benzothiophene-2-boronic acid | Furan-2-boronic acid |
| Phenylboronic acid | Pyridine-3-boronic acid |
| Pyrimidine-5-boronic acid | Thiophene-2-boronic acid |
| 2-Hydroxymethyl-5-nitrophe- | 2-Hydroxyphenylboronic acid |
| nylboronic acid | |
| 2,4-Dimethoxyphenylboronic acid | 2,6-Dimethoxypyridine-3-boronic |
| | acid |
| 4-(N,N-dimethylamino)phe- | 6-Indolylboronic acid |
| nylboronic acid | |
| trans-2-Phenylvinylboronic acid | 2-Hydroxymethyl(dimethyl)phe- |
| | nyl)boronic acid |
| Naphthalene-1-boronic acid | 3-Pyridinyl(2-hydroxymeth- |
| | yl)boronic acid |
| Quinoline-5-boronic acid | Dibenzofuran-4-boronic acid |

The boronic acid or oxaborole linker elements can also include those coumarin-containing molecules. The generic structure of the boronic acid or oxaborole moiety can be represented by the following formulas, where the line(s) crossed with the dashed line(s) illustrate the one or more possible connection points where the linker element is joined to one or more ligand elements, directly or through a connector:

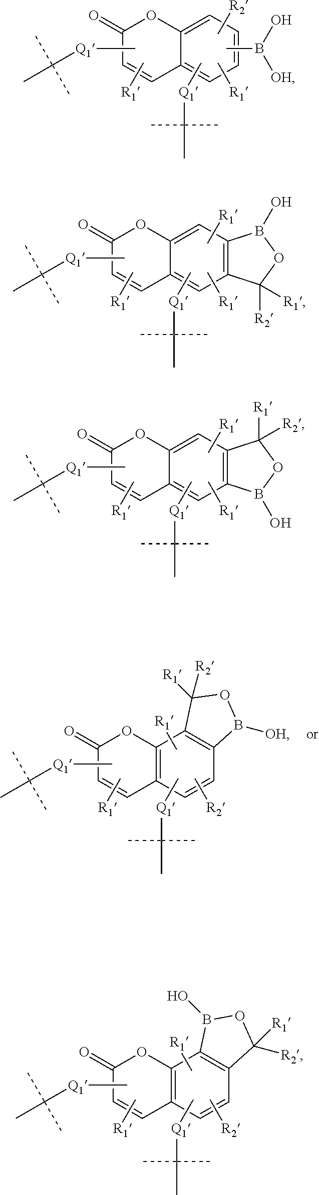

where each R₁′ and R₂′ can independently be H, substituted or unsubstituted aliphatic, substituted or unsubstituted heteroaliphatic, substituted or unsubstituted aryl, substituted or unsubstituted heteroaryl;
each Q₁′ is independently absent, substituted or unsubstituted aliphatic, substituted or unsubstituted heteroaliphatic, substituted or unsubstituted aryl, substituted or unsubstituted heteroaryl, provided that at least one Q₁′ is present, providing at least one connection point of the formula to the one or more ligand element;
or Q₁′ and R₁′ together with the atoms they attach to form a fused 5- or 6-membered aromatic or heteroaromatic ring when Q₁′ and R₁′ are adjacent;
or Q₁′ and R₂′ together with the atoms they attach to form a fused 5- or 6-membered aromatic or heteroaromatic ring when Q₁′ and R₂′ are adjacent.

In some embodiments, the boronic acid or oxaborole linker elements can be represented by the following formulas:

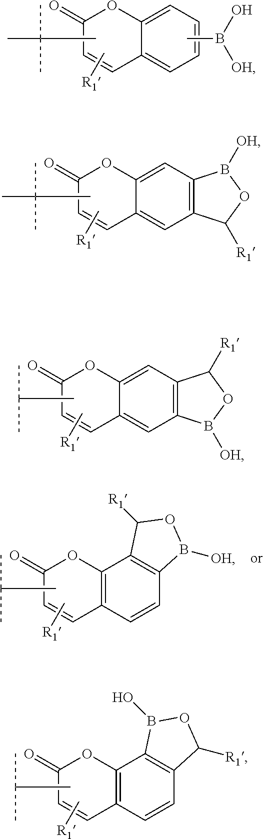

wherein each R₁′ is independently H; halogen; oxo; C₁₋₄ alkyl or phenyl optionally substituted by hydroxyl, amino, halo or thio; C₂₋₄ alkenyl; C₁₋₄ alkoxy; —S—C₁₋₄ alkyl; —CN; —COOH; —CONHR′; —NO₂ or NHR′, wherein R′ is H or C₁₋₄ alkyl.

Examples of linker elements containing diols or other linker elements that form covalent interactions with boronic acid linker elements:

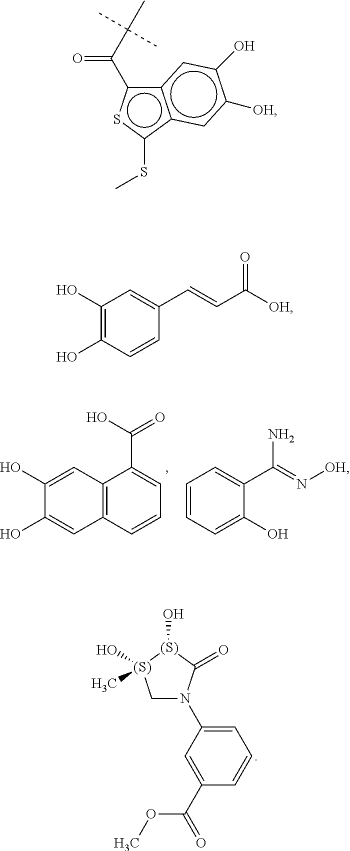

The example below shows the reaction of a boronic acid with an ortho-dihydroxy aromatic diol

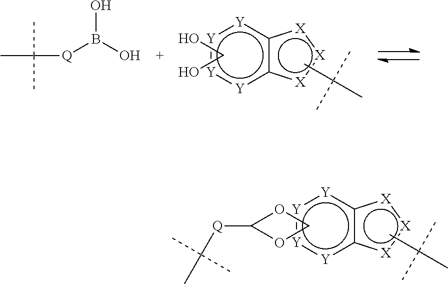

where Q is an aliphatic, alicyclic, or hetero or non-hetero aromatic moiety
where X and Y═C, N, O, or S
where the hydroxy groups emanating from the aromatic ring are ortho to each other
where the lines crossed with a dashed line illustrate the one or more bonds formed joining the one or more ligand elements, directly or through a connector.

Additional examples of diol or triol linker moieties when appropriately bearing ligand elements for a macromolecular target include but are not limited to those listed below:

| | |
|---|---|
| (±)-exo,exo-2,3-Camphanediol | (−)-Epigallocatechin gallate |
| (1R,2R,3S,5R)-(−)-Pinanediol | (3S,4R)-pyrrolidine-3,4-diol |
| 2,3,4-Trihydroxybenzophenone | 2,6-Bis(hydroxymethyl)-p-cresol |
| 3-Methyl-1,3,5-pentanetriol | 3,4-Dihydroxybenzonitrile |
| 3,4,5-Trihydroxybenzamide | 4-Methylcatechol |
| 6,7-Dihydroxy-4-methylcoumarin | 7,8-Dihydroxy-4-methylcoumarin |
| Adenosine | Alizarin Red S |
| cis-1,2-Cyclooctanediol | cis-1,2-Cyclopentanediol |
| D-(−)-Fructose | D-Sorbitol |
| Gallic acid | Gallic Acid Ethanolamide |
| Labetalol hydrochloride | meso-Erythritol |
| Methyl 3,4,5-trihydroxybenzoate | Propyl gallate |
| Pyrocatechol | Pyrogallol |
| Tricine | Triisopropanolamine |
| 1,1,1-Tris(hydroxymethyl)ethane | 1,3-Dihydroxyacetone |
| 2-(Methylamino)phenol | 2-Acetamidophenol |
| 2-Amino-2-methyl-1,3-propanediol | 2-Amino-4-methylphenol |
| 2-Hydroxy-3-methoxybenzyl | 3-Methylamino-1,2-propanediol |
| alcohol | |
| cis-1,2-Cyclohexanediol | D-(+)-Glucose |
| Hydroxypyruvic acid, Lithium salt | Pentaerythritol |
| Phenylpyruvic acid | Pinacol |
| trans-1,2-Cyclohexanediol | Tris Base (TRIZMA Base) |
| 3-Fluorocatechol | |
| 4-Nitrocatechol | 3-Methoxycatechol |
| 3,4-Dihydroxybenzonitrile | 2,3-Dihydroxynaphthalene |
| 1,8-Naphthalenediol | 2-(1-Hydroxy-1-methylethyl)phenol |

The example below shows the reaction of a boronic acid with a 1,2 or 1,3-amino alcohol.

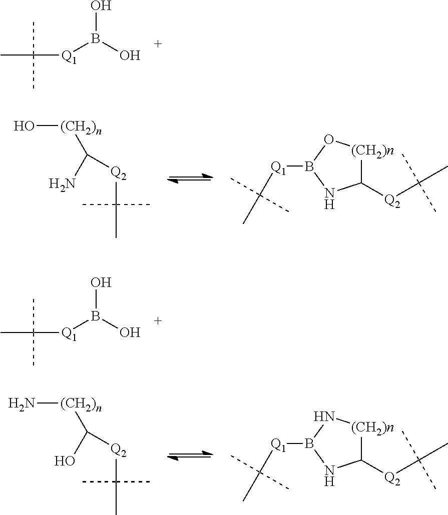

where Q₁ and Q₂ are aliphatic, alicyclic, or hetero or non-hetero aromatic moieties
where n=1 or 2
where the lines crossed with a dashed line illustrate the one or more bonds formed joining the one or more ligand elements, directly or through a connector.

The example below shows the reaction of a boronic acid with an α-hydroxy acid.

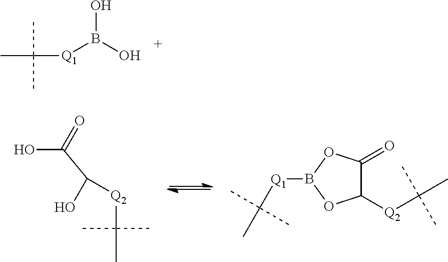

where Q₁ and Q₂ are aliphatic, alicyclic, or hetero or non-hetero aromatic moieties where the lines crossed with a dashed line illustrate the one or more bonds formed joining the one or more ligand elements, directly or through a connector.

Examples of linker elements containing α-hydroxy acids that form covalent interactions with boronic acid linker elements:

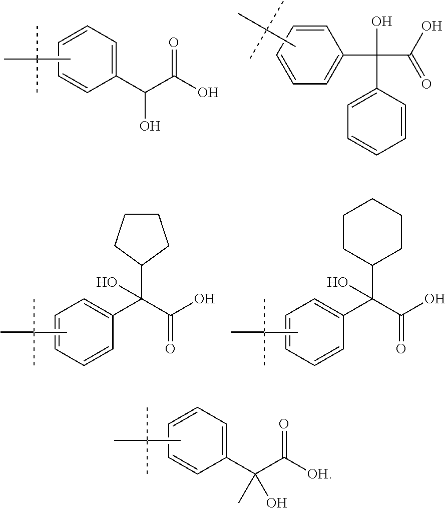

Additional examples of α-hydroxy acid linker elements include but are not limited to those listed below:

| | |
|---|---|
| Lactic acid | 2,2-Bis(hydroxymethyl)propionic |
| | acid |
| Salicylic acid | DL-Mandelic acid |
| 3,3,3-Trifluoro-2-hydroxy-2- | 3,3,3-Trifluoro-2-hydroxy-2- |
| (Trifluoromethyl)propionic acid | methylpropionic Acid |
| 3,5-Difluoromandelic acid | 2,6-Difluoromandelic acid |
| 2,6-Dihydroxybenzoic acid | 2,3-Difluoromandelic acid |
| 2,4-Difluoromandelic acid | 2,5-Difluoromandelic acid |
| 4-(Trifluoromethyl)mandelic acid | D-(−)-Quinic acid |
| Benzilic acid | 2-Fluoromandelic acid |
| DL-Atrolactic acid hemihydrate | α-Cyclohexylmandelic acid |
| α-Cyclopentylmandelic acid | α-Hydroxyisobutyric acid |
| 3-Hydroxyazetidine-3-carboxylic | 2-Hydroxy-4-methoxybenzoic acid |
| acid | |

The example below shows the reaction of a boronic acid with an α-hydroxyamide.

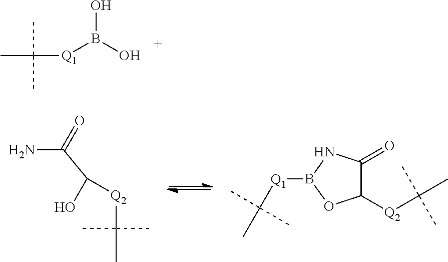

where Q₁ and Q₂ are aliphatic, alicyclic, or hetero or non-hetero aromatic moieties where the lines crossed with a dashed line illustrate the one or more bonds formed joining the one or more ligand elements, directly or through a connector.

Examples of linker elements containing α-hydroxyamides, o-hydroxyarylcarboxamides, or o-hydroxyaryl hydroxamic acids and derivatives that form covalent interactions with boronic acid linker elements:

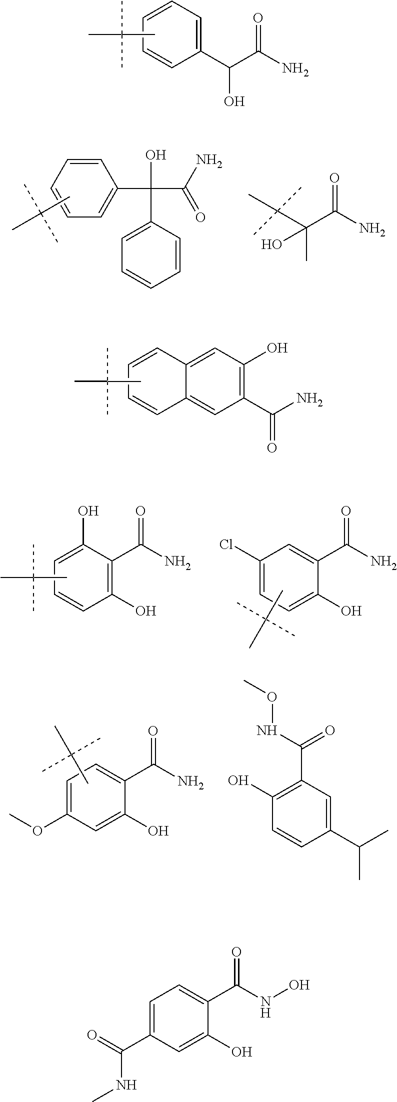

Additional examples of α-hydroxyamides or o-hydroxyarylcarboxamide linker elements include but are not limited to those listed below:

| | |
|---|---|
| 2-Hydroxy-3-naphthalenecarboxamide | N-(2-hydroxyethyl)salicylamide |
| 4-Methoxysalicylamide | Salicylamide |
| 2,6-Dihydroxybenzamide | Salicylhydroxamic acid |

The linker elements that can react with boronic acid or oxaborole linker elements can also include those N-oxide-containing compounds. The generic structure of the N-oxide-containing compound can be represented by the following formulas, where the line(s) crossed with the dashed line(s) illustrate the one or more possible connection points where the formula is joined to one or more ligand elements, directly or through a connector:

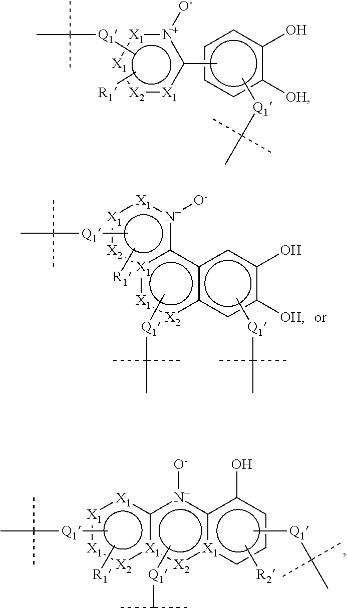

wherein:

------ represents an optional connection points where Z₁ is connected to one or more ligand elements, directly or through a connector;

each X₁ is independently C, N, O or S;

each X₂ is independently absent, C, N, O or S;

each R₁′ and R₂′ are independently H, substituted or unsubstituted aliphatic, substituted or unsubstituted heteroaliphatic, substituted or unsubstituted aryl, substituted or unsubstituted heteroaryl;

each Q₁′ is independently absent, substituted or unsubstituted aliphatic, substituted or unsubstituted heteroaliphatic, substituted or unsubstituted aryl, substituted or unsubstituted heteroaryl, provided that at least one Q₁′ is present, providing at least one connection point of the formula to the one or more ligand element;

or Q₁′ and R₁′ together with the atoms they attach to form a fused 5- or 6-membered aromatic or heteroaromatic ring when Q₁′ and R₁′ are adjacent;

or Q₁′ and R₂′ together with the atoms they attach to form a fused 5- or 6-membered aromatic or heteroaromatic ring when Q₁′ and R₂′ are adjacent.

In some embodiments, the N-oxide-containing compounds can be represented by the following formulas:

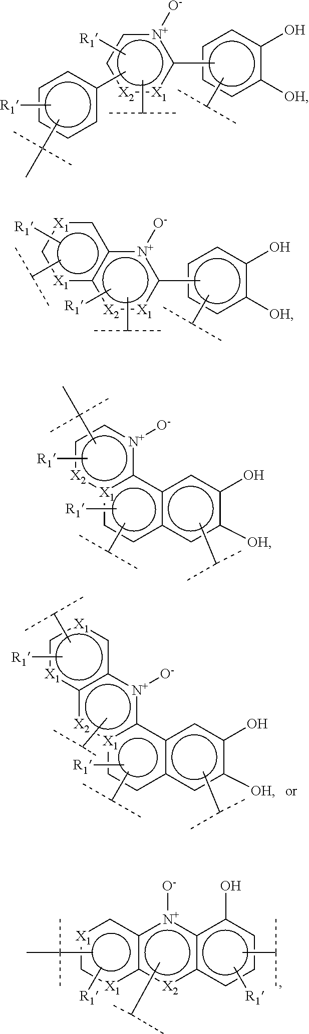

wherein

each X₁ is independently C or N;

each X₂ is independently absent, C or N;

each R₁′ is independently H; —OH; halogen; oxo; C₁₋₄ alkyl or phenyl optionally substituted by hydroxyl, amino, halo or thio; C₂₋₄ alkenyl; C₁₋₄ alkoxy; —S—C₁₋₄ alkyl; —CN; —COOH; —CONHR′; —NO₂ or NHR′, wherein R′ is H or C₁₋₄ alkyl.

Examples for the N-oxide compound are shown as below:

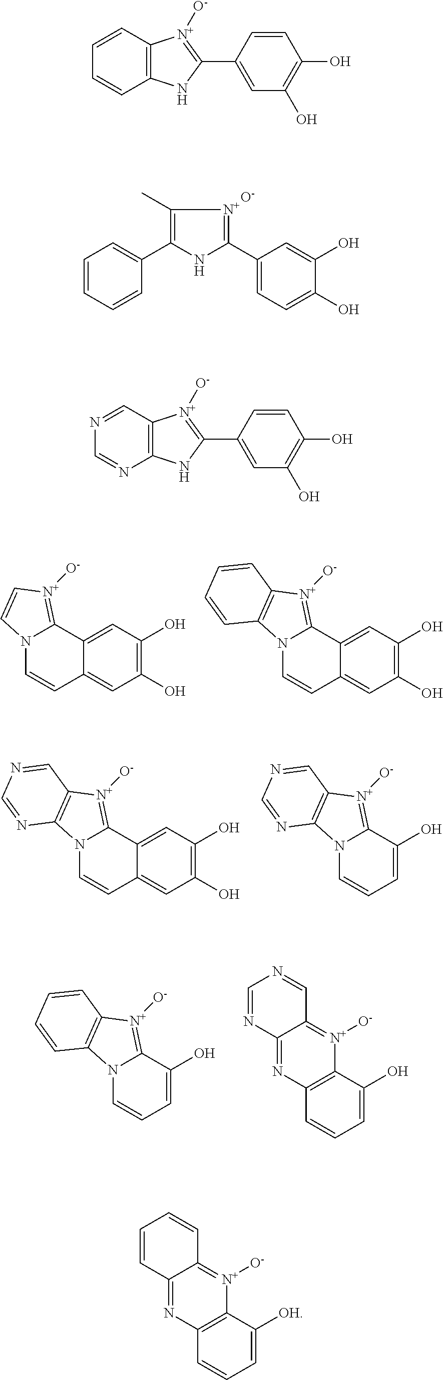

### Connectors

Connectors are used to connect the linker element to the ligand element. The connector enables the correct spacing and geometry between the linker element and the ligand element such that the cofluoron dimer or multimer formed from the monomers orients the ligand elements to allow high affinity binding of the ligand elements to the macromolecular target. The connector itself may function as a secondary ligand element by forming favorable interactions with the macromolecular target. The ideal connectors allow for modular assembly of cofluoron monomers through facile chemical reactions between reactive groups on the connector and complementary reactive groups on the linker elements and ligand elements. Additionally, connectors may be trifunctional and allow for the addition of encryption elements to allow for deconvolution of cofluoron monomers that are synthesized in a combinatorial fashion.

In one embodiment, a linker element is attached to a tri-functional connector, with one of the functionalities used to attach the connector-linker elements to a bead. Beads are distributed to unique wells, and a set of ligand elements react with the third functional group on the connector (for example 500 different aldehyde containing moieties reacted with an amino group). In this embodiment, the well the synthesis took place in identities the ligand element.

In a second embodiment (FIG. 2A), a linker element is attached to a tri-functional connector, with one of the functionalities used to attach the connector-linker element to an encoded bead. For example, Veracode™ beads (Illumina, San Diego, Calif.) or silicon particles may be used, where each bead has a unique Veracode™ or barcode pattern. The beads or particles are distributed into a set of reaction chambers (for example 10 chambers), identified in each chamber, and then reacted with a bifunctional moiety (for example, a protected amino acid). The beads are mixed, split again into the reaction chambers, and the process is repeated (split-pool synthesis). In this embodiment, repeating the process a total of 4 times will result in 10,000 ligand elements in the library. In a variation of this approach, at the end of the synthesis, the last amino acid residue is reacted with the connector to create a circular ligand element. In this version, the ligand element is identified by the code on the bead or particle.

In a third embodiment, a linker element is attached to a tri-functional connector, with one of the functionalities used to attach the connector-linker element to either a Veracode™ bead or a bar code particle. The remaining functionality is connected to a “platform” containing additional functionalities. For example, the platform may be a cyclopentane derivatized on three carbons all in the syn orientation. In this version, one of the encoding processes described in embodiments 2-5 above is used to add mono-functional moieties to the appropriate functional groups on the platform. For example, if there are 20 moieties added in each step, the resultant library will contain 8,000 ligand elements. The advantage of this approach is to guide all the diversity components in a single orientation for maximum diversity in binding surfaces.

Ligand Elements and their Targets

Cofluorons have the advantage of being able to bind the target, or to the proximate locations of target, through two or more ligands or ligand elements. In order for cofluoron to bind to the target molecules, depending on the binding mechanism, sufficient complementarity and surface area of contact such that van der Waals, hydrogen bonding, and ionic interactions may be needed for the requisite binding energy. Combination of two or more ligand elements at the binding site give cofluorons a tighter binding than would be achieved through a single ligand element. In addition, cofluorons contain a linker element (and an optional connector), which may provide additional opportunities to maximize the surface area of interaction between the cofluoron and targets.

Combinatorial chemistry approaches seek to maximize ligand elements, and such molecules are often synthesized using split and recombine or bead-based approaches. There are two general approaches used to generate a diversity library: (i) a single platform with multiple functional groups, each of which is reacted with a family of diversity reagents to create a library of surfaces and (ii) the diversity is generated using bifunctional reagents to create short linear or circular chains, such as peptides and peptide analogues.

Ligand elements may be moieties derived from molecules previously known to bind to the targets, fragments identified through NMR or crystallographic screeing efforts, molecules that have been discovered to bind to targets after performing high-throughput screening of previously synthesized commercial or non-commercial combinatorial compound libraries or molecules that are discovered to bind to targets by screening of newly synthesized combinatorial libraries.

The target molecules serve as a template to promote the binding of cofluorons to generate fluorescent signals, when cofluoron approaches the binding site or proximate. Knowing the target molecules and the binding mechanism is key to the ligand element design.

The target of interest may be chemicals (e.g., agricultural chemical, warfare chemical. etc.), proteins, peptides, nucleic acids (e.g., DNA, RNA, siRNA), nucleic acid analogs, nucleotides, oligonucleotides, nucleic acid analogues (e.g., PNA, pcPNA and LNA), enzymes, carbohydrates, lipids, aptamers, hormones, hormone antagonists, growth factors or recombinant growth factors and fragments and variants thereof, cell attachment mediators (such as RGD), cytokines, vitamins, cytotoxins, antioxidants, microbes, antibiotics or antimicrobial compounds, anti-inflammation agents, antifungals, antivirals, toxins, cells (e.g., neurons, liver cells, and immune system cells, including stem cells), organisms (e.g., fungus, viral pathogens, bacterium, viruses including bacteriophage), macromolecular associations, or combinations thereof. The targets may also be a combination of any of the above-mentioned molecules.

Ligand elements of the cofluorons can be designed to bind to at least one target biological molecule selected from the group consisting of protein, nucleic acid, cell, carbohydrate, lipid, virus, bacterial, toxin, macromolecular association and viral pathogen.

For example, the target biological molecule can be a protein tryptase. Then at least one of the ligand elements is 3-(piperidin-4-yl)phenyl]methanamine; 4-fluoro-3-(piperidin-4-yl)phenyl]methanamine; 3-(piperidin-4-yl)benzene-1-carboximidamide; 2H-spiro[1-benzofuran-3,4′-piperidine]-5-carboximidamide; or 2H-spiro[1-benzofuran-3,4′-piperidine]-5-ylmethanamine.

In certain embodiment, ligand elements of cofluoron bind to macromolecular targets such as proteins, nucleic acids, carbohydrates, and lipid. Exemplary macromolecular targets of interest also include intracellular proteins, surface proteins, viral proteins, viral structural macromolecules, bacterial proteins, or bacterial macromolecules.

In some embodiments, the target of interest are selected from the group consisting of: (1) G-protein coupled receptors; (2) nuclear receptors; (3) voltage gated ion channels; (4) ligand gated ion channels; (5) receptor tyrosine kinases; (6) growth factors; (7) proteases; (8) sequence specific proteases; (9) phosphatases; (10) protein kinases; (11) bioactive lipids; (12) cytokines; (13) chemokines; (14) ubiquitin ligases; (15) viral regulators; (16) cell division proteins; (17) scaffold proteins; (18) DNA repair proteins; (19) bacterial ribosomes; (20) histone deacetylases; (21) apoptosis regulators; (22) chaperone proteins; (23) serine/threonine protein kinases; (24) cyclin dependent kinases; (25) growth factor receptors; (26) proteasome; (27) signaling protein complexes; (28) protein/nucleic acid transporters; (29) viral capsids; and (30) bacterial surface proteins.

Many target molecules often associate to an event or activity that is of interest. Such event or activity includes, but are not limited to, association of macromolecular targets, protein interactions, protein localization, protein tracking, protein trafficking, cellular process, metabolism of cells, intracellular and extracellular compartmentalization, cell signaling, disease state, disease progression, disease prognosis, disease remission, and therapeutic molecule binding. Particularly target or event of interest include: (a) intracellular proteins, (b) protein translocations, (c) surface proteins, (d) cancer cells in the blood stream or margin tissue, (e) viral surface proteins, (f) bacterial surface proteins or macromolecules, (g) toxins and (h) organelle stains in living or fixed tissue or (i) and association of macromolecular targets.

### Cofluorons Targeted Towards Human Mast Cell β-Tryptase-II

The human mast cell β-tryptase-II is a tetrameric serine protease that is concentrated in mast cell secretory granules. The enzyme is involved in IgE-induced mast cell degranulation in an allergic response and is potentially a target for the treatment of allergic asthma, rhinitis, conjunctivitis and dermatitis. Tryptase has also been implicated in the progression of renal, pulmonary, hepatic, testicular fibrosis, and inflammatory conditions such as ulcerative colitis, inflammatory bowel disease, rheumatoid arthritis, and various other mast cell-related diseases. Hence, detections of this target have significant diagnostic values.

Cofluorons based on Linker elements containing boronic acids that form covalent interactions with diols.

An example of cofluoron monomers containing a diol and boronic acid linker elements and the dimer formed from them is shown below.

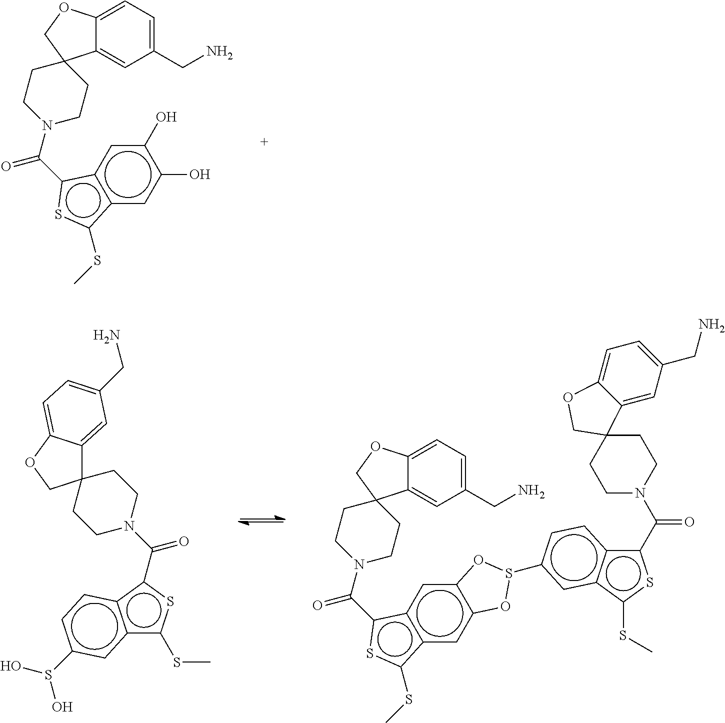

Boronic acids may form tetrahedral boronate ester complexes as shown below. Only a single stereoisomer is shown although both enantiomers may be formed.

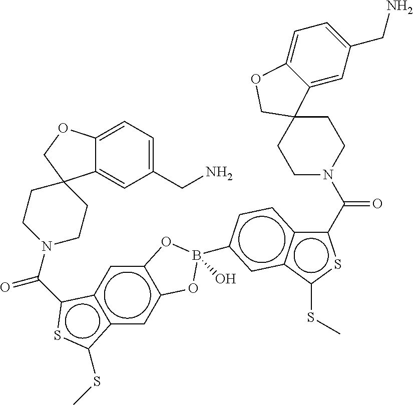

Importantly, alternative homo- and hetero-dimeric linkers such as those described in this disclosure may be employed to achieve the association to produce similar bivalent dimers. For example, amidoketo linker moieties, or heterodimeric boronic acid-diol linker moieties may also be employed to similarly present the key ligand elements.

Additional potential cofluorons and linker elements for potential cofluorons that can be targeted to tryptase may be found in PCT/US 2009/002223 and PCT/US 2010/002708, both of which are hereby incorporated by reference in their entirety.

Importantly, alternative homo- and hetero-dimeric linkers such as those described in this disclosure may be employed to achieve the association to produce similar bivalent dimers. For example, heterodimeric boronic acid-diol linker moieties may also be employed to similarly present the key ligand elements.

In some embodiments, exemplary cofluoron monomers that target on different macromolecules are listed in the following table. The general synthetic procedures for preparation of the cofluoron monomers in the table can be found in Examples 1-9.

| | | |
|---|---|---|
| Sr. | | |
| No. | Compound code | Structure |

Tryptase targets
Method-A

| | | |
|---|---|---|
| 1. | Target-31 | 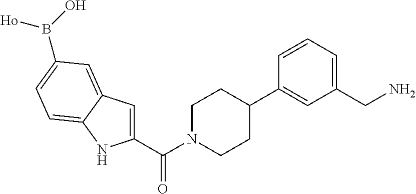 |
| 2. | Target-62 | 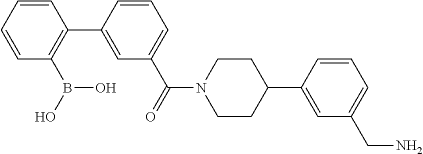 [SMILES: NCc1cccc(C2CCN(C(=O)c3cccc(-c4ccccc4B(O)O)c3)CC2)c1] |
| 3. | Target-35 | 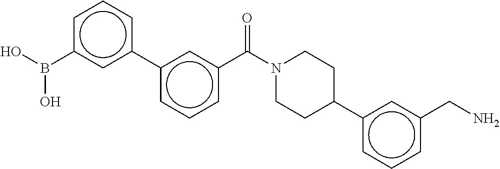 |
| 4. | Target-11F | 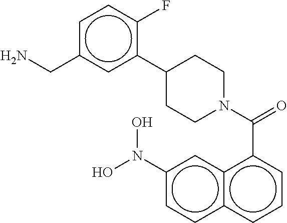 |
| 5. | Target-35F | 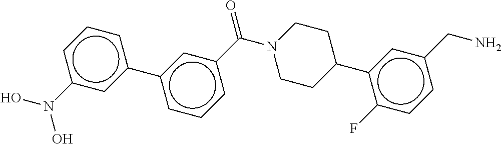 |
| 6. | Target-33 | 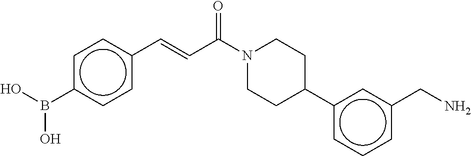 |
| 7. | Target-34 | 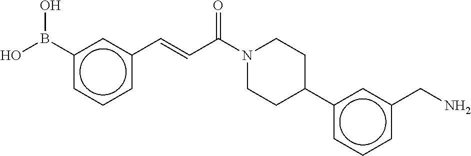 |
| 8. | Target-37 | 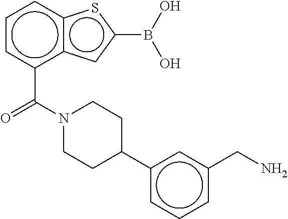 |

Tryptase targets
Method-B

| | | |
|---|---|---|
| 9. | Target-32 | 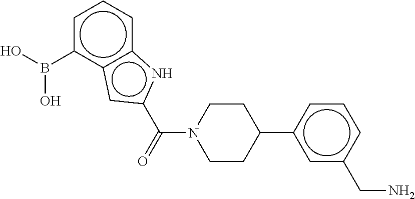 |
| 10. | Target-59 | 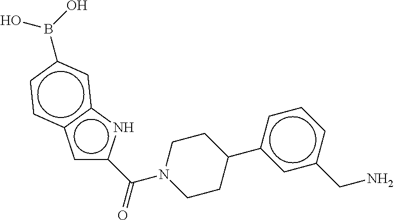 |
| 11. | Target-56 | 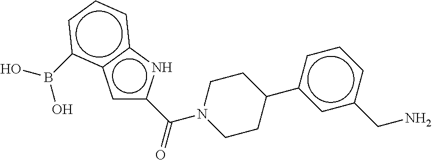 |

Tryptase targets
Method-C

| | | |
|---|---|---|
| 12. | Target-27-F | 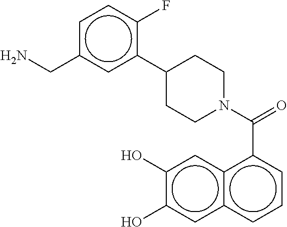 |
| 13. | Target-68 | 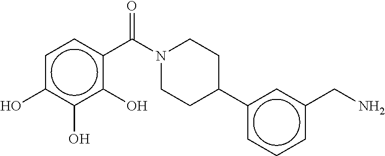 |
| 14. | Target-69 | 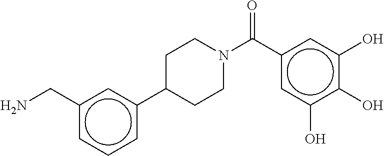 |
| 15. | Target-77 | 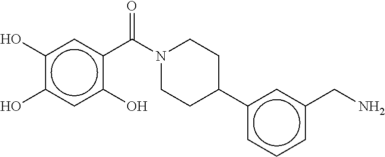 |
| 16. | Target-43 | 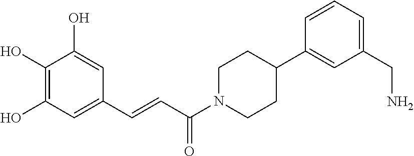 [SMILES: NCc1cccc(C2CCN(C(=O)/C=C/c3cc(O)c(O)c(O)c3)CC2)c1] |
| 17. | Target-97 | 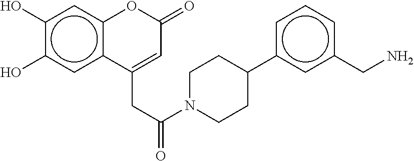 |
| 18. | Target-100 | 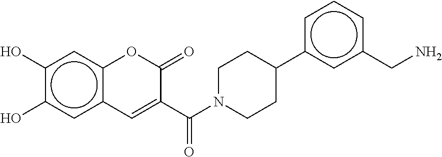 |
| 19. | Target-102 | 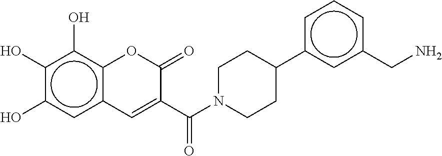 |

Tryptase targets
Method-D

| | | |
|---|---|---|
| 20. | Target-101 | 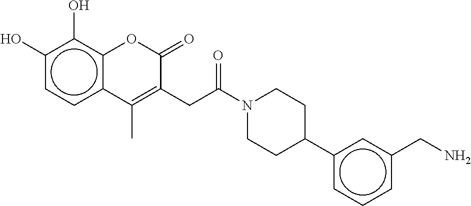 |

Tryptase targets
Method-E
Tryptase targets
Method-F

| | | |
|---|---|---|
| 21. | Target-75a | 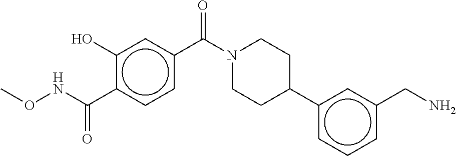 |
| 22. | Target-86 | 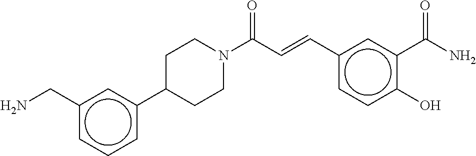 |
| 23. | Target-92 | 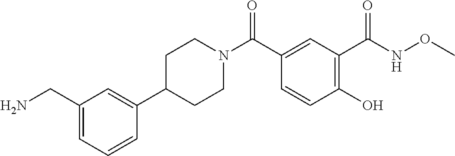 [SMILES: CONC(=O)c1cc(C(=O)N2CCC(c3cccc(CN)c3)CC2)ccc1O] |

Tryptase targets
Method-G
Tryptase targets
Method-H

| | | |
|---|---|---|
| 24. | Target-76 | 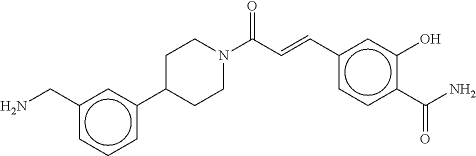 |
| 25. | Target-76a | 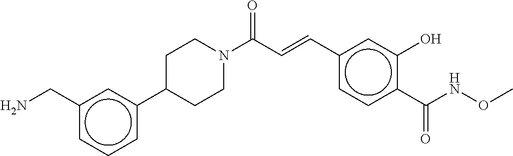 |

Tryptase targets
Method-I

| | | |
|---|---|---|
| 26. | Target-35-Spiro | 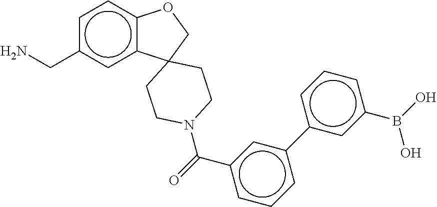 |
| 27. | Target-35-Spiro amidine | 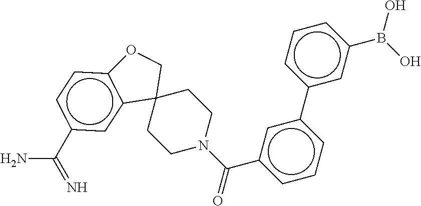 |
| 28. | T-33 Spiro amidine | 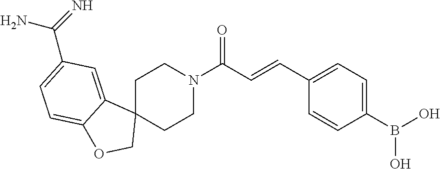 [SMILES: N=C(N)c1ccc2c(c1)C1(CCN(C(=O)/C=C/c3ccc(B(O)O)cc3)CC1)CO2] |

Tryptase targets
Method-K

| | | |
|---|---|---|
| 29. | Target-36 | 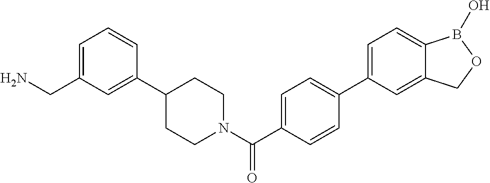 [SMILES: NCc1cccc(C2CCN(C(=O)c3ccc(-c4ccc5c(c4)COB5O)cc3)CC2)c1] |
| 30. | Target-36-meta | 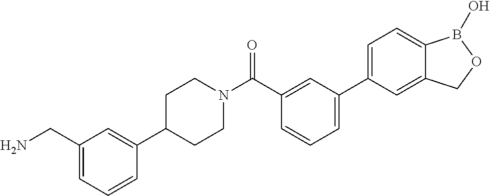 [SMILES: NCc1cccc(C2CCN(C(=O)c3cccc(-c4ccc5c(c4)COB5O)c3)CC2)c1] |

Uncategorized Target

| | | |
|---|---|---|
| 31. | Target-14 | 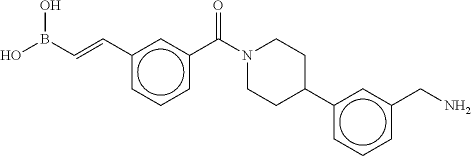 |
| 32. | Target-24 cis | 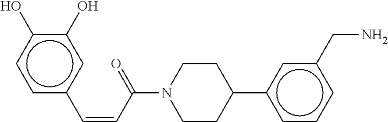 |

In some embodiments, exemplary cofluoron monomers are listed in the following table. The general synthetic procedures for preparation of the cofluoron monomers in the table can be found in Examples 14-17.

| Compound Code | Structure |
|---|---|
| 116-Spiro | 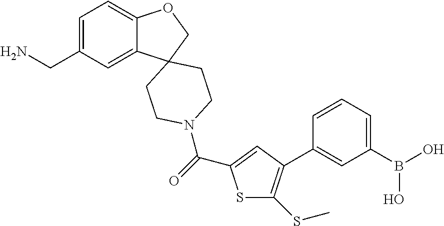 [SMILES: CSc1sc(C(=O)N2CCC3(CC2)COc2ccc(CN)cc23)cc1-c1cccc(B(O)O)c1] |
| 146 | 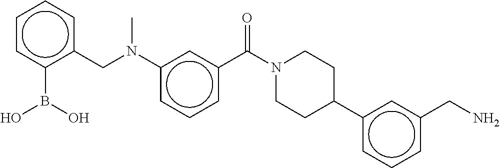 |
| 147 | 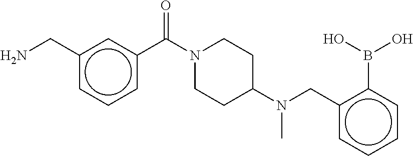 |
| 143 | 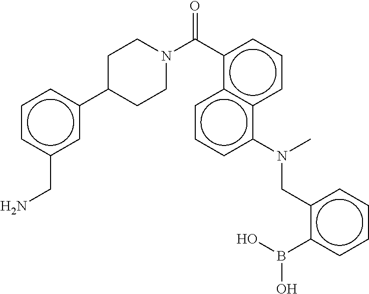 |
| 154 | 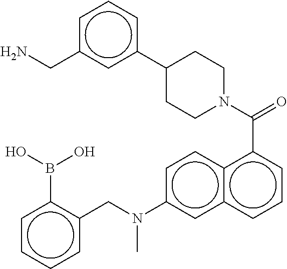 |
| 155-Spiro | 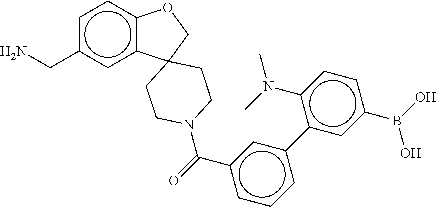 |
| 131-Spiro | 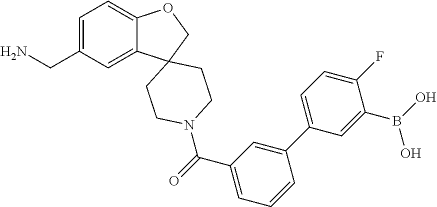 [SMILES: NCc1ccc2c(c1)C1(CCN(C(=O)c3cccc(-c4ccc(F)c(B(O)O)c4)c3)CC1)CO2] |
| 144 | 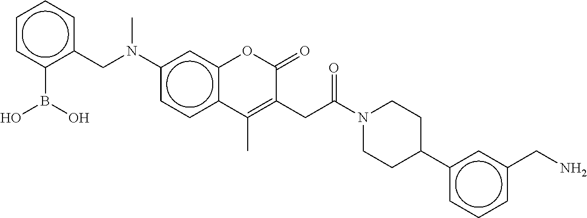 |
| 92-O-t-bu | 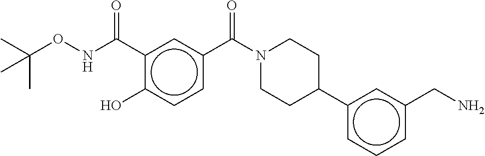 |
| 92-O-t-Bu Spiro | 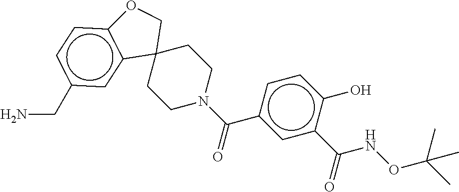 |
| 92-Spiro-O-Ph | 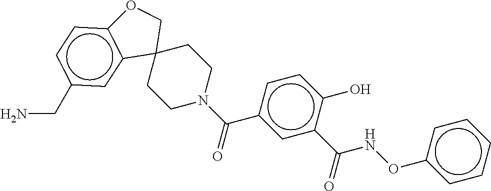 |
| 92-O-Ph | 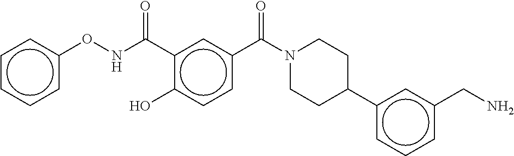 |
| 92-Spiro | 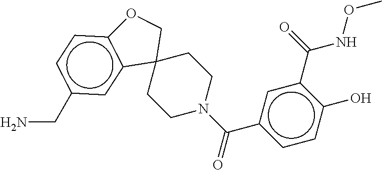 |
| 114-Spiro | 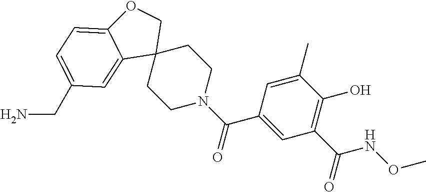 [SMILES: CONC(=O)c1cc(C(=O)N2CCC3(CC2)COc2ccc(CN)cc23)cc(C)c1O] |
| 75a-O-t-Bu | 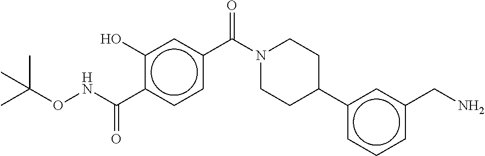 |
| 75a-O-Ph | 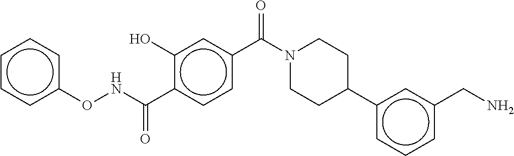 |
| 75a-O-t-Bu Spiro | 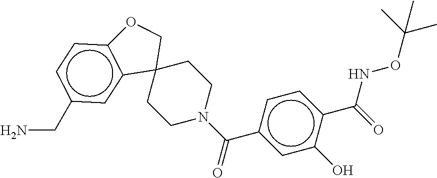 |
| 75a-O-Ph-spiro | 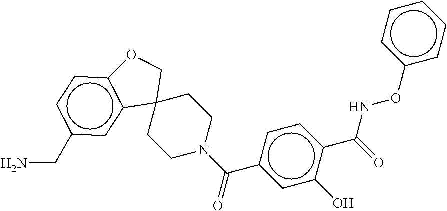 |
| 99 | 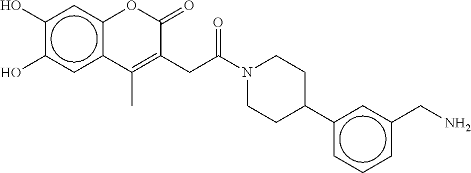 |
| 117-Spiro | 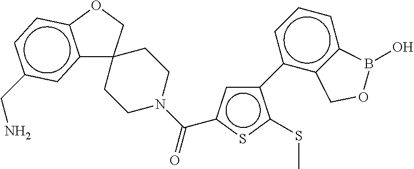 |
| 117 | 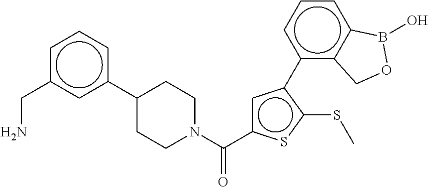 |

The present invention also relates to a multimer useful as a fluorescence reporter. The multimer comprises a plurality of covalently or non-covalently linked monomers. Each monomer comprises one or more ligand elements which are useful for binding to a target molecule with a dissociation constant less than 300 μM and a linker element being connected directly or indirectly through a connector to the one or more ligand elements. The linker element is capable of forming a bond with one or more linker elements of either the same or a different monomer of the plurality of monomers. Association of the linker elements, with their ligand elements bound to the target molecule to form a multimer, will generate a unique fluorescent signature different from that produced by those monomers either alone or in association with each other in the absence of target, when subjected to electromagnetic excitation.

The various embodiments for the monomer components in the multimer have been described herein above, similar as the embodiments and examples for the monomers provided as a collection of monomers to form cofluoron multimer reporters.

The cofluoron multimers include any multimer composition that can be provided separately, or the multimers formed in situ by self-assembling of one or more monomers either in vitro or in vivo, when using the collection of monomers.

### Target Screening

Another aspect of the present invention relates to a method of screening for combinations of monomers useful as fluorescent reporters. The method comprises providing a collection of monomers. Each of the monomers comprises one or more ligand elements, which are useful for binding to a target molecule with a dissociation constant less than 300 μM, and a linker element being connected directly or indirectly through a connector to the one or more ligand elements. The linker element is capable of forming a bond with one or more linker elements of either the same or a different monomer of the collection of monomers. Association of the linker elements, with their ligand elements bound to the target molecule to form a multimer, will generate a unique fluorescent signature different from that produced by those monomers either alone or in association with each other in the absence of target, when subjected to electromagnetic excitation. The combinations of the collection of monomers are contacted with the target molecule under conditions effective to allow the ligand elements to bind to the target molecules. The monomers are subjected to reaction conditions effective for the linker elements of either the same or different monomers to undergo bond forming to form multimers. This subjecting step can occur either before, after, or during the contacting step. As a result of the contacting and the subjecting, the combinations of monomers that form multimers and generate a fluorescent signature, which is different from that produced by those monomers either alone or in association with each other in the absence of target, are then identified.

The steps of identifying the combinations of candidate monomers, which can form cofluoron multimers useful as fluorescent reporters, can be carried out by determining which one or more of the candidate monomer pairs can produce a unique or characteristic fluorescent signals after the monomers undergoing bond forming to form multimers which binds to the target molecule.

The fluorescent signatures for each monomer of the collection of monomers alone, i.e., before contacting the monomers with the target molecule, and/or subjecting the monomers to associate with each others, if presented, can be detected and determined initially. The candidate monomer collections can be excited at a given wavelength or a set of wavelengths of electromagnetic radiation suitable to produce a fluorescent emission. If a fluorescent signature is present for the collection of the candidate monomer, fluorescence emissions of the collection of monomers can be observed at a UV, visible or IR spectrum. The fluorescent signature of individual candidate monomer can also be detected and compared with those of the multimer either in the presence or in the absence of the target.

After the candidate combination of monomers undergoes bond formation to form multimers which bind to the target molecule, the fluorescent signatures of the system, if present, can be detected and determined. If there are changes in the fluorescent signatures for the system, the one or more combination of monomers produces such, changes are then identified to be used as cofluorons for fluorescent reporting. The fluorescent signatures change can be any detectable change in the excitation and emission spectra, including an increase or a decrease or a complete quenching; a change in fluorescence excitation wavelength or fluorescence emission wavelength, including blue shift or red shift; a change in polarization of fluorescence emission; or combinations thereof.

Yet another aspect of the present invention relates to a method of screening for ligands. The method comprises providing a collection of monomers. Each of the monomers comprises one or more ligand elements having a potential to bind to a target molecule and a linker element being connected directly or indirectly through a connector to the one or more ligand elements. The linker element is capable of forming a bond with one or more linker elements of either the same or a different monomer of the collection of monomers. Association of the linker elements, with their ligand elements bound to the target molecule to form a multimer, will generate a unique fluorescent signature different from that produced by those monomers either alone or in association with each other in the absence of target, when subjected to electromagnetic excitation. The combinations of the collection of monomers are contacted with the target molecule under conditions effective to allow the ligand elements to bind to the target molecules. The monomers are subjected to reaction conditions effective for the linker elements of either the same or different monomers to undergo bond forming to form multimers. This subjecting step can occur either before, after, or during the contacting step. As a result of the contacting and the subjecting, the combinations of monomers that form multimers by binding of their ligands to the target molecule and binding of their linker elements, and that generate a fluorescent signature, which is different from that produced by those monomers either alone or in association with each other in the absence of target, are then identified.

The steps of identifying the combinations of candidate monomers, which contain the desired ligand elements for high-affinity binding to the target and binding to linker elements to form cofluoron multimers useful as fluorescent reporters, can be carried out by determining which one or more of the candidate monomer pairs can produce a unique or characteristic fluorescent signal after the monomers undergoing bond forming to form multimers which binds to the target molecule.

The fluorescent signatures for each monomer of the collection of monomers alone, i.e., before contacting the monomers with the target molecule, and/or subjecting the monomers to associate with each others, if presented, can be detected, and determined initially. The candidate monomer collections can be excited at a given wavelength or a set of wavelengths of electromagnetic radiation suitable to produce a fluorescent emission. If a fluorescent signature is present for the collection of the candidate monomer, fluorescence emissions of the collection of monomers can be observed at a UV, visible or IR spectrum. The fluorescent signature of individual candidate monomer can also be detected and compared with those of the multimers either in the presence or in the absence of the target.

After the candidate combination of monomers undergoes bond formation to form multimers which binds to the target molecule, the fluorescent signatures of the system, if present, can be detected and determined. If there are changes in the fluorescent signatures for the system, the one or more combination of monomers produces such changes are then identified to be used as cofluorons for fluorescent reporting. The fluorescent signatures change can be any detectable change in the excitation and emission spectra, including an increase or a decrease or a complete quenching; a change in fluorescence excitation wavelength or fluorescence emission wavelength, including blue shift or red shift; a change in polarization of fluorescence emission; or combinations thereof.

Screening for ligand elements having potential to bind to the target molecule can also involve determining which cofluoron dimers or multimers are more tightly bound to the target molecule. This determination can be the same as the above determination using the fluorescent signature detection. Further, the determination can also be assisted by attaching a bead barcodes to the monomers and identifying the bead barcodes. When each monomer includes an encoding element coupled to the ligand element and the linker element for each monomer, the individual components for the candidate combinations of monomers can be identified by detecting the encoding elements in the resulting multimers.

When the encoding element is a labeled bead, the steps of providing a plurality of monomers, contacting, subjecting, and identifying the monomers can be repeated to determine which of the multimers have a suitable binding affinity to the target molecule.

Additionally, mass spectrometric methods may be employed to determine the molecular weight of the high affinity dimers and the identities of the monomeric constituents. For example, the use of size-exclusion chromatographic methods may separate unbound monomeric cofluorons from dimeric cofluorons bound to the macromolecular target, followed by dissociation and detection of the cofluorons by mass spectrometry.

After the individual components of the combination of the monomers have been identified, the fluorescent reporting cofluorons, including one or more monomers resulting from the above method can be prepared by coupling the identified individual monomer components. The combination of the monomers that are composed of the identified monomers can then be used as fluorescent reporters.

Screening for the linker element of cofluoron can be subjected to dynamic combinatorial library screening for the high-affinity binding linker element pairs, and screening for the ligand elements of cofluoron can be subjected to screening for the high-affinity binding ligands to the target. Hence the screening of cofluorons for targets can include identifying and detecting the fluorescent signature changes. The detailed description for evolutionary screening methods in dynamic combinatorial library and diversity library, various scenarios for screening linker elements and ligand elements of coferons, can be found in PCT/US 2009/002223 and PCT/US 2010/002708, both of which are hereby incorporated by reference in their entirety.

### General Method for the Preparation of Cofluoron Monomers

Cofluoron monomers are comprised of one or more ligand elements, a connector and a linker element. Various linker elements provide different equilibrium properties between the monomer and dimer or multimer form, have different geometries that allow for connectors or ligand elements to be oriented in appropriate fashion, and span different distances. One approach to making cofluoron monomers for a specific target involves selecting appropriate ligand elements identified through literature or crystal structures, selecting potential linker elements pairs that may have the fluorescent properties or changes upon association based on their structures or the literature, determining the geometry and spacing required to span the distance between the ligand elements, and selecting the appropriate linker elements and connectors that provide the optimum spacing and geometry.

In silico methods can be employed to aid in the selection of permutations of ligand elements, connectors and linker elements. Virtual screening of the permutations using docking and scoring of cofluorons to known structures of the macromolecular target (e.g. from NMR or x-ray methods), either directly or in combination with ligand-based ligand elements models, can aid in selecting the most promising cofluoron designs. Alternatively, in silico methods may start from a known co-crystal structure of a ligand bound to the macromolecular target, and virtually replace regions of the ligand scaffold with novel linker elements to produce cofluoron designs. A series of candidate cofluoron monomers can then be synthesized by combining the selected ligand elements, connectors, and linker elements in a combinatorial fashion. The cofluoron monomers can then be screened against the target to determine the best candidates.

A third approach is to prepare a library of cofluoron monomers by combining various known ligand elements as well as molecules containing known and unknown ligand elements with a variety of connectors and linker elements in a combinatorial fashion. The cofluoron monomers can then be screened in a combinatorial fashion to find the best pairs of monomers as fluorescent reporters for a specific target.

### Drug Discovery and High-Throughput Screening

Cofluorons, like coferons, provide a unique opportunity for drug screening due to their combinatorial nature. The ligand elements of cofluoron may be screened for targeting specific protein surfaces or protein interaction domains and interfere or modulate activity of the target proteins. Such ligand elements can therefore be considered as a pharmacophore, and the cofluoron in this sense, can be used as fluorescent coferons for drug discovery and screening.

The unique benefit of cofluoron lies on the easy detection due to the fluorescent reporting nature of cofluorons. The ability of linker binding pairs to generate an increase in or wavelength shift in fluorescence signal provides an opportunity to rapidly detect coferon pair binding to the target protein or molecule. Therefore, cofluoron can be used to develop rapid high-throughput screening techniques to determine the binding affinities of coferon candidate pairs.

Consider two compatible linker families A and B, such that all members of the A linker family can form reversible covalent linkages to all members of the B linker family. For instance, the A and B linker families can be a family of catechols or derivatives and a family of aromatic boronic acids. For each A linker, various length and geometry connectors “C” can be attached to various pharmacophores (or ligand elements for cofluoron) “PA” and “PB” that bind to adjacent sites on the target respectively. A given coferon candidate may thus be described as Ai-Ci-PAi, or Bi-Ci-PBi, where “i” designates a number from 1-n, where n is the number of the given component available. Even for small numbers of each component, one can rapidly generate a large number of combinations for screening. For example, if An=9, Bn=8, Cn=5, PAn=4 and PBn=5, there will be 9×5×4=180 combinations of Ai-Ci-PAi, and 8×5×5=200 combinations of Bi-Ci-PBi, for a total of 36,000 combinations of cofluoron pairs subjected to screening for the ability of the right coferons to bind to and interfere or modulate activity of the target protein.

In a first embodiment, individual or mixtures of 5 of more coferon candidate pairs, having the fluorescent properties as described above, i.e., they are also cofluorons (incorporating linker elements that give rise to unique fluorescent properties when combined with an appropriate partner linker element), are mixed together in the presence of the biomolecular target. Whether the coferon candidate pairs bind to the target may be distinguished by one of several possible cofluoron effects. For example the total fluorescent signal generated by the mixture may be greater in the presence of biomolecular target than in the absence of target. Alternatively, a shift in the excitation and/or emission wavelength of the fluorescent signal to produced by the mixture is detected in the presence of target compared to in the absence of target. In addition, binding of the cofluorons to the macromolecular target may also be detected as a change in fluorescence polarization. Thus, even if some cofluoron dimerization occurs in the absence of biomolecular target, the fluorescent polarization for cofluoron dimer bound to the target will reflect the slower rotation compared to the free dimer in solution. This would allow the binding interactions of cofluorons to the target to be dectected. This approach therefore allows for distinguishing a coferon pair having higher binding affinity to targets or dimerizing in the presence of target, thus having higher potential for therapeutic candidates, from those having lower binding affinity or not significantly dimerizing in the presence of target. For instance, in a mixture of cofluoron drug candidates, a cofluoron pair that forms cofluoron multimers in the presence of the target can be distinguished from the other 4 cofluoron pairs that did not associate or dimerize in the presence of target.

High-affinity cofluoron multimers are “tailored” assemblies of two or more cofluorons incorporating linker elements that give rise to unique fluorescent properties when they combine. The optimal combinations of ligand, connector, and linker element to get the best fit to the biomolecular target can be obtained by using a selection of known ligands, and established cofluoron linker chemistries, and varying the nature of the connectors that join the linker and ligand, as well as the points of attachment (e.g. using combinatorial chemistries) and then rapidly screening the permutations to identify the functional cofluorons pairings directed to the macromolecular target.

Once high-affinity cofluorons pairs are identified for the macromolecular target, the linker moieties may be modified or replaced with other linker chemistries (such as isosteric alternatives) to produce coferons with further optimized drug properties. In addition, there is an opportunity to “tune” the fluorescent properties of the cofluorons with appropriate substituents to enhance their usefulness in detecting the presence of the biomolecular target or its association with other macromolecules.

Hence, the screening process can also include a pre-screening for best fluorescent-score wells containing cofluoron pairs with higher-potential for candidate coferon drugs, and a further screening can be cycled on those better fluorescent-score cofluoron pairs. For example, the above combinatorial library containing 36,000 combinations of cofluoron pairs, among which coferons for potential therapeutic candidates are contained, can be screened in 1,440 wells (equals 15 standard 96 well microtiter plates, or 4 of the 384 well plates), and those wells with significant fluorescent signal above background can be chosen, which can significantly reduce the number of combinations to be screened, and a small number of different coferon pairs from these chosen wells, for instance, 25 different pairs, can be re-tested individually.

In a second embodiment, one of the cofluoron pairs is immobilized on a solid surface, such as a bead. The bead may also be encoded to distinguish it from other beads bearing different cofluoron molecules. The target is added in the presence of one or more of the partner cofluorons in solution. Those beads containing cofluoron molecules that can form productive cofluoron binding pairs in the presence of the target protein or macromolecule will exhibit a higher fluorescent signal than beads containing cofluoron monomers that do not form productive binding pairs under the same conditions. The individual beads may then be identified through their codes, or the cofluoron moiety could be detached for identification, or alternatively, retested individually, with individual cofluorons. Since the majority of coferon and cofluoron linker chemistry is reversible, the above approach may strengthen the signal on individual beads over time, as dynamic combinatorial chemistry will select for the cofluoron pairs with highest binding affinity. For example, 10 different beads bearing cofluorons and 10 different cofluorons in solution (i.e., 100 different cofluoron pairs) may be tested in a single well, allowing for generation of a fluorescent signal in the presence of the target that is sufficiently above any background signal in the absence of target. In this case, the above 36,000 combinations of cofluoron pairs may be screened in 360 wells (less than 4 standard 16-well microtiter plates, or a single 384-well plate). Those wells containing beads with significant fluorescent signal above background can be chosen, and for each individual well that is chosen, the 100 different coferon pairs having higher scores can be re-screened.

Using these approaches with cofluorons, a large combinatorial library can be screened directly and rapidly, i.e., the screening process can be as simply as adding cofluorons in the samples containing targets and direct detection of fluorescent signals. Unlike the standard screening assay methods employing additional steps of labeling and washing after the ligand/receptor binding step, which add significant amount of time and cost to the methods, utilization of cofluorons for drug screening can be simple, time-saving, and cost-effective, and ultimately achieve the rapid high-throughput screening.

Similarly as screening assays for coferons, the above embodiments using cofluorons for coferon candidate drug screening are amenable to a multiple-well format and automation. Further, many commercially available high-throughput screening assay devices employing additional detection reporters, such as luminescent labeling and detection can be modified based on the inherent fluorescent properties of cofluorons upon binding to the targets. The detailed description for screening process and device designs for coferons, and different scenarios for drug screening of coferons using commercially available assays and devices can be found in PCT/US 2009/002223 and PCT/US 2010/002708, both of which are hereby incorporated by reference in their entirety.

### Fluorescent Reporting

### Target Identification and Labeling

The present invention also relates to a method of detecting the presence or absence of a target molecule in a sample. The method includes providing a sample potentially containing one or more target molecules. Also provided is a set of one to six monomers. Each monomer comprises one or more ligand elements, which are useful for binding to a target molecule with a dissociation constant less than 300 μM, and a linker element being connected directly or indirectly through a connector to the one or more ligand elements. The linker element is capable of forming a bond with one or more linker elements of either the same or a different monomer of the set of monomers. Association of the linker elements, with their ligand elements bound to the target molecule to form a multimer, will generate a unique fluorescent signature different from that produced by those monomers either alone or in association with each other in the absence of target, when subjected to electromagnetic excitation. The sample is contacted with the set of monomers under conditions effective to allow the ligand elements to bind to the target molecules, if the target molecules are present in the sample. The monomers are subjected to reaction conditions effective for the linker elements of either the same or different monomers to undergo bond forming to form multimers, if the target molecules are present in the sample. This subjecting step can occur either before, after, or during the contacting step. The presence or absence of target molecule in the sample is then detected based on the fluorescent signature of the sample subjected to the contacting and the subjecting.

The above method can further comprise a step to identify the presence or absence of target molecule in the sample as a result of an event or activity associated with the presence or absence of the target molecule labeled with the fluorescent signature of the multimer.

The sample to be tested potentially contains the target molecule of interest. While many samples will comprise targets in solution, suspension, or emulsion, solid samples that can be dissolved in a suitable solvent may also be tested. Samples of interest include biological samples which can encompass any samples of a biological origin, including, but not limited to, blood, cerebral spinal fluid, urine, sputum, plant or animal extract, lysates prepared from crops, tissue samples, etc. Samples of interest may also include environmental samples such as ground water, sea water, or mining waste, etc. The sample to be tested can contain cells, tissues, organelles, bacteria, fungus, or viruses.

The ligand elements can be designed to bind the target molecules or bind to proximate locations of the target molecules. These target molecules in turn serve as a template to promote the binding of cofluorons to generate fluorescent signals.

The target of interest may be chemicals (e.g., agricultural chemical, warfare chemical. etc.), proteins, peptides, nucleic acids (e.g., DNA, RNA, siRNA), nucleic acid analogs, nucleotides, oligonucleotides, nucleic acid analogues (e.g., PNA, pcPNA and LNA), enzymes, carbohydrates, lipids, aptamers, hormones, hormone antagonists, growth factors or recombinant growth factors and fragments and variants thereof, cell attachment mediators (such as RGD), cytokines, vitamins, cytotoxins, antioxidants, microbes, antibiotics or antimicrobial compounds, anti-inflammation agents, antifungals, antivirals, toxins, cells (e.g., neurons, liver cells, and immune system cells, including stem cells), organisms (e.g., fungus, viral pathogens, bacterium, viruses including bacteriophage), macromolecular associations, or combinations thereof. The targets may also be a combination of any of the above-mentioned molecules.

Ligand elements of the cofluorons can be designed to bind to at least one target biological molecule selected from the group consisting of protein, nucleic acid, cell, carbohydrate, lipid, virus, bacterial, toxin, macromolecular association, and viral pathogen.

For example, the target biological molecule can be a protein tryptase. In that case, suitable ligand elements include 3-(piperidin-4-yl)phenyl]methanamine; 4-fluoro-3-(piperidin-4-yl)phenyl]methanamine; 3-(piperidin-4-yl)benzene-1-carboximidamide; 2H-spiro[1-benzofuran-3,4′-piperidine]-5-Carboximidamide; or 2H-spiro[1-benzofuran-3,4′-piperidine]-5-ylmethanamine.

In certain embodiments, the ligand elements of cofluoron monomers bind to macromolecular targets such as proteins, nucleic acids, carbohydrates, and lipid. Exemplary macromolecular targets of interest also include intracellular proteins, surface proteins, viral proteins, viral structural macromolecules, bacterial proteins, or bacterial macromolecules.

In some embodiments, the target of interest are selected from the group consisting of (1) G-protein coupled receptors; (2) nuclear receptors; (3) voltage gated ion channels; (4) ligand gated ion channels; (5) receptor tyrosine kinases; (6) growth factors; (7) proteases; (8) sequence specific proteases; (9) phosphatases; (10) protein kinases; (11) bioactive lipids; (12) cytokines; (13) chemokines; (14) ubiquitin ligases; (15) viral regulators; (16) cell division proteins; (17) scaffold proteins; (18) DNA repair proteins; (19) bacterial ribosomes; (20) histone deacetylases; (21) apoptosis regulators; (22) chaperone proteins; (23) serine/threonine protein kinases; (24) cyclin dependent kinases; (25) growth factor receptors; (26) proteasome; (27) signaling protein complexes; (28) protein/nucleic acid transporters; (29) viral capsids; and (30) bacterial surface proteins.

Many target molecules often associate to an event or activity that is of interest. Such event or activity includes, but are not limited to, association of macromolecular targets, protein interactions, protein localization, protein tracking, protein trafficking, cellular process, metabolism of cells, intracellular and extracellular compartmentalization, cell signaling, disease state, disease progression, disease prognosis, disease remission, and therapeutic molecule binding. Particularly targets or events of interest include: (a) intracellular proteins, (b) protein translocations, (c) surface proteins, (d) cancer cells in the blood stream or margin tissue, (e) viral surface proteins, (f) bacterial surface proteins or macromolecules, (g) toxins and (h) organelle stains in living or fixed tissue or (i) association of macromolecular targets.

The identification of the presence or absence of the target or an event or activity associated with the target can be carried out by determining the fluorescent signals changes after the contacting and subject steps. The fluorescent signatures for each monomer of the set of monomers alone, i.e., before contacting the cofluorons with the sample, and/or subjecting the cofluorons to associate with each others, if presented, can be detected and determined initially. The cofluoron monomer set can be excited at a given wavelength or a set of wavelengths of electromagnetic radiation suitable to produce a fluorescent emission. If a fluorescent signature is present for the set, fluorescence emissions of the set can be observed in a UV, visible or IR spectrum. After the set of cofluoron monomers undergo bond formation to form cofluoron multimers which bind to the target, the fluorescent signatures of the system, if present, can again be detected and determined. If there are changes in the fluorescent signatures for the system, the target of interest is then identified to be present in the tested sample.

The fluorescent signature change can be any detectable change in the excitation and emission spectra, including an increase or a decrease or a complete quenching; a change in fluorescence excitation wavelength or fluorescence emission wavelength, including blue shift or red shift; a change in polarization of fluorescence emission; or combinations thereof. The fluorescent measurement at UV, visible, and NIR regions are carried out with instruments known to those skilled in the art.

Particularly useful is the change in polarization of fluorescent emission when detecting the presence or absence of a macromolecular target or an event or activity associated with a macromolecular target. Cofluorons, when bound to a macromolecular target that has significantly higher molecular weight than cofluoron monomers and/or dimers, (e.g., proteins, or bacterial and viral pathogen), rotate more slowly and thus change fluorescence polarization. Therefore, fluorescent polarization measurement can be used to identify and monitor binding cofluoron multimers to the macromolecular target.

In some embodiments, the cofluoron multimers, when bound to the target molecule, provide a unique fluorescent signal (i.e., the signal is different from any signal produced by the set of cofluorons in absence of target and is distinguishable from background signals from the sample of interest). As a result, the target molecule of interest is labeled with this unique fluorescent signature.

Cofluorons can be designed to generate the characteristic fluorescent signals only when a specific target is present in the sample (i.e., a target-specific fluorescent signal). This allows for detection or labeling the specific target and a target-specific event or activity. For example, the specific target for cofluorons to bind in a sample is associated with a specific tissue, organelle, or cell-type (e.g., the cofluoron can be a neuronal tracer). Alternatively, the specific target is only present in an infected cell or tissue (e.g., the cofluoron can be a disease marker).

In some further embodiments, the set of cofluorons used for identification of target or labeling can include different pairs of cofluoron where each pair, when bound to a specific target in a sample, can generate a target-specific signature different from other pairs that bind to other targets in a sample. This allows for simultaneous detection or labeling of multiple targets (i.e., a visualization of multiple targets within a single image in the sample). These methods can be used to replace the standard fluorescent labels or tags used in many assaying and screening techniques.

In addition to the detection of a target biomolecule in a range of settings, the cofluorons can also be developed to detect and potentially modulate protein-protein interactions in vitro, or their native environment in cells, biological tissues, or fluids, and even in vivo. This can be achieved through the use of ligand elements for the respective biomolecules or their interface whose association is to be targeted, that are conjoined using appropriate connectors and linker elements. This can produce a cofluoron pair that binds and dimerizes to produce a cofluorons “reporter” only when the biomolecular targets are associated. Alternatively, if one of the ligand elements of the cofluorons pair competes for binding to a site on the biomolecular target that is required for associations with other macromolecules, the cofluorons will “report” the accessibility of the site on the biomolecular target and can also inhibit the associations of the biomolecular target with its macromolecular partner. Such approaches can be used with cofluorons to detect active ligand-receptor interactions, signal transduction, protein-protein interactions, or subcellular localization, expression, or turnover of biomolecular targets.

When fluorescent emission of a cofluoron is in the infrared region, it is readily detected in live animals and in human body if the emitted signal is able to penetrate tissue deep enough for detection of target. This allows for in vivo detection of target in a living system, such as living cells.

After the target molecule in the sample is identified, the characteristic fluorescent signature changes, when cofluorons bind to the target, can be further monitored, in situ. For example, a fluorescence lifetime imaging microscopy (FLIM) can be used to detect certain bio-molecular interactions which manifest themselves by influencing fluorescence lifetimes. One example is to use confocal microscopy to detect and monitor skin cancers.

Additionally, the detection of cofluorons can be found useful in many other different applications, such as detecting microorganisms in environmental samples, detecting substances such as glucose or leukemia in blood samples, or detection of cancer invasion into margin tissue.

### Analyzing Target in the Sample

When a set of cofluoron monomers is used as a kit for detecting the presence or absence of one or more specific target molecules or quantification of the target molecules, the fluorescent signatures for the cofluoron set may be pre-determined and provided as the “reference values” with the kit. The “reference value” can be an absolute value, a relative value; a value that has an upper and/or lower limit; a range of values, an average value, a median value, a mean value, or a value as compared to a particular control or baseline value, for the parameters such as excitation wavelength, emission wavelength, and emission intensity, etc. For a specific molecular target or a set of molecular targets, the fluorescent signature change of the cofluoron set, upon binding to the target, can also be pre-determined and provided. Of course, these parameters may be closely associated with concentrations of cofluoron monomers in the set, concentrations of target in the sample, pH level, ionic strength, metal presence and concentration, and the like. These conditions can be provided in a preferred range for simple and high-quality read-out when using the set of the cofluorons.

In some embodiments, when target molecule is found to be present in the sample, the above method can be used to quantitatively analyze the target molecule or activity or event associated with the target molecule. For example, the fluorescence generated in the sample containing an unknown amount of the target molecule can be measured using the method described above with the cofluorons. This measurement can be compared with the fluorescence measured from a sample containing a known amount of the target molecule. The amount of the target molecule present in the former sample can then be determined based on the comparing. The measurement can be carried out by a technology capable of quantitating signal, such as a spectrofluorometer.

This quantification method can be found useful in many different applications such as analyzing environmental samples for the amount of microorganisms, blood samples for the amount of glucose, or other biosensing assays.

### Cell Sorting

The above method of detection of target molecules with cofluorons can also be used in cell sorting techniques to separate different cell lines. For example, when the target molecule is associated with cell surfaces, the method can further comprise sorting the cells based on the fluorescent signature of the multimer.

In some embodiments, cofluorons are used as labels in flow cytometry for cell sorting. This is a method for sorting a heterogeneous mixture of biological cells into two or more containers, one cell at a time, based upon the specific fluorescent characteristics of each cell. The set of cofluorons used can include different cofluoron pairs where each pair, when bind to a specific target in a cell, can generate a characteristic fluorescent signature different from other pairs that bound to another target in a cell. That is, each pair in the set generates a different target-specific fluorescent signature. If the target of the cofluorons is cell-specific, then different cells are sorted based on the cell-specific fluorescent signature. This approach provides a fast and objective recording of fluorescent signals from individual cells as well as physical separation of cells of particular interest. When combined with a technology capable of quantitating the fluorescent signal, the method can also provide quantitation of the sorted cells.

The following examples use cell-specific cofluorons for cell-sorting. The cell suspension mixed with the set of above-described cell-specific cofluorons is entrained in the center of a narrow, rapidly flowing stream of liquid. The flow is arranged so that there is a large separation between cells relative to their diameter. A vibrating mechanism causes the stream of cells to break into individual droplets. The system is adjusted so that there is a low probability of more than one cell per droplet. Just before the stream breaks into droplets, the flow passes through a fluorescence measuring station where the fluorescent character of interest of each cell is measured. An electrical charging ring is placed just at the point where the stream breaks into droplets. A charge is placed on the ring based on the immediately-prior fluorescence intensity measurement, and the opposite charge is trapped on the droplet as it breaks from the stream. The charged droplets then fall through an electrostatic deflection system that diverts droplets into containers based upon their charge. In some systems, the charge can be applied directly to the stream, and the droplet breaking off retains a charge of the same sign as the stream. The stream is then returned to neutral after the droplet breaks off.

### Imaging and Localization

The method of detecting of target molecule in a sample above can also further comprise the step of imaging the sample using the formed multimer as a result of the contacting and the subjecting steps.

Cofluorons can be used to trace target molecules, such as proteins in cells, organelles or tissues in their natural state. Traditional methods of visualization of proteins in living cells is an expensive and time-consuming procedure using recombinant proteins with fluorescent tags that must be introduced into the cell. Instead, cofluorons can be used as individual monomers that, depending on the molecular weight, can be designed to be cell permeable, enter the cell and combine inside the cells to form cofluoron multimers that bind to intracellular target molecules. Thus cofluorons can be used as non-invasive fluorescent reporting agents for in vitro or in vivo imaging target molecules or events or activities associated with the binding of target molecules, such as intracellular proteins and macromolecules, protein interactions, pathway analysis, protein tracking and trafficking tissues, living cells, cell types, or cellular processes. For example, cofluorons can be used in cancer diagnosis for non-invasively detecting/monitoring skin cancers by using confocal microscopy.

When fluorescent emission of the cofluoron is in the infrared region, the emitted signal may be able to penetrate tissues deep enough to detect signals generated in live animals and in human body in their natural state. Thus, the imaging methodologies can be carried out in a non-invasive manner in vivo.

When the sample to be tested is a biological sample, the method of detecting of target molecules in a sample can also include imaging and localizing the target molecule in the biological sample based on its fluorescent signature resulting from the contacting and the subjecting steps.

In one embodiment, the target molecule is localized to specific cells in the biological sample. For example, the target molecule is localized to cancer cells in the biological sample.

In another embodiment, the target molecule is localized to specific subcellular compartments in the biological sample. Such localization can be associated with a disease. Thus, cofluorons can be used to image and monitor disease state, disease progression, disease prognosis, or disease remission. Also, the target molecule localized identifies specific subcellular compartments or the metabolic state of such compartments.

The cofluorons of the present invention are designed to generate a target-specific fluorescent signal, and the specific target for cofluorons to bind in a sample is associated with a specific tissue, organelle, cell-type, or cellular processes, or the specific target is only present in an infected cell or tissue, which associates with a disease.

### Examples of Fluorescent Reporting

In some embodiments, the present invention provides a method of detecting the presence or absence of a virus, bacterium or fungus in a sample. The method includes providing a sample potentially containing one or more virus, bacterium or fungus. Also provided is a set of one to six monomers. Each monomer comprises one or more ligand elements, which are useful for binding to one or more target molecules on the surface of, or internally within the virus, bacterium or fungus, with a dissociation constant less than 300 μM, and a linker element being connected directly or indirectly through a connector to the one or more ligand elements. The linker element is capable of forming a bond with one or more linker elements of either the same or a different monomer of the set of monomers. Association of the linker elements, with their ligand elements bound to the one or more target molecules on the surface of, or internally within the virus, bacterium or fungus to form a multimer, will generate a unique fluorescent signature different from that produced by those monomers either alone or in association with each other in the absence of the virus, bacterium or fungus target, when subjected to electromagnetic excitation. The sample is contacted with the set of monomers under conditions effective to allow the ligand elements to bind to the target molecules on the surface of, or internally within the virus, bacterium or fungus, if such target molecules are present in the sample. The monomers are subjected to reaction conditions effective for the linker elements of either the same or different monomers to undergo bond forming to form multimers, if such target molecules are present in the sample. The presence or absence of the virus, bacterium, or fungus in the sample is then detected based on the fluorescent signature of the sample subjected to the contacting and the subjecting.

In a further embodiment, the ligand elements are designed to bind to surface protein targets on the virus, bacterium, or fungus, often to proximate locations of the target molecules on the surface of, or internally within the virus, bacterium, or fungus. For instance, the ligand elements of cofluorons can be designed to have affinity to the Dengue hemorrhagic fever virus, based on the 3-dimensional structure of the “E” surface protein. The E protein is important for entry into the cell and initiation of infection, as well as viral assembly and release. Two different cofluoron monomers can combine in the β-OG binding cleft on the surface of the “E” protein dimer to create a fluorescent signal. In Dengue, there are 90 copies of the surface “E” protein dimer organized into 30 triad rafts. Cofluorons may be designed to bind an E protein dimer at one or multiple sites, including adjacent non-identical sites or more widely spaced identical sites.

Cofluorons may also be designed to target multiple targets or multiple sites on a target. For example, for the aforementioned target “E” surface protein in Dengue hemorrhagic fever virus, cofluorons may be designed on the three dimers contained within a triad raft, or E protein dimers on adjacent rafts.

Exemplary cofluoron designs include: (a) cofluorons with identical ligand elements, which bind to adjacent identical binding pockets of a target protein, and combine on their linker-element portions to create a fluorescent signal; (b) cofluorons with different ligand elements, which bind to adjacent targets, and combine on their linker-element portions to create a fluorescent signal; or (c) cofluorons where a ligand element has both “donor” and “acceptor” linker elements (i.e., their geometry prevents formation of intramolecular covalent bonds), such that two or more cofluorons bind to the surface of a virus through two or more target proteins. These designs may be used to cover the surface of a virus or bacteria with multiple copies of fluorescent molecules, allowing for convenient detection of such pathogens, either in vivo or in the environment. Cofluoron targeting of pathogenic viruses has broad applicability. The structure of the virus capsid of Dengue virus is very similar for other members of the flavivirus genus, including, West Nile virus, tick-borne encephalitis virus, Japanese encephalitis virus, and Yellow Fever virus. Capsids composed of multiple copies of a coat protein are characteristic of most families of pathogenic viruses.

In some embodiments, the present invention provides a method of detecting the macromolecular association of one or more target molecules in a sample. The method includes providing a sample potentially containing one or more target molecules capable of undergoing a molecular association. Also provided is a set of one to six monomers. Each monomer comprises one or more ligand elements, which are useful for binding to the one or more target molecules capable of undergoing a molecular association with a dissociation constant less than 300 μM, and a linker element being connected directly or indirectly through a connector to the one or more ligand elements. The linker element is capable of forming a bond with one or more linker elements of either the same or a different monomer of the set of monomers. Association of the linker elements, with their ligand elements bound the one or more target molecules capable of undergoing a molecular association to form a multimer, will generate a unique fluorescent signature different from that produced by those monomers either alone or in association with each other in the absence of the one or more target molecules capable of undergoing a molecular association, when subjected to electromagnetic excitation. The sample is contacted with the set of monomers under conditions effective to allow the ligand elements to bind to the one or more target molecules capable of undergoing a molecular association, if such target molecules are present in the sample. The monomers are subjected to reaction conditions effective for the linker elements of either the same or different monomers to undergo bond forming to form multimers, if such target molecules are present in the sample. The presence or absence of the one or more target molecules capable of undergoing a molecular association in the sample is then detected based on the fluorescent signature of the sample subjected to the contacting and the subjecting.

In some further embodiments, the ligand elements are designed to bind to proximate locations of the target molecules capable of undergoing a molecular association.

The macromolecular association in this case can be a marker for a cellular process, metabolism of cells, intracellular and extracellular compartmentalization, cell signaling, disease state, disease progression, disease prognosis, disease remission, and therapeutic molecule binding.

In one embodiment, the macromolecular association of interest may be a marker for a disease state. Because cofluorons possess the binding specificity to target molecule, cofluorons provided herein can be used as reporters to trace disease-specific genetic anomalies. For example, fusion genes in cancer arise from chromosomal rearrangement, and may occur by chromosomal inversion, interstitial deletion or translocation. More than 400 proteins are known to form fusion products arising from these chromosomal modifications. BCR-ABL gene fusion, for instance, a result from the Philadelphia translocation, is commonly reported in chronic myelogenous leukemia (CML); and TMPRSS2-ERG gene fusion often occurs in prostate cancers. Other widely recognized translocations involved in a variety of cancers including in solid tumors, include ALK-EML4 and ROS1-FIG. A more complete list of chromosomal fusions that are characterized by translocations may be found on the website hosted by Wellcome Trust Sanger Institute (Genome Research Limited, Hinxton, England) at http://www.sanger.ac.uk/genetics/CGP/Census/translocation.shtml. These fusions typically match proteins which normally do not interact with each other or the fusion leads to a loss of regulatory domains.

These disease-specific genetic anomalies are attractive targets for pharmaceuticals, because the predicted side effects are minimal and normal tissue is not targeted as the fusions do not occur in the healthy population. However, certain fusions simply amplify the activity of commonly expressed genes, leading to a potential for toxicity even for some of these targets. In this regard, cofluorons against each part of the fusion proteins may be generated to selectively report or even destroy the cells containing such fusion proteins. Furthermore, screening using cofluorons for such ligands or drugs (e.g., the fusion protein products) would provide a rapid detection protocol and allow physicians to determine the likely success of the specific drugs, such as fusion-specific agents (e.g., to discover drugs like Imatinib which are suitable for treating patients with the BCR-ABL chimeric protein).

As another example, many neurodegenerative diseases arise due to misfolding of proteins that aggregate to form plaques. For example, Alzheimer's disease arises due to plaques composed of amyloid beta-peptide. Such plaques are detectable with cofluorons which are small enough to traverse the blood-brain barrier, yet large enough to combine on the surface of amyloid beta-peptide monomers and detect the formation of amyloid fibrils.

In another embodiment, the macromolecular association of interest may be a marker for a signaling pathway. Cofluorons may be used to target protein-protein interactions, interfering a signaling pathway. For example, cofluorons may target sequence-specific proteases, such as the caspases, which play a role in the apoptotic pathway.

Many proteins use protein interaction domains as modular units within their structure to achieve their desired functions. Some proteins, such as the tumor suppressor p53, are mutated in cancer cells, causing them to unfold more easily and thus not function properly. Likewise, some proteins undergo conformational changes, which may activate or deactivate enzymatic activity or additional signaling. Cofluorons may be designed to bind one or the other conformer more tightly, and thus act as a reporter of a protein function.

Cofluorons may be used to detect protein-protein-nucleic acid interactions when transcription factors bind to dsDNA or when proteins bind to RNA. Many proteins undergo modifications (i.e. phosphorylation, acetylation, methylation, sumolation, prenylation, and ubiquitination) where these modifications allow for signaling, transport, or degradation through additional protein interactions. All of these processes may be detected and monitored by judiciously designed cofluorons. Larger modifications, such as synthesis of glycoproteins provide the potential for cofluorons to bind when proteins bind to the carbohydrate moieties.

A detailed description of different macromolecular associations and cofluoron designs to target these macromolecular associations can be found in PCT/US 2010/002708, which is hereby incorporated by reference in their entirety.

### Specific Examples of Cofluorons

Examples of cofluoron monomers that can form covalent association between linker elements containing boronic acids or oxaboroles, or their binding partners such as diols, catechols, coumarins, amino alcohols, amino thiols, α-hydroxyacids, o-hydroxy arylamides are shown below.

| | |
|---|---|
| {3-[5-({4-[3- (aminomethyl)phenyl]piperidin- 1-yl}carbonyl)- 1-benzothiophen-7- yl]phenyl}boronic acid | 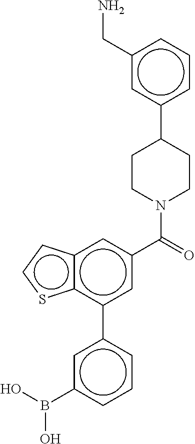 |
| [5-({4-[3- (aminomethyl)phenyl]piperidin-1- yl}carbonyl)quinolin-2- yl]boronic acid | 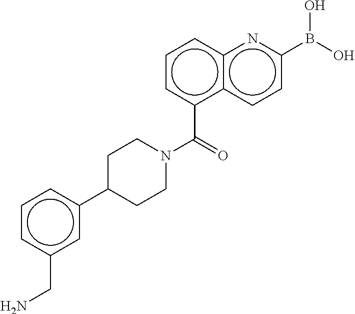 |
| [4-({4-[3- (aminomethyl)phenyl]piperidin-1- yl}carbonyl)quinolin-8- yl]boronic acid | 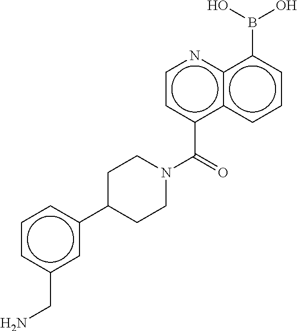 |
| [2-({4-[3-(aminomethyl)phenyl]piperidin- 1-yl}carbonyl)quinolin-5- yl]boronic acid | 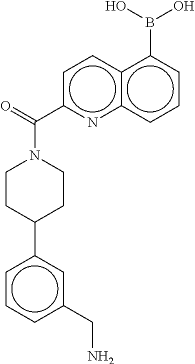 |
| [5-({4-[3-(aminomethyl)phenyl]- piperidin-1- yl}carbonyl)quinolin-8- yl]boronic acid | 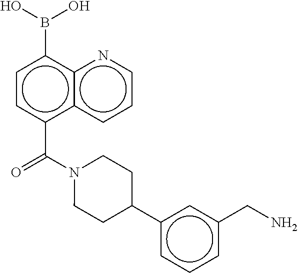 |
| [8-({4-[3- (aminomethyl)phenyl]piperidin-1- yl}carbonyl)quinolin-5- yl]boronic acid | 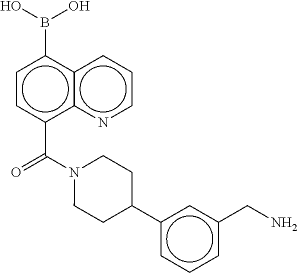 |
| [6-({4-[3- (aminomethyl)phenyl]piperidin-1-yl}- carbonyl)isoquinolin- 8-yl]boronic acid | 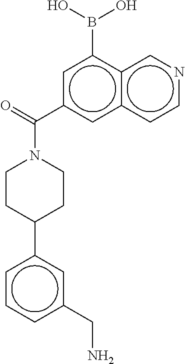 |
| [7-({4-[3-(aminomethyl)- phenyl]piperidin-1- yl}carbonyl)isoquinolin- 5-yl]boronic acid | 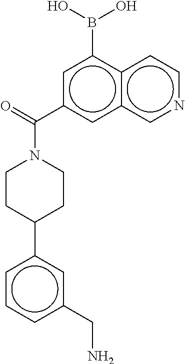 |
| [3-({4-[3- (aminomethyl)phenyl]piperidin-1-yl}- carbonyl)isoquinolin- 7-yl]boronic acid | 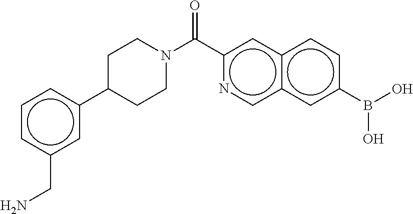 |
| [3-({4-[3-(aminomethyl)- phenyl]piperidin-1- yl}carbonyl)isoquinolin- 6-yl]boronic acid | 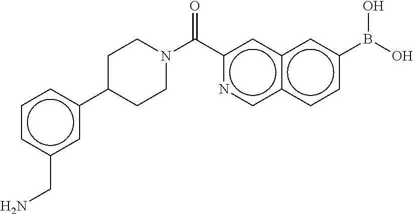 |
| [3-({4-[3- (aminomethyl)phenyl]piperidin-1- yl}carbonyl)isoquinolin-8- yl]boronic acid | 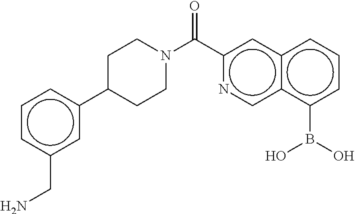 |
| [3-({4-[3- (aminomethyl)phenyl]piperidin-1-yl}- carbonyl)isoquinolin- 5-yl]boronic acid | 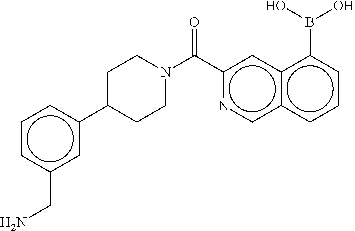 |
| [3-({4-[3- (aminomethyl)phenyl]piperidin- 1-yl}carbonyl)isoquinolin- 1-yl]boronic acid | 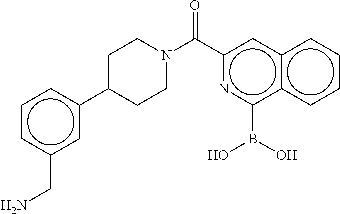 . |
| [8-({4-[3- (aminomethyl)phenyl]piperidin-1- yl}carbonyl)quinolin-6- yl]boronic acid | 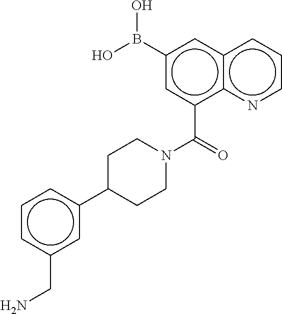 |
| [5-({4-[3- (aminomethyl)phenyl]piperidin-1- yl}carbonyl)isoquinolin- 1-yl]boronic acid | 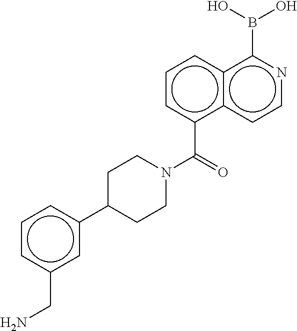 |
| [5-({4-[3- (aminomethyl)phenyl]piperidin-1- yl}carbonyl)isoquinolin- 8-yl]boronic acid | 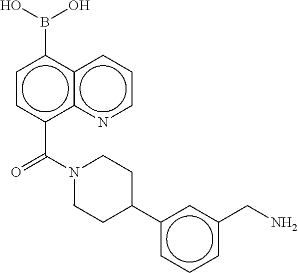 |
| [3-({4-[3- (aminomethyl)phenyl]piperidin-1- yl}carbonyl)quinolin-8- yl]boronic acid | 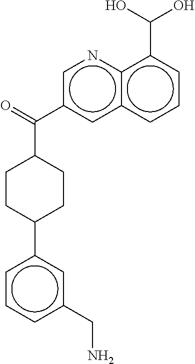 |
| [5-({4-[3- (aminomethyl)phenyl]piperidin-1- yl}carbonyl)isoquinolin- 7-yl]boronic acid | 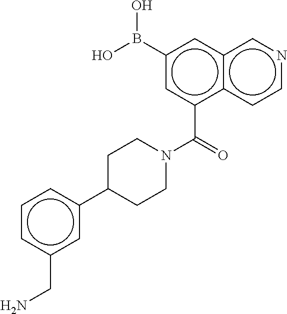 |
| [6-({4-[3- (aminomethyl)phenyl]piperidin- 1-yl}carbonyl)isoquinolin- 3-yl]boronic acid | 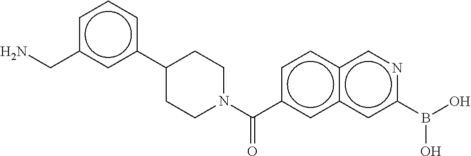 |
| [6-({4-[3- (aminomethyl)phenyl]piperidin-1- yl}carbonyl)isoquinolin- 1-yl]boronic acid | 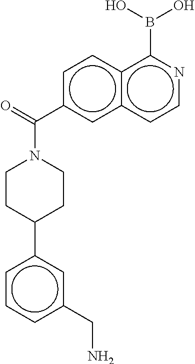 |
| [3-({4-[3- (aminomethyl)phenyl]piperidin-1- yl}carbonyl)quinolin-5- yl]boronic acid | 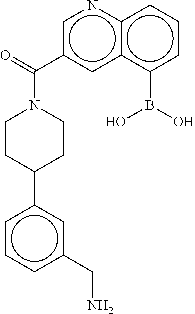 |
| [6-({4-[3- (aminomethyl)phenyl]piperidin-1- yl}carbonyl)quinolin-5- yl]boronic acid | 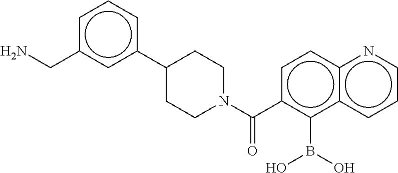 |
| [7-({4-[3- (aminomethyl)phenyl]piperidin-1- yl}carbonyl)quinolin-3- yl]boronic acid | 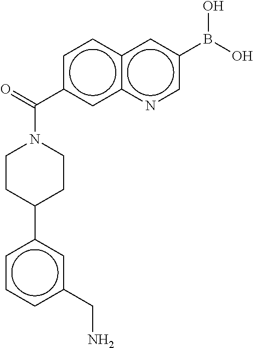 |
| [7-({4-[3- (aminomethyl)phenyl]piperidin-1- yl}carbonyl)quinolin-2- yl]boronic acid | 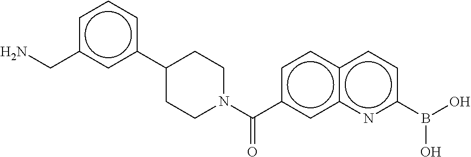 |
| [7-({4-[3- (aminomethyl)phenyl]piperidin-1- yl}carbonyl)quinolin-4- yl]boronic acid | 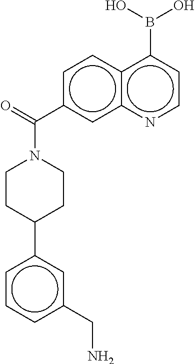 |
| [7-({4-[3- (aminomethyl)phenyl]piperidin-1- yl}carbonyl)quinolin-5- yl]boronic acid | 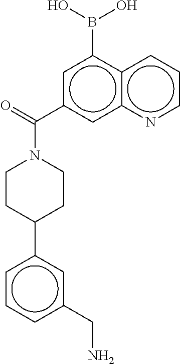 |
| [8-({4-[3- (aminomethyl)phenyl]piperidin-1- yl}carbonyl)isoquinolin- 6-yl]boronic acid | 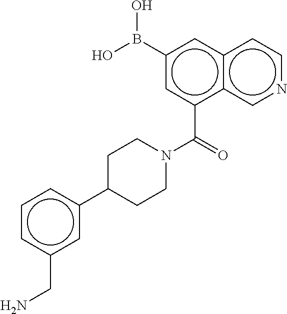 |
| [1-({4-[3- (aminomethyl)phenyl]piperidin-1- yl}carbonyl)isoquinolin- 6-yl]boronic acid | 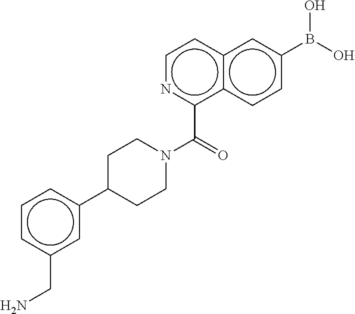 |
| [1-({4-[3- (aminomethyl)phenyl]piperidin-1- yl}carbonyl)isoquinolin- 7-yl]boronic acid | 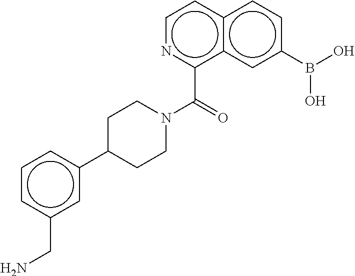 |
| [1-({4-[3- (aminomethyl)phenyl]piperidin-1- yl}carbonyl)isoquinolin- 5-yl]boronic acid | 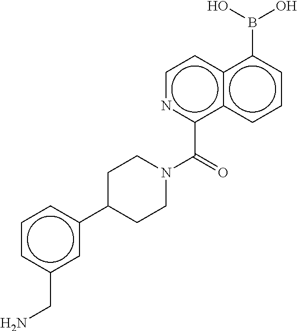 |
| [2-({4-[3- (aminomethyl)phenyl]piperidin-1- yl}carbonyl)quinolin-7- yl]boronic acid | 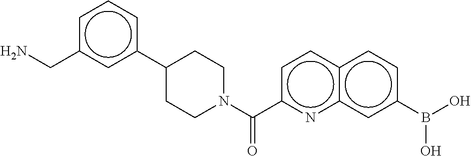 |
| [6-({4-[3- (aminomethyl)phenyl]piperidin-1- yl}carbonyl)quinolin-3- yl]boronic acid | 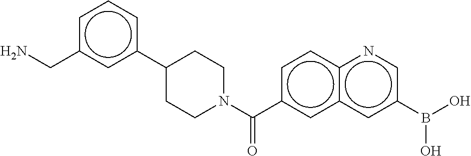 |
| [2-({4-[3- (aminomethyl)phenyl]piperidin-1- yl}carbonyl)quinolin-8- yl]boronic acid | 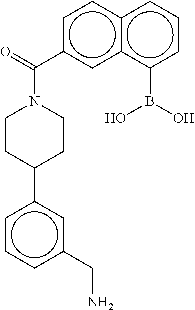 |
| [2-({4-[3- (aminomethyl)phenyl]piperidin-1- yl}carbonyl)quinolin-4- yl]boronic acid | 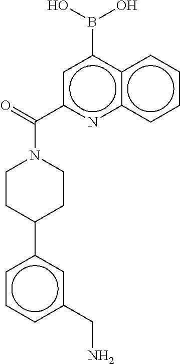 |
| [4-({4-[3- (aminomethyl)phenyl]piperidin-1- yl}carbonyl)isoquinolin- 8-yl]boronic acid | 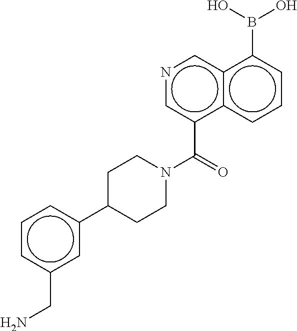 |
| [8-({4-[3- (aminomethyl)phenyl]piperidin-1- yl}carbonyl)isoquinolin- 3-yl]boronic acid | 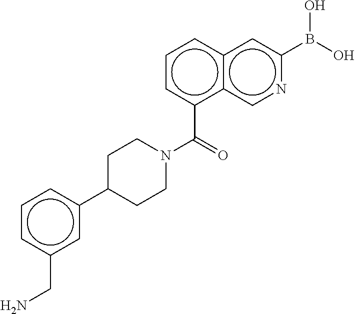 |
| [8-({4-[3- (aminomethyl)phenyl]piperidin-1- yl}carbonyl)isoquinolin- 4-yl]boronic acid | 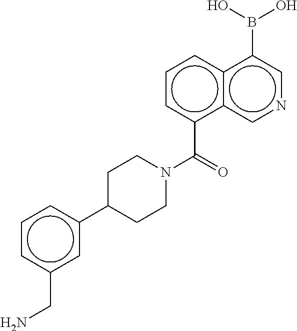 |
| [8-({4-[3- (aminomethyl)phenyl]piperidin-1- yl}carbonyl)isoquinolin- 5-yl]boronic acid | 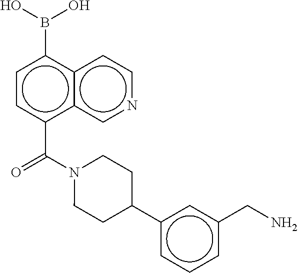 |
| [5-({4-[3- (aminomethyl)phenyl]piperidin-1- yl}carbonyl)quinolin-3- yl]boronic acid | 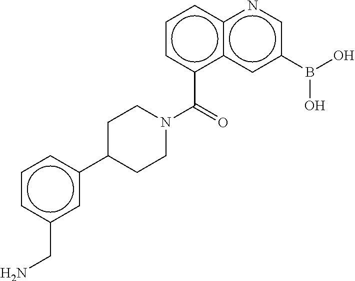 |
| [8-({4-[3- (aminomethyl)phenyl]piperidin-1- yl}carbonyl)quinolin-3- yl]boronic acid | 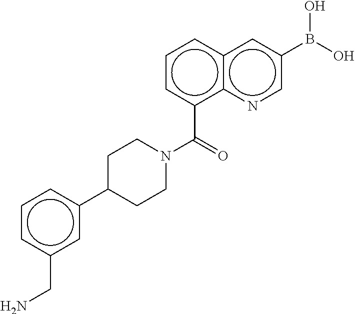 |
| [8-({4-[3- (aminomethyl)phenyl]piperidin-1- yl}carbonyl)quinolin-2- yl]boronic acid | 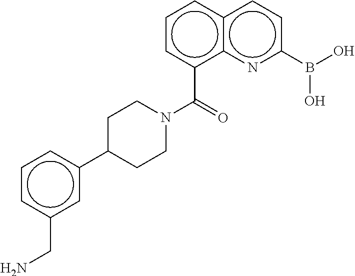 |
| [8-({4-[3- (aminomethyl)phenyl]piperidin-1- yl}carbonyl)quinolin-4- yl]boronic acid | 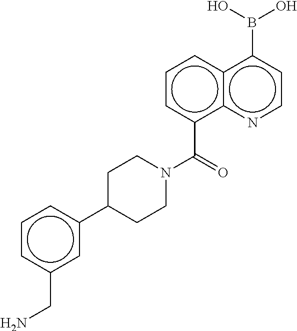 |
| [5-({4-[3- (aminomethyl)phenyl]piperidin-1- yl}carbonyl)quinolin-4- yl]boronic acid | 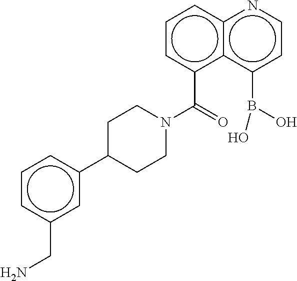 |
| [4-({4-[3- (aminomethyl)phenyl]piperidin-1- yl}carbonyl)quinolin-5- yl]boronic acid | 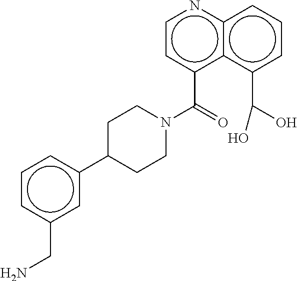 |
| [3-({4-[3- (aminomethyl)phenyl]piperidin-1- yl}carbonyl)quinolin-7- yl]boronic acid | 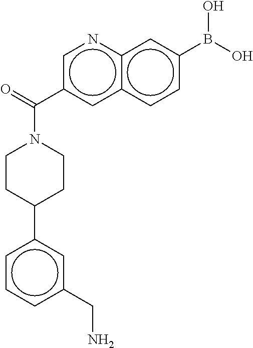 |
| [3-({4-[3- (aminomethyl)phenyl]piperidin-1- yl}carbonyl)quinolin-6- yl]boronic acid | 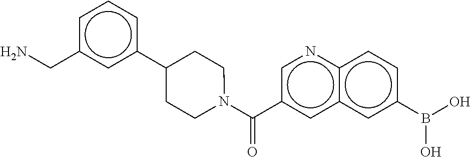 |
| [3-({4-[3- (aminomethyl)phenyl]piperidin-1- yl}carbonyl)quinolin-4- yl]boronic acid | 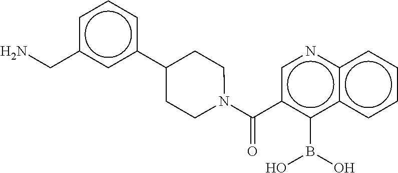 |
| [6-({4-[3- (aminomethyl)phenyl]piperidin-1- yl}carbonyl)quinolin-2- yl]boronic acid | 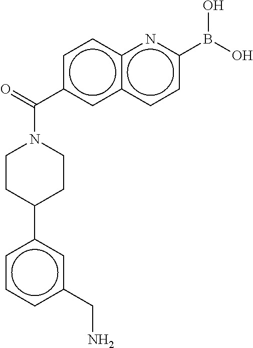 |
| [6-({4-[3- (aminomethyl)phenyl]piperidin-1- yl}carbonyl)quinolin-4- yl]boronic acid | 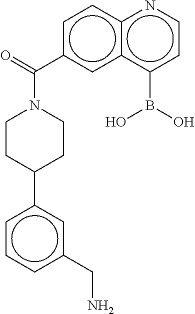 |
| [7-({4-[3- (aminomethyl)phenyl]piperidin-1- yl}carbonyl)quinolin-8- yl]boronic acid | 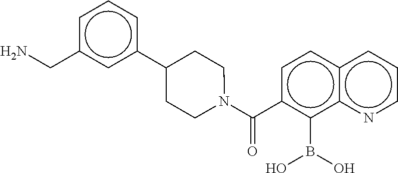 |
| [2-({4-[3- (aminomethyl)phenyl]piperidin-1- yl}carbonyl)quinolin-6- yl]boronic acid | 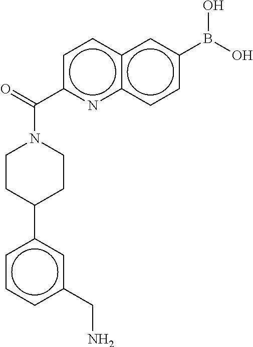 |
| [4-({4-[3- (aminomethyl)phenyl]piperidin-1- yl}carbonyl)isoquinolin- 5-yl]boronic acid | 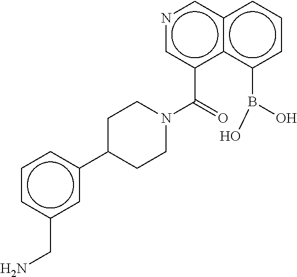 |
| [1-({4-[3- (aminomethyl)phenyl]piperidin-1- yl}carbonyl)isoquinolin- 8-yl]boronic acid | 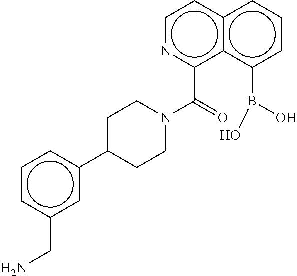 |
| [8-({4-[3- (aminomethyl)phenyl]piperidin-1- yl}carbonyl)isoquinolin- 1-yl]boronic acid | 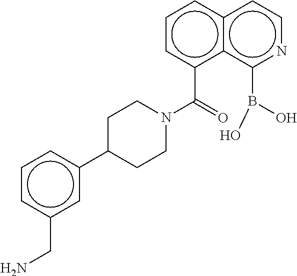 |
| [8-({4-[3- (aminomethyl)phenyl]piperidin-1- yl}carbonyl)isoquinolin- 3-yl]boronic acid | 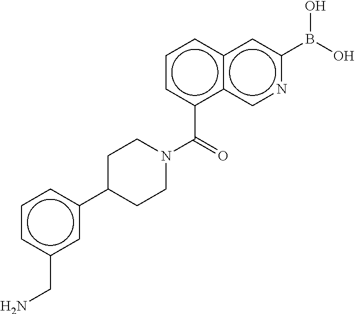 |
| [8-({4-[3- (aminomethyl)phenyl]piperidin-1- yl}carbonyl)isoquinolin- 4-yl]boronic acid | 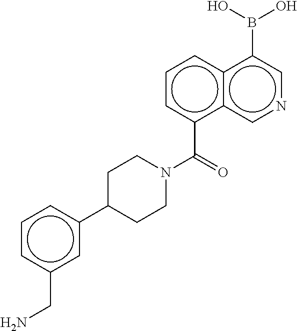 |
| [8-({4-[3- (aminomethyl)phenyl]piperidin-1- yl}carbonyl)isoquinolin- 5-yl]boronic acid | 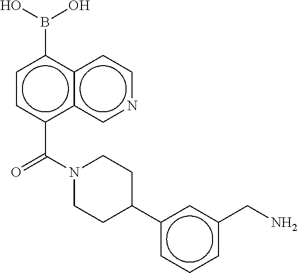 |
| [8-({4-[3- (aminomethyl)phenyl]piperidin-1- yl}carbonyl)isoquinolin- 1-yl]boronic acid | 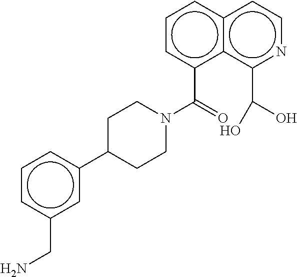 |
| [8-[{4-[3- (aminomethyl)phenyl]piperidin-1- yl}carbonyl)isoquinolin- 6-yl]boronic acid | 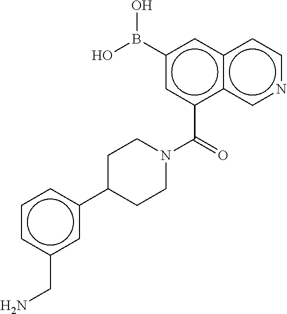 |
| [5-[{4-[3- (aminomethyl)phenyl]piperidin-1- yl}carbonyl)isoquinolin- 3-yl]boronic acid | 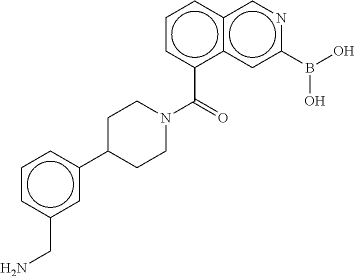 |
| [5-({4-[3- (aminomethyl)phenyl]piperidin-1- yl}carbonyl)isoquinolin- 4-yl]boronic acid | 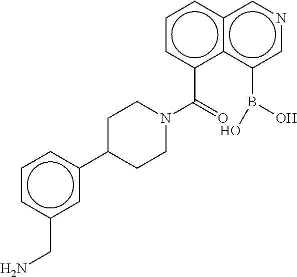 |
| [7-({4-[3- (aminomethyl)phenyl]piperidin-1- yl}carbonyl)isoquinolin- 3-yl]boronic acid | 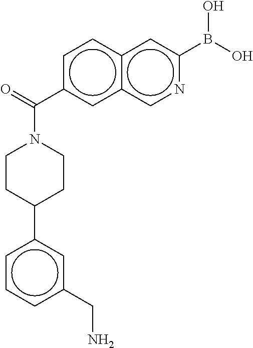 |
| [7-({4-[3- (aminomethyl)phenyl]piperidin-1- yl}carbonyl)isoquinolin- 4-yl]boronic acid | 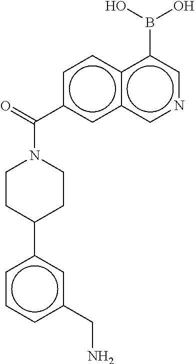 |
| [6-({4-[3- (aminomethyl)phenyl]piperidin-1- yl}carbonyl)isoquinolin- 4-yl]boronic acid | 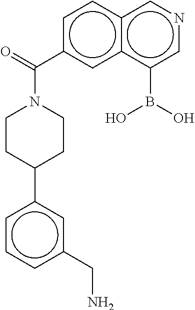 |
| [7-({4-[3- (aminomethyl)phenyl]piperidin-1- yl}carbonyl)isoquinolin- 1-yl]boronic acid | 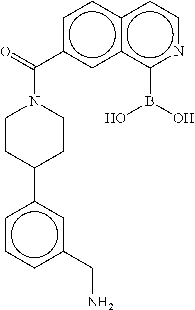 |
| [6-({4-[3- (aminomethyl)phenyl]piperidin-1- yl}carbonyl)isoquinolin- 5-yl]boronic acid | 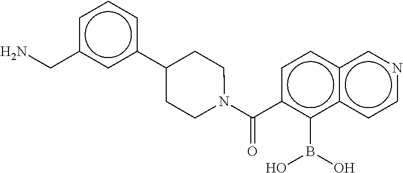 |
| [7-({4-[3- (aminomethyl)phenyl]piperidin-1- yl)carbonyl)isoquinolin- 8-yl]boronic acid | 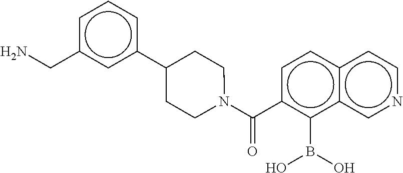 |
| [3-({4-[3- (aminomethyl)phenyl]piperidin-1- yl}carbonyl)isoquinolin- 4-yl]boronic acid | 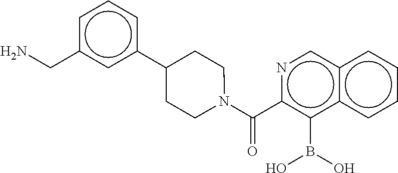 |
| 4-(2-{4-[3- (aminomethyl)phenyl]piperidin-1-yl}-2- oxoethyl)-6,7- dihydroxy-2H-chromen- 2-one | 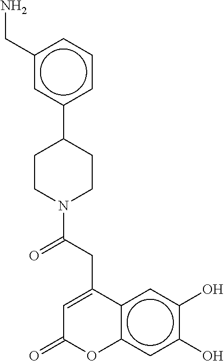 |
| 4-({4-[3- (aminomethyl)phenyl]piperidin- 1-yl}carbonyl)- 7,8-dihydroxy-2H- chromen-2-one | 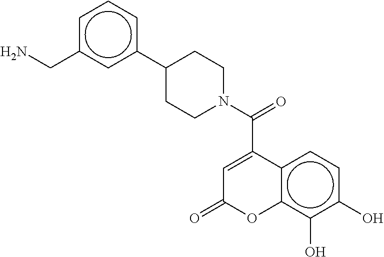 |
| 4-(2-{4-[3- (aminomethyl)phenyl]piperidin-1-yl}- 2-oxoethyl)-7,8- dihydroxy-2H-chromen- 2-one | 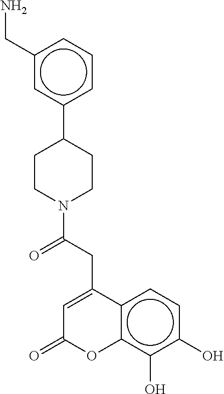 |
| 3-(2-{4-[3- (aminomethyl)phenyl]piperidin-1-yl)-2- oxoethyl)-6,7-dihydroxy-4-methyl-2H- chromen-2-one | 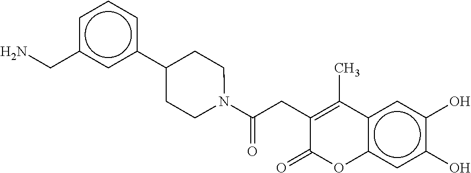 |
| 3-({4-[3- (aminomethyl)phenyl]piperidin- 1-yl}carbonyl)-6,7-dihydroxy-2H- chromen-2-one | 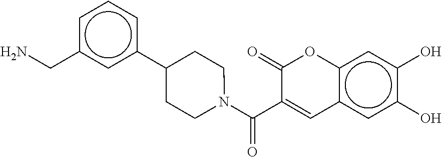 |
| 3-(2-{4-[3- (aminomethyl)phenyl]piperidin- 1-yl}-2-oxoethyl)-7,8- dihydroxy-4-methyl-2H- chromen-2-one | 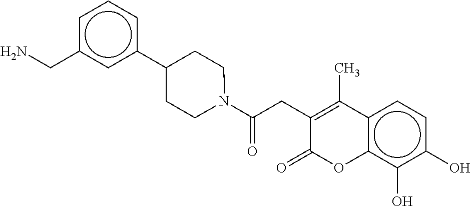 |
| 3-({4-[3- (aminomethyl)phenyl]piperidin-1- yl}carbonyl)- 7,8-dihydroxy-2H- chromen-2-one | 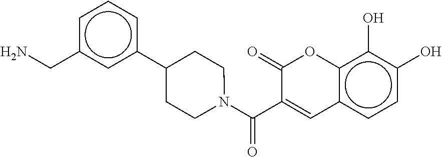 |
| 3-(2-{4-[3- (aminomethyl)phenyl]piperidin-1-yl)- 2-oxoethyl)-6,7- dihydroxy-4-methyl-2H- chromen-2-one | 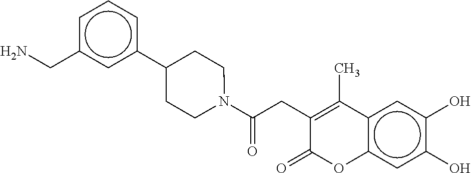 |
| 3-({4-[3- (aminomethyl)phenyl]piperidin- 1-yl}carbonyl)- 6,7-dihydroxy-2H- chromen-2-one | 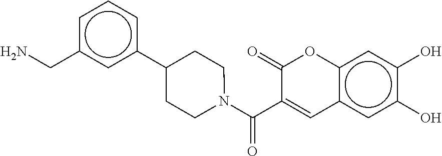 |
| 3-(2-{4-[3- (aminomethyl)phenyl]piperidin-1-yl}-2- oxoethyl)-7,8- dihydroxy-4-methyl-2H- chromen-2-one | 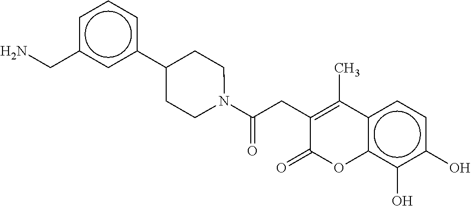 |
| 3-({4-[3- (aminomethyl)phenyl]piperidin- 1-yl}carbonyl)- 7,8-dihydroxy-2H- chromen-2-one | 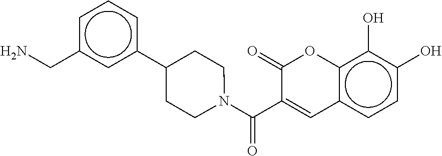 |
| 4-({4-[3- (aminomethyl)phenyl]piperidin- 1-yl}carbonyl)- 7,8-dihydroxy-2H- chromen-2-one | 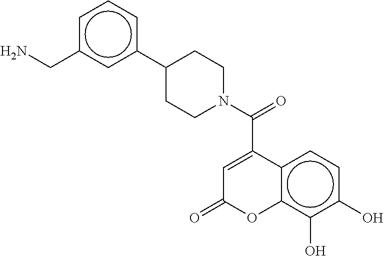 |
| 3-(2-{4-[3- (aminomethyl)phenyl]piperidin- 1-yl}-2- oxoethyl)-6,7- dihydroxy-4-methyl-2H- chromen-2-one | 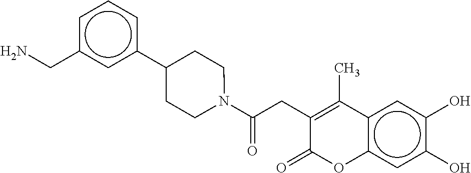 |
| 3-({4-[3- (aminomethyl)phenyl]piperidin- 1-yl}carbonyl)- 1H-indazole-5,6-diol | 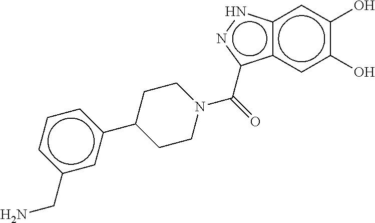 |
| 5-({4-[3- (aminomethyl)phenyl]piperidin- 1-yl)carbonyl)- 3-methoxybenzene-1,2-diol | 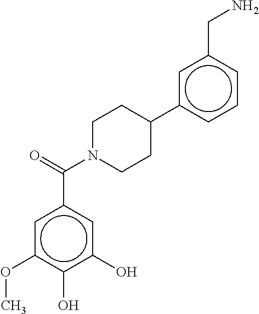 |
| 4-[(1E)-2-({[(5S)-3-[3- fluoro-4-(morpholin-4- yl)phenyl]-2-oxo-1,3- oxazolidin-5- yl]methyl}carbamoyl)eth- 1-en-1-yl]-2- hydroxybenzamide | 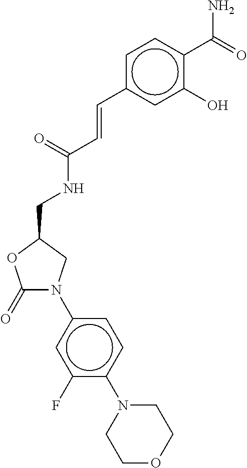 |
| 5-({4-[3- (aminomethyl)phenyl]piperidin- 1-yl}carbonyl)- 3-chloro-2-hydroxy-N- methoxybenzamide | 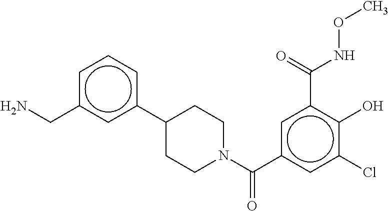 |
| 5-{[5-(aminomethyl)- 2H-spiro[1-benzofuran- 3,4′-piperidine]-1′- yl]carbonyl}-2-hydroxy- N-methoxybenzamide | 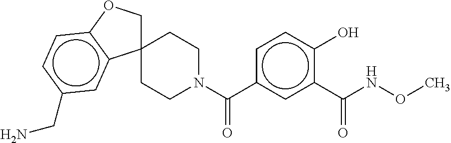 |
| 2-[(1E)-3-{4-[3- (aminomethyl)phenyl]piperidin- 1-yl}-3-oxoprop- 1-en-1-yl]-6-hydroxy-N- methoxybenzamide | 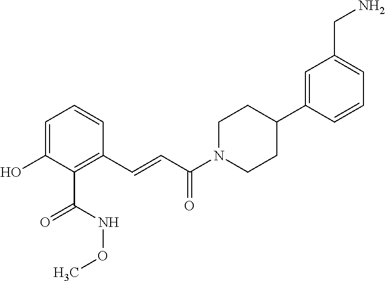 [SMILES: CONC(=O)c1c(O)cccc1/C=C/C(=O)N1CCC(c2cccc(CN)c2)CC1] |
| 5-({4-[3- (aminomethyl)phenyl]piperidin- 1-yl}carbonyl)- 2-hydroxy-N-methoxy- 3-methylbenzamide | 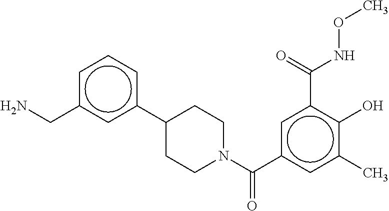 |
| [8-({4-[3- (aminomethyl)phenyl]piperidin- 1-yl}carbonyl)- 4-fluoronaphthalen-2- yl]boronic acid | 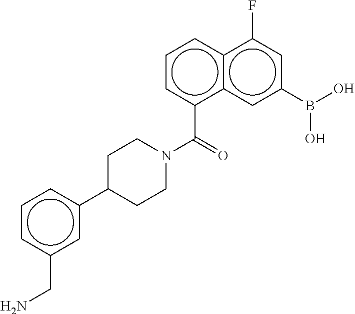 |
| {3-[5-({4-[3- (aminomethyl)phenyl]piperidin- 1-yl}carbonyl)-2,3- dimethylphenyl]phenyl}- boronic acid | 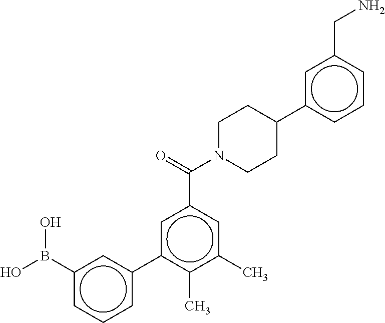 |
| {3-[5-({4-[3- (aminomethyl)phenyl]piperidin- 1-yl}carbonyl)- 2,3- dichlorophenyl]phenyl}boronic acid | 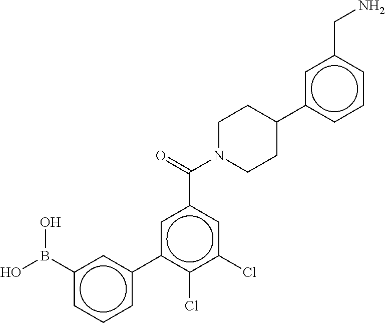 |
| (8-{[5-(aminomethyl)- 2H-spiro[1-benzofuran- 3,4′-piperidine]-1′- yl]carbonyl}naphthalen- 2-yl)boronic acid | 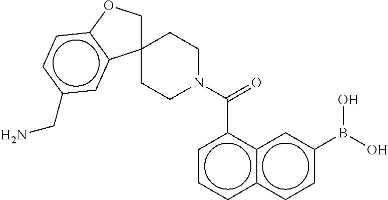 |
| (5-{[5-(aminomethyl)- 2H-spiro[1-benzofuran- 3,4′-piperidine]-1′- yl]carbonyl}naphthalen- 2-yl)boronic acid | 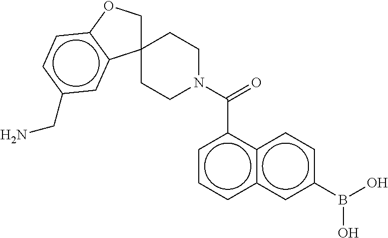 |
| [5-({4-[3- (aminomethyl)phenyl}piperidin- 1-yl}carbonyl)- 1-benzofuran-2- yl]boronic acid | 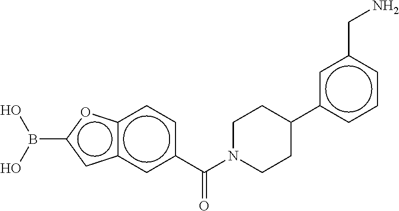 |
| [3-({4-[3- (aminomethyl)phenyl]piperidin- 1-yl}carbonyl)- 4-fluoro-1H-indazol-6- yl]boronic acid | 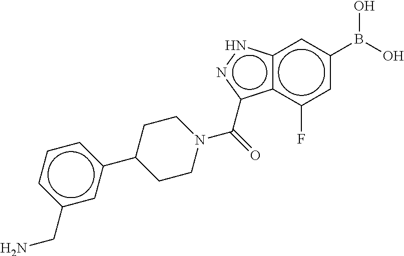 |
| {3-[5-({4-[3- (aminomethyl)phenyl]piperidin- 1-yl}carbonyl)-2,3- dimethylphenyl]phenyl}- boronic acid | 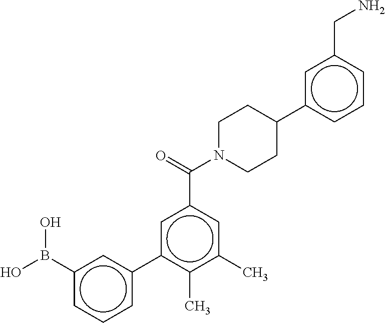 |
| {3-[3-({4-[3- (aminomethyl)phenyl]piperidin- 1-yl}carbonyl)-5- chlorophenyl]phenyl}boronic acid | 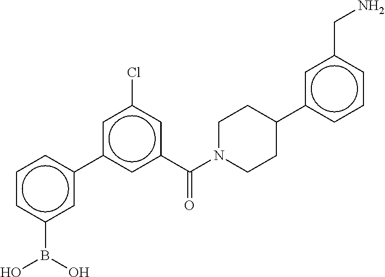 |
| {3-[5-({4-[3- (aminomethyl)phenyl]piperidin- 1-yl}carbonyl)-2- chlorophenyl]phenyl}boronic acid | 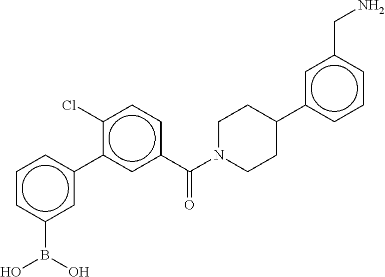 |
| {3-[5-({4-[3- (aminomethyl)phenyl]piperidin- 1-yl}carbonyl)- 2,3- dichlorophenyl]phenyl}boronic acid | 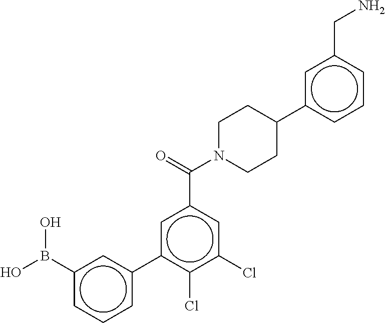 |
| {5-[3-({4-[3- (aminomethyl)phenyl]piperidin- 1-yl}carbonyl)phenyl]-2- fluorophenyl}boronic acid | 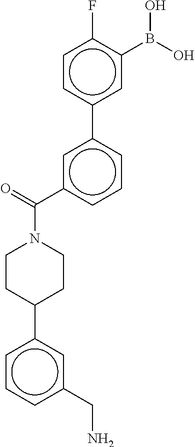 |
| {3-[3-({4-[3- (aminomethyl)phenyl]piperidin- 1-yl}carbonyl)phenyl]-5- fluorophenyl}boronic acid | 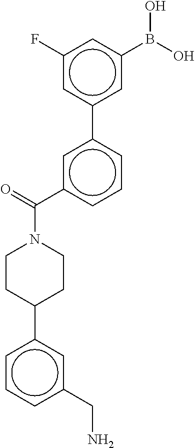 |
| 4-[3-({4-[3- (aminomethyl)phenyl]piperidin-1- yl}carbonyl)phenyl]-1,3- dihydro-2,1- benzoxaborol-1-ol | 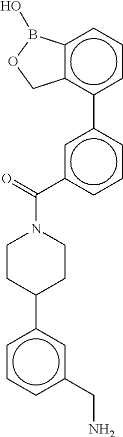 |
| {3-[3-({4-[3- (aminomethyl)phenyl]piperidin- 1-yl}carbonyl)phenyl]-2- fluorophenyl}boronic acid | 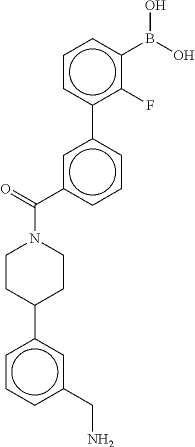 |
| [2-({[5-({4-[3- (aminomethyl)phenyl]piperidin-1- yl}carbonyl)naphthalen-1- yl)(methyl)amino}methyl)- phenyl]boronic acid | 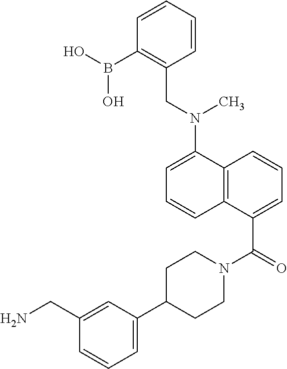 [SMILES: CN(Cc1ccccc1B(O)O)c1cccc2c(C(=O)N3CCC(c4cccc(CN)c4)CC3)cccc12] |
| [2-({[3-(2-{4-[3- (aminomethyl)phenyl]piperidin- 1-yl}-2- oxoethyl)-4-methyl-2- oxo-2H-chromen-7- yl](methyl)amino}methyl)- phenyl]boronic acid | 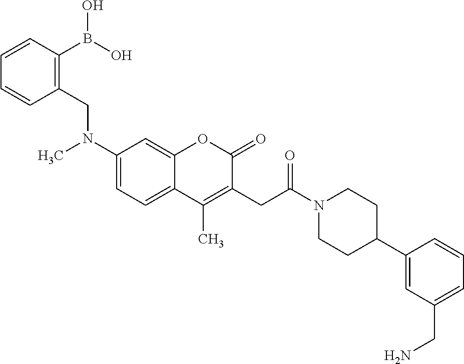 [SMILES: Cc1c(CC(=O)N2CCC(c3cccc(CN)c3)CC2)c(=O)oc2cc(N(C)Cc3ccccc3B(O)O)ccc12] |
| [2-({[4-({4-[3- (aminomethyl)phenyl]piperidin- 1-yl}carbonyl)- 2-oxo-2H-chromen-7- yl](methyl)amino}methyl)- phenyl]boronic acid | 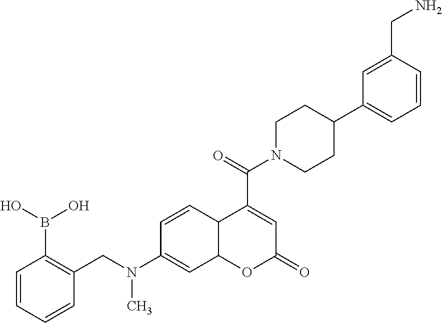 [SMILES: CN(Cc1ccccc1B(O)O)C1=CC2OC(=O)C=C(C(=O)N3CCC(c4cccc(CN)c4)CC3)C2C=C1] |
| [2-({[3-({4-[3- (aminomethyl)phenyl]piperidin- 1-yl}carbonyl)phenyl](methyl)- amino}methyl)phenyl]boronic acid | 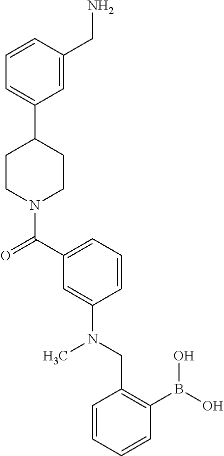 [SMILES: CN(Cc1ccccc1B(O)O)c1cccc(C(=O)N2CCC(c3cccc(CN)c3)CC2)c1] |
| [2-({[4-({4-[3- (aminomethyl)phenyl]piperidin- 1-yl}carbonyl)phenyl](methyl)- amino}methyl)phenyl]boronic acid | 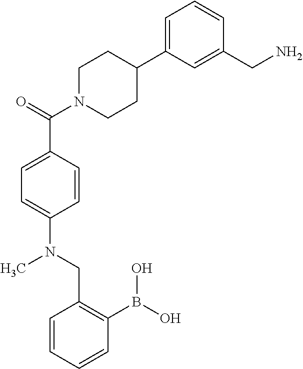 [SMILES: CN(Cc1ccccc1B(O)O)c1ccc(C(=O)N2CCC(c3cccc(CN)c3)CC2)cc1] |
| [2-({[5-({4-[3- (aminomethyl)phenyl]piperidin- 1-yl}carbonyl)naphthalen-2- yl](methyl)amino}methyl)- phenyl]boronic acid | 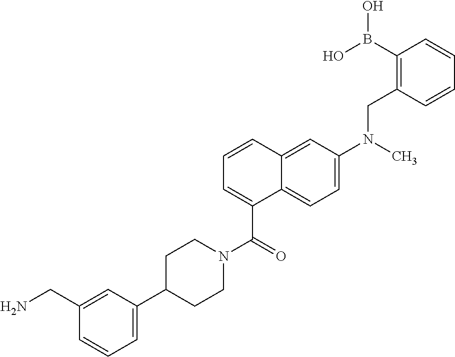 [SMILES: CN(Cc1ccccc1B(O)O)c1ccc2c(C(=O)N3CCC(c4cccc(CN)c4)CC3)cccc2c1] |

Some examples of the cofluoron dimers obtained from these sets of cofluoron monomers are can be found in coferon dimers exemplified in PCT/US 2010/002708, which is hereby incorporated by reference in its entirety.

### EXAMPLES

### Example 1

### Synthesis of Cofluoron Monomers Bearing Boronic Acid Functionality

The cofluoron monomers bearing boronic acid moieties were synthesized by either of the two methods (Method A & Method B) as below. Aryl halo carboxylic acids used in Step-1 of both Method A & Method B were either procured commercially or synthesized in house by known methods in the literature.

### Method A

Method A was carried out according to the following reaction scheme:

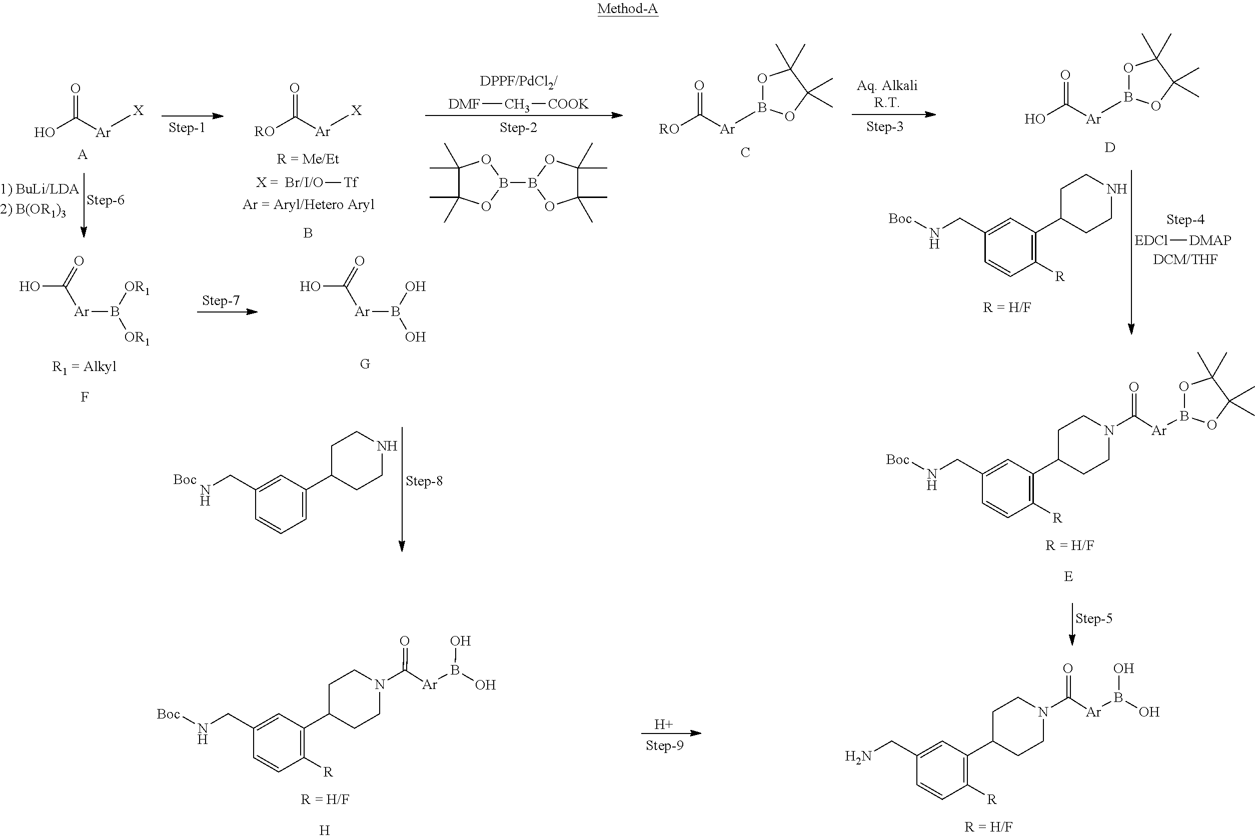

In general, aryl pinacolato boronate esters/boronic acids with carboxylic acid groups used in the reaction were synthesized and coupled with desired tert-butyl 3-(piperidin-4-yl)benzylcarbamate. The boronate ester moiety was hydrolyzed to boronic acid in acidic condition.

### Step-1

Aryl halo/hydroxy carboxylic acids were esterified by refluxing with excess methanol/ethanol in presence of catalytic sulfuric acid, or by refluxing the aryl halo/hydroxy carboxylic acid with thionyl chloride-methanol/ethanol followed by standard procedures involving distillation of excess alcohol and subsequent treatment of residue with aqueous sodium bicarbonate followed by extraction with dichloromethane/ethyl acetate. Purification was carried out by column chromatography over 100-200 mesh silica gel using hexane-ethyl acetate.

O-triflate derivatives of hydroxy esters were synthesized as per procedure described in the literature (J. Med. Chem. 53(5): 2010-2037 (2010), which is hereby incorporated by reference in its entirety).

The compounds synthesized by Step-1 and the corresponding reaction conditions are shown in the table below:

| Compound | | |
|---|---|---|
| No. | Structure | Reaction conditions |
| B-31 | 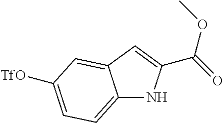 [SMILES: COC(=O)c1cc2cc(OS(=O)(=O)C(F)(F)F)ccc2[nH]1] | 1) Thionyl chloride (1.5 eq.), methanol (25 vol.), 4 h at 65°C., yield 93%; 2) As per J. Med. Chem. 53(5): 2010-2037 (2010), which is hereby incorporated by reference in its entirety, yield 81%. |
| B-62 | 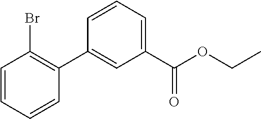 [SMILES: CCOC(=O)c1cccc(-c2ccccc2Br)c1] | Analogously as per Angew. Chem, Int. Edn. 43(40): 5331-5335 (2004), which is hereby incorporated by reference in its entirety. |
| B-35 | 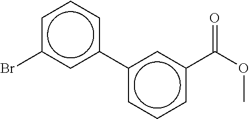 | Thionyl chloride (1.5 eq.), methanol (25 vol.), 4 h at 65°C., yield 92%. |
| B-11F | 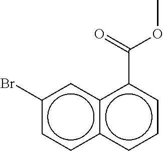 | Synthesized using literature procedures (Helvetica Chimica Acta, 21: 1519-1520 (1938); U.S. Pat. No. 4,391,816; Bull. Chem. Soc. Japan. 48: 3356-3366 (1975); and WO 2008/100480 A1, all of which are incorporated by reference in their entirety.) |

### Step-2

A solution of aryl halo/O-trifluoromethyl sulfonyl carboxylate in solvents such as toluene, dimethyl sulfoxide, dioxane was degassed with Argon, to this solution (bis-pinacolato)diboron, potassium acetate, and Pd(dppf)₂Cl₂ (dppf is referred to as 1,1′-bis(diphenylphosphanyl)ferrocene) were added at room temperature and the mixture was heated at 80-100° C. and monitored by TLC & LCMS until the starting compounds were consumed to the maximum extent. The reaction mixture was then diluted with water and extracted with ethyl acetate. Ethyl acetate extract was evaporated under vacuum to give the crude products that were purified by column chromatography over silica gel (Gradient: 0-10% ethyl acetate in hexane)

The compounds synthesized by Step-2 and the corresponding reaction conditions are shown in the table below:

| Compound | | |
|---|---|---|
| No. | Structure | Reaction conditions |
| C-31 | 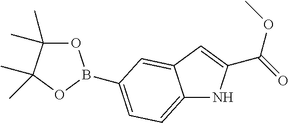 [SMILES: COC(=O)c1cc2cc(B3OC(C)(C)C(C)(C)O3)ccc2[nH]1] | Bispinacolato diboron (1.5 eq.), PdCl2 (dppf) (3 mol %), dppf (3 mol %), potassium acetate (3.0 eq.), dioxane, 20 hours at 100° C., Yield 54%. |
| C-62 | 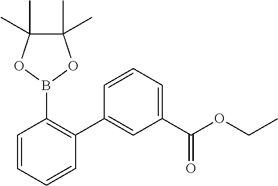 [SMILES: CCOC(=O)c1cccc(-c2ccccc2B2OC(C)(C)C(C)(C)O2)c1] | Ethyl 2′-bromo-[1,1′-biphenyl]-3-carboxylate (1 eq.), potassium acetate (3 eq.) bispinacolato diboron (10 eq.) PdCl₂ (dppf)•DCM adduct (0.03 eq.) DMSO (46 vol.), 110°C. for 5 h. Inorganics removed by column chromatography and carried forward to next step. |
| C-35 | 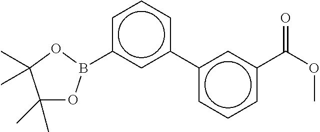 | KOAc (3 eq.), Bis Pin. Borane (10 eq.), dppf PdCl₂•DCM (6 Mol %), DMSO (12.5 vol.), 80° C. for 4 hours, yield 65%. |
| C-11F | 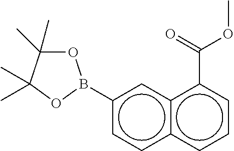 | KOAc (3 eq.), Bis Pin. Borane (10 eq.), dppf- PdCl₂•DCM (3 mol %), DMSO (10 vol.), 80° C. for 5 hours, yield 57.8%. |

### Step-3

Boronate ester from Step-2 was dissolved in mix of water and aqueous-mixable solvents such as THF (tetrahydrofuran)/methanol/Acetone. To this mixture, lithium hydroxide was added and the resulting mixture was stirred at room temperature and monitored by TLC & LCMS until the starting compound was consumed to the maximum extent (6-12 hours required). THF was then concentrated and reacted material was extracted with ethyl acetate and water. The organic layer was washed with water, and combined aqueous washings were acidified with 2N HCl and extracted with ethyl acetate. Ethyl acetate extract was dried over sodium sulphate and concentrated in vacuum to obtain crude product. In most of the cases, products were sufficiently pure to be used for carrying out the next step.

The compounds synthesized by Step-3 and the corresponding reaction conditions are shown in the table below:

| Compound | | |
|---|---|---|
| No. | Structure | Reaction conditions |
| D-31 | 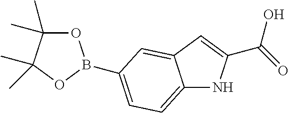 [SMILES: CC1(C)OB(c2ccc3[nH]c(C(=O)O)cc3c2)OC1(C)C] | LiOH (2.0 eq.), THF:H₂O (2:1), room temperature, yield 90%. |
| D-62 | 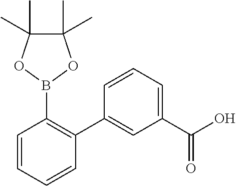 [SMILES: CC1(C)OB(c2ccccc2-c2cccc(C(=O)O)c2)OC1(C)C] | LiOH (3.0 eq.), THF:H₂O (1:1), room temperature, yield 25%. |
| D-35 | 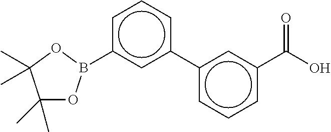 | LiOH (3.0 eq.), THF:H₂O (1:1), room temperature for 4 hours, yield 80%. |
| D-11F | 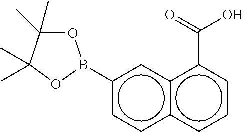 | LiOH (3.0 eq.), THF:H₂O (1:1), room temperature for 8 hours, yield 84%. Purified by column chromatography over silica gel using 0-20% ethyl acetate in n-hexane. |

### Step-4

To a stirred solution of carboxylic acid from step-3 in dichloromethane (DCM) or DMF (dimethylformamide), EDCI (1-ethyl-3-(3-dimethylaminopropyl)carbodiimide), optionally hydroxybenzotriazole (HOBt), and 4-dimethylaminopyridine (DMAP) or N,N-diisopropylethylamine (DIPEA) was added. The solution was stirred for 15 minutes at 0° C. followed by addition of desired tert-butyl 3-(piperidin-4-yl)benzylcarbamate. Stirring was continued at room temperature and reaction was monitored by LCMS until the maximum amount of starting materials was consumed. The reaction mixture was then quenched with water. The aqueous layer was extracted with dichloromethane and the combined organic layers were dried over sodium sulphate and concentrated under vacuum to afford the product. The product was used to for carrying out the next step without purification.

The compounds synthesized by Step-4 and the corresponding reaction conditions are shown in the table below:

| Compound | | |
|---|---|---|
| No. | Structure | Reaction conditions |
| E-31 | 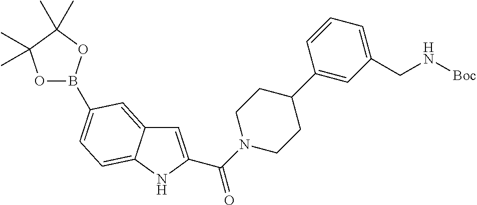 [SMILES: CC(C)(C)OC(=O)NCc1cccc(C2CCN(C(=O)c3cc4cc(B5OC(C)(C)C(C)(C)O5)ccc4[nH]3)CC2)c1] | tert-butyl 3-(piperidin-4-yl) benzyl carbamate (1.0 eq.), EDCI (1.5 eq.), HOBt (1.5 eq.), DIPEA (2.5 eq.), DMF, room temperature for 15 hours, yield 57%. |
| E-62 | 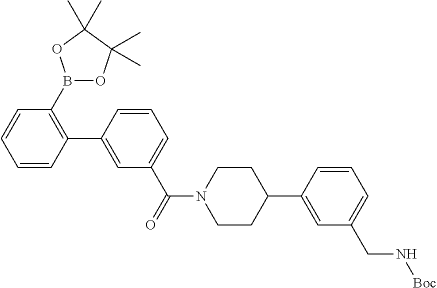 [SMILES: CC(C)(C)OC(=O)NCc1cccc(C2CCN(C(=O)c3cccc(-c4ccccc4B4OC(C)(C)C(C)(C)O4)c3)CC2)c1] | tert-butyl 3-(piperidin-4-yl) benzyl carbamate (1.1 eq.), EDCI (1.5 eq.), DMAP (0.5 eq.), DCM (125 vol.), room temperature for 24 hours, yield 48%. |
| E-35 | 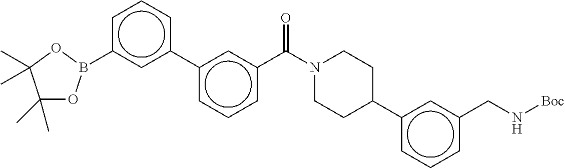 | tert-butyl 3-(piperidin-4-yl) benzyl carbamate (1.1 eq.), EDCI (1.5 eq.), DMAP (1.2 eq.), DCM (100 vol.), room temperature for 4 hours, yield 90%. Crude product used for next step. |
| E-11F | 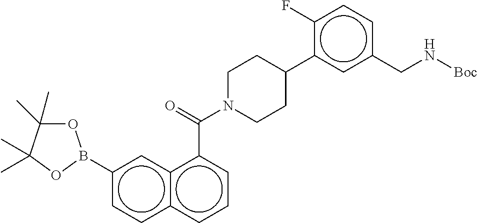 | tert-butyl 4-fluoro-3-(piperidin-4- yl) benzyl carbamate (1.3 eq.), EDCI (1.5 eq.), DMAP (1.2 eq.), DCM (100 vol.), room temperature for 4 hours, yield 86%. Crude product used for next step. |
| E-35F | 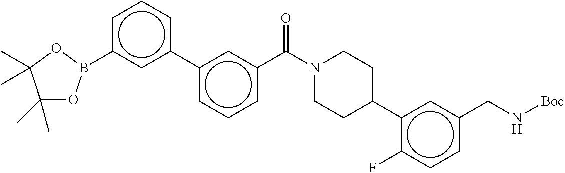 | tert-butyl 4-fluoro-3-(piperidin-4- yl)benzylcarbamate (1.2 eq.), EDCI (1.5 eq.), DMAP (1.2 eq.), DCM (100 vol.), room temperature for 4 hours, yield 92%. Crude product used for next step. |

### Step-5:

Products from Step-4 were stirred with aqueous hydrochloric acid or trifluoracetic acid (TFA) in a co-solvent such as acetonitrile, methanol, THF, and DCM. The reaction was monitored by LCMS until the maximum amount of starting materials were consumed. Reacted material was then concentrated in vacuum to remove the solvents. The residue obtained was purified by reverse phase preparative HPLC. The pure fraction of mobile phase was lyophilized to obtain the products as TFA salts. TFA salts were converted to hydrochloride salts by stirring with 2N HCl for 30 minutes under nitrogen atmosphere followed by lyophilization.

In some instances, only Boc deprotection was observed to be taking place with boronate ester functionality intact. In such cases, further hydrolysis of isolated Boc de-protected boronate esters was carried out followed by purification using preparative HPLC.

The compounds synthesized by Step-5, the corresponding reaction conditions, and analytical results are shown in the table below:

| Compound | | Reaction | |
|---|---|---|---|
| No. | Structure | conditions | Analytical data |
| Target-31 | 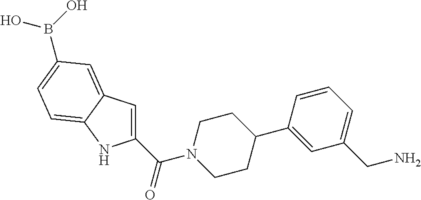 [SMILES: NCc1cccc(C2CCN(C(=O)c3cc4cc(B(O)O)ccc4[nH]3)CC2)c1] | Concentrated HCl (8.0 eq.) MeOH, 15 hours at room temperature, followed by preparative HPLC. Isolated as TFA salt of boronate ester, 50% Converted to hydrochloride by aqueous HCl (4.0 eq.), 4 hours at room temperature and lyophilization, yield 60% | Mol. Wt:-377.24 LCMS (m/z): 378 [M + l]; HPLC Purity: 93.98% ¹H NMR (400 MHz, D₂O): δ 8.22-8.12 (m, 1H), 7.54-7.28 (m, 6H), 7.04-6.96 (m, 1H), 4.66-4.52 (m, 2H), 4.24-4.10 (m, 2H), 3.50-3.34 (m, 1H), 3.20-2.94 (m, 2H), 2.10-1.70 (m, 4H). |
| Target-62 | 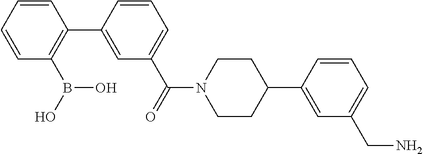 [SMILES: NCc1cccc(C2CCN(C(=O)c3cccc(-c4ccccc4B(O)O)c3)CC2)c1] | HCl (5.7 vol.), MeOH (85 vol.), 24 hours at room temperature, followed by preparative HPLC. Isolated as TFA salt, yield 26%. | Mol. Wt:-414.3 M.I. Peak observed: 415.4 ¹H NMR DMSO-d6:- ¹HNMR (400 MHz, DMSO) 1.50-1.95 (br, 4H), 2.80-2.90 (m, 1H), 3.20-3.40 (m, 4H), 3.84 (brs, 1H), 3.95-4.10 (m, 2H), 4.65 (brs, 1H), 7.25-7.55 (m, 10H), 8.00 (s, 2H), 8.10 (brs, 2H) |
| Target-35 | 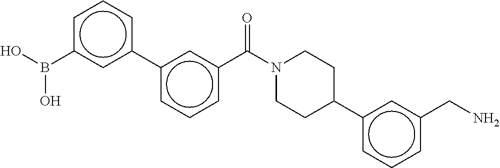 | Concentrated HCl (10 vol.), THF (66 vol.), 15 hours at room temperature, yield 14.4% | Mol. Wt:- 414.30 M.I. Peak observed: 415.05 HPLC Purity:- 94.79% ¹H NMR DMSO-d6:- ¹HNMR (400 MHz, DMSO) 1.64-1.86 (m, 4H), 2.84-2.87 (m, 2H), 3.23 (m, 2H) 3.65-3.73 (m, 1H), 3.99- 4.01 (d, 2H), 4.69 (bs, 1H), 7.29-7.57 (m, 7H), 7.70- 7.80 (m, 4H), 8.15 (bs, 1H), 8.24 (bs, 1H). |
| Target-11F | 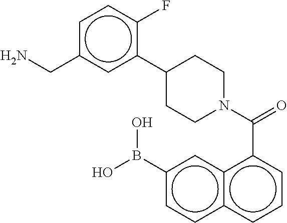 | Concentrated HCl (4 vol.), THF (66 vol.), 15 hours at room temperature, yield 12.7% | Mol. Wt:- 406.26 M.I. Peak observed: 407.30 HPLC Purity:- 96.62% ¹H NMR DMSO-d6:- ¹HNMR (400 MHz, DMSO) 1.15-1.91 (m, 4H), 2.97-3.47 (m, 3H), 3.64 (t, 1H) 4.01 (bs, 2H), 4.84, 4.87 (m, 1H), 7.21 (t, 1H) 7.37-7.61 (m, 4H), 7.93- 7.95 (m, 3H), 8.19-8.34 (m, 4H D₂O exchangable). |
| Target-35F | 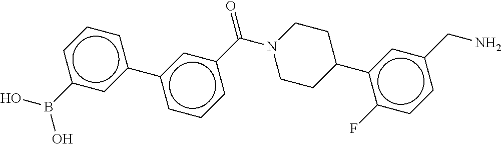 | Concentrated HCl (8 vol.), THF (25 vol.), 16 hours at room temperature, yield 25.7% | Mol. Wt:- 432.29 M.I. Peak observed: 433.40 HPLC Purity:- 98:83% ¹H NMR DMSO-d6:- ¹HNMR (400 MHz, DMSO) 1.69-1.84 (m, 4H), 2.93-3.19 (m, 3H), 3.74 (bs, 1H), 3.99-4.01 (q, 2H), 4.67 (bs, 1H), 7.21 (t, 1H), 7.34-7.47 (m, 3H), 7.54- 7.58 (m, 2H), 7.69-7.80 (m, 4H), 8.15 (bs, 2H), 8.21 (bs, 2H) |

### Step-6 & 7

Non-commercial aryl/hetero aryl carboxy boronic acids were synthesized from corresponding aryl halo carboxylic acids by reaction with LDA and tri-alkyl borate followed by hydrolysis using methods described in the literature. See, e.g., Example 20B in U.S. Patent Application Publication No. 2008/306082, which is incorporated hereby by reference in its entirety.

### Step-8

Coupling of the aryl boronic acids was carried out using the general procedures described in Step-4 above.

The compounds synthesized by Step-8, the corresponding reaction conditions, and analytical results are shown in the table below:

| Compound | | | |
|---|---|---|---|
| No. | Structure | Reaction conditions | Analytical data |
| H-33 | 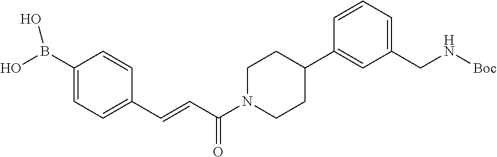 [SMILES: CC(C)(C)OC(=O)NCc1cccc(C2CCN(C(=O)/C=C/c3ccc(B(O)O)cc3)CC2)c1] | tert-butyl 3-(piperidin-4-yl) benzylcarbamate. (1.0 eq.), EDCI (1.5 eq.), HOBt (1.1 eq.), DMAP (1.1 eq.), DCM (100 vol.), DMF (2 vol.) room temperature for 2 hours, yield 88%. Crude product was used for next step. | Mol. Wt:- 464.36 M.I. Peak observed: 465.65 |
| H-34 | 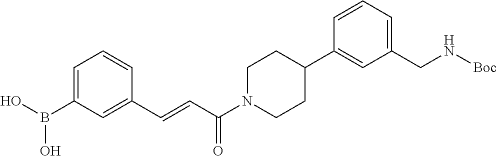 [SMILES: CC(C)(C)OC(=O)NCc1cccc(C2CCN(C(=O)/C=C/c3cccc(B(O)O)c3)CC2)c1] | tert-butyl 3-(piperidin-4-yl) benzylcarbamate. (1.0 eq.), EDCI (1.5 eq.), HOBt (1.1 eq.), DMAP (1.1 eq.), DCM (100 vol.), DMF (2 vol.), room temperature for 2 hours, yield 88%. Crude product was used for next step. | Mol. Wt:- 464.36 M.I. Peak observed: 464.85 |
| H-37 | 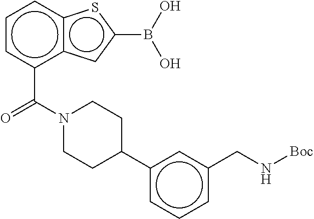 | tert-butyl 3-(piperidin-4-yl) benzylcarbamate. (1.1 eq.), EDCI (1.3 eq.), DMAP (2 eq.), DCM (50 vol.), room temperature for 2 hours, yield 50%. Crude product was used for next step. | Mol. Wt:- 494.41 M.I. Peak observed: 518.75 (M + Na) |

### Step-9

Products from Step-8 were stirred with trifluoro acetic acid in dichloromethane at room temperature and reactions were monitored by TLC & LCMS until the maximum amount of starting materials were consumed. Reacted material was concentrated in vacuum to remove excess trifluoro acetic acid and dichloromethane. Crude products obtained were purified by reverse phase preparative HPLC. The pure fraction of mobile phase was lyophilized to obtain the products as TFA salts. TFA salts were converted to hydrochloride salts by stirring with 2N HCl for 30 minutes under nitrogen atmosphere followed by lyophilization.

The compounds synthesized by Step-9, the corresponding reaction conditions, and analytical results are shown in the table below:

| Compound | | Reaction | |
|---|---|---|---|
| No. | Structure | conditions | Analytical data |
| Target-33 | 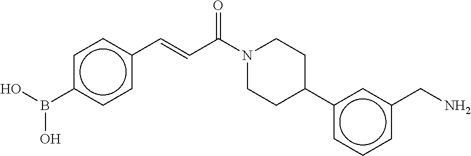 | TFA (1.5 eq), DCM (66 vol.), room temperature for 14 hours, yield 12%. | Mol. Wt: - 364.24 M.I. Peak observed: 364.90 HPLC Purity: - 97.22% ¹H NMR DMSO-d6: - ¹HNMR (400 MHz, DMSO) 1.53- 1.59 (m, 2H), 1.83 (t, 2H), 2.69- 2.88 (m, 2H), 3.20-3.23 (m, 1H), 3.97-4.01 (q, 2H), 4.42-4.47 (d, 1H), 4.64-4.67 (d, 1H), 7.27-7.39 (m, 4H), 7.48-7.52 (d, 2H), 7.67-7.69 (d, 2H), 7.80-7.82 (d, 2H), 8.23 (bs, 4H). |
| Target-34 | 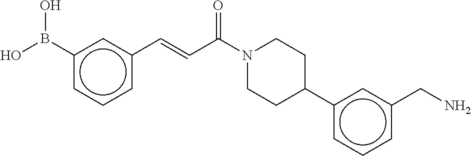 | TFA (1.5 eq), DCM (66 vol.), room temperature for 14 hours, yield 12%. | Mol. Wt: - 364.25 M.I. Peak observed: 364.90 HPLC Purity: - 95.01% ¹H NMR DMSO-d6: - ¹HNMR (400 MHz, DMSO) 1.57- 1.84 (m, 4H), 2.67-2.88 (m, 2H), 3.20-3.23 (m, 1H), 3.99-4.01 (q, 2H), 4.41-4.44 (d, 1H), 4.65- 4.68 (d, 1H), 7.27-7.39 (m, 6H), 7.48-7.52 (d, 2H), 7.76 (t, 2H), 8.08 (bs, 2H), 8.21 (bs, 2H). |
| Target-37 | 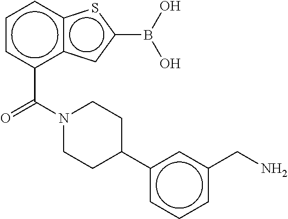 | HCl (10 vol.), THF (50 vol.), room temperature for 5 hours, yield 40%. | Mol. Wt: - 394.29 M.I. Peak observed: 395.00 HPLC Purity: - 97.24% ¹H NMR DMSO-d6: - ¹HNMR (400 MHz, DMSO) 1.49-1.91 (m, 4H), 2.81-3.19 (m, 4H), 3.42-3.55 (m, 2H), 3.99-4.00 (d, 2H), 4.78-4.80 (d, 1H), 7.30- 7.46 (m, 6H), 8.04-8.06 (d, 2H), 8.34 (bs, 2H). |

### Method B

Method B was carried out according to the following reaction scheme:

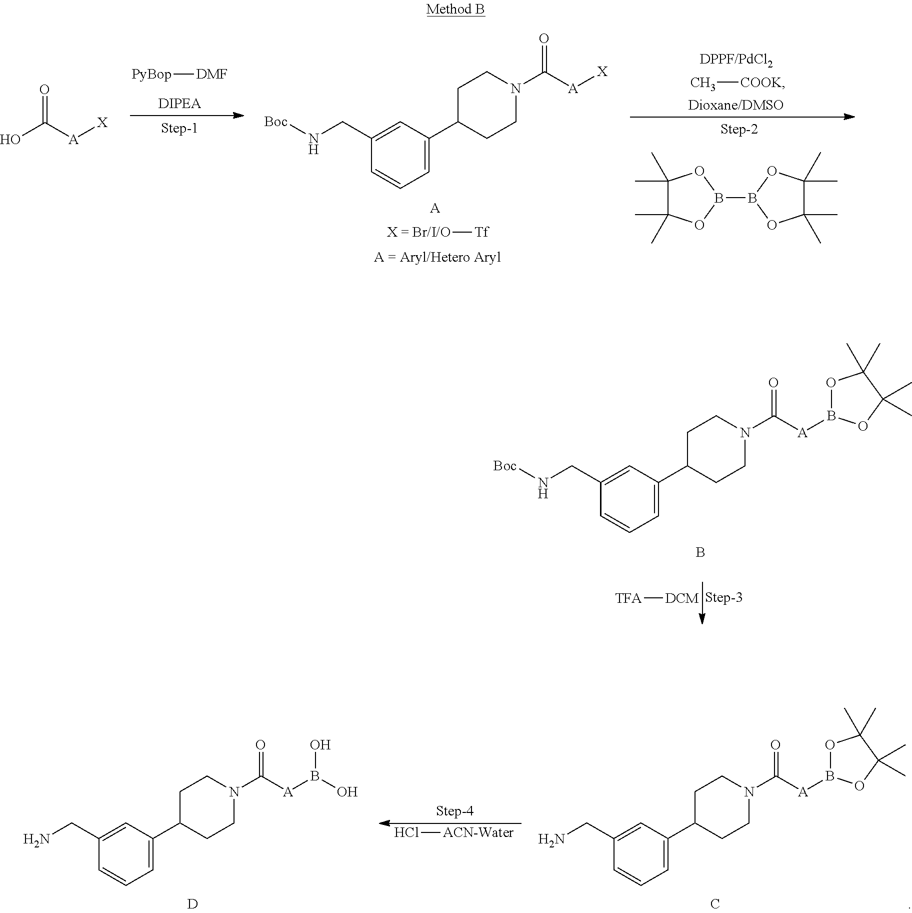

In general, halo aryl carboxylic acids were first coupled with tert-butyl 3-(piperidin-4-yl)benzylcarbamate. The coupled products were reacted with bis pinacolato diborane to obtain desired boronate esters, which were then hydrolyzed to corresponding boronic acids.

### Step-1:

Tert-butyl 3-(piperidin-4-yl)benzylcarbamate and desired aryl halo carboxylic acids were stirred with PyBop and diisopropyl ethyl amine in DMF for 24 hours at room temperature. The reaction mixture was then quenched with water and extracted with ethyl acetate. Ethyl acetate extract was dried over sodium sulfate and concentrated to obtain the crude product which was purified by column chromatography.

The compounds synthesized by Step-1 and the corresponding reaction conditions are shown in the table below:

| Compound | | |
|---|---|---|
| No. | Structure | Reaction conditions |
| A-32 | 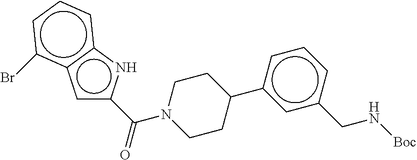 | tert-butyl 3-(piperidin-4-yl) benzyl carbamate (1 eq.) Py Bop (2 eq.) in DMF (30 vol.) & DIPEA (2.5 eq.), 24 hours at room temperature, yield 93%. |
| A-59 | 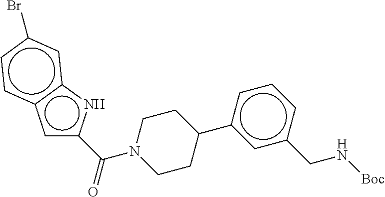 | tert-butyl 3-(piperidin-4-yl) benzyl carbamate (1 eq) Py Bop (2 eq.) in DMF (30 vol.) & DIPEA (2.5 eq.), 24 hours at room temperature, yield 71%. |
| A-56 | 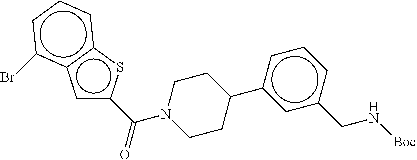 | tert-butyl 3-(piperidin-4-yl) benzyl carbamate (1 eq.) Py Bop (2 eq:) in DMF (10 vol.) & DIPEA (2.5 eq.), 24 hours at room temperature, yield 65%. |

### Step-2:

Product from Step-1 was converted to boronate ester by reacting with bis pinacolato borane in the presence of potassium acetate DPPF-PdCl₂.DCM and heated in 1,4-dioxane/dimethyl sulfoxide for 12 hours. The reacted material was then concentrated in vacuum and residue was purified by column chromatography.

The compounds synthesized by Step-2 and the corresponding reaction conditions are shown in the table below:

| Compound | | |
|---|---|---|
| No. | Structure | Reaction conditions |
| B-32 | 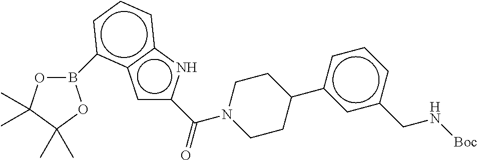 | KOAc (3 eq.), Bis Pin. Borane (10 eq.), DPPF-PdCl₂•DCM (mol. 6%), dioxane (40 vol.), reflux for 12 hours. Inorganics removed by column chromatography, and carried forward to next step. |
| B-59 | 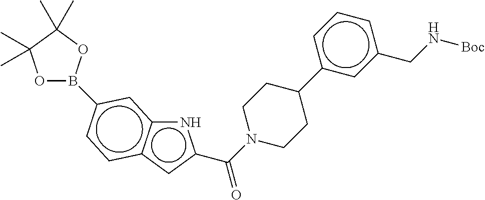 | KOAc (3 eq.), bis Pin. Borane (10 eq.), DPPF-PdCl₂•DCM (mol. 3%), DMSO (35 vol.), 80° C., 12 hours, purified column chromatography, yield 59%. |
| C-56 | 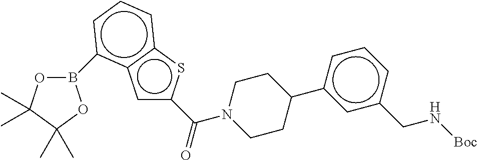 | KOAc (3 eq), bis Pin. Borane (10 eq.), DPPF-PdCl₂•DCM (mol. 3%), dioxane (200 vol.), 110° C. for 12 hours, purified by column chromatography, yield 74%. |

### Step-3:

Products from Step-2 were stirred with trifluoro acetic acid in dichloromethane at room temperature. The reacted material was then concentrated in vacuum and used for carrying out the next step without purification.

The compounds synthesized by Step-3 and the corresponding reaction conditions are shown in the table below:

| Compound | | |
|---|---|---|
| No. | Structure | Reaction conditions |
| C-32 | 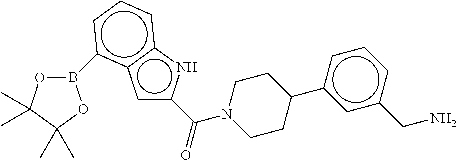 | TFA (3 vol.) DCM (100 vol.), room temperature for 24 h, subjected to next step without purification. |
| C-59 | 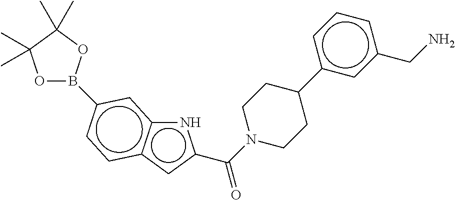 | TFA (2 vol.) DCM (200 vol.), room temperature for 24 hours, subjected to next step without purification. |
| C-56 | 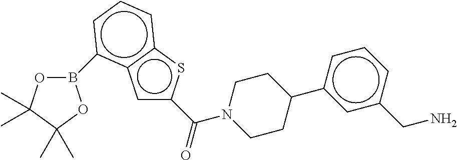 | TFA (7.5 vol.) DCM (100 vol.), room temperature for 24 hours, subjected to next step without purification. |

### Step-4:

Products from Step-3 were stirred with concentrated HCl, acetonitrile and water for about 5 hr under nitrogen atmosphere. The reacted material was concentrated in vacuum, and crude boronic acid was purified by preparative HPLC. Products were isolated either as TFA salts or acetate salts depending on the buffer used during purification by preparative HPLC.

The compounds synthesized by Step-4, the corresponding reaction conditions, and analytical results are shown in the table below:

| Compound | | | |
|---|---|---|---|
| No. | Structure | Reaction conditions | Analytical data |
| Target-32 | 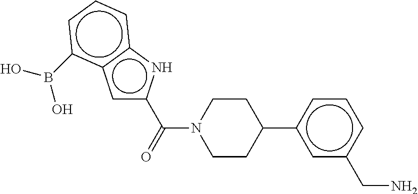 | Concentrated HCl (3 eq.) ACN:water (1:1 200 vol.), room temperature for 5 hours, and preparative HPLC purification isolated as acetate salt, yield 41%. | Mol. Wt: - 377.2 M.I. Peak observed: 378 HPLC Purity: - 96.55% (220 nm) ¹H NMR CD₃CN + D₂O: - ¹HNMR (400 MHz, CD₃CN + D₂O) 1.80-1.91 (m, 2H), 1.94 (s, 3H, acetate), 2.05 -2.15 (m, 2H), 3.00-3.10 (m, 1H), 3.20-3.50 (brm, 2H), 4.15 (s, 2H), 4.80 (brd, 2H), 7.35-7.52 (m, 6H), 7.65 (d, J = 6.8 Hz, 1H), 7.70 (d, J = 8.0 Hz, 1H). |
| Target-59 | 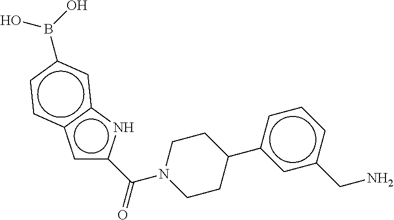 | Concentrated HCl (3 eq.) ACN:water (1:1 200 vol.), room temperature for 5 hours, and preparative HPLC purification isolated as acetate salt, yield 17%. | Mol. Wt: - 377.2 M.I. Peak observed: 378 HPLC Purity: - 97.3% (220 nm) ¹H NMR CD₃CN + D₂O: - ¹HNMR (400 MHz, CD₃CN + D₂O) 1.80-1.90 (m, 2H), 1.94 (s, 3H, acetate), 2.05-2.15 (m, 2H), 3.00-3.10 (m, 1H), 3.20-3.50 (brm, 2H), 4.17 (s, 2H), 4.75 (brd, 2H), 6.97 (s, 1H), 7.35- 7.52 (m, 4H), 7.61 (d, J = 8.0 Hz, 1H), 7.75 (d, J = 8.0 Hz, 1H), 8.06 (s, 1H) |
| Target-56 | 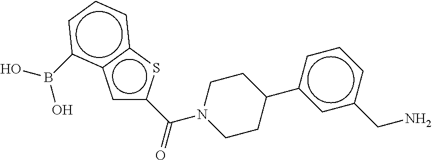 | Concentrated HCl (3 eq.) ACN:water (1:1 200 vol.), room temperature for 5 hours, and preparative HPLC purification isolated as TFA salt, yield 15%. | Mol. Wt: - 394.3 M.I. Peak observed: 395 HPLC Purity: - 97.37% (220 nm) ¹H NMR DMSO-d6: - ¹HNMR (400 MHz, DMSO) 1.60-1.95 (br, 4H), 2.85-2.95 (m, 1H), 3.25-3.40 (br, 4H), 3.95- 4.10 (m, 2H), 4.30-4.70 (br, 2H), 7.22-7.48 (m, 5H), 7.83 (d, J = 6.8 Hz, 1H), 8.05 (d, J = 8.0 Hz, 1H), 8.15 (s, 1H), 8.41 (br, 2H). |

### Example 2

### Synthesis of Cofluoron Monomers Bearing Phenolic Hydroxy Functionality

The cofluoron monomers bearing phenolic hydroxy moieties were synthesized by Method C or Method D as below.

### Method C

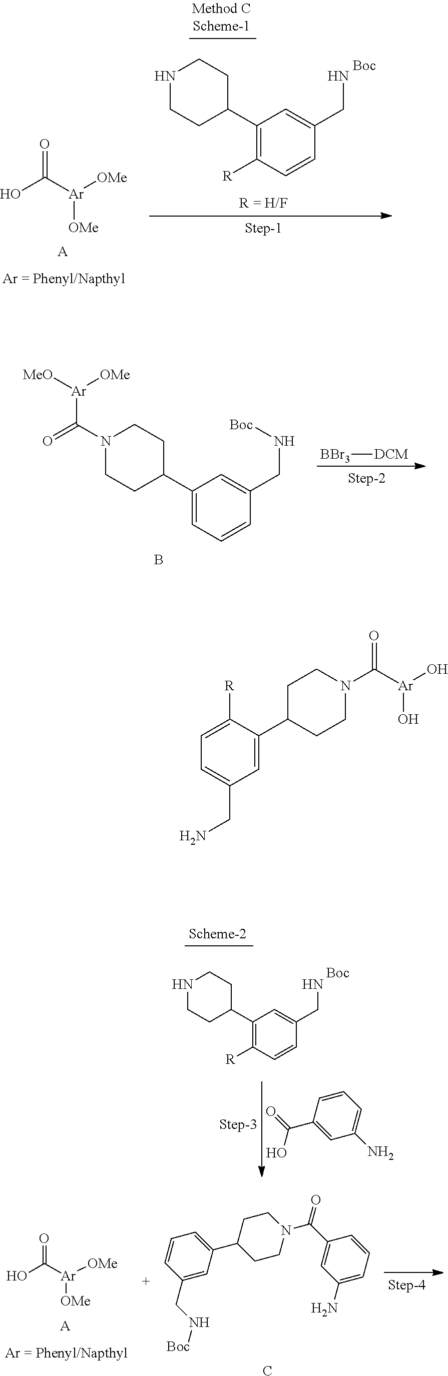

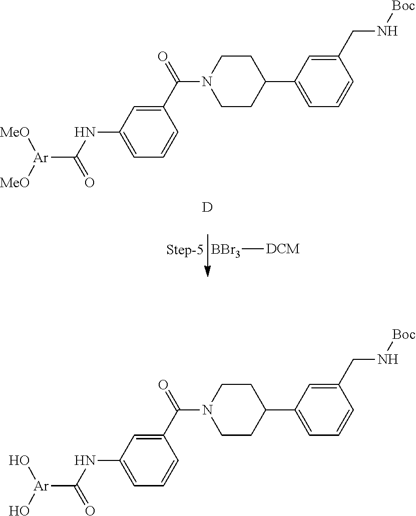

In general, desired dimethoxy analogues of carboxylic acids were first coupled with tert-butyl 3-(piperidin-4-yl)benzylcarbamate and coupled products were de-methylated using boron tribromide.

2-(6-oxo-6H-[1,3]dioxolo[4,5-g]chromen-8-yl)acetic acid, required for Target 97 was synthesized by Pechmann reaction of sesamol & diethyl 3-oxopentanedioate using toluene as a solvent and following the procedure described in the literature for analogous substrate (Chemistry Letters 2: 110-111 (2001), which is hereby incorporated by reference in its entirety).

6,7-dimethoxy-2-oxo-2H-chromene-3-carboxylic acid & 7,8-dimethoxy-2-oxo-2H-chromene-3-carboxylic acid required for Target-100 and 102 were prepared by the reaction of meldrums acid with 2-hydroxy-4,5-dimethoxybenzaldehyde or 2-hydroxy-3,4-dimethoxybenzaldehyde in water at 75° C. for 2 hours. Precipitated products were sufficiently pure to be used for carrying out the next step. Aldehydes were prepared from corresponding trimethoxy benzaldehydes by de-methylation using AlCl₃ in benzene (J. Org. Chem. 54: 4112 (1989), which is hereby incorporated by reference in its entirety).

### Step-1:

The reactions were performed using the general procedure described in Method A (Step-4) or Method B (Step-1). Crude products were used for carrying out the next step without purification.

The compounds synthesized by Step-1 and the corresponding reaction conditions are shown in the table below:

| Compound No. | Structure | Reaction conditions |
|---|---|---|
| B-27-F | 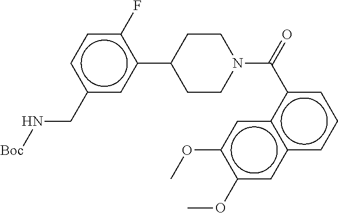 | Common core (1 eq.), EDCI (1.5 eq.), DMAP (1.2 eq.), DCM (85 vol.), room temperature for 4 hours, yield 89%. |
| B-68 | 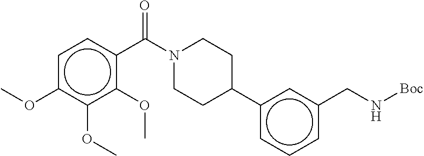 | Common core (1.1 eq.), EDCI (1.5 eq.), DMAP (1.2 eq.), DCM (85 vol.), room temperature, 4 hours, yield 93%. |
| B-69 | 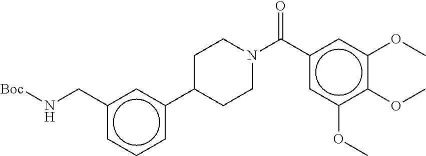 | Common core (1.1 eq.), EDCI (1.5 eq.), DMAP (1.2 eq.), DCM (85 vol.), room temperature for 4 hours, yield 96%. |
| B-77 | 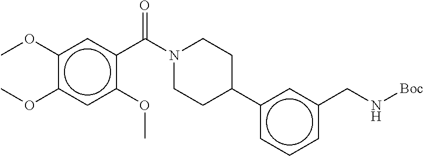 | Common core (1.1 eq.), EDCI (1.5 eq.), DMAP (1.2 eq.), DCM (85 vol.), room temperature for 4 h, yield 89.4%. |
| B-43 | 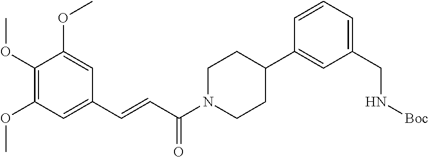 [SMILES: COc1cc(/C=C/C(=O)N2CCC(c3cccc(CNC(=O)OC(C)(C)C)c3)CC2)cc(OC)c1OC] | (E)-3-(3,4,5-trimethoxy phenyl) acrylic acid (1 eq.), tert-butyl 3-(piperidin-4-yl) benzylcarbamate (1 eq.), PyBop (2 eq.), DIPEA (2.5 eq.), DMF (5 vol.), 24 hours at room temperature, yield 63%. |
| B-97 | 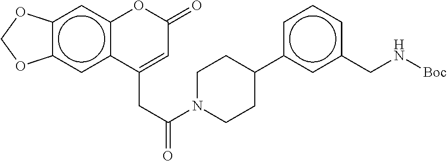 | 2-(6-oxo-6H-[l,3]dioxolo[4,5- g]chromen-8-yl)acetic acid (1 eq) tert- butyl 3-(piperidin-4-yl) benzylcarbamate (1 eq.), EDCI (2 eq.), DMAP (0.5 eq.), DCM (20 vol.), 12 hours at room temperature, yield 86%. |
| B-100 | 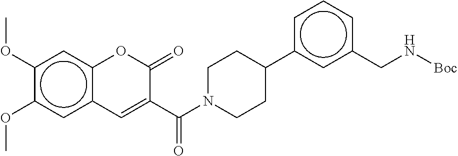 | 6,7-dimethoxy-2-oxo-2H-chromene-3- carboxylic acid (1 eq) tert-butyl 3- (piperidin-4-yl) benzylcarbamate (1 eq.), EDCI (1.5 eq.), DMAP (0.5 eq.), DCM (100 vol.), 12 hours at room temperature, yield 80%. |
| B-102 | 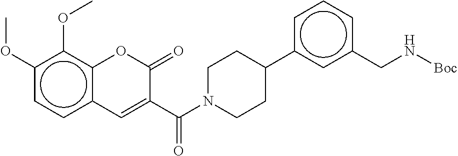 | 7,8-dimethoxy-2-oxo-2H-chromene-3- carboxylic acid (1 eq.) tert-butyl 3- (piperidin-4-yl) benzylcarbamate (1.2 eq.), EDCI (1.5 eq.), DMAP (0.5 eq.), DCM (66 vol.), 12 hours at room temperature, yield 80.6%. |

### Step-2

Product from Step-1 was dissolved in dichloromethane and the solution was cooled to 0° C. Boron tribromide (3 eq.) was added, and the reacted product was gradually warmed to room temperature. Stirring was continued at room temperature and reaction was monitored by TLC & LCMS until the maximum amount of starting materials was consumed (1-8 hours required). The reacted material was then concentrated, and excess BBr3 was removed by multiple strippings of methanol. Residue containing crude product as hydrobromide was purified by reverse phase preparative HPLC. Pure product isolated as TFA salts were converted to hydrochloride by dissolving in 2N hydrochloric acid followed by lyophilization to obtain the corresponding compounds as hydrochloride salts.

The compounds synthesized by Step-2, the corresponding reaction conditions, and analytical results are shown in the table below:

| Compound | | | |
|---|---|---|---|
| No. | Structure | Reaction conditions | Analytical data |
| Target-27-F | 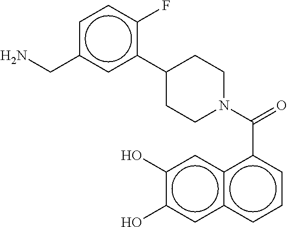 | BBr₃ (3 eq.) DCM (85 vol.), room temperature for 2 hours, yield 16% | Mol. Wt: - 394.43 M.I. Peak observed: 395.30 HPLC Purity: - 96.75 ¹H NMR DMSO-d6: - ¹HNMR (400 MHz, DMSO) 1.53- 1.88 (m, 4H), 2.91-3.33 (m, 4H), 4.0 (bs, 2H), 4.82 (m, 1H), 6.99 (s, 1H), 7.15- 7.36 (m, 5H), 7.59-7.64 (m, 2H), 7.82-7.84 (d, 1H-D₂O exchangable), 8.34-8.43 (m, 2H- D₂O exchangable). |
| Target-68 | 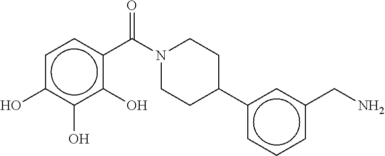 | BBr₃ (3 eq.) DCM (85 vol.), room temperature for 2 hours, yield 22% | Mol. Wt: - 342.38 M.I. Peak observed: - 343.20 HPLC Purity: - 97.94 ¹H NMR CD₃OD: - 1.75- 1.94 (m, 4H), 2.90-3.31 (m, 4H), 4.10 (s, 2H), 4.35 (bs, 1H), 6.43 (d, 1H, J = 8.4 Hz), 6.66 (d, 1H, J = 8.4 Hz), 7.29- 7.41 (m, 4H). |
| Target-69 | 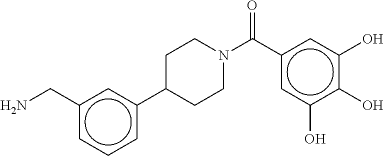 | BBr₃ (3 eq) DCM (85 vol.), room temperature for 2 hours,, yield 23.6% | Mol. Wt: - 342.38 M.I. Peak observed: - 343.25 HPLC Purity: - 97.41 ¹H NMR CD₃OD: - 1.75- 1.94 (m, 4H), 2.90-3.31 (m, 4H), 4.10 (s, 2H), 6.47 (s, 2H), 7.03- 7.38 (m, 4H). |
| Target-77 | 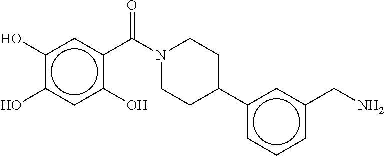 | BBr₃ (3 eq.) DCM (85 vol), room temperature for 2 hours, yield 22.7% | Mol. Wt: - 342.38 M.I. Peak observed: - 343.25 HPLC Purity: - 99.73 ¹H NMR DMSO-d6: - 1.54- 1.60 (m, 2H), 1.74-1.77 (d; 4H), 2.75-2.96 (m, 4H), 3.96- 4.14 (m, 2H), 6.34 (s, 1H), 6.54 (s, 1H), 7.24-7.39 (4H), 8.34 (bs, 3H). |
| Target-43 | 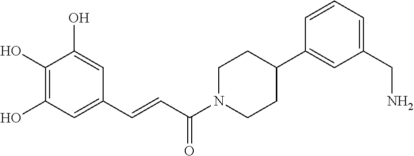 [SMILES: NCc1cccc(C2CCN(C(=O)/C=C/c3cc(O)c(O)c(O)c3)CC2)c1] | BBr3 (1 M in DCM, 5 eq.), DCM (100 vol.), 24 hours at room temperature, yield 15%. | Mol. Wt: - 368.43 M.I. Peak observed: - 369 HPLC Purity: - 99.49% ¹H NMR (400 MHz, DMSO- d6): δ 1.55 (brs, 2H), 1.80 (brs, 2H), 2.79-2.88 (m, 1H), 3.10-3.40 (br, 2H), 3.95-4.20 (m, 2H), 4.30-4.70 (br, 2H), 6.62 (s, 2H), 6.89 (d, J = 15.2 Hz, 1H), 7.20-7.40 (m, 5H), 8.11 (brs, 2H), 8.64 (brs, 1H), 8.97 (brs, 2H). |
| Target-97 | 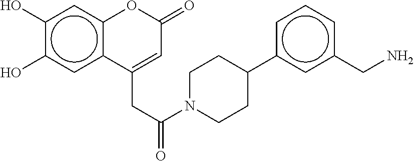 | BBr3 (1 M in DCM, 4 eq.), DCM (66 vol.), 12 hours at room temperature, yield 15%. Isolated as TFA salt, yield 20%. | Mol. Wt: - 408.45 M.I. Peak observed: - 409 HPLC: 98.37% (220 nm) ¹H NMR (400 MHz, CD₃OD): δ 1.60-2.0 (m, 4H), 2.78-3.0 (m, 2H), 3.30-3.45 (brm, 1H, merged in solvent peak), 3.90- 4.10 (m, 2H), 4.11 (s, 2H), 4.12 (br, 1H), 4.60-4.80 (brd, 1H), 6.14 (s, 1H), 6.78 (s, 1H), 7.04 (s, 1H), 7.28-7.50 (m, 4H). |
| Target-100 | 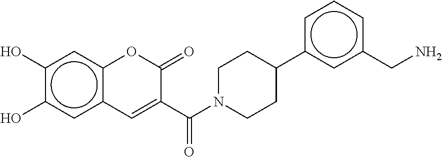 | BBr3 (1 M in DCM, 4 eq.), DCM (100 vol.), 12 hours at room temperature, isolated as TFA salt, yield 19.4%. | Mol. Wt: - 394.42 M.I. Peak observed: - 395.25 HPLC: 98.83% (220 nm) ¹H NMR (400 MHz, CD3OD): δ 1.70-2.00 (m, 4H), 2.85- 3.00 (m, 2H), 3.75-3.85 (brd, 1H), 4.10 (s, 2H), 4.70-4.80 (brd, 2H), 6.80 (s, 1H), 7.02 (s, 1H), 7.26-7.44 (m, 4H), 7.95 (s, 1H). |
| Target-102 | 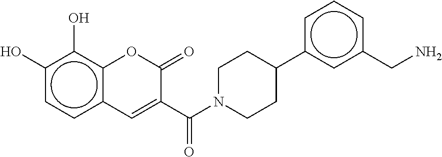 | BBr3 (1 M in DCM, 4 eq.), DCM (80 vol.), 12 hours at room temperature, isolated as TFA salt, yield 25%. | Mol. Wt: - 394.42 M.I. Peak observed: - 395.25 HPLC: 99.27% (220 nm) ¹H NMR (400 MHz, CD₃OD): δ 1.77-1.99 (m, 4H), 2.92-2.98 (m, 2H), 3.31 (brs, 1H merged in solvent peak), 3.81-3.85 (m, 1H), 4.11 (brs, 2H), 4.76 (brs, 1H, merged in solvent water peak), 6.87 (d, J = 8.4 Hz, 1H), 7.09 (d, J = 8.4 Hz, 1H), 7.29-7.42 (m, 4H), 7.98 (brs, 1H) |

### Step-3

Reaction was performed as per general procedure described in Method A (Step-4).

The compounds synthesized by Step-3 and the corresponding reaction conditions are shown in the table below:

| Compound No. | Structure | Brief Reaction conditions |
|---|---|---|
| C | 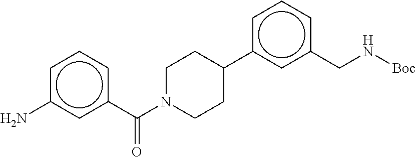 | tert-butyl 3-(piperidin-4-yl) benzyl carbamate (1 eq.), EDCI (1.5 eq.), DMAP (1.2 eq.), DCM (85 vol), room temperature for 4 hours, yield 99%. |

### Method D

Method D was carried out according to the following reaction scheme:

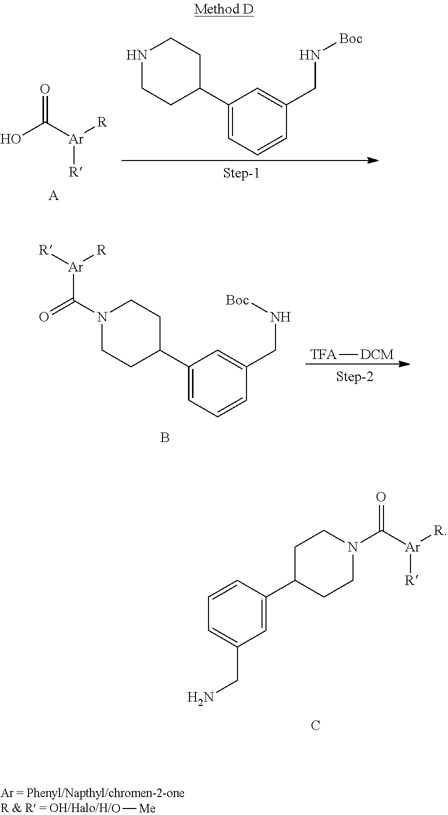

In general, desired carboxylic acid (A) was coupled with tert-butyl 3-(piperidin-4-yl)benzylcarbamate followed by deprotection of Boc functionality.

2-(7,8-dihydroxy-4-methyl-2-oxo-2H-chromen-3-yl)acetic acid required for Target-101 was synthesized by the Pechmann reaction of pyrogallol & diethyl acetyl succinate using toluene as a solvent and following the procedure described in the literature for analogous substrate, i.e., resorcinol (Chemistry Letters 2: 110-111 (2001), which is hereby incorporated by reference in its entirety).

Some halo analogues of the Boronic acids in Method A were also synthesized by this approach.

### Step-1

The reactions were carried out as general procedure in Method A (Step-4). Products were purified by column chromatography over silicagel using methanol (0-5%) in chloroform.

The compounds synthesized by Step-1 and, the corresponding reaction conditions are shown in the table below:

| Compound. | | |
|---|---|---|
| No. | Structure | Brief Reaction conditions |
| B-101 | 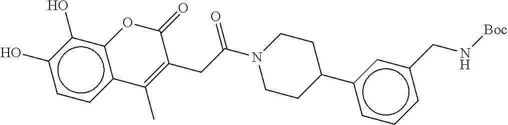 | 2-(7,8-dihydroxy-4-methyl-2-oxo- 2H-chromen-3-yl)acetic acid (1 eq.) tert-butyl 3-(piperidin-4-yl) benzyl carbamate (1.2 eq.), EDCI (1.2 eq.), HOBt (1.5 eq.), DIPEA (1.5 eq.), DMF (50 vol.), room temperature for 12 hours, yield 30%. |

### Step-2

Boc deprotection of the products from Step-1 was carried out by stirring the products with hydrochloric acid in the presence of co-solvent, such as methanol or dioxane, at room temperature. Solvents were then evaporated and residue was purified by reverse phase preparative HPLC. Products were isolated as TFA salts.

The compounds synthesized by Step-2, the corresponding reaction conditions, and analytical results are shown in the table below:

| Compound | | Brief Reaction | |
|---|---|---|---|
| No. | Structure | conditions | Analytical data |
| Target-101 | 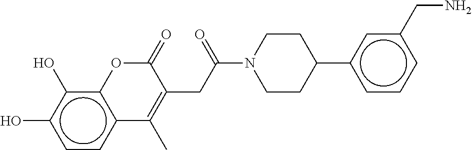 | concentrated HCl (10 V), methanol (100 V), 24 hours, room temperature, Isolated as hydrochloride in pure form after work-up Yiled:- 81.3% | Mol. Wt.: 422.47 LCMS: (M + l) 423.2 HPLC Purity: - 95.30% ¹H NMR (400 MHz, DMSO- d6): δ 1.40-1.90 (m, 4H), 2.28 (s, 3H), 2.60-2.39 (m, 2H), 3.16-3.27 (m, 1H), 4.00 (d, 2H), 4.05 (s, 2H), 4.21 (brd, 1H), 4.51 (brd, 1H), 6.84 (d, J = 8.8 Hz, 1H), 7.13 (d, J = 8.8 Hz, 1H), 7.25- 7.43 (m, 4H), 8.36 (br, 2H) |

### Example 3

### Synthesis of Cofluoron Monomers Bearing 1-Amido Phenols Functionality

### Method F

The cofluoron monomers bearing phenolic hydroxy moieties were synthesized by Method F as below, according to the following reaction scheme:

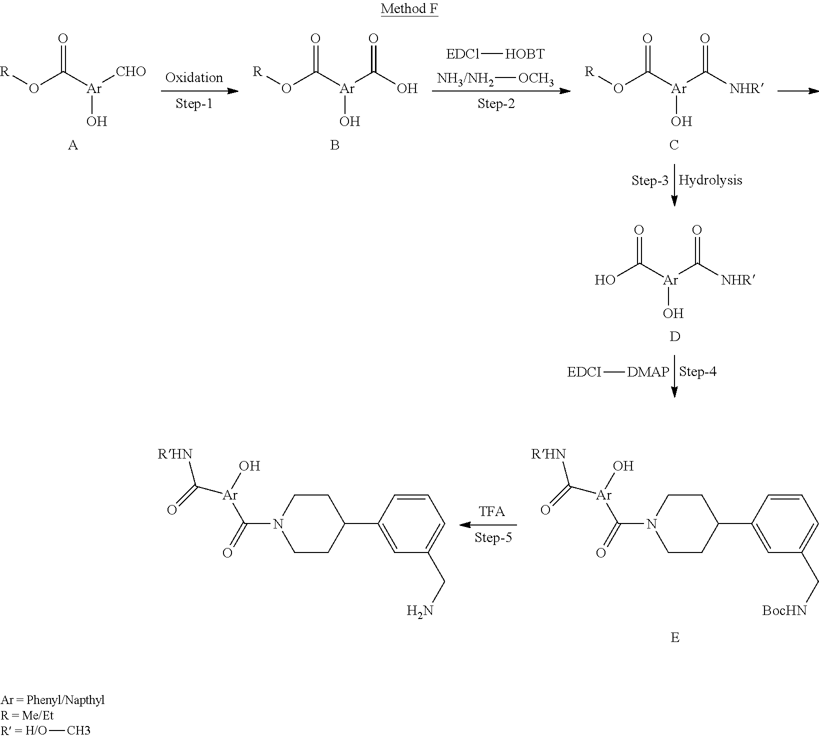

In general, ortho hydroxy aromatic aldehydes, with carbethoxy or methoxy functionality at suitable positions, were oxidized to obtain o-carboxy phenols which were then converted to amide by reaction either with ammonia/o-methyl hydroxyl amine. Ester functionality was then hydrolyzed to obtain corresponding o-hydroxy amido carboxylic acid, which upon coupling with tert-butyl 3-(piperidin-4-yl)benzylcarbamate and subsequent deprotection in acidic media produced the desired compounds.

### Step-1

Carbmethoxy hydroxy benzaldehyde was dissolved in acetonitrile and aqueous solution of di-sodium hydrogen phosphate, and 30% hydrogen peroxide was then added. The reacted material was then cooled to 0° C., and aqueous sodium chlorite was added to the reacted material drop-wise. The reacted material was allowed to warm to room temperature and stirred. Reaction was monitored by LCMS until maximum amount of starting materials was consumed. The reaction product was then concentrated, residue was acidified with aqueous HCl, and product was extracted in ethyl acetate. Ethyl acetate extract was dried over sodium sulfate and concentrated to obtain the crude product, which was sufficiently pure for carrying out the next step.

The compounds synthesized by Step-1 and the corresponding reaction conditions are shown in the table below:

| Compound No. | Structure | Reaction conditions |
|---|---|---|
| B-75 | 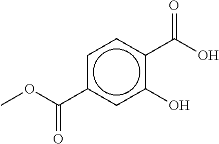 | 4-Formyl, 3-hydroxy methyl benzoate, (1 eq.) ACN (65 vol.), NaH₂PO₄•2H₂O (0.32 eq. in 11 vol. water), H₂O₂ 30% solution (5 eq.) NaClO₂ (1.4 eq. in 10 vol. water), room temperature for 2 hours, yield- 35.46%. |
| B-66 | 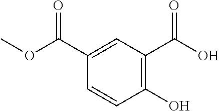 | 3-Formyl, 4-hydroxy methyl benzoate ACN (37.5 vol.), NaH₂PO₄•2H₂O (0.32 eq. in 11 vol. water), H₂O₂ 30% solution (5 eq.) NaClO2 (1.4 eq. in 10 vol. water), room temperature for 2 hours, yield 53.4%. |

### Step-2

Products from Step-1 were converted to desired amides either by conversion to acid chloride and subsequent reaction with ammonia/desired amine or by coupling reaction using EDCI-HOBT in DMF followed by the general procedures as described in Method A (Step-4). Crude products were purified by column chromatography over silica gel using methanol (0-30%) in chloroform.

Intermediates C-76 and D-76 were synthesized by the Heck reaction of the corresponding o-hydroxy-4-bromo benzamides (which was synthesized from 4-bromo-2-hydroxybenzoic acid using the general procedure described above) using ethyl acrylate and following the procedure described in the literature for analogous compounds (Bull. Korean Chem. Soc., 20: 232-236 (1999), which is hereby incorporated by reference in its entirety).

Intermediate C-86 was synthesized by the procedure described in the literature (J. Med. Chem. 43: 1670-1683 (2000), which is hereby incorporated by reference in its entirety).

The compounds synthesized by Step-2 and the corresponding reaction conditions are shown in the table below:

| Compound No. | Structure | Reaction conditions |
|---|---|---|
| C-75 | 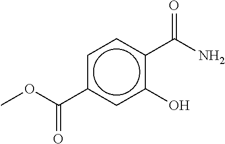 | EDCI (1.1 eq.), HOBT (1.1 eq.), aq ammonia (4 eq.), DMF (60 vol.), room temperature for 14 hours, purified by column chromatography (0-30% methanol-chloroform), yield 10%, |
| C-75a | 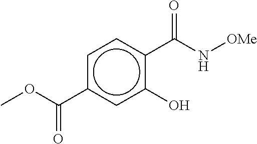 | B-75 (1 eq.), DCM (75 vol.), TEA (1.5 eq.) thionyl chloride (1.5 eq.) 0° C. for 1 h. NH₂OMe•HCl (1.5 eq.) DCM (32 vol.), TEA (2 eq.) was added and stirred for 2 hours, purified by column chromatography (0-30% methanol- chloroform), yield 64%. |
| C-92 | 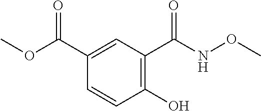 | B-66 (1 eq.), DCM (100 vol.), TEA (3 eq.) thionyl chloride (1.5 eq.) 0° C. for 1 h. NH₂OMe•HCl (1 eq.) DCM (32 vol.), TEA (2 eq.) was added and stirred for 2 h, yield 52.6%. |
| C-76 | 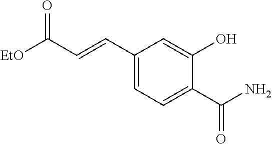 [SMILES: CCOC(=O)/C=C/c1ccc(C(N)=O)c(O)c1] | Bull. Korean Chem. Soc. 20: 232-236 (1999), which is hereby incorporated by reference in its entirety. White solid, yield 60%. |
| C-76a | 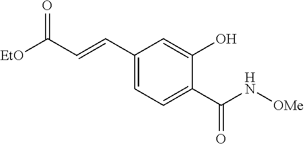 [SMILES: CCOC(=O)/C=C/c1ccc(C(=O)NOC)c(O)c1] | As above White solid; yield 93%. |
| C-86 | 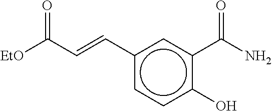 | J. Med. Chem. 43: 1670-1683 (2000), which is hereby incorporated by reference in its entirety. Yield 71.4%. |

### Step-3

Hydrolysis of Step-2 products was carried out using the general procedure followed in Method A (Step-3). Crude products were used for carrying out the next step unless specified.

The compounds synthesized by Step-3 and the corresponding reaction conditions are shown in the table below:

| Compound No. | Structure | Reaction conditions |
|---|---|---|
| D-75a | 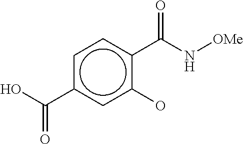 | LiOH (3.0 eq.), THF:H2O (1:1), room temperature, 4 hours, Yield: - 86%. |
| D-92 | 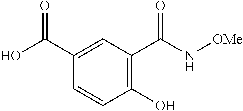 [SMILES: CONC(=O)c1cc(C(=O)O)ccc1O] | Acetone (25 Vol) 1 N NaOH (25 Vol), room temperature stir 12 hours. Crude product contaminated with Sodium chloride was used for next step without purification. |
| D-76 | 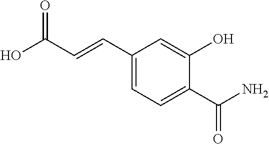 [SMILES: NC(=O)c1ccc(/C=C/C(=O)O)cc1O] | LiOH (4.0 eq.), MeOH:H2O (4:1), room temperature, 4 hours, Acidified with aq. Citric acid instead of HCl during work-up. Yield: - 75%. |
| D-76a | 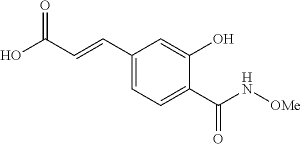 [SMILES: CONC(=O)c1ccc(/C=C/C(=O)O)cc1O] | LiOH (4.0 eq.), MeOH:H2O (4:1), room temperature, 4 hours, Acidified with aq. Citric acid instead of HCl during work-up. Yield: - 70%. |
| D-86 | 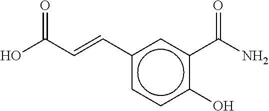 | Acetone (25 Vol) 1 N NaOH (25 Vol), room temperature stir 12 hours. Crude product was taken for further step. |

### Step-4

The coupling reactions of Step-3 products with tert-butyl 3-(piperidin-4-yl)benzylcarbamate were carried out using the general procedures described in Method-A (Step-4). Crude products were used for carrying out the next step without further purification, unless specified.

The compounds synthesized by Step-4 and the corresponding reaction conditions are shown in the table below:

| Compound No. | Structure | Reaction conditions |
|---|---|---|
| E-75a | 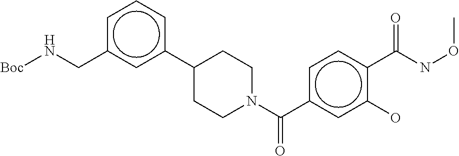 | tert-butyl-3-(piperidin-4-yl) benzylcarbamate (1.1 eq.), EDCI (1.5 eq.), DMAP (1.2 eq.), DCM (85 vol.), room temperature for 4 hours, yield 61%. |
| E-92 | 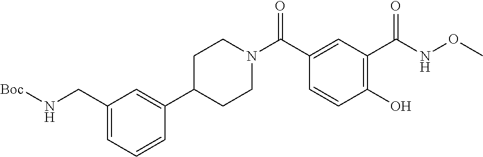 [SMILES: CONC(=O)c1cc(C(=O)N2CCC(c3cccc(CNC(=O)OC(C)(C)C)c3)CC2)ccc1O] | tert-butyl-3-(piperidin-4-yl) benzylcarbamate (1.2 eq.) DCM (100 vol.), DMAP (0.5 eq), EDCI (1.5 eq.), room temperature for 12 hours, purified by column chromatography on silic gel using methanol (0-10%) in chloroform, yield 61%. |
| E-76 | 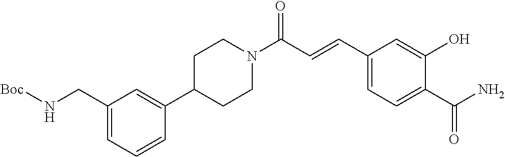 [SMILES: CC(C)(C)OC(=O)NCc1cccc(C2CCN(C(=O)/C=C/c3ccc(C(N)=O)c(O)c3)CC2)c1] | tert-butyl-3-(piperidin-4-yl) benzylcarbamate (1.2 eq.) DMF (10 vol.), HOBt (1.5 eq.), EDCI (1.5 eq.) DIEA (2.5 eq.), room temperature for 12 hours, yield 71%. |
| E-76a | 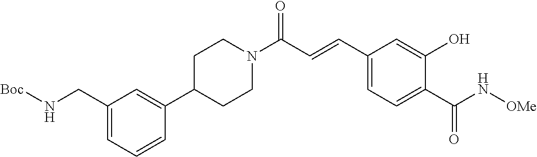 [SMILES: CONC(=O)c1ccc(/C=C/C(=O)N2CCC(c3cccc(CNC(=O)OC(C)(C)C)c3)CC2)cc1O] | tert-butyl-3-(piperidin-4-yl) benzylcarbamate (1.2 eq.) DMF (10 vol.), HOBt (1.5 eq.), EDCI (1.5 eq.) DIEA (2.5 eq.), room temperature for 12 hours, yield 36%. |
| E-86 | 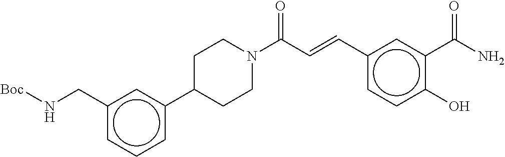 | tert-butyl-3-(piperidin-4-yl) benzylcarbamate (1.2 eq.) DCM (60 vol.), DMAP (0.5 eq.), EDCI (1.5 eq.), room temperature for 12 hours, purified by column chromatography on silic gel using methanol (0-10%) in chloroform, yield 45%. |

### Step-5

Boc deprotection of Step-4 products was carried out by stirring the products in aqueous hydrochloric acid-methanol or methanolic HCl at room temperature. Crude products were purified by reverse phase preparative HPLC and isolated as TFA salts.

The compounds synthesized by Step-5, the corresponding reaction conditions, and analytical results are shown in the table below:

| Compound | | Reaction | |
|---|---|---|---|
| No. | Structure | conditions | Analytical data |
| Target-75a | 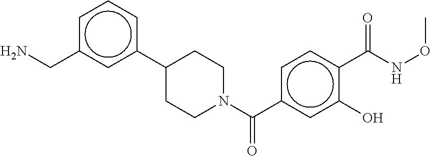 | Methanolic HCl (25 vol.), 4 hours at room temperature, purified by preparative HPLC. Isolated as TFA Salt, yield 11%. | Mol. Wt: - 383.44 M.I. Peak observed: - 384.20 HPLC Purity: - 97.05 ¹H NMR DMSO-d6: - 1.61- 1.83 (m, 4H), 2.08 (m, 2H), 3.15- 3.16 (m, 2H), 3.76 (s, 1H), 4.00 (s, 2H), 4.58 (m, 1H), 6.89-6.92 (m, 2H), 7.26-7.36 (m, 4H), 7.67- 7.69 (d, 1H). |
| Target-92 | 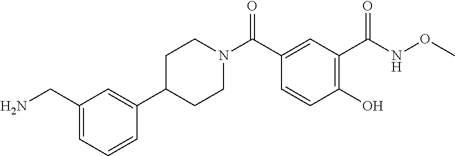 [SMILES: CONC(=O)c1cc(C(=O)N2CCC(c3cccc(CN)c3)CC2)ccc1O] | Methanol (100 vol.), concentrated HCl (10 vol.), room temperature for 12 hours, purified by preparative HPLC. Isolated as TFA salt, yield 70%. | Mol. Wt: - 383.44 M.I. Peak observed: - 384.2 HPLC: 99.5% (220 nm) ¹H NMR (400 MHz, DMSO-d6): δ 1.50-1.90 (m, 4H), 2.75-2.90 (m, 1H), 2.91-3.30 (br, 2H), 3.50-3.60 (br, 2H), 3.73 (s, 3H), 4.00-4.10 (m, 2H), 6.99 (d, J = 8.4 Hz, 1H), 7.20-7.40 (m, 4H), 7.47 (d, J = 8.4 Hz, 1H), 7.77 (s, 1H), 8.20 (br, 2H), 11.7 (br, 1H), 11.9 (br, 1H). |
| Target-76 | 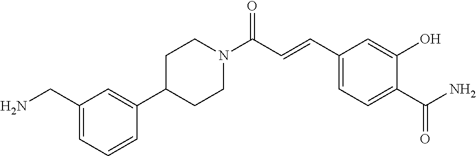 [SMILES: NCc1cccc(C2CCN(C(=O)/C=C/c3ccc(C(N)=O)c(O)c3)CC2)c1] | Methanol (30 vol.), concentrated HCl (1 vol.), room temperature for 3 hours. Isolated as hydrochloride salt in pure form after work-up, yield 45%. | Mol. Wt.: 379.45 M.I. Peak observed: - 380 [M + l] HPLC Purity: 95.60% ¹H NMR (400 MHz, CD₃OD): 7.80 (d, J = 8.0 Hz, 1H), 7.51 (d, J = 15.6 Hz, 1H), 7.45-7.30 (m, 4H), 7.26 (d, J = 15.6 Hz, 1H), 7.20-7.12 (m, 2H), 4.82-4.74 (m, 1H), 4.48-4.36 (m, 1H), 4.10 (s, 2H), 3.00-2.82 (m, 2H), 2.04-1.90 (m, 2H), 1.80-1.62 (m, 2H) |
| Target-76a | 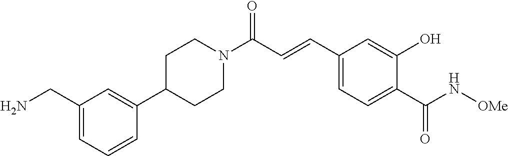 [SMILES: CONC(=O)c1ccc(/C=C/C(=O)N2CCC(c3cccc(CN)c3)CC2)cc1O] | Methanol (30 vol.), concentrated HCl (1 vol.), room temperature for 3 hours. Isolated as hydrochloride salt in pure form after work-up, yield 72%. | Mol. Wt.: 409.48 M.I. Peak observed: - 410 [M + l] HPLC Purity: 96.93% ¹H NMR (400 MHz, CD₃OD): 7.69 (d, J = 8.4 Hz, 1H), 7.50 (d, J = 15.2 Hz, 1H) 7.43-7.18 (m, 6H), 7.16 (s, 1H), 4.82-4.74 (m 1H), 4.46-4.36 (m, 1H), 4.10 (s, 2H), 3.83 (s, 3H), 3.02-2.84 (m, 2H), 2.06-1.90 (m, 2H), 1.80-1.64 (m, 2H). |
| Target-86 | 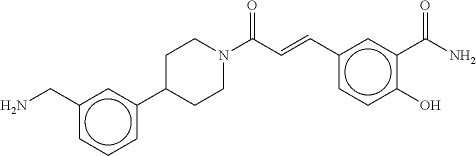 | Methanol (100 vol.), concentrated HCl (10 vol.), room temperature for 12 hours. Isolated as hydrochloride salt in pure form after work-up, yield 92.30%. | Mol. Wt: - 379.45 M.I. Peak observed: - 380.2 HPLC: 94.8% (220 nm) ¹H NMR (400 MHz, DMSO-d6): δ 1.40-1.70 (br, 2H), 1.84 (br, 2H), 2.60-2.91 (m, 2H), 3.10-3.30 (m, 1H), 3.98 (d, J = 5.6 Hz, 2H), 4.40-4.70 (br, 2H), 6.91 (d, J = 8.4 Hz, 1H), 7.20-7.50 (m, 6H), 7.78 (d, J = 8.8 Hz, 1H), 8.07 (br, 1H), 8.32 (br, 2H), 8.37 (br, 2H), 8.65 (br, 1H). |

### Example 4

### Synthesis of Cofluoron Monomers Bearing \acute{α}-Hydroxy Carboxylic Acids Functionality

### Method H

The cofluoron monomers bearing \acute{α}-hydroxy carboxylic acids moieties were synthesized by Method H as below, according to the following reaction scheme:

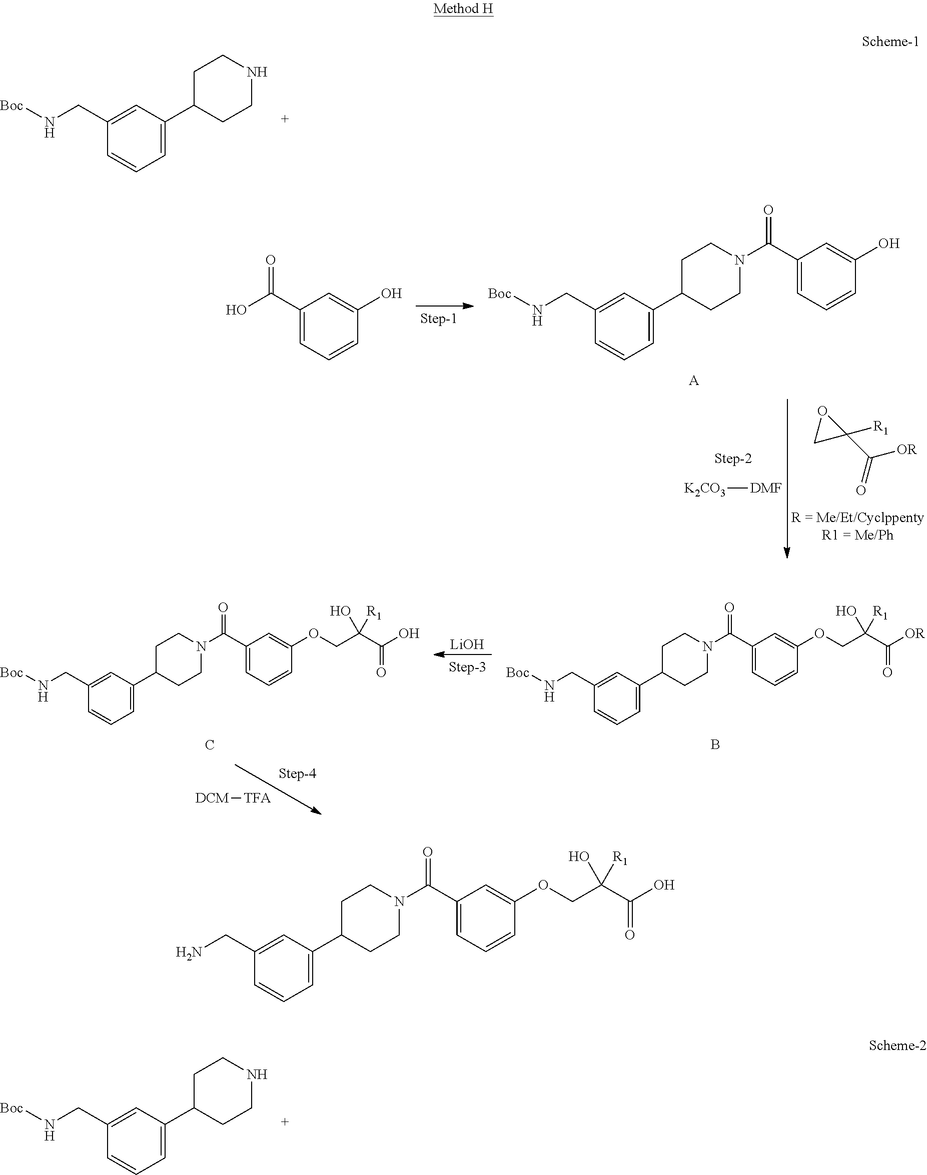

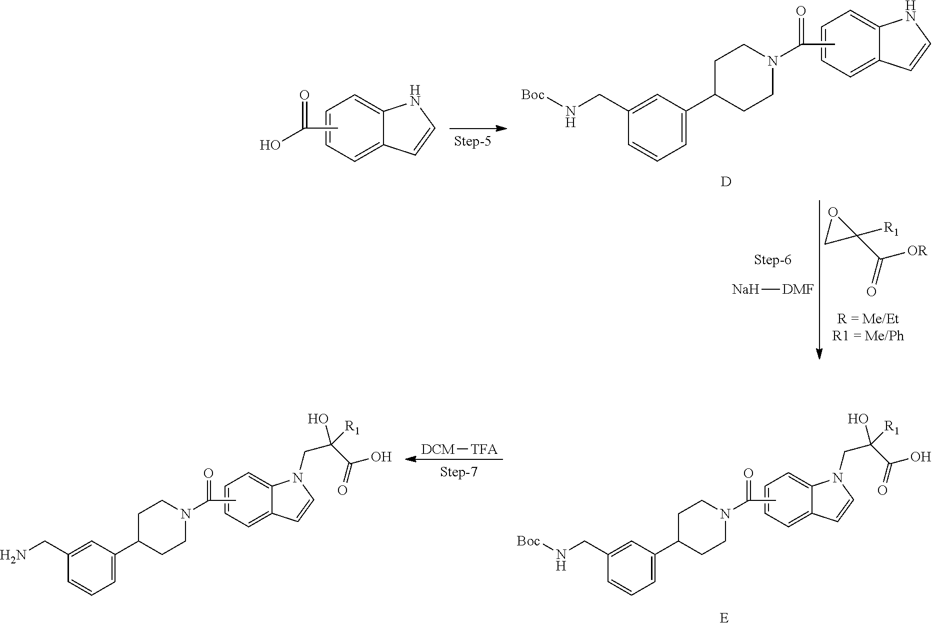

In general, alpha-hydroxy carboxylic acids were synthesized by reacting the desired epoxide with tert-butyl 3-(1-(3-hydroxybenzoyl)piperidin-4-yl)benzyl carbamate in the presence of base to yield alpha-hydroxy carboxylic esters that were hydrolyzed and de-protected to obtain the desired compounds (Scheme-1).

Similarly, indole 5/6 carboxylic acids were coupled with tert-butyl 3-(piperidin-4-yl)benzyl carbamate and resulting coupled products were treated with desired epoxides. Alpha hydroxy esters formed in the reaction were hydrolyzed to yield alpha-hydroxy acids, which were subjected to Boc de-protection to obtain the desired compounds (Scheme-2).

### Step-1 and 5:

Coupling of desired carboxylic acid was carried out with tert-butyl 3-(piperidin-4-yl)benzylcarbamate following the general procedure described in Method-A (Step-4).

### Step-2:

In a stirred suspension of the product from step-1 (intermediate A) in dimethyl formamide, potassium carbonate was added followed by desired epoxide. The reacted material was heated to 100° C., and the reaction monitored by LCMS until the maximum amount of starting materials was consumed. Then the reacted material was cooled to room temperature and diluted with water and extracted with ethyl acetate. Ethyl acetate extract was washed with water, dried over sodium sulfate and concentrated in vacuum to obtain the crude product. The crude product was purified by column chromatography over silica gel using 0-25% ethyl acetate in hexane. Epoxide required for preparation of Target-103 was synthesized following the procedure described in the literature (J. Am. Chem. Soc. 113: 3096-3106 (1991), which is hereby incorporated by reference in its entirety).

### Step-3:

Hydroxy ester from Step-2 was hydrolyzed to acid following general procedure in Method-A, Step 3. The products were purified by column chromatography over silica gel using methanol (1-15%) in chloroform.

### Step-4:

Boc deprotection of the Step-3 products was carried out by stirring with methanolic HCl at room temperature. Reactions were monitored by LCMS and, after reaction completion, solvents were evaporated in vacuum. Residue was purified by reverse phase preparative HPLC to obtain the products as TFA salts.

### Step-6:

The stirred suspension of coupled product from Step-1 in THF was added with sodium hydride. Stirring continued for 30 minutes and desired epoxy ester was added to the suspension. Stirring continued at room temperature, and the reaction was monitored by LCMS until LCMS showed the peak of the corresponding carboxylic acid instead of the ester. After completion of the reaction, the reacted material was concentrated in vacuum and quenched with ice. The pH of the reacted material was then adjusted to 3-4 by potassium hydrogen sulfate and extracted with ethyl acetate. Ethyl acetate extract was dried over sodium sulfate and concentrated in vacuum to obtain the crude product, which was used for carrying out the next step without purification.

### Step-7:

Boc de-protection of product from step-6 was carried out following the general procedure described in Method-A, Step-9.

### Example 5

### Synthesis of Spiro Analogues for Cofluoron Monomers

### Method J

Method J was carried out according to the reaction scheme as follows:

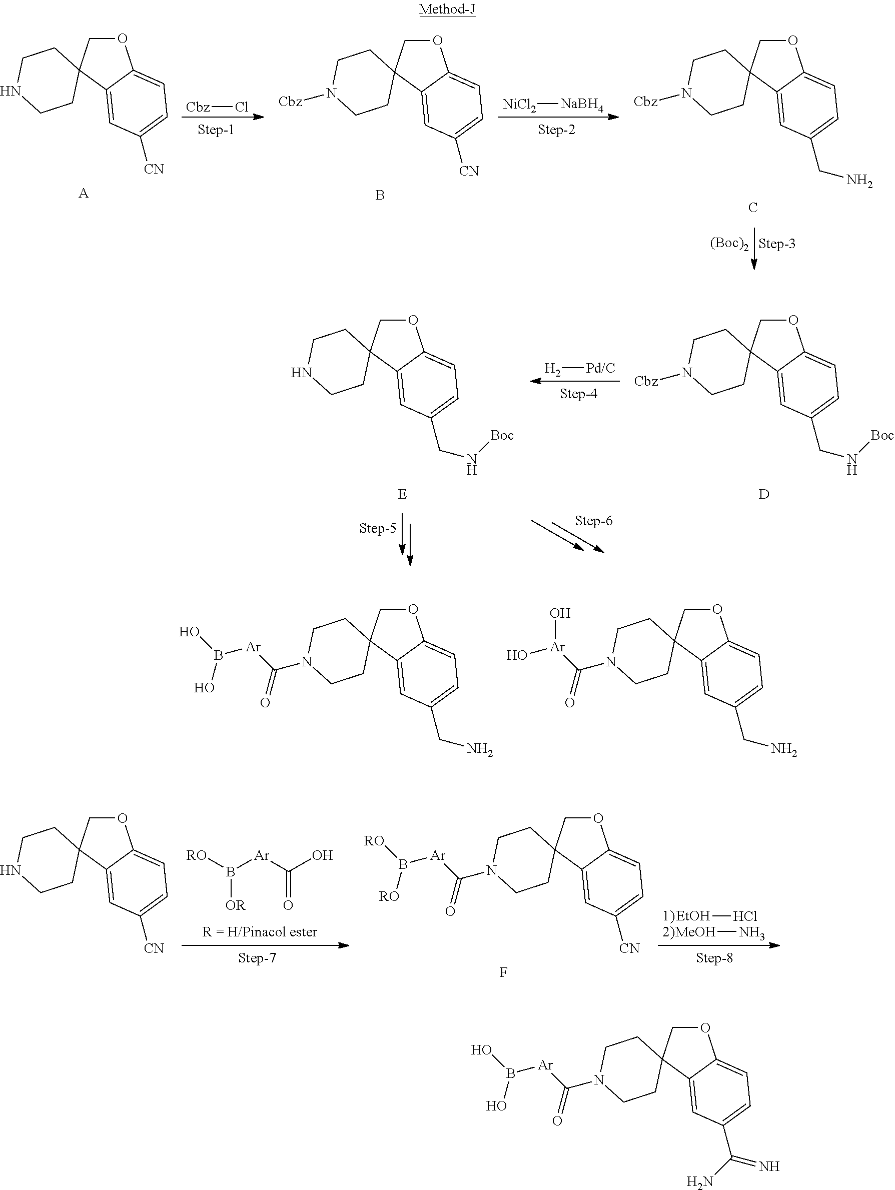

In general, spiro key intermediate (E) was synthesized from 2H-spiro [benzofuran-3,4′-piperidine]-5-carbonitrile through the reactions described in the above scheme below. Also see U.S. Patent Application Publication No. 2009/0163527; and B. org. Med. Chem. Lett. 18: 2114-2121 (2008), both of which are hereby incorporated by reference in their entirety). Boronic acids or hydroxy compounds were synthesized through the similar reaction schemes described in Method A and Method C. Spiro amidines were synthesized following Steps 7 and 8 in the above reaction scheme, as described in details below.

### Experimental Procedures:

### Step-1

To a stirred solution of 2H-spiro[benzofuran-3,4′-piperidine]-5-carbonitrile (5 g, 0.023 mol) in THF (10 vol.) and aqueous sodium bicarbonate (10 vol.), benzyl chloroformate (1.3 eq. 0.030 mol.) was added at 0-5° C. and the reaction mixture was stirred for 3 hours at same temperature. The reaction mixture was then warmed to room temperature and stirring continued for additional 2 hours. Solvents were then evaporated under reduced pressure, and aqeuous layer was extracted with ethyl acetate. Ethyl acetate extracts were dried over sodium sulphate and concentrated to obtain the crude product. The crude product was purified by column chromatography over silica gel using ethyl acetate (0-20%) in hexane to obtain the pure product. (Yield: 60%; Mol. Wt: 348.40; M.I peak observed: 348.95)

### Step-2 and 3

To a stirred solution of benzyl-5-cyano-2H-spiro[benzofuran-3,4′-piperidine]-1′-carboxylate (4 g, 0.011 mol) in methanol (10 vol), Boc anhydride (5.01 g, 2.0 eq. 0.022 mol.) and NiCl₂ (0.372 g, 0.25 eq, 0.0028 mol) was added at 0-5° C. Sodium borohydride (0.869 g, 2.0 eq., 0.22 mol.) was then added portion-wise maintaining the temperature. Reaction mixture was allowed to warm to room temperature and stirring continued for 3 hours thereafter. Solvents were evaporated under reduced pressure. Residue was diluted with water (˜20 vol.) and extracted with ethyl acetate. Ethyl acetate extract was dried over sodium sulphate and concentrated to obtain the crude product. The crude product was purified by column chromatography over silica gel using ethyl acetate (0-40%) in hexane to obtain the pure product. (Yield: 3.2 g (62.7%); Mol. Wt: 438.52; M.I peak observed: −475.55 (M+Na))

### Step-4

To a stirred solution of benzyl5-((tert-butoxycarbonyl)amino)-2H-spiro[benzofuran-3,4′-piperidine]-1′-carboxylate (3 g, 0.0066 mol) in methanol (15 vol), 10% Pd/C (500 mg) was added at room temperature under nitrogen atmosphere. The mixture was then stirred under hydrogen pressure (˜10 Kg) at room temperature in an autoclave until no more hydrogen was consumed and LCMS indicated formation of product and the maximum consumption of starting materials (˜4 hours required). The reaction vessel was depressurized and the reacted material was filtered through celite. Solvent was evaporated in vacuum, and the residue was purified by column chromatography to obtain pure product, which was characterized by LCMS. (Yield: 63%; Mol. Wt: 318.41; M.I peak observed: 319.05)

### Step-5:

The procedure described in Method-A, Steps-4 and 5 was followed:

The compounds synthesized by Step-5, the corresponding reaction conditions, and analytical results are shown in the table below:

| Compound | | | |
|---|---|---|---|
| No. | Structure | Reaction conditions | Analytical data |
| Target-35- Spiro | 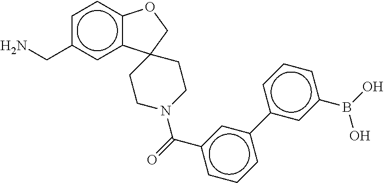 | 1) tert-butyl ((2H- spiro[benzofuran-3,4′- piperidin]-5- yl)methyl)carbamate (1.1 eq.), EDCI (1.5 eq.), DMAP (1.2 eq.), DCM (100 vol.), room temperature for 12 hours, yield 81%; 2) TFA (10 eq.) Acetonitrile (65 vol.), room temperature for 12 hours, yield 57%. | Mol.Wt: - 442.31 M.I. Peak observed: 443.40 HPLC Purity: - 95.81% ¹H NMR DMSO-d6: - ¹HNMR (400 MHz, DMSO): - 1.67-1.79 (m, 4H), 3.16-3.29 (m, 2H), 3.92-3.93 (q, 4H), 4.50 (s, 3H), 6.81-6.83 (d, 1H), 7.23-7.25 (d, 1H), 7.40-7.58 (m, 4H), 7.71-7.81 |
| | | | (m, 4H), 8.15 |
| | | | (s, 1H), 8.25 |
| | | | (bs, 2H). |

### Step-7:

The reactions were carried out following procedures described in Method-A (Step-4 or Step-8).

The compounds synthesized by Step-7 and the corresponding reaction conditions are shown in the table below:

| Compound No. | Structure | Reaction conditions |
|---|---|---|
| F-35-Spiro amidine | 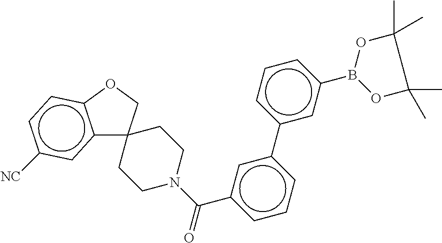 | 2H-spiro[benzofuran-3,4′-piperidine]- 5-carbonitrile (1.1 eq.), EDCI (1.5 eq.), DMAP (1.2 eq.), DCM (100 vol), room temperature, 12 hours, purified by column chromatography, over silica gel using 0-40% ethyl acetate in hexane. Yield - 75% |
| F-33 spiro amidine | 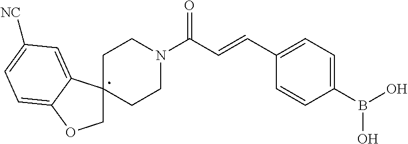 | 2H-spiro[benzofuran-3,4′-piperidine]- 5-carbonitrile (1.1 eq.), EDCI (1.5 eq.), DMAP (1.2 eq.), DCM (100 vol), room temperature, 12 hours, column chromatography, over silica gel using 0-40% ethyl acetate in hexane, Yield - 52% |

### Step-8:

Products from Step-7 were treated with ethanolic HCl at ambient temperature followed by methanolic ammonia in a sealed bottle to obtain the desired compounds, which were isolated by preparative HPLC as TFA salts. The TFA salts were converted to hydrochloride salts by stirring with 2N HCl for 30 minutes and by subsequent lipophilization.

The compounds synthesized by Step-8, the corresponding reaction conditions, and analytical results are shown in the table below:

| Compound | | | |
|---|---|---|---|
| No. | Structure | Reaction conditions | Analytical data |
| Target-35- Spiro amidine | 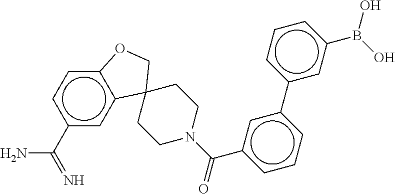 | 1) Ethanolic HCl (5 vol) room temperature, r hours, purified by column chromatography, over silica gel using 0-40% methanol in chloroform Yield-75% 2) methanolic ammonia (10 vol), heating in sealed tube, 12 hours, purified by preparative HPLC, isolated as TFA salt, converted to HCl salt. Yield - 43% | Mol. Wt: - 455.31 M.I. peak observed: - 456.20 HPLC Purity: - 98.66% 1H NMR DMSO-d6: - 1.703- 1.847 (m, 4H), 3.057 (m, 2H), 3.657 (m, 2H), 4.657 (s, 2H), 7.038-7.018 (d, 1H), 7.413- 7.457 (m, 2H), 7.570 (t, 1H), 7.790 (t, 1H), 7.866 (s, 1H), 8.142 (s, 1H), 7.675-7.746 (m, 4H), 8.746 (bs, 2H), 9.079 (s, 2H), 8.142 (s, 1H). |
| T-33 spiro amidine | 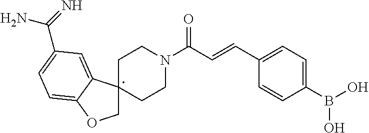 | 1) Ethanolic HCl (5 vol) room temperature, 4 hours, purified by column chromatography, over silica gel using 0-40% methanol in chloroform Yield-61% 2) methanolic ammonia (10 vol), heating in sealed tube, 12 hours, purified by | Mol. Wt: - 405.25 M.I. peak observed: - 406.10 HPLC Purity: - 98.36% 1H NMR DMSO-d6: - 1.784- 1.815 (m, 4H), 2.900 (m, 1H), 4.340-4.493 (m, 2H), 4.862 (s, 2H), 7.029-7.050 (d, 1H), 7.364-7.403 (d, 1H J = 15.6 Hz), 7.513-7.551 (d, 1H, J = 15.2 Hz), 7.689-7.822 (d, 5H), |
| | | preparative HPLC, | 7.804 (s, 1H), 9.050 (bsa, 2H), |
| | | isolated as TFA salt, | 8.710 (bs, 2H), 8.134 (bs, 2H). |
| | | converted to HCl salt. | |
| | | Yield - 13% | |

### Example 6

### Synthesis of Cofluoron Monomers Bearing Benzo Oxaborol-1-ol Functionality

### Method K

The cofluoron monomers bearing benzo oxaborol-1-ol moieties were synthesized by Method K as below, according to the following reaction scheme:

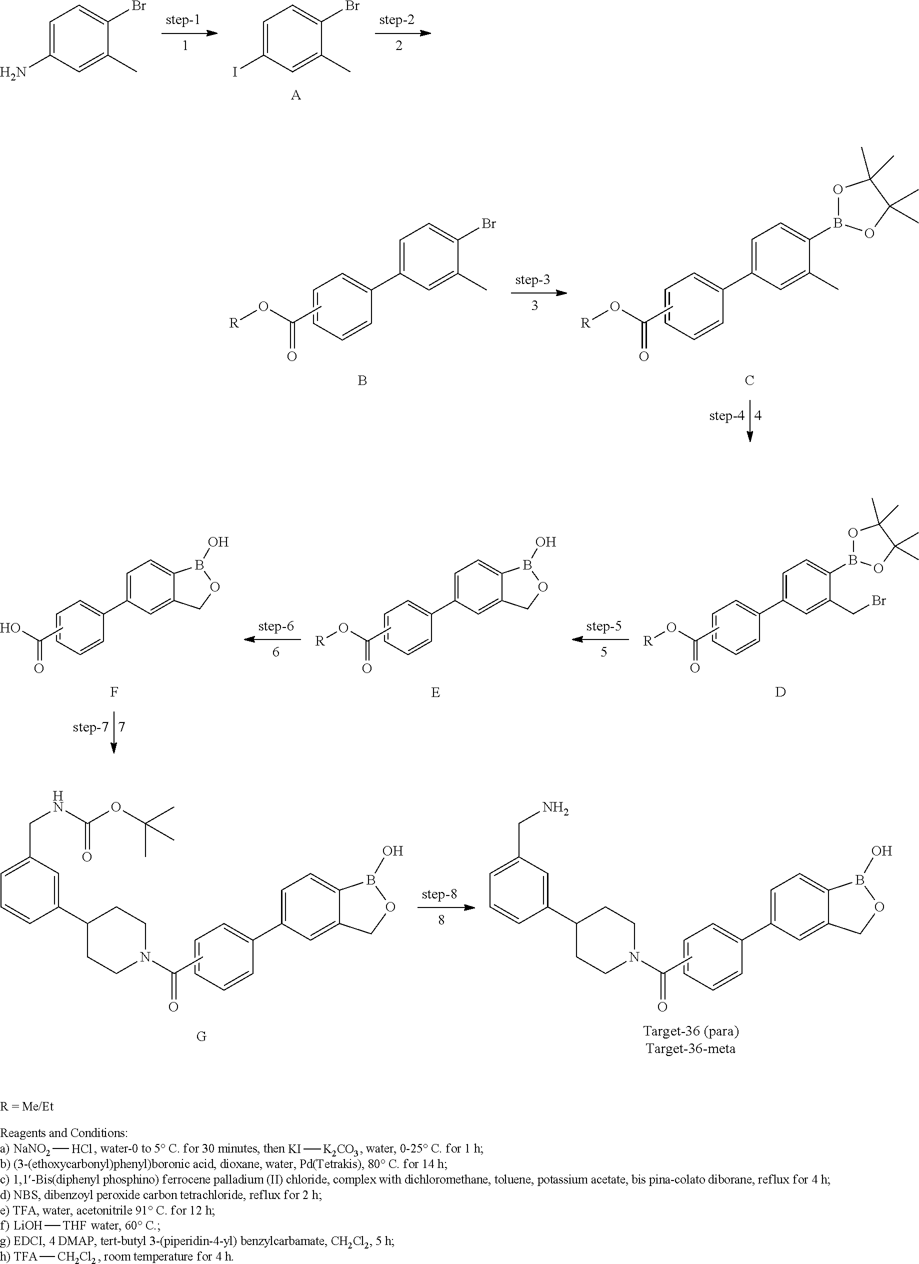

### Step-1:

1-Bromo-4-iodo-2-methylbenzene was synthesized following the procedures available in the literature (Bioorganic and Medicinal Chemistry 16: 6764-6777 (2008); J. Am. Chem. Soc. 122: 6871-6883 (2000), both of which are hereby incorporated by reference in their entirety).

### Step-2:

Suzuki coupling of Step-1 product with meta/para carbethoxy/methoxy phenyl boronic acid was carried out in the presence of palladium (0) tetrakis(triphenyl phosphene) in dioxane and sodium carbonate as base. After completion of reaction, the reaction mixture was filtered through celite pad and filtrate was concentrated under reduced pressure residue was diluted with water and extracted with ethyl acetate to obtain crude products. Crude products obtained were purified by column chromatography over silica gel using 5-10% ethyl acetate in hexane.

The compounds synthesized by Step-2 and the corresponding reaction conditions are shown in the table below:

| Compound No. | Structure | Reaction conditions |
|---|---|---|
| B-36 | 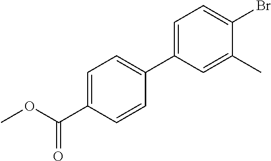 | Boronic acid (1.2 eq.), Water (5 vol) dioxane (20 vol), Pd-Tetrakis (10 mol %), Sodium carbonate (2 eq.), 80° C., 15 hours. Yield 64.8% |
| B-36-meta | 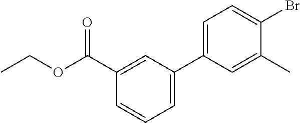 [SMILES: CCOC(=O)c1cccc(-c2ccc(Br)c(C)c2)c1] | Same as above Yield: - 60% |

### Step-3

Stirred suspension of Step-2 products in toluene was degassed with argon and to this were added potassium acetate, PdCl₂-DPPF—CH₂Cl₂ and bis(pinacolato)diborane. The reacted material was heated to reflux and monitored by LCMS until the maximum amount of starting materials was consumed. The mixture was then filtered through celite pad and filtrate was concentrated under reduced pressure to yield the crude product. The crude product was purified by column chromatography over silica gel using 1-5% ethyl acetate in hexane.

The compounds synthesized by Step-3 and the corresponding reaction conditions are shown in the table below:

| Compound No. | Structure | Reaction conditions |
|---|---|---|
| C-36 | 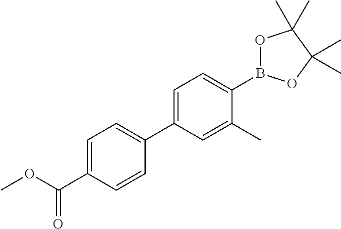 | Bispinacolato diborane (2.5 eq.), PdCl₂ (dppf) (5 mol %), dppf (3 mol %), Potassium acetate (3.0 eq.), Toluene (30 vol), Reflux, 5 hours., Yield 50% |
| C-36-meta | 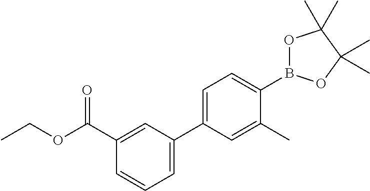 [SMILES: CCOC(=O)c1cccc(-c2ccc(B3OC(C)(C)C(C)(C)O3)c(C)c2)c1] | Same as above Yield: - 75% |

### Step-4

To a stirred solution of Step-3 product in carbon tetrachloride, dibenzoyl peroxide and N-bromo succinamide were added. The resulting mixture was heated to 75° C. and reaction was monitored by LCMS. After consumption of maximum starting materials, the reaction mixture was diluted with water and extracted with dichloromethane. The organic phase was again washed with water followed by brine, dried over anhydrous sodium sulfate, and concentrated under reduced pressure to obtain the crude product. The crude product was purified by column chromatography over silica gel using 1-5% ethyl acetate in hexane.

The compounds synthesized by Step-4 and the corresponding reaction conditions are shown in the table below:

| Compound No. | Structure | Reaction conditions |
|---|---|---|
| D-36 | 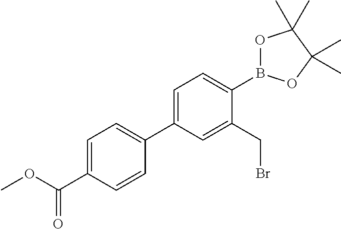 | benzoyl peroxide (0.2 eq.), NBS (1.2 eq.) CCl₄ (20 vol.), 75° C. for 3 hours. Yield 60%. |
| D-36-meta | 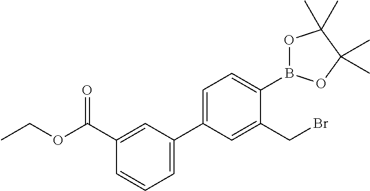 [SMILES: CCOC(=O)c1cccc(-c2ccc(B3OC(C)(C)C(C)(C)O3)c(CBr)c2)c1] | Same as above Yield 65%. |

### Step-5

To a stirred solution of Step-4 product in acetonitrile, trifluoro acetic acid and water were added and mixture was heated to 91° C. and monitored by LCMS. After the maximum amount of starting material was consumed, the reaction mixture was concentrated and residue obtained was diluted with water and extracted with ethyl acetate. Concentration of ethyl acetate layer yielded crude product, which was purified by column chromatography over silica gel using 10-35% ethyl acetate in hexane.

The compounds synthesized by Step-5 and the corresponding reaction conditions are shown in the table below:

| Compound No. | Structure | Reaction conditions |
|---|---|---|
| E-36 | 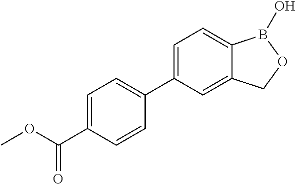 [SMILES: COC(=O)c1ccc(-c2ccc3c(c2)COB3O)cc1] | Acetonitrile (30 vol), TFA (10 vol) Water (5 Vol), 91° C. 14 hours, Yield: - 50% |
| E-36-meta | 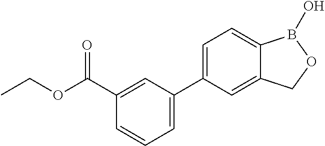 [SMILES: CCOC(=O)c1cccc(-c2ccc3c(c2)COB3O)c1] | Same as above Yield: - 50% |

### Step-6

A mixture of Step-5 product, lithium hydroxide, THF, and water was heated to 60° C. The reaction was monitored by LCMS until the maximum amount of starting materials was consumed. The reaction mixture was concentrated and diluted with water. pH of the reacted material was then adjusted to ˜2 using concentrated HCl. Precipitated product was filtered, washed with water, and dried in vacuum oven.

The compounds synthesized by Step-6, the corresponding reaction conditions, and analytical results are shown in the table below:

| Compound | | Reaction | |
|---|---|---|---|
| No. | Structure | conditions | Analytical data |
| F-36 | 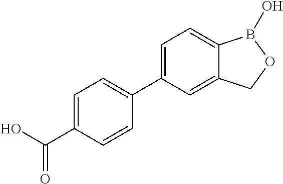 [SMILES: O=C(O)c1ccc(-c2ccc3c(c2)COB3O)cc1] | LiOH (10 eq), THF (10 vol), Water (20 Vol), 60° C., 2 hours. Yield: - 60% | Ionization not observed in LCMS/ESMS 1H NMR DMSO-d6: - 5.054 (s, 2H), 7.141-7.165 (d, 1H, J = 9.6 Hz), 7.531- 7.551 (d, 1H, J = 8 Hz), 7.778-7.846 (m, 2H), 7.992-8.058 (m, 2H), 7.084 (s, 1H). |
| F-36-meta | 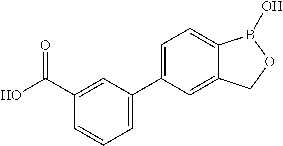 [SMILES: O=C(O)c1cccc(-c2ccc3c(c2)COB3O)c1] | Same as above Yield: - 75% | 1H NMR DMSO-d6: - 5.051 (s, 2H), 7.523-7.543 (d, 1H, J = 8 Hz), 7.618- (t, 1H), 7.812-7.832 (d, 1H), 7.922-7.955 (d, 2H), 8.076 (s, 1H), 8.216 (s, 1H), 9.275 (s, 1H), 13.10 (s, 1H) |

### Step-7

The reactions were carried out following the general procedure described in Method-A, Step-4. DMF was used as co-solvent. pH of the reacted material was adjusted to ˜5 by adding dilute HCl prior to the extraction.

The compounds synthesized by Step-7 and the corresponding reaction conditions are shown in the table below:

| Compound No. | Structure | Reaction conditions |
|---|---|---|
| G-36 | 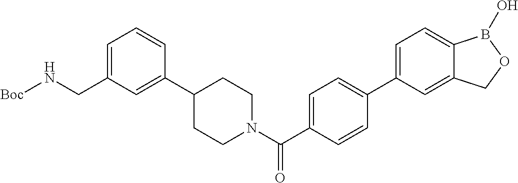 [SMILES: CC(C)(C)OC(=O)NCc1cccc(C2CCN(C(=O)c3ccc(-c4ccc5c(c4)COB5O)cc3)CC2)c1] | tert-butyl 3-(piperidin-4-yl) benzyl carbamate (1.3 eq.), EDCI.HCl (1.5 eq.), DMAP (2 eq.), DCM (20 vol), DMF (10 vol), room temperature, 4 hours, Yield: - 50%, |
| G-36-meta | 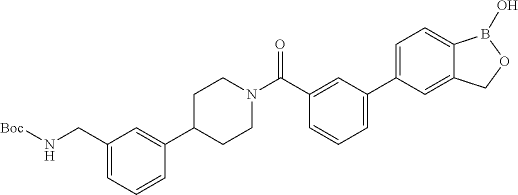 [SMILES: CC(C)(C)OC(=O)NCc1cccc(C2CCN(C(=O)c3cccc(-c4ccc5c(c4)COB5O)c3)CC2)c1] | Same as above Yield: - 70% |

### Step-8

Boc de-protection of product from Step-6 was carried out following the general procedure described in Method-A, Step-9.

The compounds synthesized by Step-8, the corresponding reaction conditions, and analytical results are shown in the table below:

| Compound | | Reaction | |
|---|---|---|---|
| No. | Structure | conditions | Analytical data |
| Target-36 | 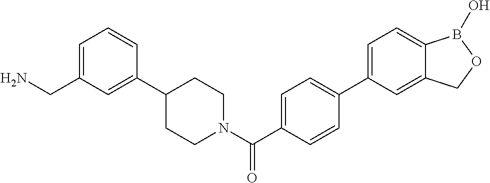 [SMILES: NCc1cccc(C2CCN(C(=O)c3ccc(-c4ccc5c(c4)COB5O)cc3)CC2)c1] | TFA (20 eq.), dichloromethane (20 vol), room temperature, 4 hours. Preparative HPLC. isolated as TFA salt converted to hydrochloride Yield: - 12.76% | Mol.Wt: - 426.32 M.I. peak observed: - 427.05 HPLC Purity: - 99.10% 1H NMR DMSO-d6: - 1.646-1.769 (m, 4H), 4.653-4.681 (m, 1H), 2.822-2.881 (m, 2H), 3.104-3.218 (m, 2H), 3.997-4.011 (d, 2H), 5.047 (s, 2H), 7.458 (s, 1H), 8.055 (s, 1H), 7.535-7.554 (m, 2H), 7.298-7.375 (m, 3H), 7.726-7.746 |
| | | | (d, 2H, J = 8 Hz), 7.816- |
| | | | 7.796 (d, 2H, J = 8 Hz) |
| | | | 8.372 (s, 3H). |
| Target-36- meta | 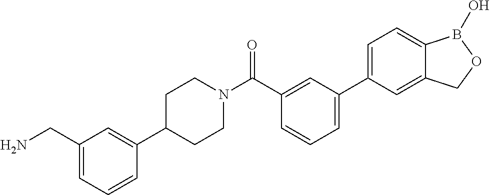 [SMILES: NCc1cccc(C2CCN(C(=O)c3cccc(-c4ccc5c(c4)COB5O)c3)CC2)c1] | Same as above isolated as TFA salt & converted to hydrochloride Yield: - 50% | Mol. Wt: - 426.32 M.I. peak observed: - 427.06 HPLC Purity: - 99.47% ¹H NMR DMSO-d6: - 1.670- 2.070 (m, 4H), 2.813-2.873 (m, 2H), 3.166-3.230 (m, 1H), 3.989-4.003 (d, 2H), 3.578- 3.558 (m, 1H), 5.046 (s, 2H), 7.469 (s, 1H), 7.669, (s, 1H), 8.095 (s, 1H), 7.288-7.340 (m, 3H), 7.819-7.816 (d, 1H, J = 8 Hz), 7.757-7.737 (d, 1H, J = 8 Hz), 7.433-7.414 (d, 1H, J = 7.6 Hz), 7.512-7.532, |
| | | | (d, 1H, J = 8 Hz), 8.406 |
| | | | (s, 3H) |

### Example 7

### Synthesis of (E)-(3-(4-(3-(aminomethyl)phenyl)piperidine-1-carbonyl)styryl)boronic acid Hydrochloride (Target-14)

(E)-(3-(4-(3-(aminomethyl)phenyl)piperidine-1-carbonyl)styryl)boronic acid hydrochloride was synthesized according to the following reaction scheme:

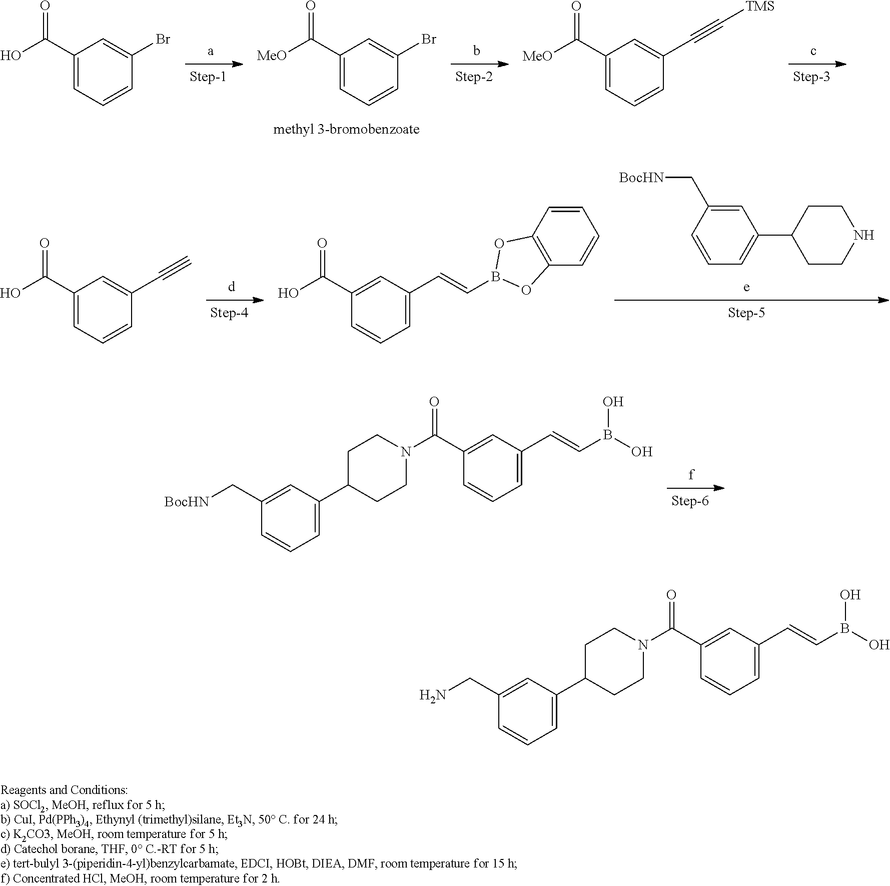

Methyl 3-bromobenzoate was synthesized from 3-bromobenzoic acid by esterification with thionyl chloride in methanol. Further Sonogashira coupling was carried out with ethynyl(trimethyl)silane to afford methyl 3-((trimethylsilyl)ethynyl)benzoate, which, upon hydrolysis with lithium hydroxide in methanol, yielded 3-ethynylbenzoic acid, following the general procedures as described in PCT/US 2010/002708, which is hereby incorporated by reference in its entirety.

### Step-4: Synthesis of (E)-3-(2-(benzo[d][1,3,2]dioxaborol-2-yl)vinyl)benzoic acid

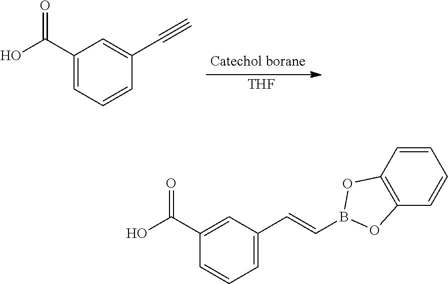 [SMILES: C#Cc1cccc(C(=O)O)c1 ; O=C(O)c1cccc(/C=C/B2Oc3ccccc3O2)c1 ; "THF"]

To a cold solution of 3-ethynylbenzoic acid (0.2 g, 1.37 mmol) in anhydrous THF (10 mL), was added catechol borane (0.15 mL, 1.37 mmol). The reaction mixture was stirred at room temperature for 2 h. The resulting solution was poured into cold water and extracted with EtOAc. The organic layer was washed with H₂O, dried over Na₂SO₄ and evaporated under vacuum. The crude product was purified by silica gel column chromatography (0-20%, EtOAc in hexane) to afford (E)-3-(2-(benzo[d][1,3,2]dioxaborol-2-yl)vinyl)benzoic acid as a white solid. (Yield: 0.27 g (75%); ¹H NMR (400 MHz, Acetone-d₆): δ 8.18-8.12 (m, 1H), 7.95 (d, J=7.2 Hz, 1H), 7.92-7.86 (m, 1H), 7.76 (d, J=7.2 Hz, 1H), 7.51 (t, J=7.2 Hz, 1H), 7.43 (d, J=18.2 Hz, 1H), 7.12-7.02 (m, 1H), 6.84-6.76 (m, 1H), 6.70-6.62 (m, 1H), 6.32 (d, J=18.2 Hz, 1H))

### Step-5: Synthesis of (E)-(3-(4-(3-(((tert-butoxycarbonyl)amino)methyl)phenyl)piperidine-1-carbonyl)styryl)boronic acid

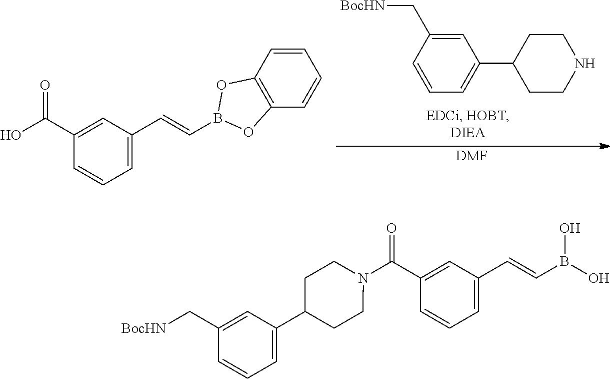

To a solution of (E)-3-(2-(benzo[d][1,3,2]dioxaborol-2-yl)vinyl)benzoic acid (0.1 g, 0.38 mmol) in anhydrous DMF (5 mL) at 0° C., was added HOBt (0.077 g, 0.57 mmol). The reaction mixture was stirred for 10 minutes and EDCI (0.11 g, 0.57 mmol), tert-butyl 3-(piperidin-4-yl)benzylcarbamate (0.11 g, 0.38 mmol) and DIEA (0.13 mL, 0.76 mmol) were added. The resulting solution was stirred at room temperature for overnight. The reaction mixture was then diluted with EtOAc and was washed with H₂O. The organic layer was dried over Na₂SO₄ and evaporated under vacuum. The crude product was purified by silica gel column chromatography (0-15%, EtOAc in hexane) to afford (E)-(3-(4-(3-(((tert-butoxycarbonyl)amino)methyl)phenyl)piperidine-1-carbonyl)styryl)boronic acid. (White solid; Yield: 0.06 g (35%); Mol. Wt.: 464.36; LCMS (m/z): 465 [M+1])

### Step-6: Synthesis of (E)-(3-(4-(3-(aminomethyl)phenyl)piperidine-1-carbonyl)styryl)boronic acid hydrochloride

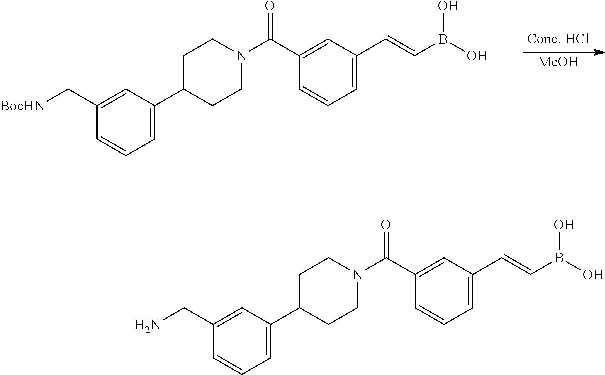 [SMILES: "MeOH" ; CC(C)(C)OC(=O)NCc1cccc(C2CCN(C(=O)c3cccc(/C=C/B(O)O)c3)CC2)c1 ; NCc1cccc(C2CCN(C(=O)c3cccc(/C=C/B(O)O)c3)CC2)c1]

To a stirred solution of (E)-(3-(4-(3-(((tert-butoxycarbonyl)amino)methyl)phenyl)piperidine-1-carbonyl)styryl)boronic acid (0.05 g, 0.09 mmol) in MeOH (3 mL) was added 2 N HCl (0.05 mL) at room temperature. The resulting solution was stirred at room temperature for 5 h. The reaction mixture was evaporated under vacuum and the resulting residue was triturated with diethyl ether to afford (E)-(3-(4-(3-(aminomethyl)phenyl)piperidine-1-carbonyl)styryl)boronic acid hydrochloride, as a white solid. (Yield: 0.02 g (64%); Mol. Wt.: 364.25; LCMS (m/z): 365 [M+1], 387 [M+Na]; HPLC Purity: 94.17%; ¹H NMR (400 MHz, CD3OD): δ 7.62-7.54 (m, H), 7.52-7.46 (m, 1H), 7.42-7.18 (m, 7H), 6.37 (d, J=18.0 Hz, 1H), 4.02 (s, 3H), 3.82-3.70 (m, 1H), 3.20-3.10 (m, 1H), 2.95-2.76 (m, 2H), 1.95-1.82 (m, 1H), 1.80-1.52 (m, 3H).)

### Example 8

### Synthesis of (Z)-1-(4-(3-(aminomethyl)phenyl)piperidin-1-yl)-3-(3,4-dihydroxyphenyl)prop-2-en-1-one Hydrochloride (Target-24 cis)

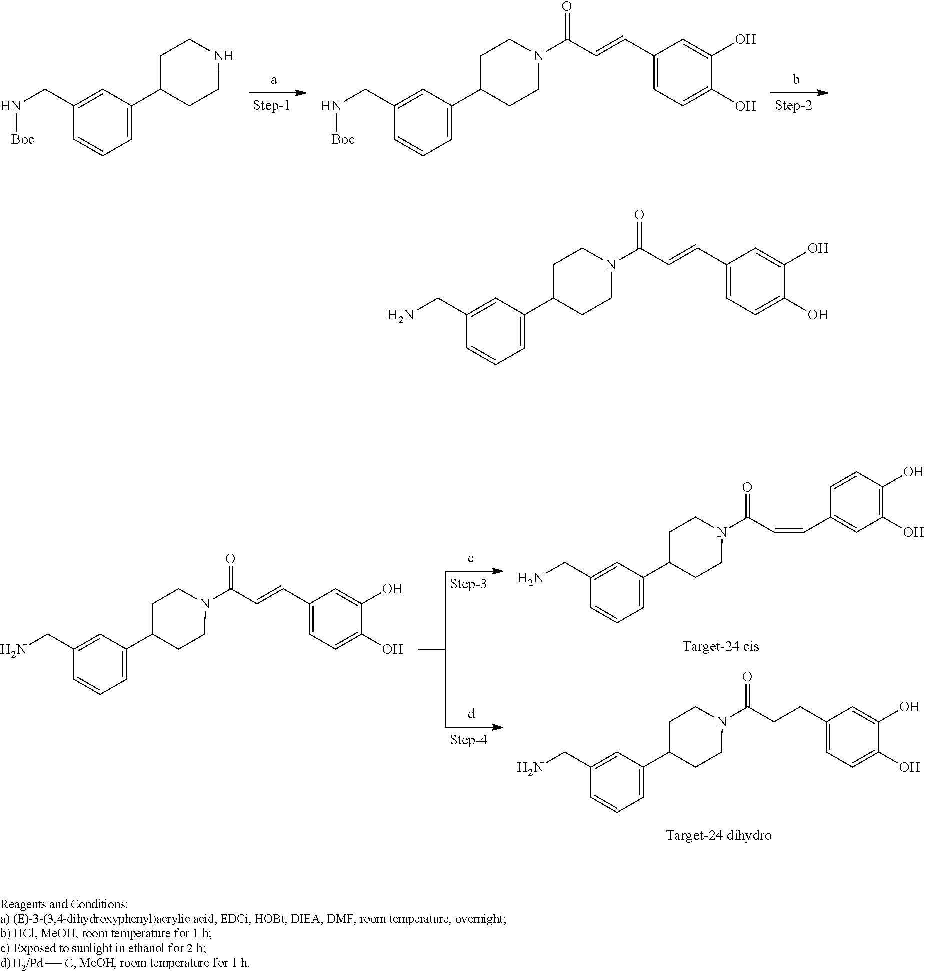

Synthesis of (E)-1-(4-(3-(aminomethyl)phenyl)piperidin-1-yl)-3-(3,4-dihydroxyphenyl)prop-2-en-1-one hydrochloride can follow the general procedures as described in PCT/US 2010/002708, which is hereby incorporated by reference in its entirety.

### Step-3: Synthesis of (Z)-1-(4-(3-(aminomethyl)phenyl)piperidin-1-yl)-3-(3,4-dihydroxyphenyl)prop-2-en-1-one hydrochloride

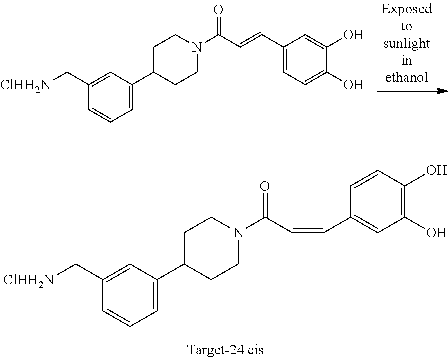

(E)-1-(4-(3-(Aminomethyl)phenyl)piperidin-1-yl)-3-(3,4-dihydroxyphenyl)prop-2-en-1-one hydrochloride was taken in ethanol (3.0 mL) and exposed to sunlight for 2 h. The organic layer was concentrated under vacuum to afford (Z)-1-(4-(3-(aminomethyl)phenyl)piperidin-1-yl)-3-(3,4-dihydroxyphenyl)prop-2-en-1-one hydrochloride as a white solid. (Yield: 0.018 g, (90%); Mol. Wt.: 352.43; LCMS (m/z): 353 [M+1], 375 [M+Na]; HPLC Purity: 89.73%; ¹H NMR (400 MHz, CD3OD): δ 7.33 (t, J=7.6 Hz, 1H), 7.25 (d, J=7.6 Hz, 1H), 7.16 (d, J=7.6 Hz, 1H), 7.10 (s, 1H), 6.94 (d, J=1.8 Hz, 1H), 6.80 (d, J=8.2 Hz, 1H), 6.74 (dd, J=8.2, 1.8 Hz, 1H), 6.67 (d, J=12.0 Hz, 1H), 5.91 (d, J=12.0 Hz, 1H), 4.80-4.70 (m, 1H), 4.13 (ABq, J=13.6 Hz, 2H), 4.08-3.98 (m, 1H), 3.08-2.95 (m, 1H), 2.80-2.65 (m, 2H), 1.82-1.74 (m, 1H), 1.68-1.54 (m, 1H), 1.45-1.36 (m, 1H), 0.80-0.65 (m, 1H))

Additional examples for synthesizing linker elements or cofluorons containing boronic acid family and their binding partners may be found in PCT/US 2010/002708, which is hereby incorporated by reference in its entirety.

### Example 9

### Fluorescence Measurements of Cofluoron Models

The fluorescence properties of cofluorons were measured in 0.1M HEPES ((4-(2-hydroxyethyl)-1-piperazineethanesulfonic acid) or 0.1M phosphate buffer at pH 7.4 in 96- or 384-well black plates using a Molecular Dynamics SpectraMax M5 plate reader (Molecular Devices, Inc., Sunnyvale, Calif.). Cofluoron model samples were excited at discrete wavelengths and the fluorescence emission was scanned across a range of wavelengths (between 400 and 750 nm). The fluorescence properties of individual monomers, as well as the fluorescent properties of multimers formed by association of different combinations of monomers, were tested and compared.

Boronic acids paired with various partner partners, for instance, catechols, diols, o-hydroxyarylamides, alpha hydroxy carboxylic acids and amides, o-hydroxy arylhydroxamic acids and hydroxamates, were used as a model system of linker elements for cofluorons. These linker elements are suitable for appending to ligand elements to generate full-length cofluorons.

Among these linker elements, at least several combinations of the linker elements generated either substantially greater signal or a red or blue shift in the emission wavelength, or both greater signal or shift in emission wavelength occurred. The use of co-solvents, such as DMSO, which mimic binding of the linker elements to a more hydrophobic surface, can enhance the fluorescence intensity.

### Example 10

### Enhancement in Fluorescence Emission Intensity when Forming Multimers

A. Multimers Formed by Binding 3,4,5-Trihydroxybenzamide with 2-Fluorophenylboronic Acid

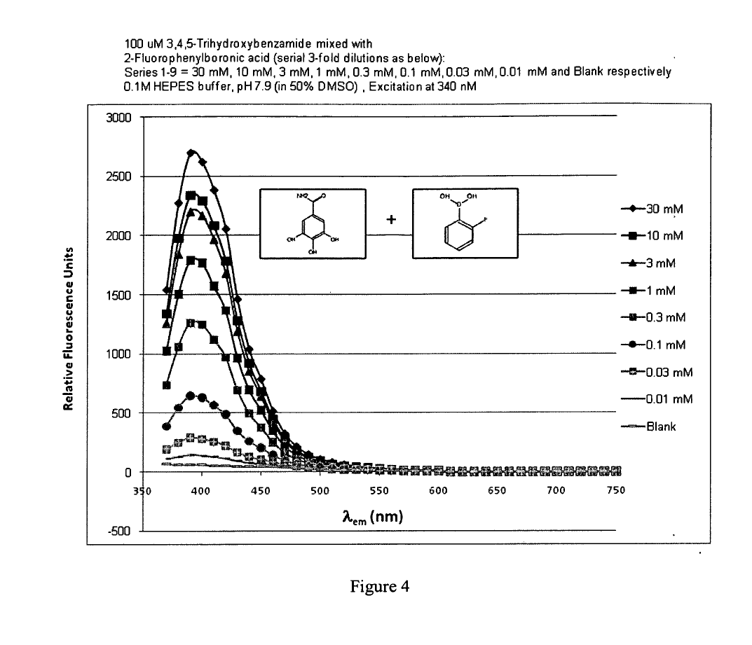

FIG. 4 shows the results of fluorescent measurements on the monomer 3,4,5-trihydroxybenzamide and the multimers formed by mixing 3,4,5-trihydroxybenzamide with different concentrations of 2-fluorophenylboronic acid. The multimers were formed by mixing 100 μM 3,4,5-trihydroxybenzamide with 2-fluorophenylboronic acid having concentrations as follows: 30 mM, 10 mM, 3 mM, 1 mM, 0.3 mM, 0.1 mM, 0.03 mM, 0.01 mM, and blank, respectively. Fluorescence signals were measured on samples in 0.1M HEPES buffer at pH 7.9 (in 50% DMSO), when excited at 340 nm.

Increased intensities of fluorescence emission were observed for cofluoron multimers formed when mixing 3,4,5-trihydroxybenzamide with different concentrations of 2-fluorophenylboronic acid. The multimer formed, when combining these two linker elements at 100 μM each, generated a fluorescent emission 390 nm with an intensity 10-fold higher compared with that produced by 3,4,5-trihydroxybenzamide alone.

B. Multimers Formed by Binding 7,8-Dihydroxy-4-Methylcoumarin with Various Boronic Acids

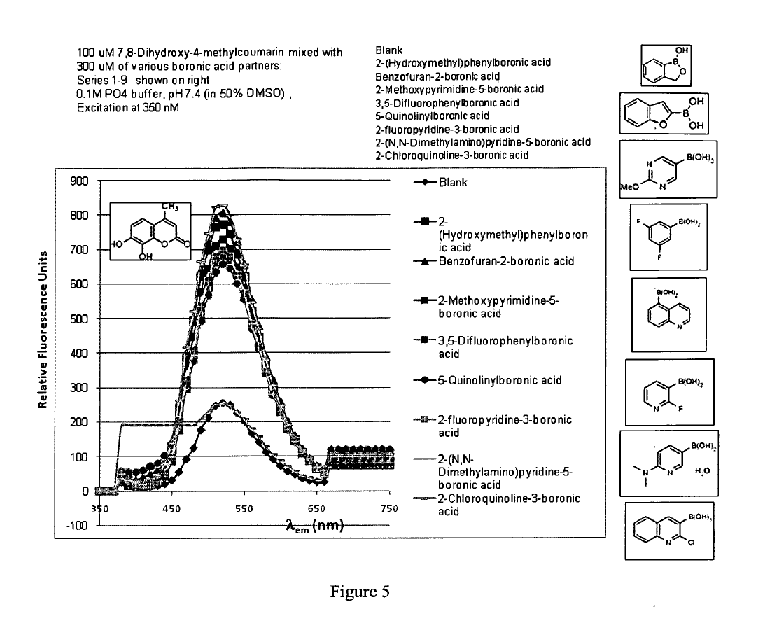

FIG. 5 shows the results of fluorescent measurements on the monomer containing a dihydroxy moiety and multimers formed by mixing the dihydroxy compound with various boronic acid binding partners. The multimers were formed by mixing 100 μM 7,8-dihydroxy-4-methylcoumarin with 300 μM of various boronic acid binding partners as follows: 2-(hydroxymethyl)phenylboronic acid, benzofuran-2-boronic acid, 2-methoxypyrimidine-5-boronic acid, 3,5-difluorophenylboronic acid, 5-quinolinylboronic acid, 2-fluoropyridine-3-boronic acid, 2-(N,N-dimethylamino)pyridine-5-boronic acid, and 2-chloroquinoline-3-boronic acid, respectively. Fluorescence signals were measured on samples in 0.1M phosphate buffer at pH 7.4 (in 50% DMSO), when excited at 350 nm.

The results showed that majority of the above boronic acids, when binding to the dihydroxy compound, can strongly enhance the fluorescence emission intensity of the dihydroxy compound. The dihydroxy compound, 7,8-dihydroxy-4-methylcoumarin, exhibited at least a 3-fold increase in fluorescence emission intensity (excitation wavelength=350 nm, and emission wavelength=520 nm) when forming covalent oligomers with the following boronic acid partners: 2-(hydroxymethyl)phenylboronic acid, benzofuran-2-boronic acid, 2-methoxypyrimidine-5-boronic acid, 3,5-difluorophenylboronic acid, 5-quinolinylboronic acid, 2-fluoropyridine-3-boronic acid, and 2-chloroquinoline-3-boronic acid. However, 2-(N,N-dimethylamino)pyridine-5-boronic acid did not provide any enhanced signal.

### Example 11

### Shift in Fluorescence Emission Wavelength when Forming Multimers

The fluorescent signatures generated by the cofluoron multimers formed by cofluoron monomer and its binding pairs can also include a shift in fluorescence emission wavelength either in the red or blue direction, compared to those produced by the cofluoron monomers. This allows for an increased ability to distinguish the target-induced fluorescent signal arising from cofluoron multimers formed by covalently linked cofluoron monomers from the background fluorescence from each individual cofluoron monomers.

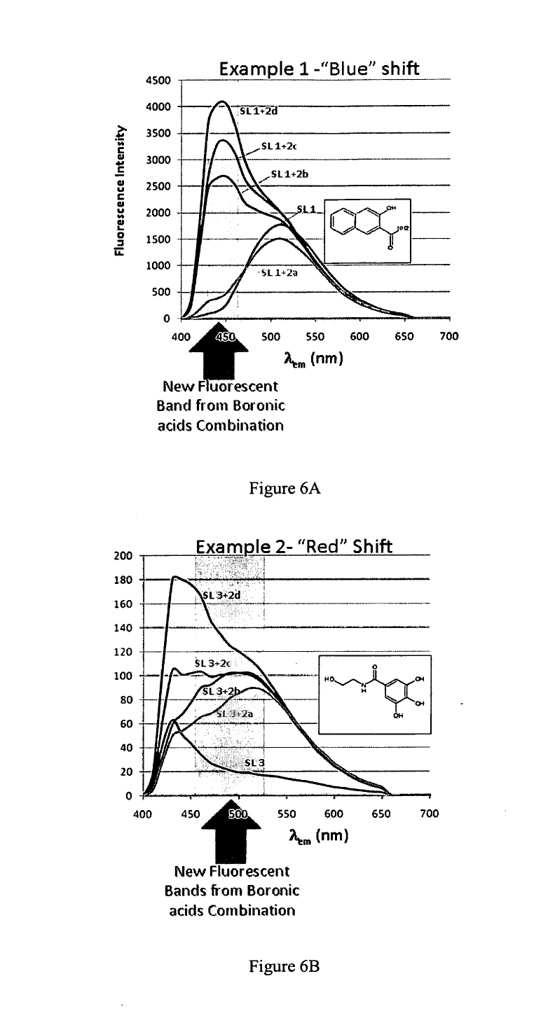

FIG. 6 illustrates the wavelength shifts in fluorescence emission for linker elements when binding to their binding partners. Linker elements SL1 and SL3 each are binding partners of boronic acid compounds, and each linker element alone can produce fluorescent signal. FIG. 6A shows that addition of 3 different boronic acid linker elements (2b, 2c, and 2d) to the linker element SL1 produced a stronger fluorescent signal, as well as a fluorescent emission wavelength shift to a lower wavelength (i.e. blue shift); while FIG. 6B shows that addition of 3 different boronic acid linker elements (2b, 2c, and 2d) to the linker element SL3 produced a stronger fluorescent signal, as well as a fluorescent emission wavelength shift to a higher wavelength (red shift).

A. Multimers Formed by Binding 2-Hydroxy-3-Naphthalenecarboxamide with Various Boronic Acids

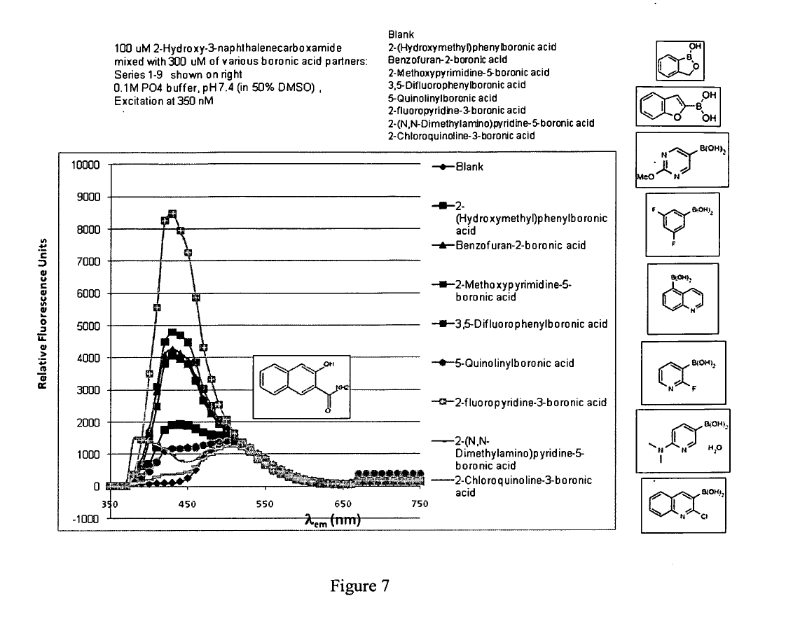

FIG. 7 shows the results of fluorescent measurements on the monomer 2-hydroxy-3-naphthalenecarboxamide and the multimers formed by mixing 2-hydroxy-3-naphthalenecarboxamide with various boronic acid binding partners. The multimers were formed by mixing 100 μM 2-hydroxy-3-naphthalenecarboxamide with 300 μM of various boronic acid binding partners as follows: 2-(hydroxymethyl)phenylboronic acid, benzofuran-2-boronic acid, 2-methoxypyrimidine-5-boronic acid, 3,5-difluorophenylboronic acid, 5-quinolinylboronic acid, 2-fluoropyridine-3-boronic acid, 2-(N,N-dimethylamino)pyridine-5-boronic acid, and 2-chloroquinoline-3-boronic acid, respectively. Fluorescent signals were measured on samples in 0.1M phosphate buffer at pH 7.4 (in 50% DMSO), when excited at 350 nm.

As a monomer, 2-hydroxy-3-naphthalenecarboxamide showed a fluorescent emission maximum at 500 nm. When forming multimers with various boronic acids, however, the maximum shifted to about 420 to 430 nm. When forming a multimer with 2-fluoropyridine-3-boronic acid, the fluorescent emission signal at 420 nm was enhanced more than 80-fold than that of the monomer 2-hydroxy-3-naphthalenecarboxamide. When forming multimers with benzofuran-2-boronic acid, 2-methoxypyrimidine-5-boronic acid, or 3,5-difluorophenylboronic acid, the fluorescent emission signals at 420 nm were enhanced about 40-fold than that of the monomer 2-hydroxy-3-naphthalenecarboxamide.

B. Solvent Effect on Fluorescent Measurements

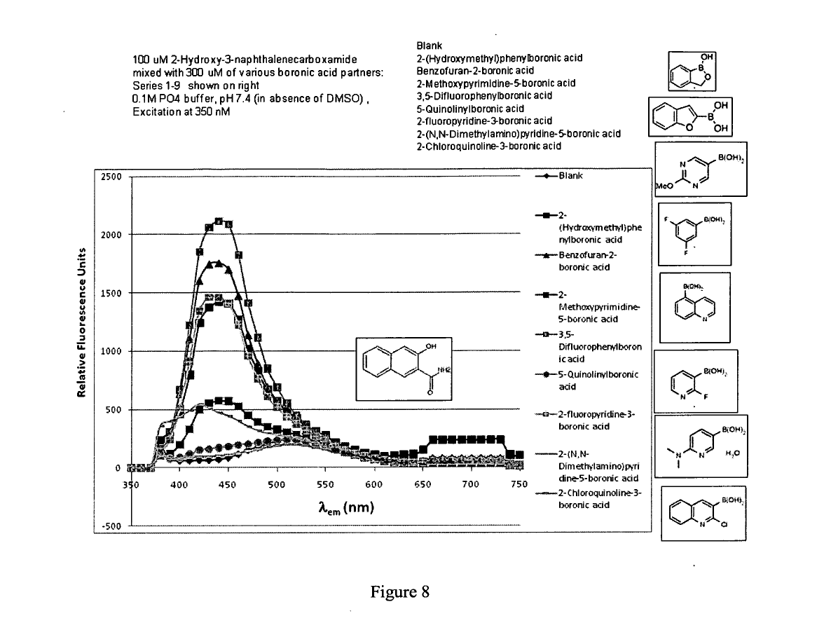

FIG. 8 shows the results of fluorescent measurements on the monomer 2-hydroxy-3-naphthalenecarboxamide and the multimers formed by mixing 2-hydroxy-3-naphthalenecarboxamide with various boronic acid binding partners. The multimers were formed similarly as the above experiment in Example 11A. Fluorescent signals were measured on samples in similar conditions as the above experiment in Example 11A, except that the experiments were carried out in the absence of DMSO.

In the absence of DMSO, a similar “blue shift” occurred for 2-hydroxy-3-naphthalenecarboxamide monomer when forming multimers with various boronic acids: a fluorescent emission maximum of about 510 nm for the monomer was shifted to peaks at about 440 nm for various boronate hetero-multimers of this naphthalene derivative. Overall, the total fluorescent emission signals were about 4-fold lower in aqueous solution than in 50% DMSO. In the absence of DMSO, the multimer formed by binding with 3,5-difluorophenylboronic acid appeared to produced the highest fluorescent signal (30-fold enhancement), among the tested boronic acids; and other binding partners that produced strong fluorescent signal included benzofuran-2-boronic acid, 2-methoxypyrimidine-5-boronic acid, and 2-fluoropyridine-3-boronic acid (20 to 35-fold enhancement).

C. pH Effect on Fluorescent Measurements

Fluorescence signatures can also be modulated by changing pH. The above experiments in Examples 11A and 11B were repeated using 100 μM 2-hydroxy-3-naphthalenecarboxamide monomer mixed with 100 μM of various boronic acid partners as follows: benzofuran-2-boronic acid, 3,5-difluorophenylboronic acid, and 2-fluoropyridine-3-boronic acid, in both DMSO and aqueous solutions.

When the pH for reactions carried out in 50% DMSO was increased from 6.4 to 7.4 and to 8.4, the best fluorescence enhancement, among all multimers formed, went from 38-fold to 29-fold, and to 8-fold, respectively, as the overall fluorescence intensity dropped from 6000 to 3600, and to 700 at 440 nm, respectively. Simultaneously, the fluorescent emission peaks for the formed multimers shifted from 440 nm to 510 nm, but there was no enhancement for fluorescent signals at 510 nm.

In contrast, as the pH for reactions carried out in aqueous conditions was increased from 6.4 to 7.4, and to 8.4, the best fluorescence enhancement, among all multimers formed, went from 10-fold to 21-fold, and to 23-fold, respectively, as the overall intensity increased from 600 to 1300, and to 1500 at 440 nm, respectively. At the same time, the fluorescent emission peaks for the formed multimers stayed at 440-450 nm, and the fluorescent emission peak for the monomer stayed at 510-520 nm.

D. Multimers Formed by Binding Methyl 3,4,5-Trihydroxybenzoate with Various Boronic Acids

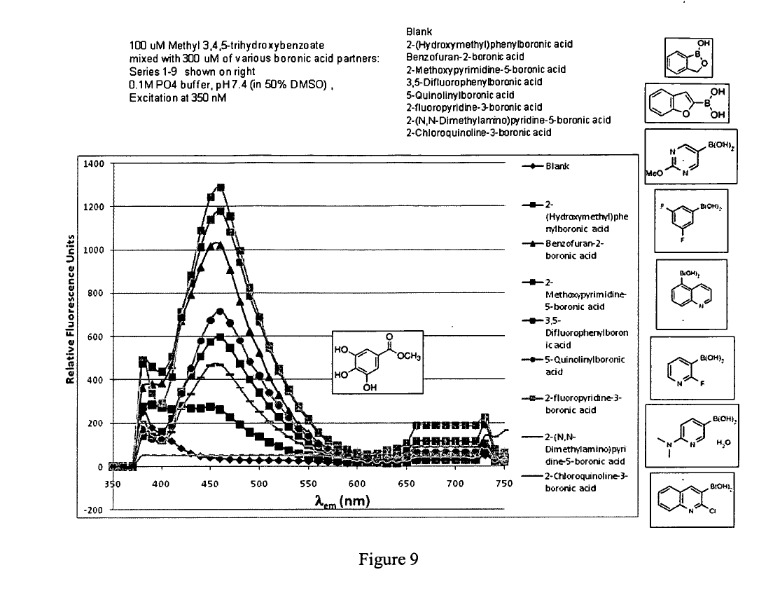

FIG. 9 shows the results of fluorescent measurements on the monomer methyl 3,4,5-trihydroxybenzoate and the multimers formed by mixing methyl 3,4,5-trihydroxybenzoate with various boronic acid binding partners. The multimers were formed by mixing 100 μM methyl 3,4,5-trihydroxybenzoate with 300 μM of various boronic acid binding partners as follows: 2-(hydroxymethyl)phenylboronic acid, benzofuran-2-boronic acid, 2-methoxypyrimidine-5-boronic acid, 3,5-difluorophenylboronic acid, 5-quinolinylboronic acid, 2-fluoropyridine-3-boronic acid, 2-(N,N-dimethylamino)pyridine-5-boronic acid, and 2-chloroquinoline-3-boronic acid, respectively. Fluorescent signals were measured on samples in 0.1M phosphate buffer at pH 7.4 (in 50% DMSO), when excited at 350 nm.

When forming multimers with various boronic acids, the fluorescent emission peak of methyl 3,4,5-trihydroxybenzoate shifted from about 390 nm to about 460 nm. When forming a multimer with 2-fluoropyridine-3-boronic acid, the fluorescent emission signal at 420 nm was enhanced more than 80-fold than that of the monomer 2-hydroxy-3-naphthalenecarboxamide. When forming multimers with 2-fluoropyridine-3-boronic acid, benzofuran-2-boronic acid, or 3,5-difluorophenylboronic acid, the fluorescent emission signals at 460 nm were enhanced about 28- to 35-fold than that of the monomer methyl 3,4,5-trihydroxybenzoate. Again, the fluorescent signal changes when forming multimers also depend on the boronic acid partner of the monomer. For example, when forming multimers with 2-(hydroxymethyl)phenylboronic acid, the fluorescent emission signals at 460 nm were enhanced about 7-fold, although not as high as the other binding partners tested herein.

E. Multimers Formed by Binding 3,4,5-Trihydroxybenzamide with Various Boronic Acids

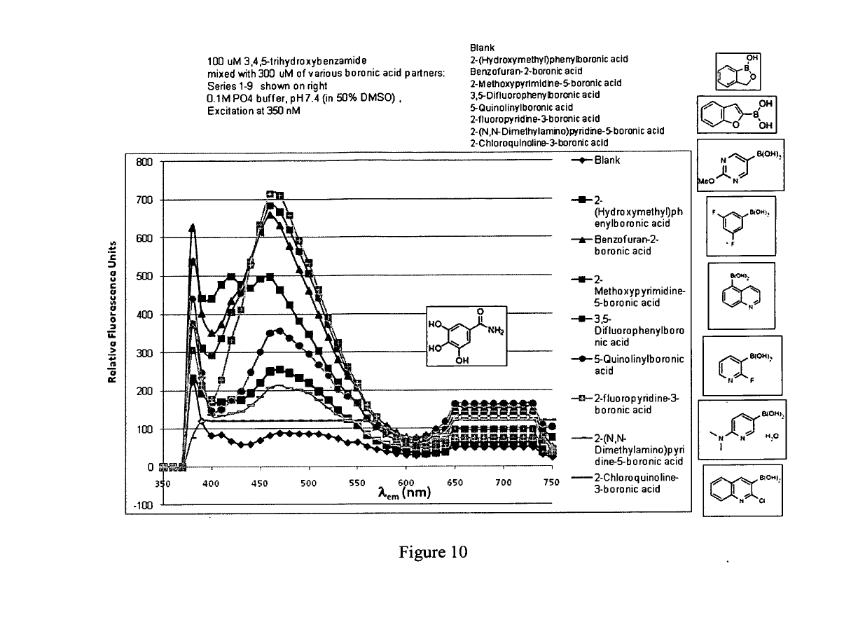

FIG. 10 shows the results of fluorescent measurements on the monomer 3,4,5-trihydroxybenzamide and the multimers formed by mixing 3,4,5-trihydroxybenzamide with various boronic acid binding partners similar as those in the above experiment in Example 11D. Fluorescent signals were measured on samples under similar conditions as the above experiment in Example 11D.

As in Example 11D, using 3,4,5-trihydroxybenzamide, which has a structure closely related to methyl 3,4,5-trihydroxybenzoate, also produced a red shift when forming multimers with various boronic acids. 3,4,5-trihydroxybenzamide also showed a similar binding preference with 2-fluoropyridine-3-boronic acid, benzofuran-2-boronic acid, or 3,5-difluorophenylboronic acid, as reflected from the higher fold on fluorescent enhancement when binding to these boronic acids than the others binding partners tested herein. However, because the monomer 3,4,5-trihydroxybenzamide alone is capable of producing a strong fluorescent signal, the fluorescent enhancement, when binding to the above boronic acid partners, was about 7- to 8-fold.

When the above experiments for binding 3,4,5-trihydroxybenzamide with various boronic acids were repeated in aqueous buffer, in the absence of DMSO, the fluorescent emission intensities of the formed multimers were less than those formed in the presence of DMSO, but the red shifts were still observed.

### Example 12

### Fluorescence Measurements of Cofluorons

The fluorescence properties of cofluorons were measured in 0.1M HEPES or 0.1M phosphate buffer at pH 7.4 in 96- or 384-well black plates using a Molecular Dynamics SpectraMax M5 plate reader. Cofluoron samples were excited at discrete wavelengths and the fluorescence emission was scanned across a range of wavelengths (between 400 and 750 nm). The fluorescence properties of individual cofluoron monomers, as well as the fluorescent properties of cofluoron multimers formed by association of different combinations of cofluoron monomers, were tested and compared.

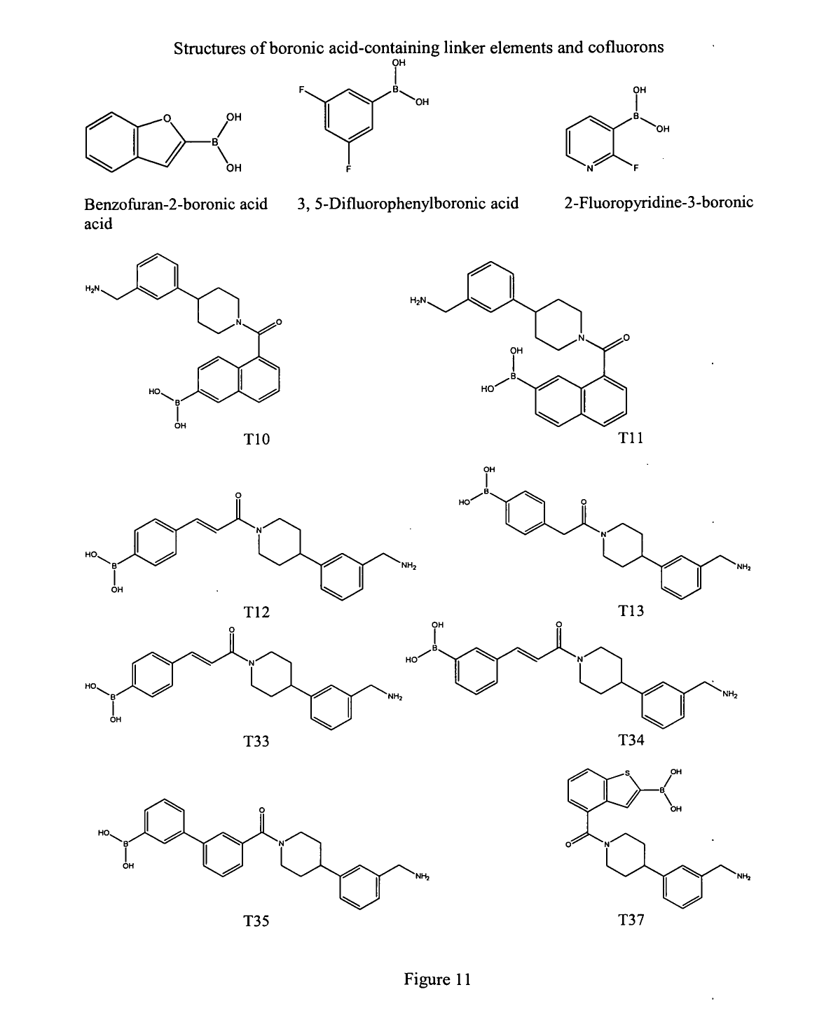

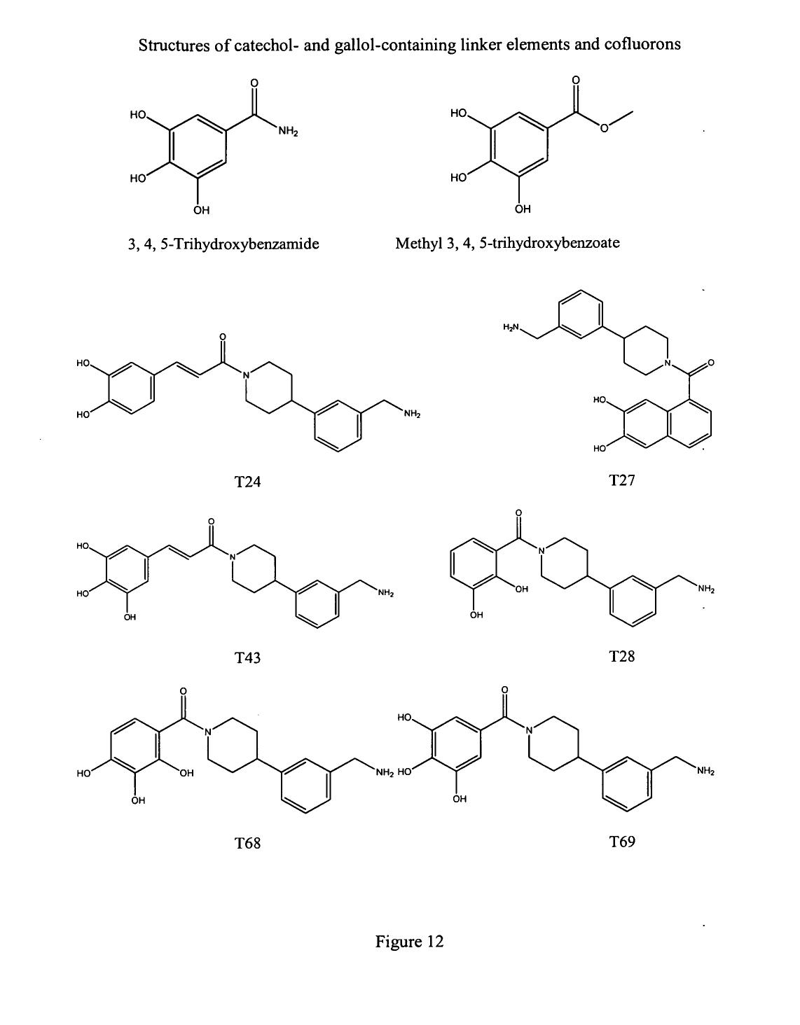

The binding target chosen for the fluorescent measurements of cofluorons in this example was human mast cell β2-tryptase, a serine protease that is released upon degranulation and that plays a role in response to foreign antigens. Inhibition of this enzyme may ameliorate the effects of an overactive immune response, leading to allergic rhinitis, conjunctivitis, dermatitis, anaphylaxis, and even ulcerative colitis. Cofluorons were designed and synthesized to bind to Tryptase as monomers with an IC₅₀ ranging from about 0.5 μM to about 10 μM. FIGS. 11 and 12 demonstrate various designs of linker elements and potential cofluoron monomers that contain boronic acid (FIG. 11) and its binding partners catechol and gallol (FIG. 12).

A. Cofluoron Dimers Formed by Binding Monomer T27 with its Various Binding Partners

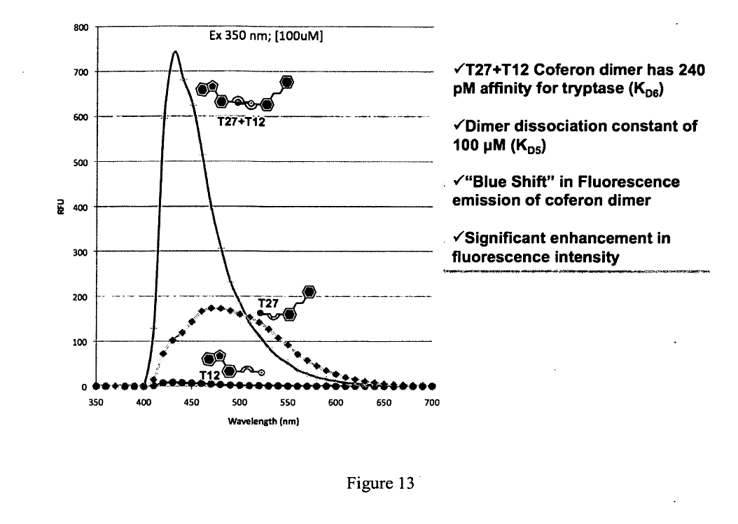

FIG. 13 shows the results of fluorescent measurements on the cofluoron multimers formed by binding cofluoron monomer T12 and T27 compared to those measurements on individual cofluoron monomers alone. The multimers were formed by mixing 100 μM T27 and 100 μM of cofluoron monomers T10, T11, T12, and T13. Fluorescent signals were measured on samples excited at 350 nm.

When binding cofluoron monomer T27 with its various binding partners, the fluorescent emission peaks for all formed cofluoron multimers had a blue shift, as well as an enhancement on peak intensity. Among all formed cofluoron multimers, T12 and T27 cofluoron dimer had a significantly higher enhancement in fluorescent emission intensity than the other cofluoron dimers.

B. Cofluoron Dimers Formed by Binding Monomers T11 and T24

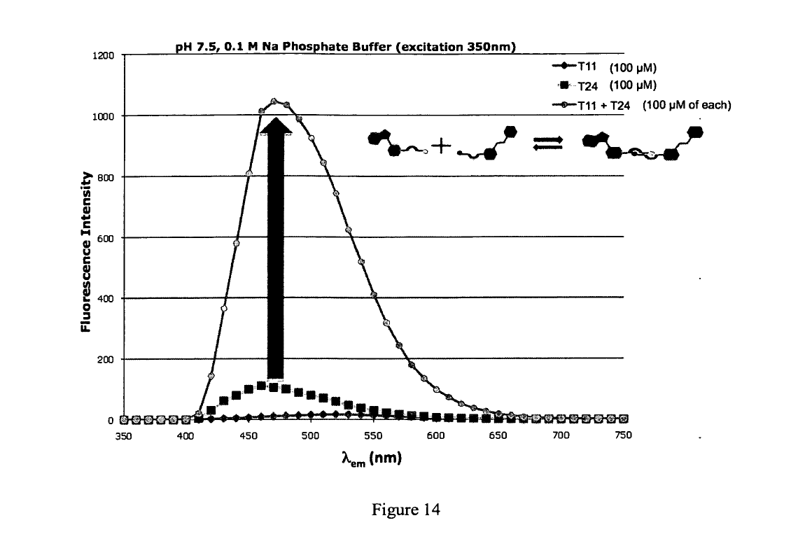

FIG. 14 shows the results of fluorescent measurements on another cofluoron dimer that had a significantly high fluorescence enhancement upon binding. The cofluoron dimer formed by binding cofluoron monomers T11 and T24 produced an 11-fold enhancement in fluorescent emission intensity compared to those of individual cofluoron monomers.

C. Cofluoron Dimers Formed by Binding Monomer T43 with its Various Binding Partners

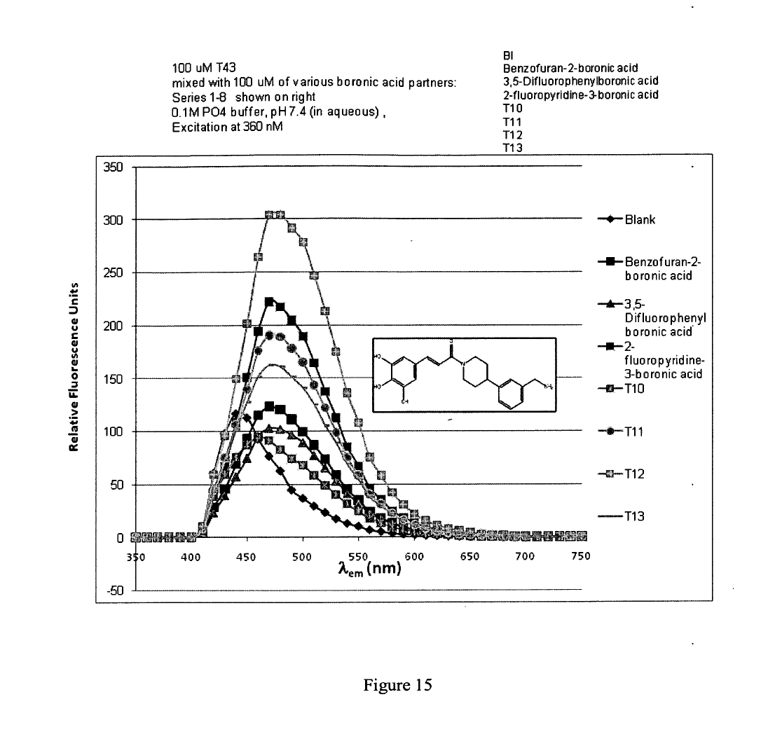

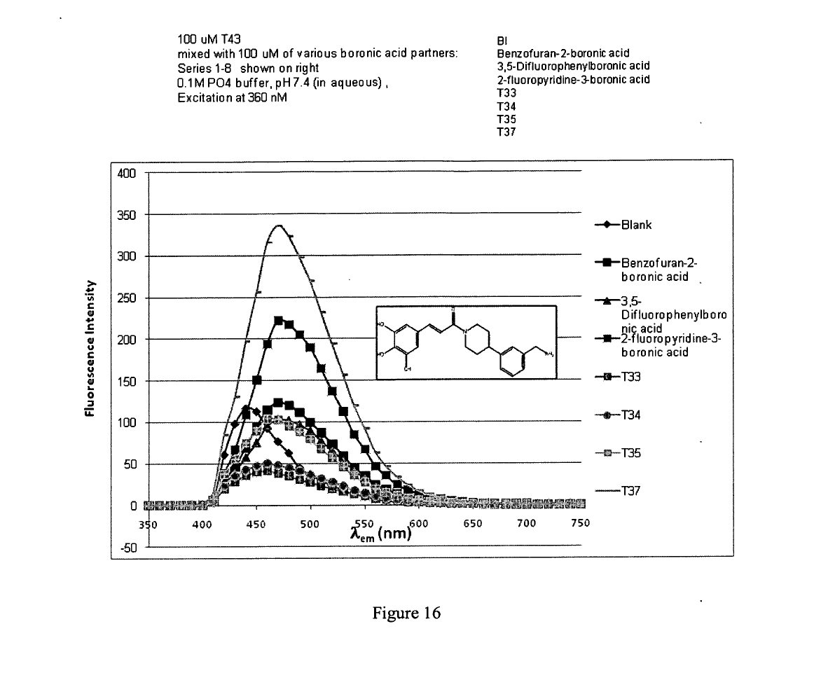

FIGS. 15 and 16 show the results of fluorescent measurements on the cofluoron monomer T43 and on the cofluoron multimers formed by binding T43 with various boronic acid binding partners and with various cofluoron monomers. The multimer was formed by mixing 100 μM T43 with 100 μM of various binding partners as follows: blank, benzofuran-2-boronic acid, 3,5-difluorophenylboronic acid, 2-fluoropyridine-3-boronic acid, T10, T11, T12, T13 (FIG. 15), T33, T34, T35, and T37 (FIG. 16), respectively. Fluorescent signals were measured on samples in 0.1M phosphate buffer at pH 7.4 (in aqueous), when excited at 360 nm.

For the boronate-T43 multimers, all the complexes formed showed a slight “red shift” from an emission peak of 440 nm for the cofluoron monomer T43 to an emission peak of 470 nm. The strongest fluorescent signal enhancements observed, among all multimers tested herein, were for cofluoron multimers formed by covalently linking cofluoron monomer T43 with two other cofluoron monomers, T12 and T37.

### Example 13

### Intracellular Fluorescence of Cofluorons

Human mast cell β2-tryptase containing cells, grown in standard media, were incubated with 100 μM monomer 1 for 15 minutes at room temperature, followed by the addition of 100 μM monomer 2. Stock concentrations of cofluorons, 50 mM in DMSO, were diluted in media immediately before use. For localization studies, 10.5% PEG and 0.0005% berberine were added to costain mast cell granules. Fluorescent signals were detected using an inverted CKX41 Olympus microscope under UV excitation and an 100× oil immersion lens. Images were recorded using an Olympus DP20 digital camera.

The fluorescence emission measurements on cofluorons binding to the target tryptase were measured in 0.1M HEPES or 0.1M phosphate buffer at pH 7.4 in 96- or 384-well black plates using a Molecular Dynamics SpectraMax M5 plate reader. Cofluoron samples were excited at discrete wavelengths and the fluorescence emission was scanned across a range of wavelengths (between 400 and 750 nm). The fluorescence properties of cofluoron monomers and cofluoron multimers in the presence of 3 μM tryptase as well as in the absence of tryptase were measured and compared.

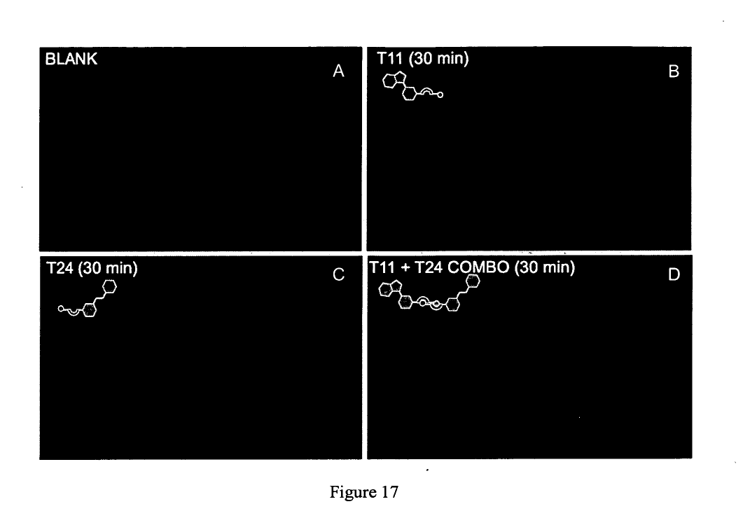

FIGS. 17A-17D are fluorescent images demonstrating the permeation of cofluoron monomers T11 and T24 into a human mast cell line and the detection of formation of cofluoron dimer T11-T24 inside the cells, by enhanced fluorescent signals. FIG. 17A is an image of untreated cells as a control showing background staining under the excitation of UV wavelength. FIG. 17B shows a faint staining after cells were treated with 100 μM cofluoron monomer T11; and FIG. 17C shows a somewhat brighter staining after cells were individually treated with 100 μM cofluoron monomer T24. FIG. 17D shows a remarkable increase in fluorescence signals in the cells after both cofluoron monomers were added (100 μM each).

These results demonstrate that cofluorons can permeate the cells as monomers, and that after entering the cells, the cofluoron monomers have combined to form cofluoron dimers which showed significantly enhanced fluorescence signals inside the mast cell line. Counterstaining with berberine shows colocalization with the cofluorons T11+T24 staining, which suggest that the cofluorons are within the granules.

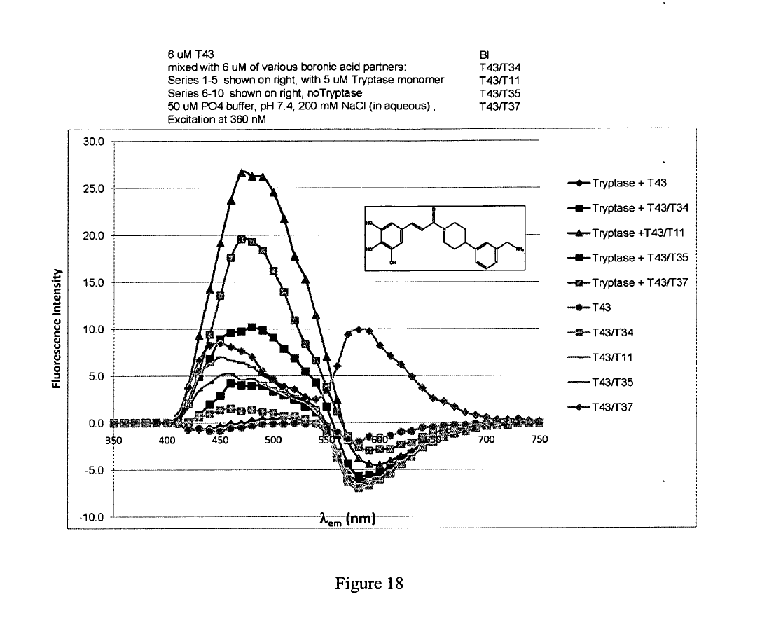

To test whether the cofluorons retained their fluorescence when binding to the target Tryptase, fluorescence were measured on the cofluoron multimers formed by mixing 6 μM T43 with 6 μM T34, T11, T35, and T37, respectively, in the presence or absence of 5 μM Tryptase, in aqueous 50 μM phosphate buffer (pH 7.4) containing 200 mM NaCl. The results are shown in FIG. 18. Excitation was at 360 nm, and the portion of emission containing meaningful signal is between 400 nm and 550 nm.

The cofluorons were added in about a 2-fold in excess of the tryptase so that low level of multimer formation in the absence of target can also be observed, and hence that a fluorescent emission change due to the binding of cofluoron dimer to the target, if it occurs, would be recorded. Cofluoron monomer T43, in the absence of a boronic acid partner, exhibited essentially no fluorescent signal between 400 nm and 550 nm. When cofluoron monomer T43 was linked with cofluoron T34 to form a dimer in the absence of tryptase, there was hardly any fluorescent signal, while the signal was enhanced by 3-fold in the presence of tryptase.

Among all the cofluoron pairs tested herein, the most intense fluorescent signal was observed for the T43/T11 cofluoron pair at 490 nm. T43/T11 cofluoron pair also produced the highest fluorescent enhancement (6-fold) in the presence of tryptase versus in the absence of tryptase. Both T43/35 and T43/T37 cofluoron pairs generated a fluorescent signal that was enhanced 2-fold and 3-fold, respectively, in the presence of tryptase.

While these increases in fluorescent emission intensity of cofluorons in the presence of tryptase may be modest, they do illustrate the ability of tryptase to shift the equilibrium towards the formation of the cofluoron multimers on the enzyme target.

### Example 14

### Synthesis of Cofluorons with Boronic Acid Functionality

Eight targets with boronic acid functionality were synthesized. These compounds were synthesized by two approaches.

In Approach-1, the aryl boronic acids or their pinacolato boronate esters with carboxylic acid were coupled to a protected core (Core-1 or Core-4 shown in synthetic scheme). Product was deprotected to obtain the target boronic acids.

In Approach-2, desired halo aryl carboxylic acids were coupled to the appropriate protected core. The boronate ester/acid was then introduced on the coupled product and deprotected to give the desired target boronic acids.

The desired aryl halo carboxylic acids in Step-1 of both the approaches were either procured commercially or synthesized in-house by known methods in the literature. The details of the synthesis of these targets are given below.

### Approach-1

Desired aryl boronic acids or their pinacolato boronate esters with carboxylic acid groups were synthesized and coupled with a protected core (Core-1 or Core-4 shown in synthetic scheme). Coupled products were deprotected. During deprotection reaction of intermediates containing boronate ester functionality, either partial or complete hydrolysis of boronate esters to boronic acids occurred. Mixture of boronate ester and boronic acid was then subjected to purification by preparative HPLC under acidic condition, during which, remainder of the boronate ester was converted to boronic acid.

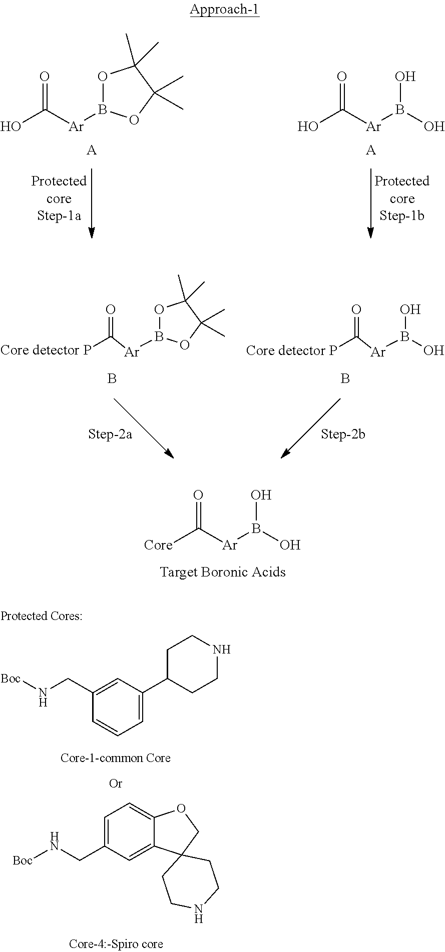

### A. Synthesis of Boronate Ester or Boronic Acid Precursors

The details of intermediates sourced/synthesized as per literature methods/synthesized by adapted methods are given below.

| Compound | |
|---|---|
| Code | Structure |
| A-116 | 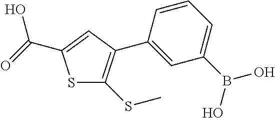 [SMILES: CSc1sc(C(=O)O)cc1-c1cccc(B(O)O)c1] 4-(3-boronophenyl)-5-(methylthio)thiophene- 2-carboxylic acid |
| A-131 | 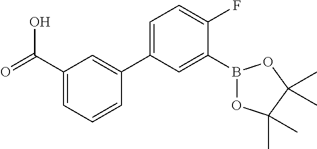 [SMILES: CC1(C)OB(c2cc(-c3cccc(C(=O)O)c3)ccc2F)OC1(C)C] 4′-fluoro-3′-(4,4,5,5-tetramethyl-1,3,2-dioxaborolan- 2-yl)-[1,1′-biphenyl]-3-carboxylic acid |
| A-143 | 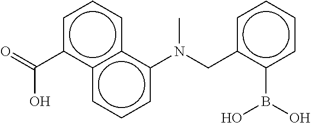 5-((2-boronobenzyl)(methyl)amino)-1-naphthoic acid |
| A-146 | 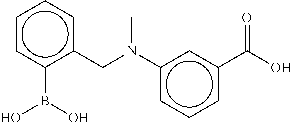 3-((2-boronobenzyl)(methyl)amino)benzoic acid |
| A-147 | 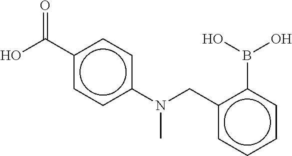 4-((2-boronobenzyl)(methyl)amino)benzoic acid |
| A-154 | 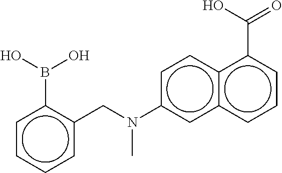 6-((2-boronobenzyl)(methyl)amino)-1-naphthoic acid |
| A-155 | 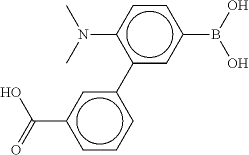 5′-borono-2′-(dimethylamino)-[1,1′-biphenyl]-3- carboxylic acid |

### Synthesis of 4-(3-boronophenyl)-5-(methylthio)thiophene-2-carboxylic acid (A-116)

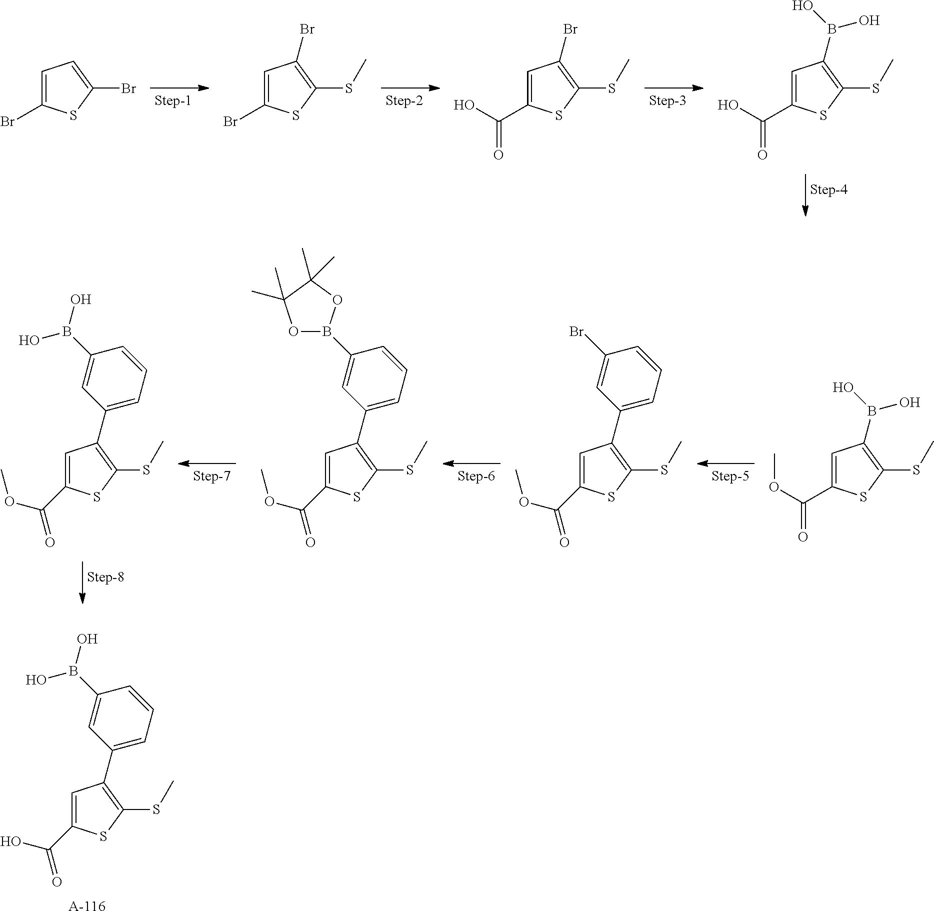

### Step-1:

2,4-dibromo-5-methylthio thiophene was synthesized as per procedures available in the literature (Kano et al., Heterocycles 20(10): 2035-37 (1983).

### Step-2:

Lithiation of 2,4-dibromo-5-methylthio thiophene (28.13 g, 97.7 mmol) was done by adding n-BuLi (7.46 g, 116.64 mmol) at −78° C. in THF (562 ml) under stirring. At same temperature dry-ice was carefully added, during which, the temperature of the reaction mixture was allowed to rise to room temperature. The reaction mixture was then quenched with dilute HCl and concentrated. The residue obtained was diluted with HCl, filtered, and washed with methanol to obtain the product. Yield: 17.2 g, 70%. MS (ES+): m/z=253.20/255.20 [MH+].

### Step-3:

Lithiation of 4-bromo-5-(methylthio)thiophene-2-carboxylic acid (the product of Step-2; 14.99 g, 59.25 mmol), was done by adding n-BuLi (11.37 g, 177.76 mmol) in THF (300 mL) at −78° C. under stirring. After 30 minutes, at same temperature tri-isopropyl borate (32.53 g, 177.76 mmol) was carefully added drop wise, during which, and the temperature of the reaction mixture was allowed to raise to room temperature. The reaction mixture was quenched with dilute HCl and concentrated in vacuo. The residue obtained was diluted with dilute HCl, filtered, washed with water, re-dissolved in aqueous NaOH, and re-precipitated by acidifying with dilute HCl to obtain pure product. Yield: 10.36 g, 80%. MS (ES+): m/z=219.10 [MH⁺].

### Step-4:

To ice cold methanol (30 vol.) was added concentrated sulphuric acid (2 vol.) and then 4-borono-5-(methylthio)thiophene-2-carboxylic acid (the product of Step-3; 9.9 g, 45.85 mmol), was added. The reaction mixture was heated to reflux until completion of the reaction. After completion, the reaction mixture was concentrated to its 25% volume and poured on crushed ice. The solid precipitated was filtered and washed with water to obtain pure product. Yield: 7.45 g, 70%. MS (ES+): m/z=233.25 [MH⁺].

### Step-5:

Suzuki coupling of (5-(methoxycarbonyl)-2-(methylthio)thiophen-3-yl)boronic acid (the product of Step-4; 5 g, 21.54 mmol) with 3-bromo iodobenzene (7.31 g, 25.85 mmol) was carried out in presence of Palladium (0) tetrakis(triphenyl phosphine) (10 mol %) in dioxane (20 vol.), water (5 vol.), and sodium carbonate (4.56 g, 43.08 mmol), and heated at 80° C. for 15 hours. After completion of the reaction, the reaction mixture was filtered through a pad of celite, and filtrate was concentrated in vacuo. The residue was diluted with water and extracted with ethyl acetate to obtain crude product. Crude product obtained was purified by column chromatography over silica gel eluting with 5-10% ethyl acetate in hexane. Yield: 3.69 g, 50%. MS (ES+): m/z=343/345.10 [MH⁺].

### Step-6

Stirred suspension of methyl 4-(3-bromophenyl)-5-(methylthio)thiophene-2-carboxylate (the product of Step-5; 2.6 g, 7.8 mmol) in toluene (30 mL) was degassed with argon, and charged with potassium acetate (3 eq.), PdCl₂-DPPF—CH₂Cl₂ (5 mol %) and bis(Pinacolato)diborane (4.93 g, 19.5 mmol), dppf (3 mol %). Reaction was heated to reflux, and monitored by LCMS until most of the starting material was consumed. The mixture was filtered through a pad of celite. The filtrate was concentrated under reduced pressure to yield the crude product. The crude product was purified by column chromatography over silica gel eluting with 1-5% ethyl acetate in hexane. Yield: 2.14 g, 70%. MS (ES+): m/z=391.15 [MH⁺].

### Step-7:

To ice cold methanol (30 mL) was added concentrated sulfuric acid (2 mL), and then methyl 5-(methylthio)-4-(3-(4,4,5,5-tetramethyl-1,3,2-dioxaborolan-2-yl)phenyl)thiophene-2-carboxylate (the product of Step-6; 2.1 g, 5.38 mmol) was added at 0° C. Reaction mixture was heated to reflux until completion of reaction. After completion, the reaction mixture was concentrated to 25% of its volume and poured over crushed ice. The precipitate was filtered and washed with water to obtain pure product. Yield: 1.3 g, 80%. MS (ES+): m/z=309.20 [MH⁺].

### Step-8:

A mixture of Step-7 product (1.29 g, 4.21 mmol), potassium hydroxide (2.36 g, 42.13 mmol), THF (10 mL) and water (20 mL) was heated to 60° C. for 2 h. The reaction was monitored by LCMS until most of the starting was consumed. The reaction mixture was concentrated in vacuo and diluted with water. The pH of the reaction mixture was then adjusted to ˜2 using concentrated HCl resulting in a precipitate. The precipitate was filtered, washed with water and dried in vacuum oven. Yield: 744 mg, 60%. MS (ES+): m/z=295.20 [MH⁺].

### Synthesis of 4′-fluoro-3′-(4,4,5,5-tetramethyl-1,3,2-dioxaborolan-2-yl)-[1,1′-biphenyl]-3-carboxylic acid (A-131)

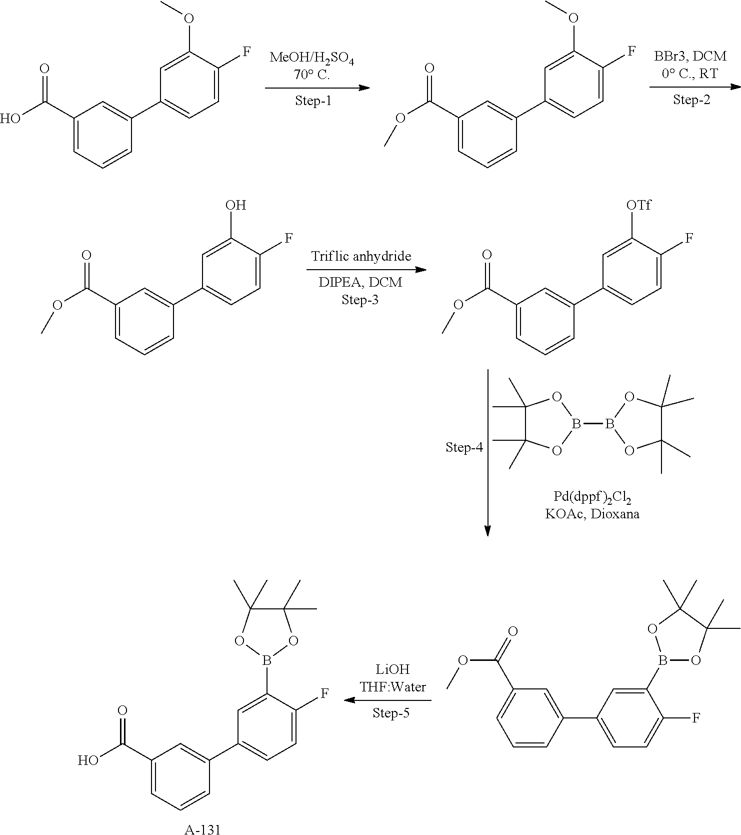

### Step-1:

4′-fluoro-3′ methoxy biphenyl-3-carboxylic acid (1 g, 4.865 mmol) was dissolved in methanol (25 mL and the solution was cooled to 0° C. Thionyl chloride (0.8 ml, 12.19 mmol) was added drop wise and then refluxed at 70° C. overnight. The methanol was concentrated in vacuo and the residue was diluted with ethyl acetate. The organic layer was washed with water (1×25 mL) and 10% NaHCO₃ solution, and then separated from aqueous layer. The organic layer was dried over sodium sulfate, filtered, and concentrated in vacuo to obtain pure product (off white solid). Yield: 1.01 g (95%). MS (ES+): m/z=261 [MH⁺]. ¹H NMR (400 MHz, CDCl₃): δ 8.22 (s, 1H), 8.02 (d, J=7.7 Hz, 1H), 7.73 (d, J=87.6 Hz, 1H), 7.51 (t, J=7.6 Hz, 1H), 7.22-7.08 (m, 3H), 3.96 (d, J=6.2 Hz, 6H).

### Step-2:

A stirred solution of methyl-4′-fluoro-3′-methoxy-[1,1′-biphenyl]-3-carboxylate (900 mg, 3.46 mmol) in dichloromethane (25 mL) was cooled to 0° C. and dropwise charged with boron tribromide (1.0 ml, 10.38 mmol) under a nitrogen atmosphere, and stirred at room temperature for 5 hours. The reaction mixture was cooled, quenched with methanol, and then concentrated in vacuo. The steps of charging with methanol and being concentrated in vacuo are repeated several times to remove excess of bromine. Yield: 800 mg (94%). ¹H NMR (400 MHz, DMSO-d₆): δ 8.08 (t, J=1.9 Hz, 1H), 7.92 (d, J=87.7 Hz, 1H), 7.87-7.82 (m, 1H), 7.59 (t, J=7.8 Hz, 1H), 7.25 (d, J=6.0 Hz, 2H), 7.10 (ddd, J=8.3, 4.3, 2.4 Hz, 1H), 3.86 (s, 3H).

### Step-3:

A stirred solution of methyl-4′-fluoro-5′-hydroxy-[1,1′-biphenyl]-3-carboxylate (800 mg, 3.25 mmol) in dichloromethane (30 mL) was charged with DIPEA (1.7 ml, 9.76 mmol) at 0° C. then charged with triflic anhydride (1.67 ml, 9.76 mmol) and stirred at room temperature for 6 hr. The reaction mixture was quenched with water followed by wash with 1N HCl (25 mL) and brine solution. The organic layer was separated and dried over Na₂SO₄, filtered, and concentrated in vacuo resulting in crude product in DIPEA as yellow oil. The crude compound was further purified by column chromatography on silica gel eluting with (n-hexane-ethyl acetate 9:1) to give 850 mg pure product as white solid. Yield: 850 mg (85%). ¹H NMR (400 MHz, DMSO-d₆): δ 8.24-8.19 (m, 1H), 8.13 (dd, J=7.1, 2.3 Hz, 1H), 8.01-7.97 (m, 2H), 7.91 (ddd, J=8.9, 4.8, 2.5 Hz, 1H), 7.75-7.62 (m, 2H), 3.90 (d, J=1.4 Hz, 3H).

### Step-4:

A solution of methyl-4′-fluoro-3′-(((trifluoromethyl)sulfonyl)oxy)-[1,1′-biphenyl]-3-carboxylate (500 mg, 1.322 mmol), potassium acetate (444 mg, 4.629 mmol), bis pinacolato diborane (3.34 g, 13.22 mmol) in anhydrous dioxane (15 mL) was degassed for 15 minutes under argon. To this mixture, Pd(dppf)Cl₂ (64.7 mg, 0.0793 mmol), dppf (43.4 mg, 0.0793 mmol) were added and again degassed for 10 minutes, and stirred at 80° C. for 12-14 hr. The reaction mixture was filtered through a pad of celite. The filtrate was concentrated in vacuo. The residue was diluted with ethyl acetate and washed with water followed by brine. The organic layer was dried over sodium sulfate, filtered, and concentrated in vacuo to obtain crude product. The crude product was further purified by column chromatography on silica gel eluting with (n-hexane-ethyl acetate 8:2) to obtain 650 mg product contaminated with some bis pinacolato diborane. Yield: 600 mg. ¹H NMR (400 MHz, DMSO-d₆): δ 1.33 (s, 12H), 3.89 (s, 3H), 7.36-7.23 (m, 1H), 8.02-7.79 (m, 3H), 8.15-8.07 (m, 1H), 7.70-7.57 (m, 1H).

### Step-5:

To a solution of 4′-fluoro-3′-(4,4,5,5-tetramethyl-1,3,2-dioxaborolan-2-yl)-[1,1′-biphenyl]-3-carboxylate (600 mg, 1.685 mmol) in THF:water (10 mL) was added lithium hydroxide (212 mg, 5.056 mmol) and stirred at room temperature overnight. The solvent was concentrated in vacuo and the pH of residue was adjusted up to 2. Major product spot was isolated by acid base work-up. Yield: 200 mg. ¹H NMR (400 MHz, DMSO-d₆): δ 13.12 (brs, 1H), 8.12 (t, J=1.9 Hz, 1H), 7.97-7.84 (m, 4H), 7.61 (t, J=7.7 Hz, 1H), 7.29 (t, J=8.7 Hz, 1H), 1.33 (s, 12H).

### Synthesis of 5-((2-boronobenzyl)(methyl)amino)-1-naphthoic acid (A-143)

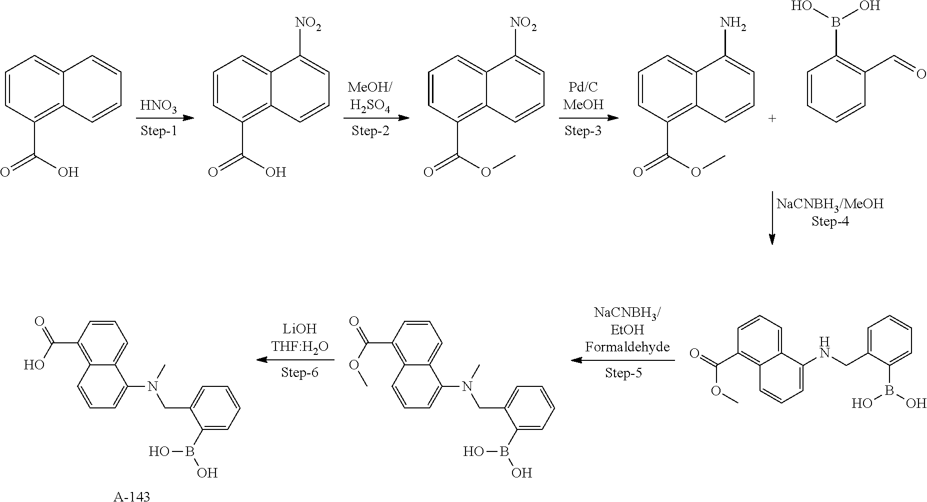

### Step-1:

To cold fuming nitric acid (3 ml, 660 mmol) at 0-5° C. was charged with α-naphthoic acid (1 gm, 5.8 mmol) portion-wise over a 15-minute period. The reaction mixture was stirred at 0-5° C. for 30 minutes and then at room temperature for an additional 2 hr. The reaction mixture was poured into 20 ml ice-cold water upon which a precipitate formed. The precipitate was filtered and washed with 10 ml water. The solid obtained was dissolved in 10 ml 8% sodium carbonate and stirred for 10 minutes and filtered. Filtrate was acidified using 10% HCl (pH=2) and the precipitate was filtered and re-crystallized from ethanol, filtered and dried under vacuum to obtain a yellow solid. Yield: 1.14 g, 90.47%, HPLC Purity: 98.09%. ¹H NMR (400 MHz, DMSO-d₆): δ 13.57 (s, 1H), 9.19 (d, J=8.8 Hz, 1H), 8.54-8.21 (m, 3H), 7.85 (dt, J=16.3, 7.8 Hz, 2H).

### Step-2:

A stirred solution of Step-1 product (1 g, 4.60 mmol) in methanol (15 ml) was charged with concentrated sulfuric acid and heated to reflux at 70° C. for 24 hours. The solvent was concentrated in vacuo and the residue was basified to pH=8 using 10% sodium bicarbonate and extracted with ethyl acetate (3×20 ml). The combined organic layer was washed with brine (2×10 ml), dried over sodium sulfate, filtered and concentrated in vacuo resulting in crude product which was purified by column chromatography on silica gel to obtain a pale yellow color solid. Yield: 150 mg, 14.15%, HPLC Purity: 77.57%. ¹H NMR (400 MHz, CDCl₃): δ 9.26 (d, J=8.7 Hz, 1H), 8.67 (d, J=8.8 Hz, 1H), 8.30 (d, J=7.3 Hz, 1H), 8.21 (d, J=7.5 Hz, 1H), 8.18-8.07 (m, 1H), 7.71 (dt, J=22.9, 8.1 Hz, 1H), 4.05 (d, J=8.5 Hz, 3H).

### Step-3:

A stirred solution of 10% Pd—C (8 mg) in dry methanol (2 mL) was charged with a solution of Step-2 product (80 mg, 0.340 mmol) in methanol (10 ml) under nitrogen. The reaction was charged with a hydrogen pressure (bladder) for 24 hours at room temperature. The reaction mixture was filtered through a pad of celite. The filtrate was concentrated in vacuo to obtain yellow oil. Yield: 60 mg, 86.95%. MS (ES+): m/z=202.05 [MH⁺].

### Step-4:

A stirred solution of Step-3 product (120 mg, 1 eq.) in methanol (20 mL) was charged with 2-formyl phenyl boronic acid (89 mg, 1 eq.). The reaction was stirred at room temperature for 30 minutes, then charged with sodium cyanoborohydride (150 mg, 4 eq.), and stirred at room temperature for an additional 48 hours. The solvent was concentrated in vacuo then partitioned between DCM (20 mL) and water (2×15 mL) and separated. The organic layer was dried over sodium sulfate, filtered, and concentrated in vacuo to obtain the crude product. The crude product was purified by column chromatography on silica gel to obtain yellow color oil. Yield: 80 mg, 40%; HPLC Purity: 82.57%. MS (ES+): m/z=336.15 [MH⁺].

### Step-5:

A stirred solution of the Step-4 product (100 mg, 0.590 mmol) in ethanol (6 mL), water (2 mL), and acetic acid (2 mL) was charged with p-formaldehyde (14 mg, 0.590 mmol), stirred at room temperature for 15 minutes, and then charged with sodium cyanoborohydride (75 mg, 2.30 mmol) portion-wise over a 15-minute period, and stirred at room-temperature for 24 hours. The solvent was concentrated in vacuo. The residue was charged with water (10 mL), acidified to pH=2 using 1N KHSO₄, and extracted with ethyl acetate (3×10 mL). The combined organic layers were washed with brine (2×10 mL), dried over sodium sulfate, filtered, and concentrated in vacuo to obtain a yellow solid. Yield: 100 mg, 97.15%. MS (ES+): m/z=350.10 [MH⁺].

### Step-6:

A stirred solution of the Step-5 product (100 mg, 1 eq.) in THF (3 mL) and water (3 mL) was charged with solid lithium hydroxide (14 mg, 2 eq.). The reaction mixture was stirred at room temperature for 24 hours. The THF was concentrated in vacuo and the aqueous portion was acidified to pH=2 using 1N KHSO₄, and extracted with ethyl acetate (3×15 mL). The combined organic layers were washed with brine (2×10 mL), dried over sodium sulfate, filtered, and concentrated in vacuo to obtain. brown solid. Yield: 80 mg, 83.33%; HPLC Purity: 32.52%. MS (ES+): m/z=336.10 [MH⁺].

### Synthesis of 3-((2-boronobenzyl)(methyl)amino)benzoic acid (A-146)

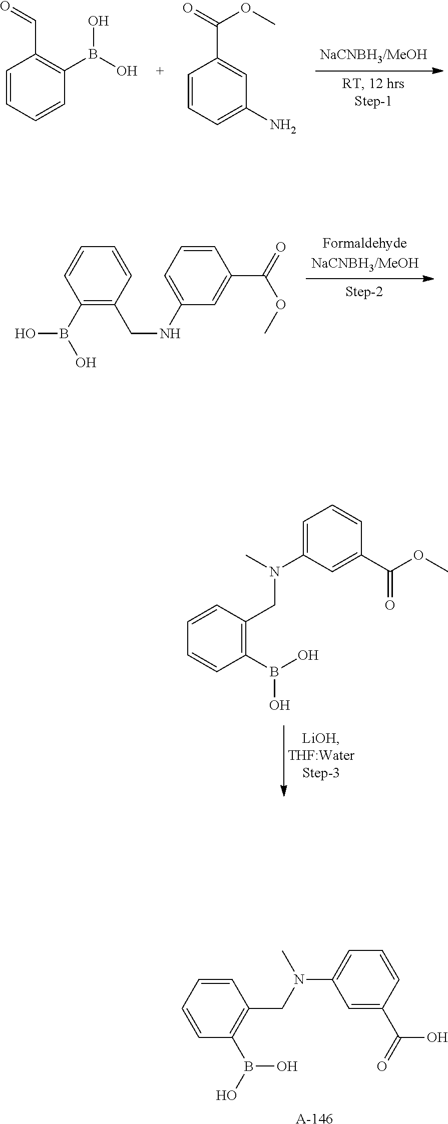 [SMILES: O=Cc1ccccc1B(O)O ; COC(=O)c1cccc(N)c1 ; COC(=O)c1cccc(NCc2ccccc2B(O)O)c1 ; COC(=O)c1cccc(N(C)Cc2ccccc2B(O)O)c1 ; CN(Cc1ccccc1B(O)O)c1cccc(C(=O)O)c1 ; "A-146"]

### Step-1:

A stirred solution of methyl-3-amino benzoate (200 mg, 1.52 mmol) in methanol (5 mL) was charged with 2-formyl phenyl boronic acid (198 mg, 1.32 mmol), stirred at room temperature for 10 minutes, and then charged with sodium cyano borohydride (332 mg, 5.29 mmol) portion-wise over a 15-minute period, and stirred at room temperature for 24 hours. The solvent was concentrated in vacuo. The residue was dissolved in DCM (20 mL) and washed with water (2×15 mL), brine (2×15 mL), dried over sodium sulfate, filtered, and concentrated in vacuo to obtain the crude product. The crude product was purified by column chromatography on silica gel to obtain a brown solid. Yield: 250 mg, 66.31%. MS (ES+): m/z=286.15 [MH⁺]. ¹H NMR (400 MHz, DMSO-d₆): δ 7.55-7.39 (m, 1H), 7.37-7.26 (m, 4H), 7.23-7.10 (m, 4H), 4.58 (s, 2H), 4.12-3.99 (m, 1H), 3.83 (d, J=30.9 Hz, 3H), 1.99 (s, 2H).

### Step-2:

A stirred solution of the Step-1 product (250 mg, 0.87 mmol) in ethanol (15 mL), water (5 mL), and acetic acid (5 mL) was charged with p-formaldehyde (40 mg, 1.30 mmol) and stirred at room temperature for 15 minutes. The reaction mixture was charged with sodium cyanoborohydride (220 mg, 3.50 mmol) portion-wise over a 15-minute period and stirred at room-temperature for 24 hours. The solvent was concentrated in vacuo. The residue was charged with water (10 mL), acidified to pH=2 using 1N KHSO₄ and extracted with ethyl acetate (3×20 mL). The combined organic layers were washed with brine (2×20 mL), dried over sodium sulfate, filtered, and concentrated in vacuo resulting in yellow solid (Qty-160 mg). Yield: 160 mg, 61.06%; HPLC Purity: 85.56%. MS (ES+): m/z=300.00 [MH⁺].

### Step-3:

To a stirred solution of the Step-2 product (160 mg, 0.53 mmol) in THF (5 mL) and water (2 mL) was charged with lithium hydroxide (26 mg, 1.00 mmol). The reaction mixture was stirred at room temperature for 24 hours. The solvent was concentrated in vacuo. The residue was acidified to pH 2 using 1N KHSO₄ and extracted with ethyl acetate (3×15 mL). The combined organic layer was washed with brine (2×10 mL), dried over sodium sulfate, filtered, and concentrated in vacuo resulting in a pale yellow solid. Yield: 150 mg, 98.68%. MS (ES+): m/z=286.15 [MH⁺].

### Synthesis of 4-((2-boronobenzyl)(methyl)amino)benzoic acid (A-147)

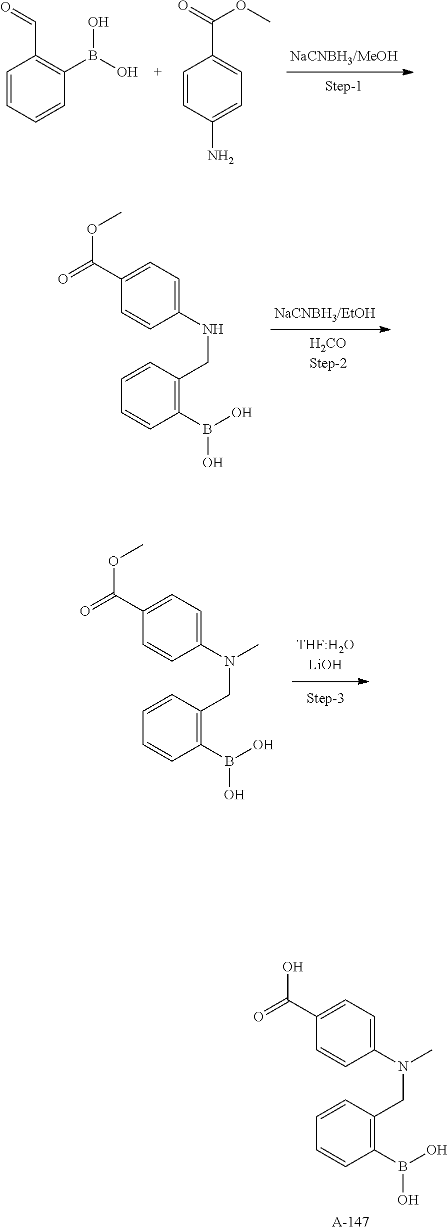 [SMILES: O=Cc1ccccc1B(O)O ; COC(=O)c1ccc(N)cc1 ; COC(=O)c1ccc(NCc2ccccc2B(O)O)cc1 ; COC(=O)c1ccc(N(C)Cc2ccccc2B(O)O)cc1 ; CN(Cc1ccccc1B(O)O)c1ccc(C(=O)O)cc1 ; "A-147"]

### Step-1:

A stirred solution of methyl-4-amino benzoate (200 mg, 1.52 mmol) in methanol (5 mL) was charged with 2-formyl phenyl boronic acid (198 mg, 1.32 mmol), stirred at room temperature for 10 minutes, and then charged with sodium cyano borohydride (332 mg, 0.529 mmol) portion-wise over a 15-minute period, and stirred at room temperature for an additional 24 hours. The solvent was concentrated under vacuum. The residue was dissolved in DCM (20 mL), washed with water (2×15 mL), brine (2×15 mL), dried over sodium sulfate, filtered, and concentrated in vacuo resulting in the crude product. The crude product was purified by column chromatography to obtain an off-white color solid. Yield: 270 mg, 71.61%. MS (ES+): m/z=286.15 [MH⁺]. ¹H NMR (400 MHz, DMSO-d₆): δ 7.89 (t, J=8.7 Hz, 2H), 7.67 (dd, J=13.5, 8.6 Hz, 2H), 7.46 (d, J=4.1 Hz, 2H), 7.38-7.24 (m, 2H), 4.59 (s, 2H), 4.10 (q, J=5.2 Hz, 1H), 3.81 (s, 3H), 1.23 (s, 2H).

### Step-2:

A stirred solution of the Step-1 product (50 mg, 0.175 mmol) in ethanol (3 mL), water (1 mL) and acetic acid (1 mL) was charged with p-formaldehyde (8 mg, 0.26 mmol) and stirred at room temperature for 15 minutes. The reaction mixture was then charged with sodium cyanoborohydride (44 mg, 0.70 mmol) portion-wise over a 15-minute period and stirred at room temperature for 24 hours. The solvent was concentrated in vacuo. The residue was diluted in water (10 mL), was acidified to pH=2 using 1N KHSO₄, and extracted with ethyl acetate (3×10 mL). The combined organic layer was washed with brine (2×10 mL), dried over sodium sulfate, filtered, and concentrated in vacuo resulting in an off-white solid. Yield: 50 mg, 96.15%. MS (ES+): m/z=300.00 [MH⁺].

### Step-3:

A stirred solution of the Step-2 product (250 mg, 0.83 mmol) in THF (10 mL) and water (4 mL) was charged with lithium hydroxide (40 mg, 1.6 mmol) and stirred at room temperature for 24 hours. The solvent was concentrated in vacuo. The residue was acidified to pH=2 using 1N KHSO₄ and extracted with ethyl acetate (3×15 mL). The combined organic layers were washed with brine (2×10 mL), dried over sodium sulfate filtered, and concentrated in vacuo resulting in yellow solid. Yield: 210 mg, 88.23%; HPLC Purity: 82.94%. MS (ES+): m/z=286.15 [MH⁺].

### Synthesis of 6-((2-boronobenzyl)(methyl)amino)-1-naphthoic acid (A-154)

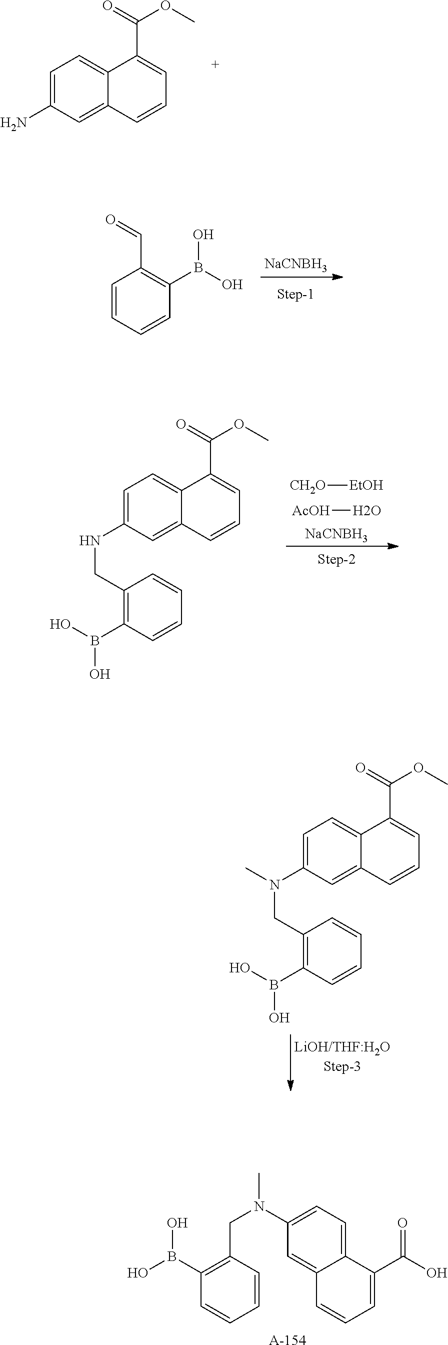

### Step-1:

A stirred solution of methyl 6-amino-1-naphthoate (500 mg, 2.48 mmol) in methanol (20 mL) was charged with 2-formyl phenyl boronic acid (373 mg, 2.48 mmol) and stirred at room temperature for 30 minutes. The reaction mixture was then charged with sodium cyanoborohydride (625 mg, 9.9 mmol) and stirred at room temperature for an additional 48 hours. The solvent was concentrated in vacuo and residue was diluted with DCM (20 mL), washed with water (2×15 mL), dried over sodium sulfate, filtered, and concentrated in vacuo to obtain the crude product. The crude product was purified by column chromatography to obtain yellow solid. Yield: 600 mg, 72.02%. MS (ES+): m/z=336.10 [MH⁺]. HPLC Purity: 99.59%. ¹H NMR (400 MHz, DMSO-d₆): δ 9.66-9.60 (m, 1H), 8.67 (d, J=9.5 Hz, 1H), 8.36 (dd, J=9.7, 2.8 Hz, 2H), 8.06 (d, J=8.3 Hz, 2H), 7.92 (dd, J=15.9, 7.3 Hz, 2H), 7.74 (s, 1H), 7.49 (dq, J=15.2, 7.7 Hz, 3H), 7.35 (t, J=7.2 Hz, 1H), 4.69 (s, 2H), 4.00-3.88 (m, 3H).

### Step-2:

A stirred solution of the Step-1 product (600 mg, 1.79 mmol) in ethanol (36 mL), water (12 mL), and acetic acid (12 mL) was charged with p-formaldehyde (81 mg, 2.68 mmol) and stirred at room temperature for 15 minutes. The reaction mixture was charged with sodium cyanoborohydride (450 mg, 7.16 mmol) portion-wise over a 15-minute period and stirred at room-temperature for 24 hours. The solvent was concentrated in vacuo. The residue was diluted with water (10 mL) and acidified to pH=2 using 1N KHSO₄ and extracted with ethyl acetate (3×20 mL). The combined organic layer was washed with brine (2×20 mL), dried over sodium sulfate, filtered, and concentrated in vacuo resulting in a yellow solid (680 mg crude) which was used in the next step without further purification.

Crude product was used as such for next step: HPLC Purity: 93.25%; MS (ES+): m/z=350.15 [MH⁺]; ¹H NMR (400 MHz, DMSO-d₆): δ 8.52 (d, J=9.6 Hz, 1H), 8.14 (s, 1H), 7.88 (d, J=8.4 Hz, 1H), 7.77 (d, J=7.1 Hz, 1H), 7.53 (d, J=6.9 Hz, 1H), 7.40 (t, J=7.7 Hz, 1H), 7.32 (dd, J=9.4, 2.9 Hz, 1H), 7.21 (dq, J=15.0, 7.0 Hz, 3H), 7.05 (t, J=5.9 Hz, 2H), 4.80 (s, 2H), 3.89 (s, 3H), 3.07 (s, 3H).

### Step 3:

A stirred solution of the Step-2 product (670 mg, 1.9 mmol) in THF (20 mL) and water (20 mL) was charged with lithium hydroxide (92 mg, 3.8 mmol) and stirred at room temperature for 24 hours. The solvent was concentrated in vacuo. The residue was acidified to pH=2 using 1N KHSO₄ and extracted with ethyl acetate (3×25 mL). The combined organic layer was washed with brine (2×20 mL), dried over sodium sulfate, filtered, and concentrated in vacuo resulting in a yellow solid. Yield: 600 mg, 93.33%. MS (ES+): m/z=336.10 [MH⁺]. HPLC Purity: 80.32%. ¹H NMR (400 MHz, DMSO-d₆): δ 12.84 (s, 1H), 8.63 (d, J=9.5 Hz, 1H), 7.81 (dd, J=27.3, 7.8 Hz, 2H), 7.52 (d, J=7.1 Hz, 1H), 7.42-7.14 (m, 4H), 7.06 (d, J=6.1 Hz, 2H), 4.79 (s, 2H), 4.08-3.86 (m, 2H), 3.09 (d, J=23.7 Hz, 3H).

### Synthesis of 5′-bromo-2′-(dimethylamino)-[1,1′-biphenyl]-3-carboxylic acid (A 155)

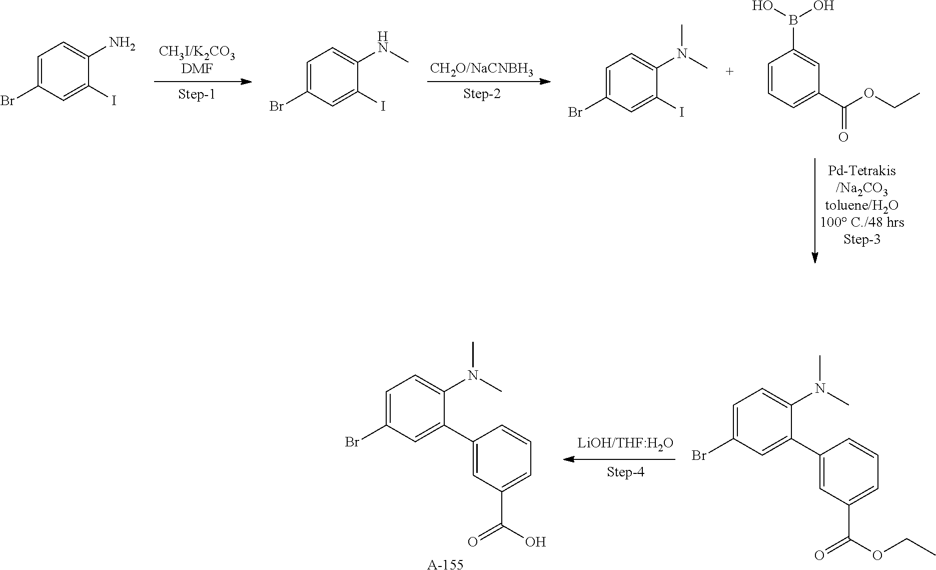

### Step-1:

A stirred solution of 4-bromo-2-iodo aniline (2 g, 6.71 mmol) and potassium carbonate (1.4 g, 10.14 mmol) in DMF (20 mL) was cooled to 0° C. Iodomethane (1.9 g, 13.0 mmol) was charged to the reaction mixture drop wise a 20-minute period while keeping the temperature between 0-5° C., stirred at 0° C. for 1 hour and then stirred at room temperature for 48 hours. The reaction mixture was charged with water (30 mL) and was extracted with ethyl acetate (3×25 mL). The combined organic layers were washed with brine (2×25 mL), dried over sodium sulfate, filtered, and concentrated in vacuo to obtain the crude product which was purified by column chromatography to obtain a brown oil. Yield: 1.8 g, 82.19%. HPLC Purity: 68.15%. MS (ES+): m/z=312/314 [MH⁺].

### Step 2:

A stirred solution of the step-1 product (1.8 g, 5.70 mmol) in ethanol (108 mL), water (36 mL), and acetic acid (36 mL) was charged with p-formaldehyde (260 mg, 8.60 mmol) and stirred at room-temperature for 15 minutes. The reaction mixture was portion-wise charged over a 20-minute period with sodium cyanoborohydride (1.45 g, 23 mmol) and stirred at room-temperature for 24 hr. The solvent was concentrated in vacuo and the residue was diluted in water (20 mL) and was acidified to pH=2 using 1N KHSO₄ and extracted with ethyl acetate (3×30 mL). The combined organic layer was washed with brine (2×20 mL), dried over sodium sulfate, filtered, and concentrated in vacuo resulting in a yellow oil. Yield: 1.7 g, 90.42%. HPLC Purity: 99.70%. MS (ES+): m/z=326. [MH⁺].

### Step 3:

A stirred solution of step-2 product (1.7 g, 5.20 mmol) in toluene (50 mL) was charged with a solution of sodium carbonate (1.11 g, 10.04 mmol) in water (15 mL), 3-ethoxycarbonyl phenyl boronic acid (939 mg, 5.20 mmol) and the reaction was degassed with argon for 1 hr and then charged with tetrakis (340 mg, 20w/w) and heated to 100° C. for 24 hours. The reaction was allowed to cool to room-temperature and charged with water (20 mL) and was extracted with ethyl acetate (3×20 mL). The combined organic layer was washed with brine (2×20 mL), dried over sodium sulfate, filtered, and concentrated in vacuo to obtain 750 mg of the crude product which was purified by column chromatography to obtain desired product as colorless oil. Yield: 150 mg, 8.33%. HPLC Purity: 96.76%. MS (ES+): m/z=348/350 [MH⁺].

### Step 4:

A stirred solution of the step-3 product (100 mg, 0.28 mmol) in THF (5 mL) and water (3 mL) was charged with lithium hydroxide (10 mg, 0.43 mmol) and stirred at room-temperature for 24 hours. The solvent was concentrated in vacuo and the residue was acidified to pH=2 using 1N KHSO₄ and extracted with ethyl acetate (3×10 mL). The combined organic layer was washed with brine (2×10 mL), dried over sodium sulfate, filtered, and concentrated in vacuo resulting in an off-white solid. Yield: 70 mg, 76.92%. HPLC Purity: 95.09%. MS (ES+): m/z=321/323 [MH⁺].

### B. Coupling of Boronate Ester or Boronic Acid Precursors (A) to the Appropriate Protected Core (Step-1a & b):

To a stirred solution of carboxylic acid in DCM or DMF was added DMAP, DIPEA, EDCI, or HOBt (in some cases). The solution was stirred for 15 minutes at a temperature range of 0° C. to room temperature followed by addition of protected 4-(3-aminomethyl phenyl)piperidine or 5-aminomethyl Spiro[benzofuran-3,4′-piperidine]. Stirring was continued at room temperature and reaction was monitored by LCMS until most of the starting materials were consumed. Reaction mixture was then quenched with water, and the aqueous layer was extracted twice with dichloromethane. The combined organic layer was dried over sodium sulfate, filtered, and concentrated in vacuo to afford the crude product which was used for next step without further purification.

The details of compounds synthesized by Step-1a are as below:

| Compound | | Brief Reaction | Analytical |
|---|---|---|---|
| No. | Structure | conditions | data |
| B-131- Spiro | 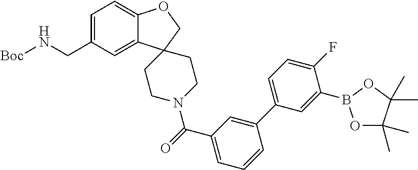 [SMILES: CC(C)(C)OC(=O)NCc1ccc2c(c1)C1(CCN(C(=O)c3cccc(-c4ccc(F)c(B5OC(C)(C)C(C)(C)O5)c4)c3)CC1)CO2] tert-butyl ((1′-(4′-fluoro-3′-(4,4,5,5-tetramethyl-1,3,2-dioxaborolan- 2-yl)-[1,1′-biphenyl]-3-carbonyl)-2H-spiro[benzofuran-3,4′- piperidin]-5-yl)methyl)carbamate | A-131 (1 eq.), Spiro core (1 eq.), EDCI (1.5 eq.), DMAP (0.5 eq.), in Dichloromethane (50 vol.), room temperature, 12 hours. Yield: 62% after acid-base work- up. | Yield: 62%; Mol.Wt: 642.56; MS (ES+): m/z = 587 [M − t-Bu]. |

The details of compounds synthesized by Step-1b are as below.

| Compound | | Brief Reaction | Analytical |
|---|---|---|---|
| No. | Structure | conditions | data |
| B-116 Spiro | 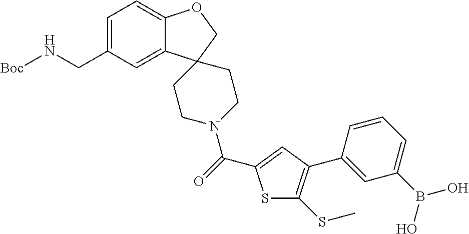 [SMILES: CSc1sc(C(=O)N2CCC3(CC2)COc2ccc(CNC(=O)OC(C)(C)C)cc23)cc1-c1cccc(B(O)O)c1] (3-(5-(5-(((tert-butoxycarbonyl)amino)methyl)-2H-spiro[benzofuran-3,4′- piperidin]-1′-ylcarbonyl)-2-(methylthio)thiophen-3-yl)phenyl)boronic acid | tert-butyl ((2H- spiro[benzofuran- 3,4′-piperidin]-5- yl)methyl)carbamate (1.3 eq.), EDCI.HCl (1.5 eq.), DMAP (2 eq.), DCM (20 vol.), room temperature, 4 hours, | Yield: 60%; Mol. Wt: 594.55; MS (ES+): m/z = 595.70 [MH⁺]. |
| B-146 | 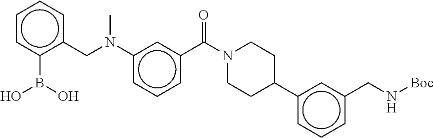 (2-(((3-(4-(3-(((tert-butoxycarbonyl)amino)methyl)phenyl)piperidine-1- carbonyl)phenyl)(methyl)amino)methyl)phenyl)boronic acid | A-146, tert-butyl 3- (piperidin-4-yl) benzyl carbamate (1 eq), EDCI 1.5 eq. DMAP 1.1 eq., HOBt, 1.1 eq. in Dichloromethane (70 vol.), room temperature, 12 hours. | Yield: 50%; Mol. Wt: 557.49; MS (ES+): m/z = 558.40. |
| B-147 | 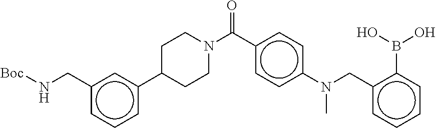 (2-(((4-(4-(3-(((tert-butoxycarbonyl)amino)methyl)phenyl)piperidine-1- carbonyl)phenyl)(methyl)amino)methyl)phenyl)boronic acid | A-147, tert-butyl 3- (piperidin-4-yl) benzyl carbamate (1 eq), EDCI 1.5 eq. DMAP 1.1 eq., HOBt, 1.1 eq. in Dichloromethane (70 vol.), room temperature, 12 hours | Yield: 50%; Mol. Wt: 557.49; MS (ES+): m/z = 558.40 [MH⁺]. |
| B-143 | 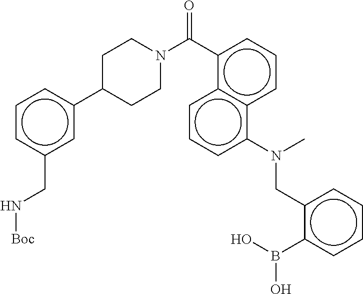 (2-(((5-(4-(3-(((tert-butoxycarbonyl)amino)methyl)phenyl) piperidine-1-carbonyl)naphthalen-1-yl)(methyl)amino) methyl)phenyl)boronic acid | A-143, tert-butyl 3- (piperidin-4-yl) benzyl carbamate 1 eq., EDCI 1.5 eq. DMAP 1.1 eq., HOBt, 1.1 eq. in Dichloromethane (125 vol.), room temperature, 12 hours. | Yield: 96.5%; Mol. Wt: 607.5; MS (ES+): m/z = 608.40 [MH⁺]. |
| B-154 | 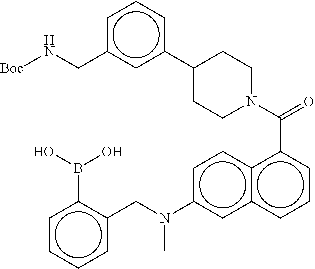 (2-(((5-(4-(3-(((tert-butoxycarbonyl)amino)methyl) phenyl)piperidine-1-carbonyl)naphthalen-2-yl) (methyl)amino)methyl)phenyl)boronic acid | A-154, tert-butyl 3- (piperidin-4-yl) benzyl carbamate 1 eq., EDCI 1.5 eq. DMAP 1.1 eq., HOBt, 1.1 eq. in Dichloromethane (125 vol.), room temperature, 12 hours. | Yield: 97.34%; Mol. Wt: 607.5; MS (ES+): m/z = 608.35 [MH⁺]; ¹H NMR (400 MHz, dmso- d₆): δ 8.13 (s, 2H), 7.69-7.62 (m, 3H), 7.51 (dd, J = 15.3, 8.2 Hz, 5H), 7.39-6.89 (m, 5H), 4.88-4.70 (m, 2H), 4.16 (s, 2H), 3.16- 2.61 (m, 8H), 2.05-1.49 (m, 6H), 1.27 (s, 9H). |

C. Deprotection of the Protected Amide (B) with Boronate Functionality (Step-2a) or Boronic Acid Functionality (Step-2b):

### (Step-2a):

Products from Step-1a were stirred with aqueous hydrochloric acid or trifluoracetic acid (TFA) in a co-solvent like dioxane, acetonitrile, methanol, THF, DCM etc. Reaction was monitored by LCMS until most of the starting material was consumed. Reaction mass was then concentrated in vacuo to remove the solvents, and the residue obtained was purified by reverse phase preparative HPLC. The pure fraction of mobile phase was lyophilized to obtain the products as TFA salts.

In most of the cases, boronate esters were hydrolyzed partly to obtain mixture of desired product and corresponding boronate esters. In such cases, mixture was subjected to preparative HPLC purification under acidic conditions, during which, most of the boronate esters were converted to target boronic acids. Multiple purifications were needed in such cases to isolate pure boronic acid.

In some cases, TFA salts were converted to hydrochloride salts by stirring with 2N HCl for 30 minutes under nitrogen atmosphere followed by lyophilization.

The details of compounds synthesized by Step-2a are as below. All reactions were done on 100-200 mg scale.

| Compound | | Brief Reaction | Analytical |
|---|---|---|---|
| No. | Structure | conditions | data |
| 131-Spiro | 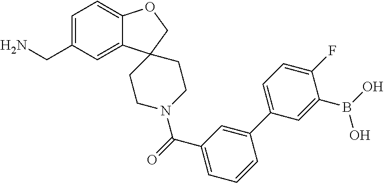 [SMILES: NCc1ccc2c(c1)C1(CCN(C(=O)c3cccc(-c4ccc(F)c(B(O)O)c4)c3)CC1)CO2] (3′-(5-(aminomethyl)-2H-spiro[benzofuran-3,4′-piperidin]- 1′-ylcarbonyl)-4-fluoro-[1,1′-biphenyl]-3-yl)boronic acid | Acetonitrile (20 vol.), TFA (10 vol.), water (3 vol.), 80° C., 12 hours. | Mol. Wt: 460.3; MS (ES+): m/z = 461 [MH⁺]; HPLC Purity: 99.6%;. ¹H NMR (400 MHz, dmso- d₆ (D₂O): δ 1.66-1.77 (m, 4H), 3.07-3.38 (m, 4H) 3.93 (s, 2H), 4.41-4.49 (m, 2H), 6.81 (d, J = 8.2 Hz, 1H), 7.20 (t, J = 8.8 Hz, 2H), 7.39 (d, J = 9.0 Hz, 2H), 7.55 (t, J = 7.7 Hz, 1H), 7.65 (s, 1H), 7.73 (d, J = 7.9 Hz, 2H), 7.85 (d, J = 5.3 Hz, 1H). |

### Step-2b:

Products from Step-1b were stirred with aqueous hydrochloric acid or trifluoracetic acid (TFA) in a co-solvent like dioxane, acetonitrile, methanol, THF, DCM etc. Reaction was monitored by LCMS until most of the starting material was consumed. Reaction mass was concentrated under vacuum. The residue obtained was purified by reverse phase preparative HPLC. The pure fraction of mobile phase was lyophilized to obtain the products as TFA salts.

In some cases, TFA salts were converted to hydrochloride salts by stirring with 2N HCl for 30 minutes under nitrogen atmosphere followed by lyophilization;

The details of compounds synthesized by above method Step-2b are as below. All reactions were done on 100-200 mg scale.

| Compound | | Brief Reaction | Analytical |
|---|---|---|---|
| No. | Structure | conditions | data |
| 116 Spiro | 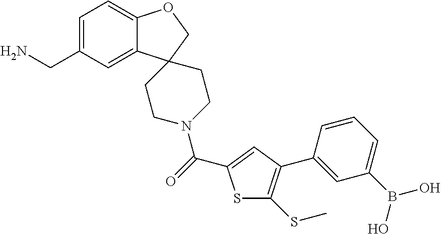 [SMILES: CSc1sc(C(=O)N2CCC3(CC2)COc2ccc(CN)cc23)cc1-c1cccc(B(O)O)c1] (3-(5-(5-(aminomethyl)-2H-spiro[benzofuran-3,4′-piperidin]-1′- ylcarbonyl)-2-(methylthio)thiophen-3-yl)phenyl)boronic acid | TFA (20 eq.), dichloromethane (20 vol.), room temperature, 4 hours. Preparative HPLC. isolated as TFA salt. | Yield: 24%; Mol. Wt: 494.15; MS (ES+): m/z = 494.95 [MH⁺] ¹H NMR (400 MHz, dmso) δ 8.17 (s, 4H), 7.95 (s, 1H), 7.78 (d, J = 7.3 Hz, 1H), 7.60 (d, J = 7.5 Hz, 1H), 7.46 (dd, J = 15.9, 8.2 Hz, 3H), 7.24 (d, J = 8.2 Hz, 1H),, 6.84 (d, J = 8.2 Hz, 1H), 4.52 (s, 2H), 4.27 (d, J = 13.2 Hz, 2H), 3.92 (d, J = 5.8 |
| | | | Hz, 2H). 2.58- |
| | | | 2.45 (m, 3H), |
| | | | 1.90-1.72 |
| | | | (m, 4H). |
| 146 | 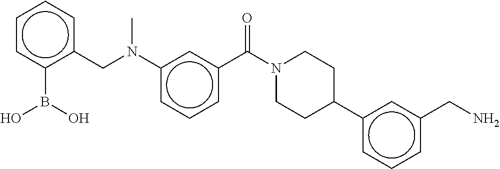 (2-(((3-(4-(3-(aminomethyl)phenyl)piperidine-1-carbonyl)phenyl)(methyl)amino) methyl)phenyl)boronic acid | Dichloromethane (70 vol.), TFA (2 eq added at 0° C.). Stirred at room temperature, for 24 hours. Purification by preparative HPLC. | Yield: 10.34%; Mol. Wt: 457.37; MS (ES+): m/z = 458.25 [MH⁺], HPLC Purity: 97.56% ¹H NMR (400 MHz, DMSO-d₆) δ 8.11 (s, 2H), 7.51 (d, J = 7.3 Hz, 1H), 7.35 (d, J = 6.8 Hz, 2H), 7.22 (ddt, J = 31.5, 15.2, 6.9 Hz, 5H), 7.00 (d, |
| | | | J = 7.7 Hz, 1H), |
| | | | 6.77 (d, J = 8.1 |
| | | | Hz, 1H), 6.69- |
| | | | 6.60 (m, 2H), |
| | | | 4.64 (d, J = 39.2 |
| | | | Hz, 2H), 4.15- |
| | | | 3.80 (m, 6H), |
| | | | 2.99 (s, 3H), 2.78 |
| | | | (t, J = 12.0 Hz, |
| | | | 1H), 1.62 (t, J = |
| | | | 67.8 Hz, 6H). |
| 147 | 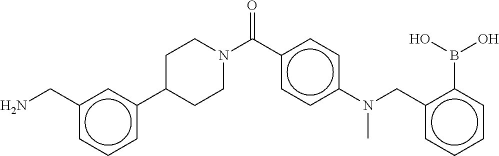 (2-(((4-(4-(3-(aminomethyl)phenyl)piperidine-1-carbonyl)phenyl)(methyl) amino)methyl)phenyl)boronic acid | dichloromethane (70 mL), TFA (2 eq. added at 0° C.). Stirring at room temperature for 24 hours. Purification by preparative HPLC. | Yield: 10.34%, Mol. Wt: 457.37; MS (ES+): m/z = 458.30 [MH⁺], HPLC Purity: 98.83% ¹H NMR (400 MHz, DMSO-d₆) δ 8.11 (s, 2H), 7.51 (d, J = 7.3 Hz, 1H), 7.35 (d, J = 6.8 Hz, 2H), 7.22 (ddt, J = 31.5, 15.2, 6.9 |
| | | | Hz, 5H), 7.00 (d, |
| | | | J = 7.7 Hz, 1H), |
| | | | 6.77 (d, J = 8.1 |
| | | | Hz, 1H), 6.69- |
| | | | 6.60 (m, 2H), |
| | | | 4.64 (d, J = 39.2 |
| | | | Hz, 2H), 4.15- |
| | | | 3.80 (m, 6H), |
| | | | 2.99 (s, 3H), 2.78 |
| | | | (t, J = 12.0 Hz, |
| | | | 1H), 1.62 (t, J = |
| | | | 67.8 Hz, 6H). |
| 143 | 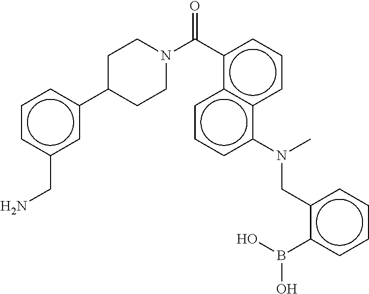 (2-(((5-(4-(3-(aminomethyl)phenyl)piperidine-1-carbonyl) naphthalen-1-yl)(methyl)amino)methyl)phenyl)boronic acid | Dichloromethane (70 mL), TFA (3 eq. added at 0° C.) stirring at room temperature for 24 hours. Purification by preparative HPLC after concentrating in vacuum. | Yield: 10.34%; Mol. Wt: 507.43; MS (ES+): m/z = 508.30 [MH⁺] HPLC Purity: 99.5% ¹H NMR (400 MHz, DMSO-d₆) δ 8.36-8.28 (m, 1H), 8.13 (s, 2H), 7.51 (q, J = 11.5, 8.7 Hz, 4H), 7.32 (tdd, J = 27.9, 17.0, 7.6 Hz, 8H), 4.84 (t, J = 13.6 Hz, 1H), 4.39 (s, 2H), 4.02 (q, J = 5.9 Hz, 2H), 3.43- 2.77 (m, 5H), 2.70 (s, 3H), 2.03- 1.28 (m, 6H). |
| 154 | 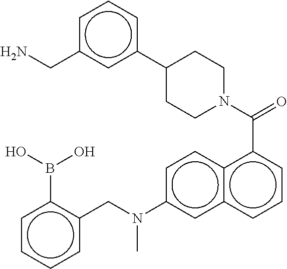 (2-(((5-(4-(3-(aminomethyl)phenyl)piperidine-1- carbonyl)naphthalen-2-yl)(methyl)amino)methyl) phenyl)boronic acid | Dichloromethane (45 vol.), TFA (3 eq. added at 0° C.) Stirred at room temperature for 24 hours. Purification by preparative HPLC after concentrating in vacuum. | Yield: 19.07%; Mol. Wt: 507.43; MS (ES+): m/z = 508.25 [MH⁺], HPLC Purity: 97.10% ¹H NMR (400 MHz, DMSO-d₆): δ 8.10 (s, 3H), 7.68 (d, J = 8.7 Hz, 1H), 7.52 (dd, J = 15.8, 8.2 Hz, 2H), 7.40- 7.17 (m, 4H), 7.14 (d, J = 6.8 Hz, 2H), 7.05 (dd, J = 13.4, 5.3 Hz, 2H), 4.79 (s, 2H), 4.02 (s, 2H), 3.06 (s, 3H), 2.82 (m, 5H), 1.95- 1.62 (m, 6H). |

### Approach-2

Desired halo aryl carboxylic acids were first coupled with tert-butyl 3-(piperidin-4-yl)benzyl carbamate. The coupled products were reacted with Bis Pinacolato diborane to obtain boronate esters which were hydrolyzed to corresponding boronic acids.

### A. Synthesis of Halo Carboxylic Acid Precursors:

The details of intermediates halo aryl carboxylic acids (A) sourced/synthesised as per literature methods/synthesised by developed methods are described above in Approach 1 A of this example.

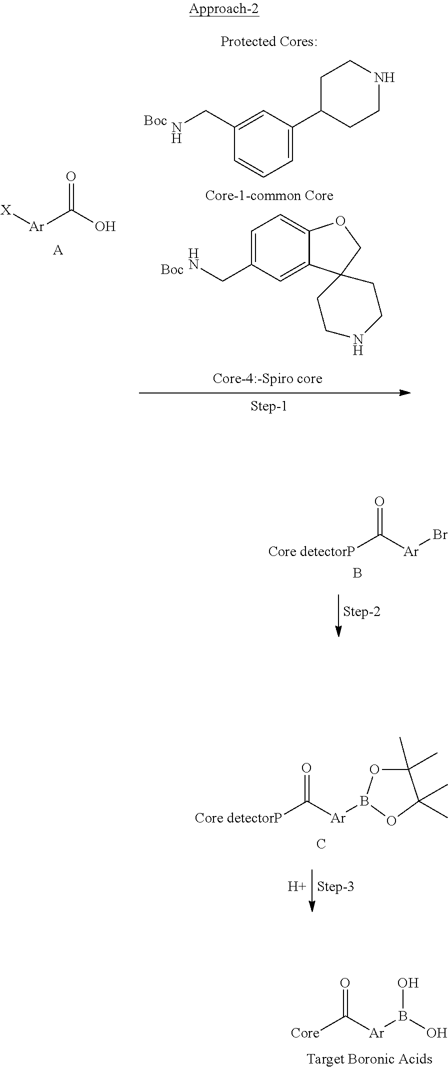

### B. Coupling of Halo Carboxylic Acid Precursors (A) to the Appropriate Protected Core to Obtain the Halo Amides:

### Step-1:

To a stirred solution of carboxylic acid in DCM or DMF was added DMAP, DIPEA, EDCI, or HOBt (in some cases). The solution was stirred for 15 minutes at a temperature range of 0° C. to room temperature followed by addition of Core-1 or Core-4 as shown in the above synthetic scheme. Stirring was continued at room temperature, and reaction was monitored by LCMS until most of the starting materials were consumed. Solvents were concentrated under vacuum. The reaction mixture was then quenched with water. The aqueous layer was extracted twice with dichloromethane/ethyl acetate. The combined organic layers were optionally washed with dilute HCl whenever DIPEA was used, dried over sodium sulfate, and concentrated under vacuum to afford the product which was purified by column chromatography.

The details of compounds synthesized by Step-1 are as below.

| Compound | | Brief Reaction | |
|---|---|---|---|
| No. | Structure | conditions | Analytical data |
| B-144 | 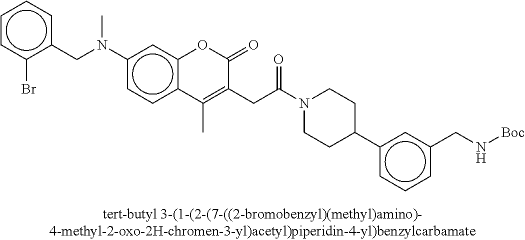 | Carboxylic acid (0.34 g, 0.19 mmol) in DCM (~90 mL), HOBt (1.5 eq.), EDCI (1.5 eq.), DMAP (0.5 eq.) and tert-butyl 3- (piperidin-4-yl) benzyl carbamate (1.2 eq.) was stirred at room temperature for 12 hours. Yield: 77% after chromatographic purification. | Mol.Wt: 687.23; MS (ES+): m/z = 588 [M − Boc]. |
| B-155- Spiro | 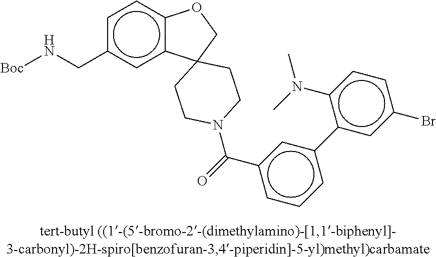 | A-155, spiro core (1 eq.), EDCI (1.5 eq.), DMAP (1.1 eq.), HOBt (1.1 eq.) in dichloromethane (75 mL), room temperature, 12 hours. | Yield: 90.20%; Mol.Wt: 620.58; MS (ES+): m/z = 620.30 [MH⁺]. |

### C. Boronation of Halo Amides (Step-1) to Obtain Desired Boronate Esters (C):

### Step-2:

The product of Step-1 was converted to boronate ester by palladium(0) catalyzed reaction with his pinacolato borane in 1,4-dioxane using potassium acetate as base. Reaction was monitored by LCMS until most of the starting material was consumed. After completion of the reaction, the reaction mixture was filtered through celite and concentrated. Product was extracted in ethyl acetate, and ethyl acetate layer was washed with water. The organic layer was separated, dried over sodium sulfate concentrated and purified by column chromatography using hexane/ethyl acetate to yield the boronate esters contaminated with his pinacolato borane. This crude product was characterized by LCMS and subjected to the next step without further purification

The details of compounds synthesized by Step-2 are as below.

| Compound | | Brief Reaction | |
|---|---|---|---|
| No. | Structure | conditions | Analytical data |
| C-144 | 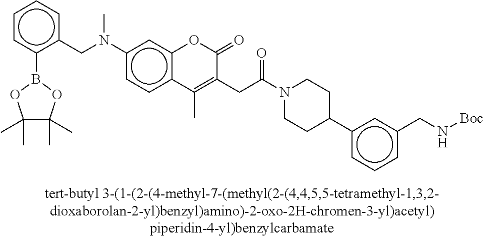 | B-144 (50 mg), Pd(OAc)₂ (1 eq.), TPP (4 eq.), potassium acetate (3 eq.), bis pinacolato diborane (10 eq.) in dioxane, 90° C. for 16 hours. | Mol.Wt: 735.41; MS (ES+): m/z = 758 [M + Na]. |
| C-155- Spiro | 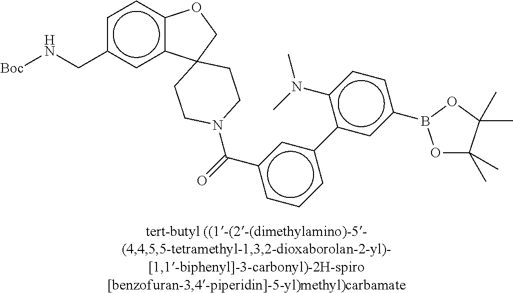 | B-155, bis pinacolato diborane (5 eq.), KOAc (3.5 eq.), Pd(dppf)Cl₂ (0.06 eq.), DMSO (60 mL), 80° C., 6 hours. | Yield: crude; Mol.Wt: 667.64; MS (ES+): m/z = 668.50 [MH⁺]. |

### D. Deprotection of Boronate Esters (Step-2) to Obtain the Target Boronic Acids

### Step-3:

Products of Step-2 were stirred with dioxane and concentrated HCl at room temperature overnight, when LCMS indicated complete consumption of starting. The reaction mixture was concentrated, and purified by Preparative HPLC.

The details of compounds synthesized are below. All reactions were done on 100-200 mg scale.

| Compound | | Brief Reaction | |
|---|---|---|---|
| No. | Structure | conditions | Analytical data |
| 144 | 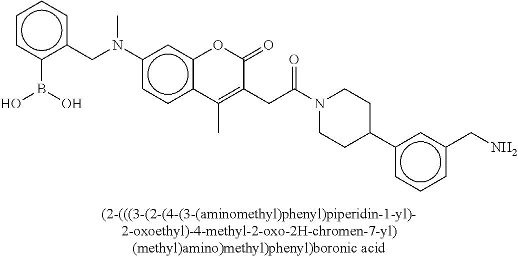 | Dioxane (100 vol.) 30% HCl (2 vol.), room temperature, overnight. Isolated as TFA salt by preparative HPLC. Yield: 26%. | Mol. Wt: 553.27; MS (ES+): m/z = 554 [MH⁺]; HPLC Purity: 96.4%; ¹H NMR (400 MHz, DMSO-d₆): δ 1.40- 1.70 (br, 2H), 1.84 (br, 2H), 2.60-2.91 (m, 2H), 3.10-3.30 (m, 1H), 3.98 (d, J = 5.6 Hz, 2H), 4.40-4.70 (br, 2H), 6.91 (d, J = 8.4 Hz, 1H), 7.20-7.50 (m, 6H), 7.78 (d, J = 8.8 Hz, 1H), 8.07 (br, 1H), 8.32 (br, 2H), 8.37 (br, 2H), 8.65 (br, 1H). |
| 155-Spiro | 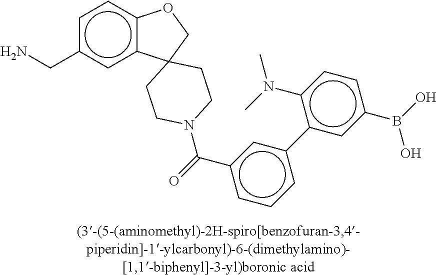 | C-155, Acetonitrile (80 vol.), 2NHCl (30 vol.), room temperature, 12 hours. | Mol. Wt: 485.3; MS (ES+): m/z = 486.35 [MH⁺]; HPLC Purity: 96.72% ¹H NMR (400 MHz, DMSO-d₆): δ 8.02 (s, 2H), 7.75-7.66 (m, 1H), 7.64 (s, 1H), 7.56 (m, 3H), 7.42 (s, 1H), 7.36 (d, J = 7.5 Hz, 1H), 7.22 (d, J = 8.3 Hz, 1H), 7.04 (dd, J = 8.0, 4.3 Hz, 1H), 6.84 (d, J = 8.2 Hz, 1H), 4.57-3.89 (m, 2H), 3.69 (m, 2H), 3.19 (d, J = 62.5 Hz, 4H), 2.5 (s, 6H), 1.90-1.64 (m, 3H), 1.21 (d, J = 37.9 Hz, 3H). |

### Example 15

### Synthesis of Cofluorons with Amido Phenol Functionality

13 Final targets with amido phenol functionality were synthesized.

### Approach-1

Suitably substituted 2-hydroxy aromatic amides with carboxylic acid functionality were synthesized and coupled with the protected core followed by the deprotection of Boc protection on amino methyl functionality as in the reaction scheme below:

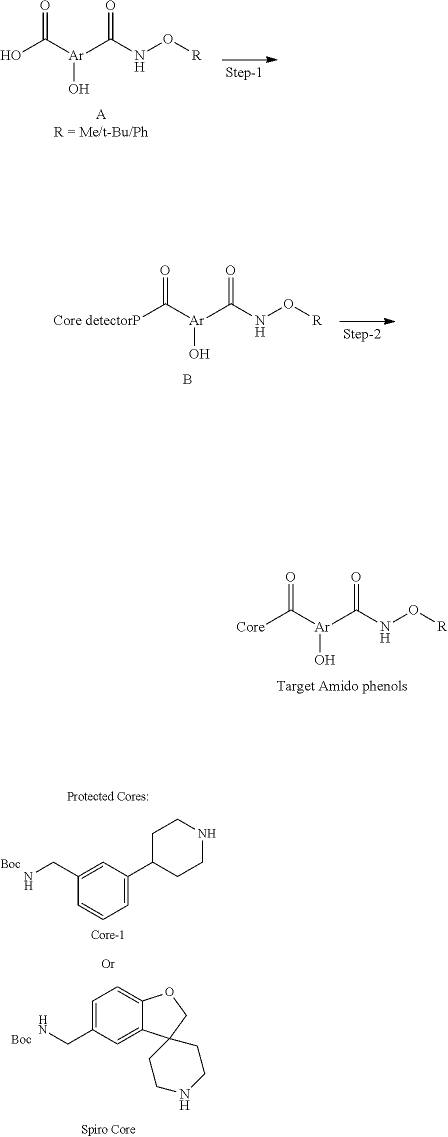

### A. Synthesis of Intermediate (A)

The details of syntheses of intermediates (A) are given as below:

| Target | Structure |
|---|---|
| A-75a-O—t-Bu | 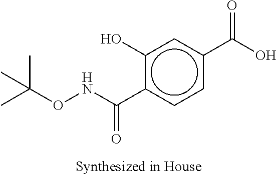 |
| A-75a-O—Ph | 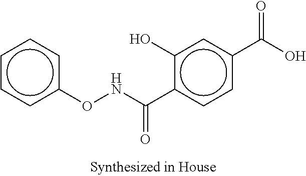 |
| A-92-O—t-Bu | 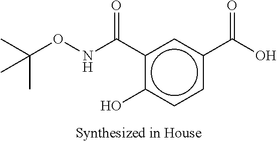 |
| A-114 Spiro | 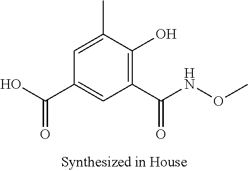 [SMILES: CONC(=O)c1cc(C(=O)O)cc(C)c1O] |

### 4-(tert-butoxycarbamoyl)-3-hydroxybenzoic acid (A-75-O-t-bu)

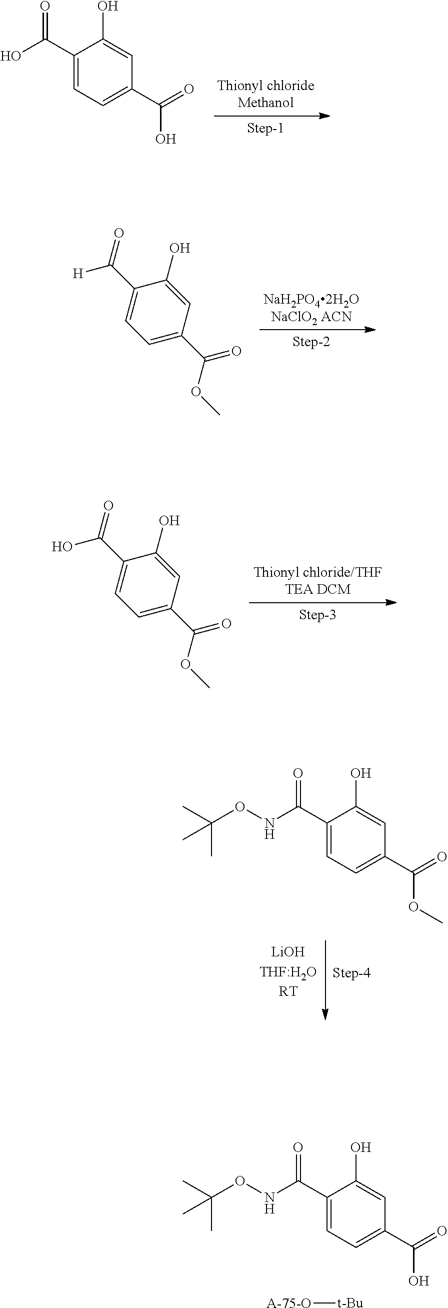

### Step-1:

A solution of 4-formyl-3-hydroxy benzoic acid (0.1 g, 0.6 mmol) in methanol (50 mL) was cooled to 0° C. and charged with thionyl chloride (0.097 g, 0.72 mmol) and heated at reflux for 6 hours. Thin layer chromatography (TLC) (Mobile phase 5% methanol in chloroform) indicated absence of starting material (Rf 0.1, “retention factors”) along with new spot (Rf 0.5). The reaction mixture was cooled to room temperature and concentrated in vacuo. The residue was partitioned between ethyl acetate and water and separated. The organic layer was dried over sodium sulfate concentrated, filtered, and concentrated in vacuo resulting in 95 mg desired product: Yield: (95 mg, 87.9%). NMR: NMR (400 MHz, DMSO-d₆): δ 3.93 (s, 3H), 7.4 (d, J=8.0 Hz, 1H), 7.5 (s, 1H), 7.7 (d, J=8.0 Hz, 1H).

### Step-2:

A solution of methyl 4-formyl-3-hydroxybenzoate (0.05 g, 0.27 mmol) and NaH₂PO₄.2H₂O (0.11 g, 0.69 mmol) in DMSO: water, 2:1 (7.5 ml) was charged with sodium chlorite (0.075 g, 0.66 mmol) at 0° C. The reaction mixture was allowed to stir at room temperature for 12 hours. The reaction mixture was acidified with 1N HCl until pH=2. The precipitated white solid was filtered, washed with water several times and dried to give 2-hydroxy-4-(methoxycarbonyl)benzoic acid. Yield: (0.035 g, 65%). Molecular Weight: 196. MS (ES+): m/z=197.2 [MH⁺].

### Step-3:

A solution of 2-hydroxy-4-(methoxycarbonyl)benzoic acid (0.20 g, 1 mmol) in THF (10 mL) was charged with thionyl chloride (0.121 g, 10 mmol) at 0° C. The reaction mixture was heated to 45° C. for 4 hours. The reaction mixture was concentrated in vacuo. The residue was diluted in dry DCM (5 ml), and charged with a solution of o-t-butyl amine.HCl (0.512 g, 4 mmol) and TEA (0.412 g, 4 mmol) in DCM (15 ml) at 0° C. The reaction mixture was charged with 1N HCl solution (15 ml) and separated. The organic layer dried over sodium sulfate, filtered, and concentrated in vacuo to obtain 0.205 g crude product. The crude product was purified by column chromatography on silica gel using hexane-ethyl acetate as eluent to give methyl 4-(benzoyloxy)-3-formylbenzoate. Yield: (0.16 g, 58.8%). Molecular Weight: 267. MS (ES+): m/z=268.05 [MH⁺].

### Step-4:

A solution of step-3 product (0.160 g, 0.59 mmol) in THF:water (2:1) (15 mL) was charged with LiOH (0.043 g, 1.7 mmol) and stirred at room temperature for 6 hours. The reaction mixture was concentrated in vacuo and the aqueous layer was and acidified with 1N HCl until pH=2. A solid precipitated product was filtered and dried to give 4-(tert-butoxycarbamoyl)-3-hydroxybenzoic acid. Yield: (0.015 g, 44%). Molecular Weight: 253. MS (ES+): m/z=254.0 [MH⁺].

### 3-hydroxy-4-(phenoxycarbamoyl)benzoic acid (A-75-O-ph)

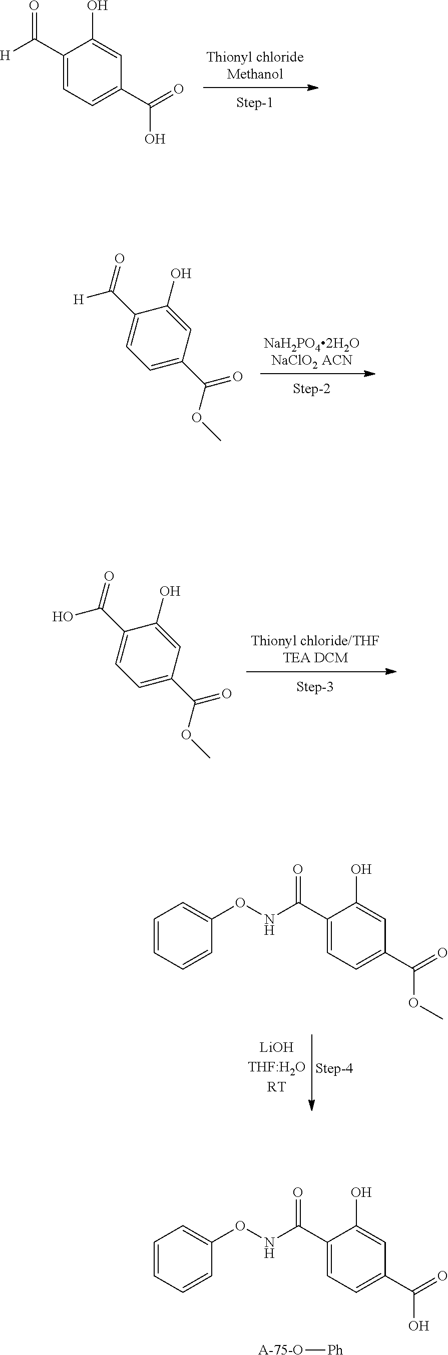

### Step-1:

A solution of 4-formyl-3-hydroxy benzoic acid (0.1 g, 0.6 mmol) in methanol (50 mL) at 0° C. was charged with thionyl chloride (0.097 g, 0.72 mmol) and the reaction mixture was heated at reflux for 6 hr. The reaction mixture was cooled and concentrated in vacuo and partitioned between ethyl acetate and water and separated. The organic layer was dried over sodium sulfate, filtered, and concentrated in vacuo resulting in 95 mg of the desired product. Yield: (0.095 g, 87.9%). ¹H NMR (400 MHz, DMSO-d₆): δ 3.93 (s, 3H), 7.4 (d, J=8.0 Hz, 1H), 7.5 (s, 1H), 7.7 (d, J=8.0 Hz, 1H)

### Step-2:

A solution of methyl 4-formyl-3-hydroxybenzoate (0.05 g, 0.27 mmol) and NaH₂PO₄.2H₂O (0.11 g, 0.69 mmol) in DMSO:water, 2:1 (7.5 ml) was cooled to 0° C. and charged with sodium chlorite (0.075 g, 0.66 mmol). The reaction mixture was allowed to stir at room temperature for 12 hr. then acidified to pH 2 with 1N HCl. The precipitated white solid was filtered, washed with water several times and dried to give 2-hydroxy-4-(methoxycarbonyl)benzoic acid. Yield: (0.035 g, 65%). Molecular Weight: 196. MS (ES+): m/z=197.2 [MH⁺].

### Step-3:

A solution of 2-hydroxy-4-(methoxycarbonyl)benzoic acid (0.05 g, 0.25 mmol) in THF (10 mL) was cooled to 0° C. and charged with thionyl chloride (0.303 g, 2.5 mmol) then the reaction mixture was heated to 45° C. for 4 hr. The reaction mixture was concentrated in vacuo and the residue was diluted with dry DCM (5 ml) and charged with a solution of o-phenyl amine.HCl (0.055 g, 0.38 mmol), NaHCO₃ (0.038 mg, 0.45 mmol) and in DCM (15 ml) at 0° C. then the reaction was charged with 1N HCl solution (15 ml) and the organic was separated, dried over sodium sulfate, filtered, and concentrated in vacuo resulting in 0.07 g of crude product which was purified by column chromatography on silica gel eluting with hexane-ethyl acetate resulting in methyl 3-hydroxy-4-(phenoxycarbamoyl)benzoate. Yield: (0.5 g, 68%). Molecular Weight: 287. MS (ES+): m/z=288.1 [MH⁺].

### Step-4:

A solution of methyl 3-hydroxy-4-(phenoxycarbamoyl)benzoate (0.05 g, 0.17 mmol) in THF:water (2:1) (7.5 mL) was charged with LiOH (0.012 g, 0.51 mmol) and stirred at room temperature for 6 h. The reaction mixture was concentrated and the aqueous was acidified to pH 2 with 1N HCl and the precipitate was filtered and dried to give 3-hydroxy-4-(phenoxycarbamoyl)benzoic acid. Yield: (0.03 g, 63.8%). Molecular Weight: 273. MS (ES+): m/z=274.0 [MH⁺].

### Synthesis of 3-(tert-butoxycarbamoyl)-4-hydroxybenzoic acid (A-92-O-t-bu)

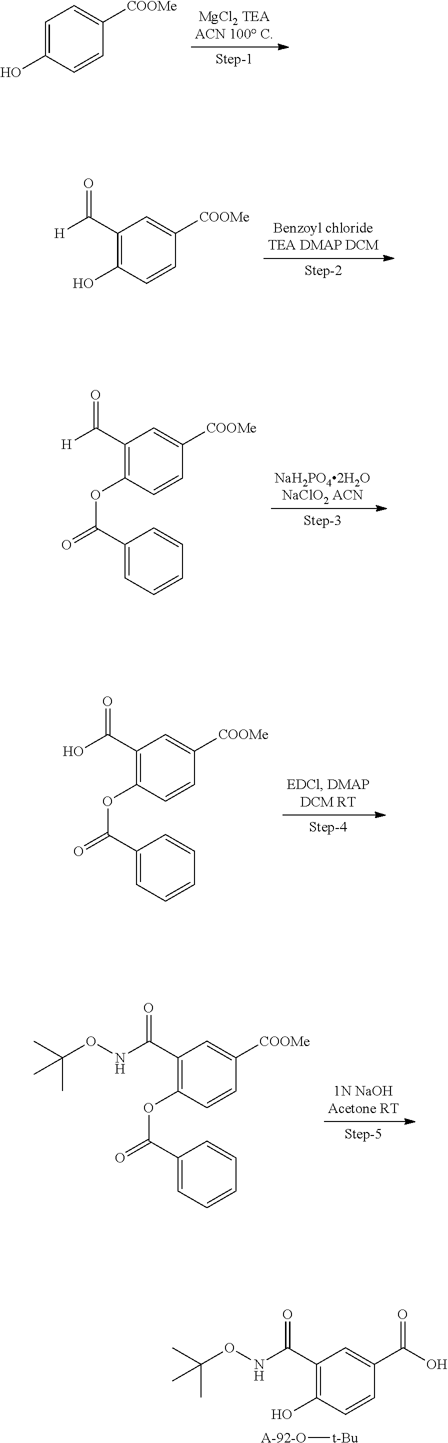

### Step-1:

A solution of methyl-4-hydroxy benzoate (2 g, 13.15 mmol) and anhydrous magnesium chloride (1.87 g, 19.7 mmol) in acetonitrile (100 mL) was charged with triethyl amine (7 mL, 49.9 mmol). The reaction mixture was then charged with para formaldehyde (8 g, 89.4 mmol) in a single portion and the reaction mixture was heated at reflux for 24 hours. The reaction mixture was cooled and quenched with 1N HCl and extracted with ethyl acetate. The organic layer was washed with water and separated dried over sodium sulfate, filtered, and concentrated in vacuo. The crude material was purified by column chromatography using hexane ethyl acetate as eluent to give methyl 3-formyl-4-hydroxybenzoate as white solid. Yield: (0.51 g, 22%). ¹H NMR (400 MHz, CDCl₃): δ 3.93 (s, 3H), 7.04 (d, J=8.8 Hz, 1H), 8.18-8.20 (dd, J=1.6 Hz, J=8.8 Hz, 1H), 8.32 (s, 1H), 9.56 (s, 1H), 11.39 (s, 1H).

### Step-2:

A solution of methyl 3-formyl-4-hydroxybenzoate (1.8 g, 0.01 mol) in dichloromethane (120 mL) was cooled to 0° C. and charged with DMAP (0.12 g, 0.001 mol), triethylamine (5.5 mL, 0.04 mol) and benzoyl chloride (2.3 mL, 0.02 mol). The reaction mixture was allowed to stir at room temperature overnight. The reaction mixture was quenched with water. The organic layer was separated and washed with water and the organic layer was separated, dried over sodium sulfate, filtered, and concentrated in vacuo. The crude material was purified by column chromatography using hexane/ethyl acetate as eluent resulting in methyl 4-(benzoyloxy)-3-formylbenzoate. Yield: (1.4 g, 49.2%). ¹H NMR (400 MHz, DMSO-d₆): δ 3.83 (s, 3H), 7.06 (d, J=8.8 Hz, 1H), 8.02-8.07 (dd, J=1.6 and 8.6 Hz, 1H), 8.38 (d, J=1.2 Hz, 1H).

### Step-3:

A solution of methyl 4-(benzoyloxy)-3-formylbenzoate (0.05 g, 0.17 mmol) and NaH₂PO₄.2H₂O (0.068 g, 0.44 mmol) in 2:1 of DMSO:H₂O (6 mL) was charged with sodium chlorite (0.038 g, 0.42 mmol). The reaction mixture was stirred at room temperature for 2 hours and acidified to pH 2 with 1N HCl. The white precipitate was filtered, washed with water several times, and dried to give 2-(benzoyloxy)-5-(methoxycarbonyl)benzoic acid as the desired product. Yield: (0.05 g, 96.1%). ¹H NMR (400 MHz, DMSO-d₆): δ 3.83 (s, 3H), 7.06 (d, J=8.8 Hz, 1H), 8.02-8.07 (dd, J=1.6, 8.6 Hz, 1H), 8.38 (d, J=1.2 Hz, 1H).

### Step-4:

A solution of 2-(benzoyloxy)-5-(methoxycarbonyl)benzoic acid (0.3 g, 1.00 mmol) in DCM (15 mL) was charged with DMAP (0.061 g, 0.5 mmol), EDCI (0.28 g, 1.5 mmol) and o-(tert-butyl)hydroxylamine hydrochloride (0.18 g, 1.5 mmol) and the mixture was stirred at room temperature for 2 hours. The reaction mixture was washed with water (3×), 2N HCl (3×), and separated. The organic layer was dried over sodium sulfate, filtered, and concentrated in vacuo resulting in crude material. The crude product was purified by column chromatography on silica gel using hexane/ethyl acetate as eluent to give methyl 4-(benzoyloxy)-3-(tert-butoxycarbamoyl)benzoate. Yield: (0.2 g, 54%). MS (ES+): m/z=372 [MH⁺].

### Step-5:

A solution of methyl 4-(benzoyloxy)-3-(tert-butoxycarbamoyl)benzoate (0.05 g, 0.13 mmol) in acetone (1.2 mL) was charged with 1N NaOH (1.2 mL) and the reaction mixture was stirred at room temperature overnight. The reaction mixture was concentrated in vacuo and the aqueous was acidified to pH 2 using 1N HCl. A solid precipitated out and was filtered and dried to give 3-(tert-butoxycarbamoyl)-4-hydroxybenzoic acid. Yield: (0.015 g, 44%). MS (ES+): m/z=254 [MH⁺].

### Synthesis of 4-hydroxy-3-(methoxycarbamoyl)-5-methylbenzoic acid (A-114)

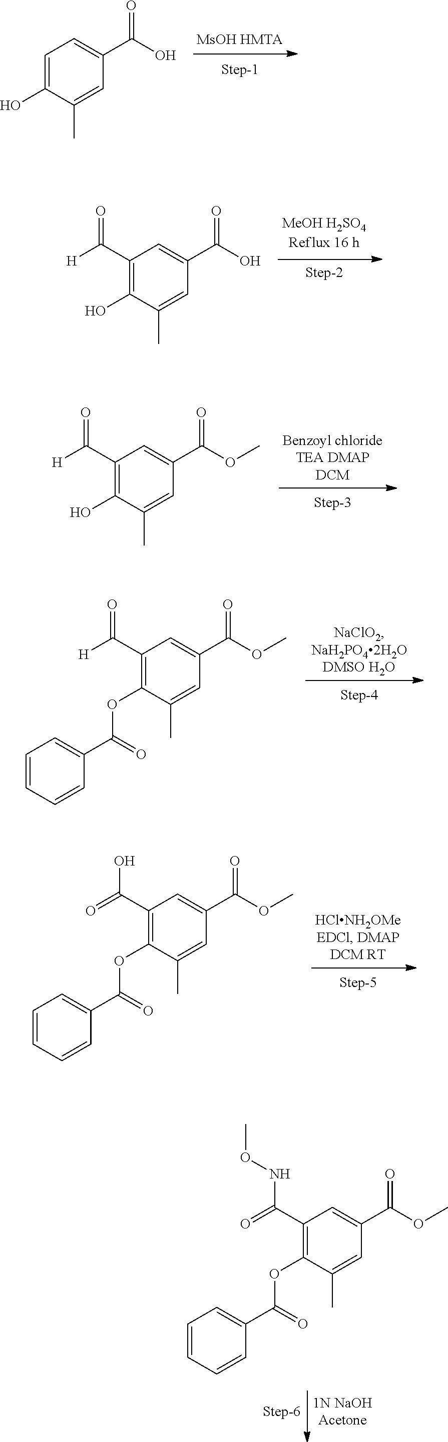 [SMILES: Cc1cc(C(=O)O)ccc1O ; Cc1cc(C(=O)O)cc(C=O)c1O ; COC(=O)c1cc(C)c(O)c(C=O)c1 ; COC(=O)c1cc(C)c(OC(=O)c2ccccc2)c(C=O)c1 ; COC(=O)c1cc(C)c(OC(=O)c2ccccc2)c(C(=O)O)c1 ; CONC(=O)c1cc(C(=O)OC)cc(C)c1OC(=O)c1ccccc1]

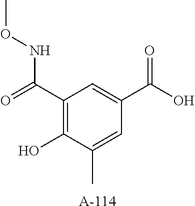

### Step-1:

A suspension of 4-hydroxy-3-methylbenzoic acid (1 g, 6.57 mmol) suspended in methanesulfonic acid (5 mL) was cooled to 0° C. and portion-wise charged with hexamethylenetetramine (1.84 g, 13.15 mmol). The reaction mixture was warmed to room temperature followed by heating at 90° C. for 5 hours, then cooled to room temperature and stirred overnight. The reaction mixture was poured into ice cooled water and the compound was extracted in ethyl acetate. The organic layer was washed with water, dried over sodium sulfate, filtered, and concentrated in vacuo to give 3-formyl-4-hydroxy-5-methylbenzoic acid as yellow solid. Yield: (0.5 g, 42.3%). ¹H NMR (400 MHz, CDCl₃): δ 3.93 (s, 3H), 7.04 (d, J=8.8 Hz, 1H), 8.19 (dd, 1H, J=1.6 Hz, 8.8 Hz, 1H), 8.32 (s, 1H), 9.56 (s, 1H), 11.39 (s, 1H).

### Step-2:

A solution of 3-formyl-4-hydroxy-5-methylbenzoic acid (0.2 g, 1.11 mmol) in methanol (4 mL) was charged with concentrated sulfuric acid (0.14 mL) and refluxed for 16 hours. The reaction mixture was concentrated and the aqueous layer was extracted in ethyl acetate. The combined organic layer was washed with saturated solution of sodium bicarbonate, dried over sodium sulfate, filtered, concentrated in vacuo to give methyl 3-formyl-4-hydroxy-5-methylbenzoate as an off white solid. Yield: (0.18 g, 85.7%). ¹H NMR (400 MHz, DMSO-d₆): δ 3.83 (s, 3H), 7.06 (d, J=8.8 Hz, 1H), 8.02-8.07 (dd, J=1.6, 8.6 Hz, 1H), 8.38 (d, J=1.2 Hz, 1H).

### Step-3:

A solution of methyl 3-formyl-4-hydroxy-5-methylbenzoate (0.5 g, 2.57 mmol) in dichloromethane (50 mL) was cooled to 0° C. and charged with DMAP (0.031 g, 0.25 mmol), triethylamine (1.4 mL, 1.03 mmol), and benzoyl chloride (0.6 mL, 5.15 mmol). The reaction mixture was stirred at room temperature overnight, and then quenched with water. The organic layer was separated and washed with water. The organic layer was dried over sodium sulfate, filtered, and concentrated in vacuo. The crude product was purified by column chromatography on silica gel using hexanes/ethyl acetate as eluent to give methyl 4-(benzoyloxy)-3-formyl-5-methylbenzoate. Yield: (0.5 g, 65.7%). ¹H NMR (400 MHz, CDCl₃): δ 3.91 (s, 3H), 3.92 (s, 3H), 7.03 (d, J=8.8 Hz, 1H), 8.05-8.09 (dd, J=1.8, 8.6 Hz, 1H), 8.16 (s, 1H), 9.48 (s, 1H), 12.2 (s, 1H).

### Step-4:

A solution of methyl 4-(benzoyloxy)-3-formyl-5-methylbenzoate (0.5 g, 1.67 mmol) and NaH₂PO₄.2H₂O (0.65 g, 4.19 mmol) in DMSO:water (2:1, 30 mL) was charged with sodium chlorite (0.36 g, 4.02 mmol). The reaction mixture was stirred at room temperature for 2 hours, and then acidified to pH=2 with 1N HCl, upon which a white precipitate formed. The precipitate was filtered, washed with water several times and dried to give 2-(benzoyloxy)-5-(methoxycarbonyl)-3-methylbenzoic acid as the desired product. Yield: (0.4 g, 77%). ¹H NMR (400 MHz, DMSO-d₆): δ 3.70 (s, 3H), 7.05 (d, J=8.4 Hz, 1H), 7.89-7.93 (dd, J=1.4, 8.6 Hz, 1H), 8.26 (s, 1H).

### Step-5:

A solution of 2-(benzoyloxy)-5-(methoxycarbonyl)-3-methylbenzoic acid (0.2 g, 0.63 mmol), DMAP (0.077 g, 0.63 mmol), EDCI (0.18 g, 0.95 mmol) in DCM (20 mL) was charged with o-methyl hydroxylamine hydrochloride (0.08 g, 0.95 mmol) and stirred at room temperature for 2 hours. The reaction mixture was washed with water (3×), 2N HCl (3×), and separated. The combined organic layer was dried over sodium sulfate, filtered, and concentrated in vacuo and the crude was further purified by column chromatography on silica gel using hexanes/ethyl acetate as eluent to give methyl 4-(benzoyloxy)-3-(methoxycarbamoyl)-5-methylbenzoate. Yield: (0.12 g, 57.1%). ¹H NMR (400 MHz, CDCl₃): δ 1.46 (s, 9H), 1.64-2.00 (m, 4H), 2.70-2.82 (m, 1H), 2.90-3.40 (br, 2H), 4.29 (s, 2H), 4.50-5.00 (br, 2H), 6.97 (d, J=8.4 Hz, 1H), 7.00-7.20 (m, 4H), 7.26-7.30 (m, 1H), 7.42 (d, J=8.4 Hz, 1H), 7.70 (s, 1H), 10.7 (s, 1H), 12.1 (s, 1H).

### Step-6:

A solution of methyl 4-(benzoyloxy)-3-(methoxycarbamoyl)-5-methylbenzoate (0.12 g, 0.34 mmol) in acetone (2.5 mL) was charged with 1N NaOH (2.5 mL) and stirred at room temperature overnight. The reaction mixture was concentrated in vacuo and the aqueous was acidified to pH 2 with 1N HCl. Upon acidification, a precipitate formed. The precipitate was filtered and dried to give 4-hydroxy-3-(methoxycarbamoyl)-5-methylbenzoic acid. Yield: (0.03 g, 38.4%). MS (ES+): m/z=226 [MH⁺].

B. Synthesis of Intermediate Amides and Final Targets with their Respective Approaches

### Step-1:

Couplings of desired suitably substituted carboxylic acids were carried out with protected 4-(3-aminomethyl phenyl)piperidine or 5-aminomethyl spiro[benzofuran-3,4′-piperidine] as per conditions described in the table below. Work-up of reactions was carried out as described below in General Procedures for Examples 14-17.

Details of the compound are given in the table as below.

| Compound | | Brief Reaction | |
|---|---|---|---|
| No. | Structure | conditions | Analytical data |
| B-75a-O—t-Bu | 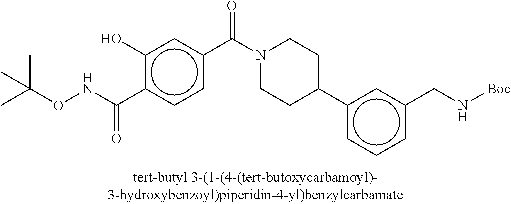 | EDCI (1.5 eq.), DMAP (1.2 eq), DCM (~200 vol.), phenyl piperidine core (1 eq.). Stirred at room temperature for 3 hours. Crude product used for next step without purification. | Yield: 96%; Mol.Wt.: 525.64; MS (ES+): m/z = 526.35 [MH⁺]. |
| B-75a-O—t-Bu Spiro | 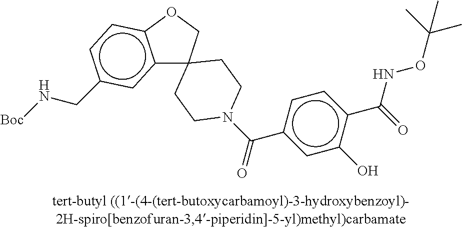 | EDCI (1.5 eq.), DMAP (1.2 eq.), DCM (~200 vol.), phenyl piperidine core (1 eq.). Stirred at room temperature for 3 hours. Crude product used for next step without purification. | Yield: 82.5%; Mol.Wt.: 553.65; MS (ES+): m/z = 586.350 [M + Na]. |
| B-75a-O—Ph | 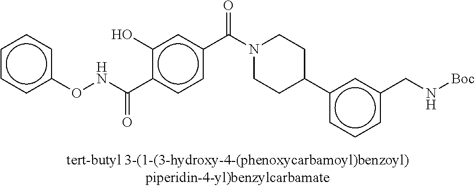 | EDCI (1.5 eq.), DMAP (1.2 eq.), DCM (~200 vol.), phenyl piperidine core (1 eq.). Stirred at room temperature for 3 hours. Crude product used for next step without purification. | Yield: 90%; Mol.Wt.: 545.63; MS (ES+): m/z = 558.3 [M + Na]. |
| B-75a-O—Ph- spiro | 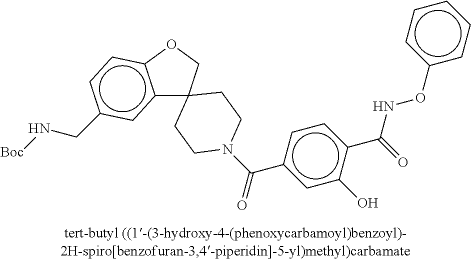 | EDCI (1.5 eq.), DMAP (1.2 eq.), DCM (~300 vol.), phenyl piperidine core (1 eq.). Stirred at room temperature for 3 hours. Crude product used for next step without purification. | Yield: 96%; Mol.Wt.: 573.64; MS (ES+): m/z = 596.20 [M + Na]. |
| B-92-O—t-Bu | 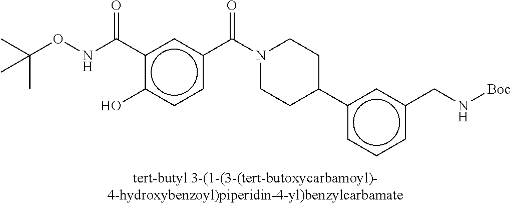 | EDCI (1.5 eq.), DMAP (0.5 eq.), DCM (~300 vol.), phenyl piperidine core (1 eq.). Stirred at room temperature for 4 hours. Crude product used for next step without purification. | Yield: 64.5%; Mol.Wt.: 525.64; MS (ES+): m/z = 426 [M − Boc]. |
| B-92-O—t-Bu spiro | 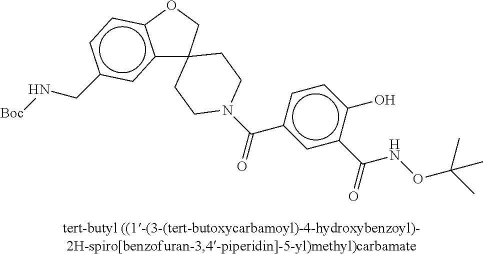 | EDCI (1.5 eq.), DMAP (0.5 eq.), DCM (~300 vol.), Spiro core (1.2 eq.). Stirred at room temperature for 4 hours. Crude product used for next step without purification. | Yield: 89.8%; Mol.Wt.: 553.65; MS (ES+): m/z = 576 [M + Na]. |
| B-114 Spiro | 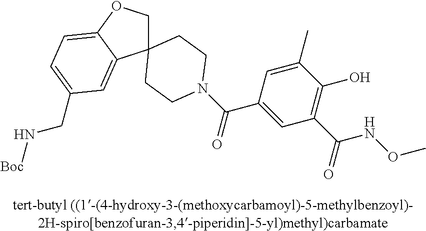 [SMILES: CONC(=O)c1cc(C(=O)N2CCC3(CC2)COc2ccc(CNC(=O)OC(C)(C)C)cc23)cc(C)c1O] | EDCI (1.5 eq.), DMAP (0.5 eq.), DCM (~150 vol.), Spiro core (1.2 eq.). Stirred at room temperature for 4 hours, Crude product purified by column chromatography using hexane ethyl acetate. | Yield: 71.4%; Mol.Wt.: 525.59; MS (ES+): m/z = 526 [MH⁺]. |

### Step-2:

Products of Step-1 were deprotected as per conditions described in the table below. The details of the compounds synthesized are as below. All reactions were done on 100-200 mg scale.

| Compound | | Brief Reaction | |
|---|---|---|---|
| No. | Structure | conditions | Analytical data |
| 75a-O—t-Bu | 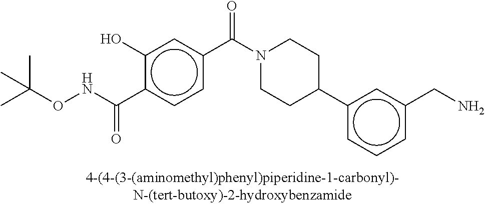 | DCM (~175 vol.), TFA (6 Vol.). Stirred at room temperature for 3 hours, followed by concentration and purification by preparative HPLC. | Yield: 41%;. Mol.Wt. 425.52; MS (ES+): m/z = 426.25 [MH⁺]; HPLC: 97.9% (200-400 nm); ¹H NMR (400 MHz, DMSO-d₆, D₂O): δ 7.76 (s, 1H), 7.74 (d, J = 8.2 Hz, 1H), 7.49 (d, J = 6.7 Hz, 1H), 7.30 (dt, J = 25.5, 8.2 Hz, 1H), 7.07 (dd, J = 13.7, 7.5 Hz, 2H), 6.95 (d, J = 8.4 Hz, 1H), 4.01 (q, J = 5.6 Hz, 2H) 3.20 (m, 3H), 2.86 (s, 2H), 1.91- 1.53 (m, 4H), 1.25 (s, 9H). |
| 75a-O—Ph | 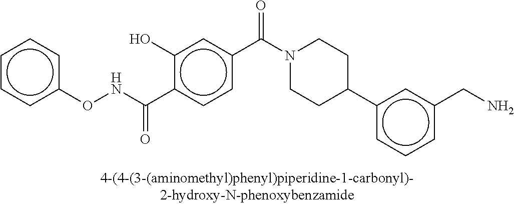 | DCM (~50 vol.), TFA (6 vol.). Stirred at room temperature for 3 hours, followed by concentration and purification by preparative HPLC. | Yield: 10%; Mol. Wt. 445.41; MS (ES+): m/z = 446.20 [MH⁺]; HPLC: 96.68% (200-400 nm); ¹H NMR (400 MHz, DMSO-d₆, D₂O): δ 7.94 (s, 1H), 7.81 (s, 1H), 7.74 (d, J = 8.2 Hz, 1H), 7.49 (d, J = 6.7 Hz, 1H), 7.30 (dt, J = 25.5, 8.2 Hz, 5H), 7.07 (dd, J = 13.7, 7.5 Hz, 2H), 6.95 (d, J = 8.4 Hz, 1H), 3.97 (s, 2H), 3.20 (m, 3H), 2.86 (s, 2H), 1.91-1.53 (m, 4H). |
| 75a-O—t-Bu Spiro | 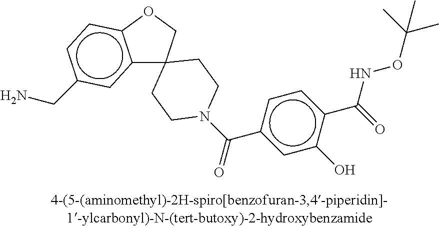 | DCM (~100 vol.), TFA (6 vol.). Stirred at room temperature for 3 hours, followed by concentration and purification by preparative HPLC. | Yield: 48%; Mol. Wt. 453.53; MS (ES+): m/z = 517.20 [M + Na + AcN]; HPLC: 99.13% (200-400 nm); ¹H NMR (400 MHz, DMSO-d₆): δ 11.09 (s, 1H), 8.05 (d, J = 16.0 Hz, 3H), 7.77 (d, J = 7.9 Hz, 1H), 7.43 (d, J = 2.0 Hz, 1H), 7.23 (dd, J = 8.3, 1.9 Hz, 1H), 6.96-6.84 (m, 2H), 4.50 (d, J = 4.5 Hz, 2H), 4.36 (d, J = 13.3 Hz, 1H), 3.95 (q, J = 5.6 Hz, 2H), 3.29-3.01 (m, 4H), 1.76 (q, J = 22.4, 20.8 Hz, 4H), 1.25 (s, 9H) |
| 75a-O—Ph- spiro | 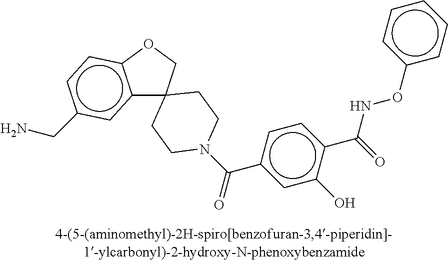 | DCM (~50 vol.), TFA (6 Vol). Stirred at room temperature for 3 hours, followed by concentration and purification by preparative HPLC. | Yield: 29%; Mol. Wt. 473.52; MS (ES+): m/z = 474.20 [MH⁺]; HPLC: 99.80% (200-400 nm); ¹H NMR (400 MHz, DMSO-d₆): δ 8.03 (s, 3H), 7.79 (d, J = 7.9 Hz, 1H), 7.43 (s, 1H), 7.35 (t, J = 7.8 Hz, 1H), 7.23 (d, J = 7.9 Hz, 1H), 7.12 (d, J = 8.2 Hz, 1H), 7.06 (t, J = 7.3 Hz, 2H), 7.01-6.85 (m, 3H), 6.84 (s, 1H), 4.51 (d, J = 3.7 Hz, 2H), 4.36 (s, 1H), 3.95 (q, J = 5.5 Hz, 2H), 3.58 (s, 2H), 3.10 (s, 2H), 1.74 (d, J = 37.8 Hz, 4H). |
| 92-O—t-Bu | 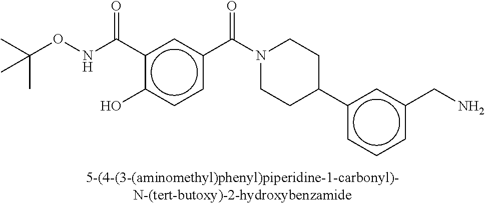 | Dioxane (~200 vol.), Concentrated HCl (3.5 vol.). Stirred at room temperature for 4 hours, followed by concentration and purification by preparative HPLC. | Yield: 50%; Mol. Wt. 425.52; MS (ES+): m/z = 448 [M + Na]; HPLC: 94.5% (220 nm); ¹H NMR (400 MHz, DMSO-d₆): δ 1.50- 1.90 (m, 4H), 2.75- 2.90 (m, 1H), 2.91- 3.30 (br, 2H), 3.50- 3.60 (br, 2H), 3.73 (s, 3H), 4.00-4.10 (m, 2H), 6.99 (d, J = 8.4 Hz, 1H), 7.20- 7.40 (m, 4H), 7.47 (d, J = 8.4 Hz, 1H), 7.77 (s, 1H), 8.20 (br, 2H), 11.7 (br, 1H), 11.9 (br, 1H). |
| 92-O—t-Bu spiro | 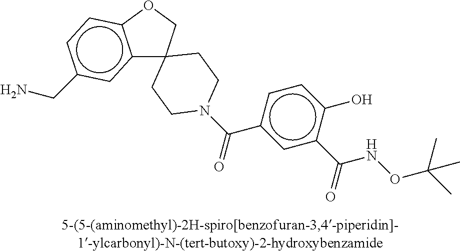 | Dioxane (~75 vol.), Concentrated HCl (3.5 vol.). Stirred at room temperature for 3 hours, followed by concentration and purification by preparative HPLC. | Yield: 38%; Mol. Wt. 453.53; MS (ES+): m/z = 476 [M + Na]; HPLC: 99.0% (220 nm); ¹H NMR (400 MHz, DMSO-d₆): δ 1.50- 1.90 (m, 4H), 2.75- 2.90 (m, 1H), 2.91- 3.30 (br, 2H), 3.50- 3.60 (br, 2H), 3.73 (s, 3H), 4.00-4.10 (m, 2H), 6.99 (d, J = 8.4 Hz, 1H), 7.20- 7.40 (m, 4H), 7.47 (d, J = 8.4 Hz, 1H), 7.77 (s, 1H), 8.20 (br, 2H), 11.7 (br, 1H), 11.9 (br, 1H). |
| 114 Spiro | 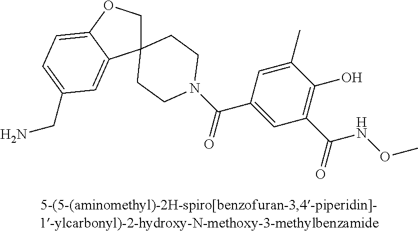 [SMILES: CONC(=O)c1cc(C(=O)N2CCC3(CC2)COc2ccc(CN)cc23)cc(C)c1O] | Dioxane (~100 vol.), Concentrated HCl (4 vol.). Stirred at room temperature for 4 hours, followed by concentration and purification by preparative HPLC. | Yield: 33.3%; Mol Wt.: −425.48; MS (ES+): m/z = 448 [M + Na]; HPLC: 95.98% (220 nm); ¹H NMR (400 MHz, DMSO-d₆): δ 1.50- 1.90 (m, 4H), 2.75- 2.90 (m, 1H), 2.91- 3.30 (br, 2H), 3.50- 3.60 (br, 2H), 3.73 (s, 3H), 4.00-4.10 (m, 2H), 6.99 (d, J = 8.4 Hz, 1H), 7.20- 7.40 (m, 4H), 7.47 (d, J = 8.4 Hz, 1H), 7.77 (s, 1H), 8.20 (br, 2H), 11.7 (br, 1H), 11.9 (br, 1H). |

### Approach-2

Carboxy O-methyl salicylaldehydes/Protected salicylic acids were first coupled with the protected core. Subsequent O-methylation and oxidation (in case of aldehydes) or deprotection (in case of protected salicylic acids) of the coupled product yielded carboxylic acid, which was coupled with suitable amine. Boc protection on amino methyl functionality was then carried out to obtain the desired products. In case of O-Methyl compounds, O-de-methylation and Boc deprotection was carried out together using boron tribromide as in the reaction scheme showing below:

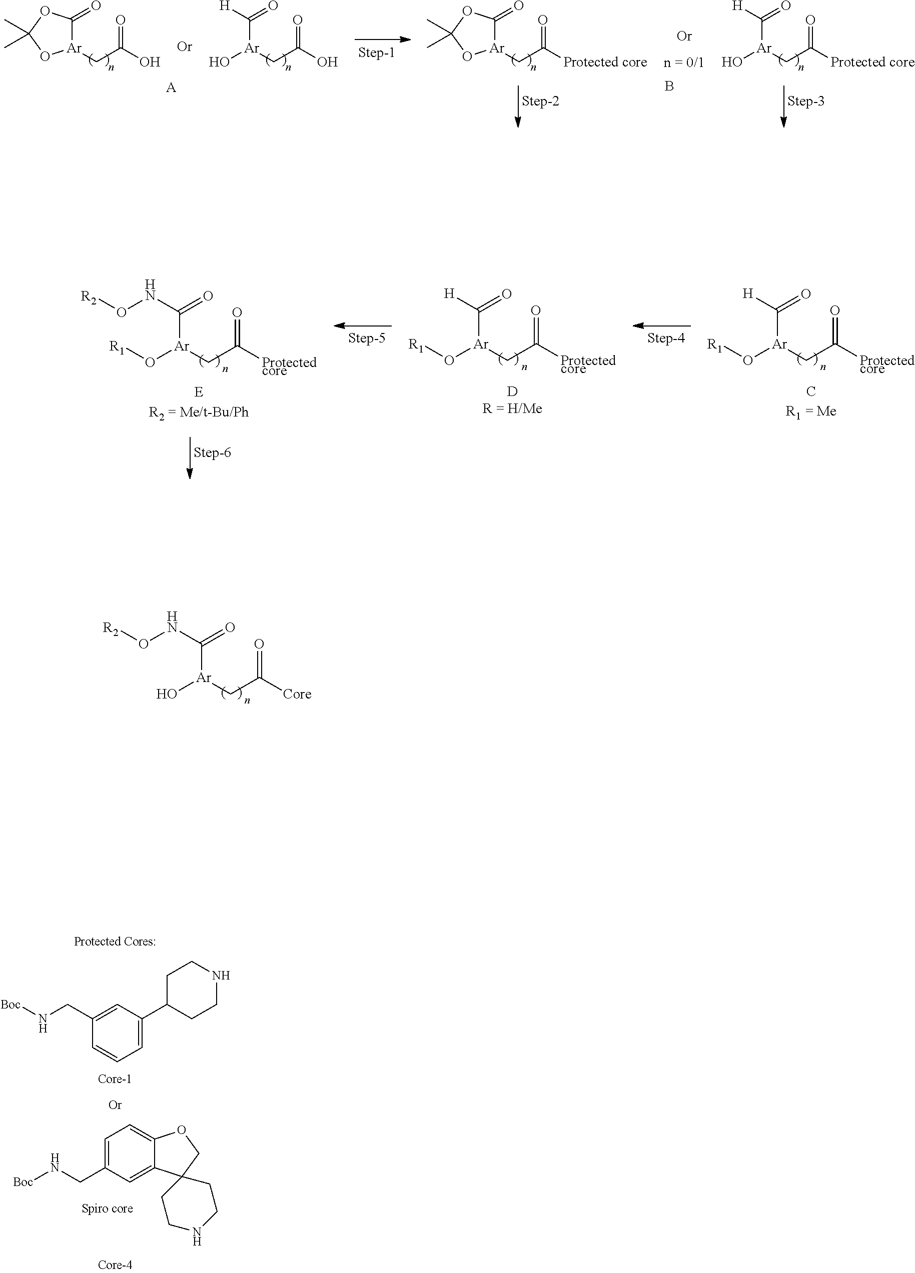

Step-1: Coupling of Protected Salicylic Acids/Salicylaldehydes with Appropriate Core (Core-1/Core-4 as Shown in Synthetic Scheme Above)

A stirred solution of protected salicylic acid/salicylaldehyde in DCM was charged with EDCI, HOBt (in some cases) and DMAP or DIPEA. The solution was stirred for 15 minutes at 0° C. followed by addition of protected core. Stirring was continued at room temperature, and reaction was monitored by LCMS until most of the starting materials were consumed. Reaction mixture was then quenched with water. Aqueous layer was extracted with dichloromethane. Combined organic layers were dried over sodium sulfate, filtered, and concentrated under vacuum to afford the crude product, which were sufficiently pure to be used for next step.

| Compound | | Brief Reaction | |
|---|---|---|---|
| No. | Structure | conditions | Analytical data |
| B-92-Spiro-O—Ph | 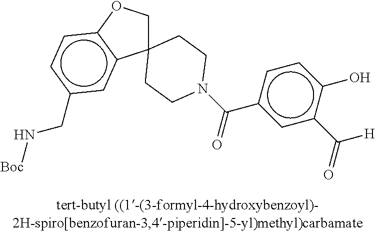 | EDCI (1.5 eq.), DMAP (0.5 eq), DCM (100 Vol), Spiro core (1.2 eq.). Stirred at room temperature for 4 hours. Crude product used for next step without purification. | Yield: 53.5%; Mol. Wt.: 466.53 MS (ES+): m/z = 489 [M + Na]. |
| B-92-O—Ph | 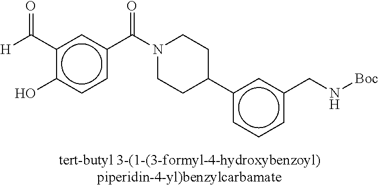 | EDCI (1.5 eq.), DMAP (0.5 eq.), DCM (100 vol.), Phenyl piperidine core (1 eq.). Stirred at room temperature for 4 hours. Crude product used for next step without purification. | Yield: 72%; Mol. Wt.: 438.52 MS (ES+): m/z = 502 [M + Na + AcN]. |

### Step-2: O-Methylation of Step-1 Product:

A solution of Step-1 product and potassium carbonate in acetone was charged with methyl iodide and heated at 70° C. for 4 hours. The reaction mixture was filtered and concentrated in vacuo and the compound was extracted in dichloromethane and washed with water. The organic layer was washed with water, dried over sodium sulfate, filtered, and concentrated to afford Step-2 product. The crude product was used as such for the next step without purification.

| Compound | | Brief Reaction | |
|---|---|---|---|
| No. | Structure | conditions | Analytical data |
| D-92-Spiro-O—Ph | 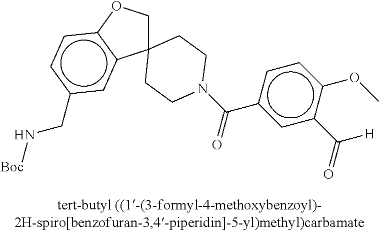 | Acetone (60 vol.), Potassium carbonate (3 eq.), methyl iodide (1.2 eq), 70° C., 4 hours, Isolated by distillation of solvent, dilution with water and extraction with dichloromethane and concentration. Crude product used for next step. | Yield: 95% (Crude); Mol. Wt.: 480.55 LCMS (m/z): 503 [M + Na]. |
| D-92-O—Ph | 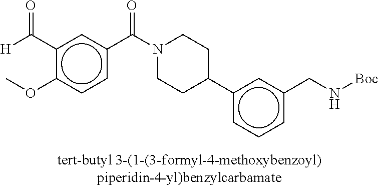 | Acetone (60 vol.), Potassium carbonate (3 eq.), Methyl iodide (2 eq.), 70° C., 4 hours. Isolated by distillation of solvent, dilution with water and extraction with dichloromethane and concentration. Crude product used for next step. | Yield: 100% (Crude); ¹H NMR (400 MHz, CDCl₃): δ 3.93 (s, 3H), 7.04 (d, J = 8.8 Hz, 1H), 8.18-8.20 (dd, J = 1.6 Hz, J = 8.8 Hz, 1H), 8.32 (s, 1H), 9.56 (s, 1H), 11.39 (s, 1H). |

### Step-3: Oxidation of Step-2 Product:

A solution of Step-2 product and NaH₂PO₄.2H₂O in DMSO:water was charged with sodium chlorite and allowed to stir at room temperature for 2 hours. The reaction mixture was acidified to pH=2 with 1N HCl upon which a precipitate formed. The white precipitate was filtered, washed with water several times and dried to afford Step-3 product.

| Compound | | Brief Reaction | |
|---|---|---|---|
| No. | Structure | conditions | Analytical data |
| D-92-Spiro-O—Ph | 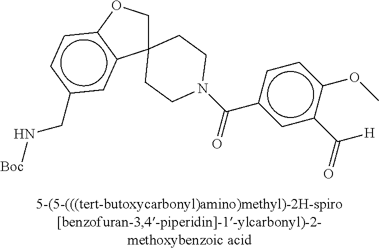 | Sodium chlorite (2.4 eq.), Sodium dihydrogen phosphate dehydrate (2.5 eq.), DMSO (40 vol.), Water (20 vol.). Stirred at room temperature for 2 hours, followed by acidification with 1N HCl to pH-2. Filtration to obtain solid product which was sufficient pure to be used for next step. | Yield: 88.2%; ¹H NMR (400 MHz, DMSO-d₆): δ 3.70 (s, 3H), 7.05 (d, J = 8.4 Hz, 1H), 7.89-7.93 (dd, J = 1.4, 8.6 Hz, 1H), 8.26 (s, 1H). |
| D-92-O—Ph | 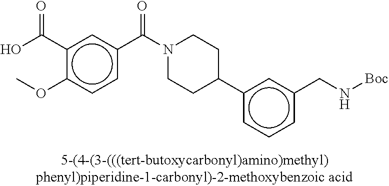 | Sodium chlorite (2.4 eq.), Sodium dihydrogen phosphate dehydrate (2.5 eq.), DMSO (20 vol.), Water (10 vol.). Stirred at room temperature for 2 hours, followed by acidification with 1N HCl to pH-2. Filtration to obtain solid product which was sufficient pure to be used for next step. | Yield: 57%; ¹H NMR (400 MHz, CDCl₃): δ 3.93 (s, 3H), 7.04 (d, 1H, J = 8.8 Hz), 8.18- 8.20 (dd, J = 1.6 Hz, 8.8 Hz, 1H), 8.32 (s, 1H), 9.56 (s, 1H), 11.39 (s, 1H). |

Step-4: Amide Coupling of Step-3 Products with O-Phenyl and O-Methyl Hydroxyl Amines:

A solution of Step-3 products in dioxane/pyridine, and Boc anhydride was charged with O-phenylhydroxylamine and stirred at room temperature overnight. Reaction mixture was concentrated in vacuo and given for preparative purification to give Step-4 product.

| Compound | | Brief Reaction | |
|---|---|---|---|
| No. | Structure | conditions | Analytical data |
| D-92-Spiro-O—Ph | 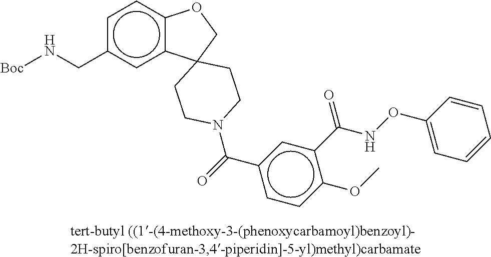 | Dioxane (20 vol.), pyridine (1 eq.), Boc anhydride (1.3 eq.) O-phenyl hydroxylamine (1.3 eq.). Stirred room temperature for 12 hours. Purified by preparative HPLC after concentration in vacuum. | Yield: −17.8%; Mol. Wt.: 587.66; MS (ES+): m/z = 488 [M − Boc]. |
| D-92-O—Ph | 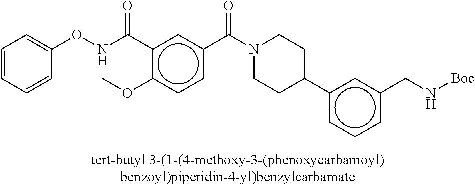 | Dioxane (25 vol.), pyridine (1 eq.), Boc anhydride (1.3 eq.) O-phenyl hydroxylamine (1.3 eq.). Stirred room temperature for 12 hours. Purified by preparative HPLC after concentration in vacuum. | Yield: 26.7%; Mol. Wt.: 559.65; MS (ES+): m/z = 460 [M − Boc]. |

### Step-5:—Deprotection of Protected Core:

A solution of Step-4 product in dichloromethane was charged with BBr₃ in DCM. The reaction mixture was stirred at room temperature for 3 hours. The reaction mixture was concentrated and purified by preparative HPLC to afford final target compounds.

| Compound | | Brief Reaction | |
|---|---|---|---|
| No. | Structure | conditions | Analytical data |
| 92-Spiro-O—Ph | 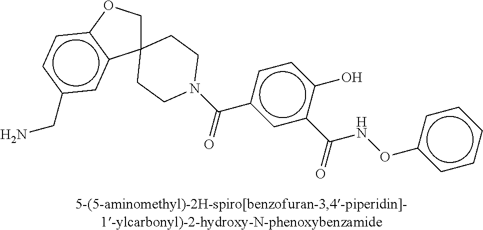 | 1M BBr₃ in DCM (1.5 eq.). Stirred room temperature for 3 hours. Purified by preparative HPLC after concentration in vacuum. | Yield: 24%; Mol. Wt.: 473.52; MS (ES+): m/z = 474[MH⁺]; HPLC: 96.1% (220 nm); ¹H NMR (400 MHz, DMSO-d₆): δ 1.50-1.90 (m, 4H), 2.75-2.90 (m, 1H), 2.91-3.30 (br, 2H), 3.50- 3.60 (br, 2H), 3.73 (s, 3H), 4.00-4.10 (m, 2H), 6.99 (d, J = 8.4 Hz, 1H), 7.20-7.40 (m, 4H), 7.47 (d, J = 8.4 Hz, 1H), 7.77 (s, 1H), 8.20 (br, 2H), 11.7 (br, 1H), 11.9 (br, 1H). |
| 92-O—Ph | 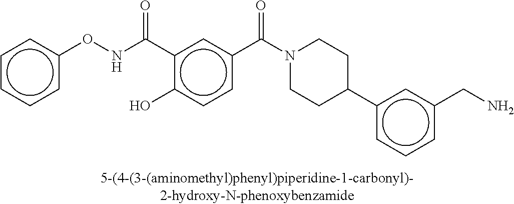 | 1M BBr₃ in DCM (1.5 eq.). Stirred room temperature for 3 hours. Purified by preparative HPLC after concentration in vacuum. | Yield:: −10%, Mol. Wt. 445.51 MS (ES+): m/z = 446 [MH⁺] HPLC: 86.8% (220 nm) ¹H NMR (400 MHz, DMSO-d₆): δ 1.50-1.90 (m, 4H), 2.75-2.90 (m, 1H), 2.91-3.30 (br, 2H), 3.50- 3.60 (br, 2H), 3.73 (s, 3H), 4.00-4.10 (m, 2H), 6.99 (d, J = 8.4 Hz, 1H), 7.20-7.40 (m, 4H), 7.47 (d, J = 8.4 Hz, 1H), 7.77 (s, 1H), 8.20 (br, 2H), 11.7 (br, 1H), 11.9 (br, 1H). |

### Example 16

### Synthesis of Cofluorons with Phenolic and Hydroxymethyl Phenol Functionality

11 Final Targets with phenolic and hydroxymethyl phenol functionality were synthesized. Title compounds were synthesized by two different approaches as described below.

### Approach-1:

Functionalized dihydroxy aromatic carboxylic acids were coupled with the required core and coupled product was deprotected as described in the scheme below.

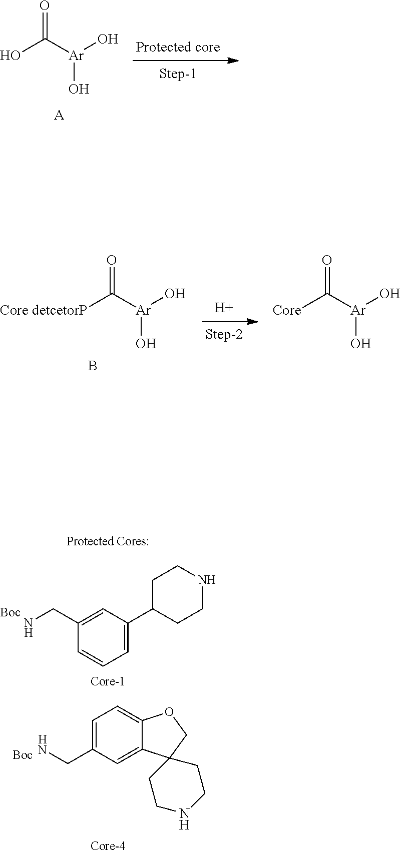

### Step-1:

Coupling of carboxylic acids (A) was carried out with Core-1 or Core-4 as shown in general synthetic scheme above. Work-up of reactions were carried out as described below in General Procedures for Examples 14-17.

The details of the compounds synthesized are shown as below. Reactions were done on 100-200 mg scale.

| Compound | | Brief reaction | |
|---|---|---|---|
| No. | Structure | conditions | Analytical data |
| B-99 | 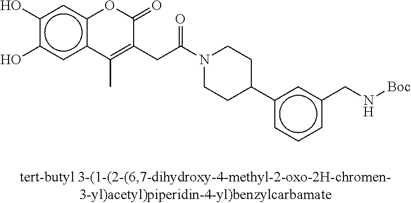 | Carboxylic acid (1 eq.), DMF (~25 vol.), EDCI (1.5 eq.), HOBt (1.5 eq.), Core (1.0 eq.), and DIPEA (4.0 eq.). Stirred at room temperature for 12 hours. Purified by column chromatography. | Yield: 0.1 g, 28%; MS (ES+): m/z = 557 [M + Na]. |

### Step-2:

Products of Step-1 were deprotected as per conditions described in the table below. The details of the compounds synthesized are shown as below. Reactions were done on 100-200 mg scale

| Compound | | Brief reaction | |
|---|---|---|---|
| No. | Structure | conditions | Analytical data |
| 99 | 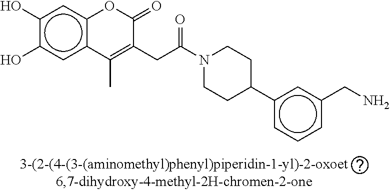 | Dichloromethane (~100 vol.), BBr₃, room temperature −0° C., 12 hours, trituration with methanol followed by preparative HPLC. | Yield: 33%; MS (ES+): m/z = 423 [MH⁺]; HPLC: 99.92% (220 nm); ¹H NMR (400 MHz, CD₃OD): δ 7.43-7.28 (m, 4H), 7.12 (s, 1H), 6.75 (s, 1H), 4.67 (d, J = 12.8 Hz, 1H), 4.33 (d, J = 13.3 Hz, 1H), 4.11 (s, 2H), 3.91- 3.70 (m, 2H), 2.92 (tt, J = 12.1, 3.7 Hz, 2H), 2.79 (td, J = 13.4, 12.7, 2.7 Hz, 1H), 2.37 (s, 3H), 2.04-1.79 (m, 3H), 1.66 (qd, J = 12.9, 4.2 Hz, 1H). |

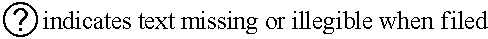

The details of syntheses of intermediates are described below.

| Target | Structure |
|---|---|
| A-99 | 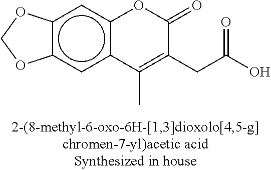 |

### Synthesis 2-(8-methyl-6-oxo-6H-[1,3]dioxolo[4,5-g]chromen-7-yl)acetic acid (A-99)

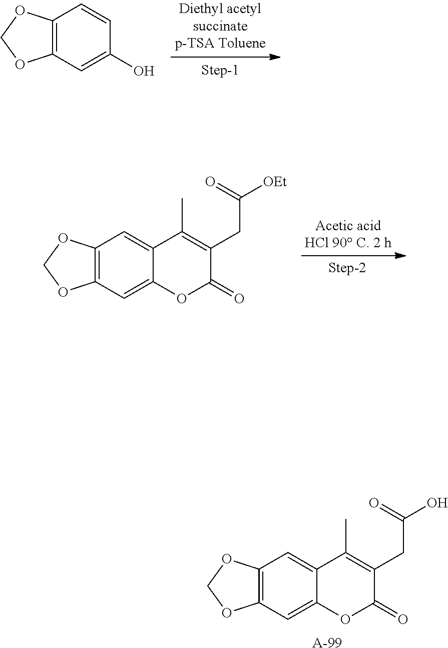

### Step-1:

A solution of sesamol (0.5 g, 3.62 mmol) in toluene (10 mL) and diethyl acetyl succinate (0.87 mL, 4.30 mmol) in toluene (10 mL) was charged with p-TSA.H2O (0.34 g, 1.79 mmol) and heated at 80° C. overnight. TLC (Mobile phase 50% ethyl acetate in n-hexane) indicated the absence of starting material (Rf 0.6) and product formation (Rf 0.4). The reaction mixture was concentrated and the compound was extracted in ethyl acetate, washed with brine. The organic layer was separated, dried over sodium sulfate, filtered, and concentrated in vacuo and purified by column chromatography on silica gel eluting with hexanes/ethyl acetate resulting in ethyl 2-(8-methyl-6-oxo-6H-[1,3]dioxolo[4,5-g]chromen-7-yl)acetate. Yield: (0.6 g, 57%). MS (ES+): m/z=313 [M+Na].

### Step-2:

A solution of ethyl 2-(8-methyl-6-oxo-6H-[1,3]dioxolo[4,5-g]chromen-7-yl)acetate (0.2 g, 0.68 mmol) in acetic acid (6 mL) was charged with concentrated HCl (2 mL) and heated at 90° C. for 2 hours. The reaction mixture was concentrated in vacuo to obtain a solid, which was washed with pentane and dried to give 2-(8-methyl-6-oxo-6H-[1,3]dioxolo[4,5-g]chromen-7-yl)acetic acid. The product was used in the next step without further purification. Yield: (0.17 g, crude). ¹H NMR (400 MHz, DMSO-d₆): δ 7.34 (d, J=2.4 Hz, 1H), 7.10 (d, J=2.4 Hz, 1H), 6.17 (d, J=2.5 Hz, 2H), 3.57 (s, 2H), 2.64 (s, 3H).

### Example 17

### Synthesis of Cofluorons with Benzooxaborol-1-ol Functionality

Final targets with benzoxaborol functionality were synthesized 112 Spiro, T-117 Spiro, T-117 Spiro methyl and T-117-gem mono methyl, were synthesized with benzoxaborol functionality. Synthetic approaches for every target is described with its respective scheme and procedure as shown below.

### Synthesis of Target 112spiro

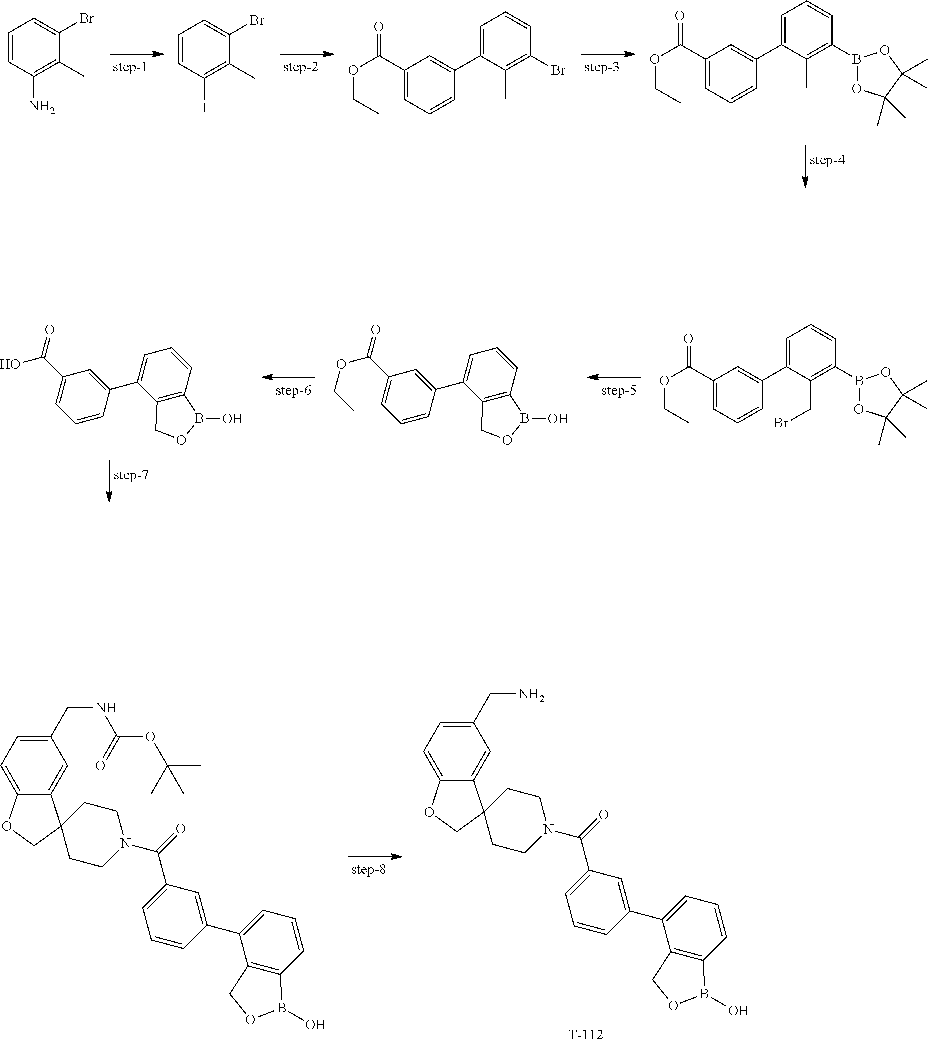

### Step-1:

1-bromo-6-iodo-2-methylbenzene was synthesized as per procedures available in the literature (Bioorganic and Medicinal Chemistry, 16: 6764-77 (2008); J. Am. Chem. Soc., 122:6871-83 (2000)).

### Step-2:

Suzuki coupling of Step-1 product (8.5 g, 28.6 mmol) with m-carbethoxy phenyl boronic acid (6.65 g, 34.32 mmol)) was carried out in presence of palladium (0) tetrakis(triphenyl phosphine) (10 mol %) in dioxane (20 vol) and sodium carbonate (6.06 g, 57.2 mmol) as the base. After completion of the reaction, the reaction mixture was filtered through a pad of celite and the filtrate was concentrated in vacuo. The residue obtained was partitioned between ethyl acetate and water and separated. The aqueous layer was re-extracted with ethyl acetate. The combined organic fractions were dried over sodium sulfate, filtered and concentrated in vacuo. The crude product obtained was purified by column chromatography over silica gel eluting with 5-10% ethyl acetate in hexanes. Yield: 80%. Molecular Weight: 319.19. MS (ES+): m/z=321.2 [MH⁺⁺] (bromo pattern).

### Step-3

A stirred suspension of Step-2 (7.0 g, 21.9 mmol) in toluene (30 vol.) was degassed with argon, and then charged with potassium acetate (6.47 g, 65.7 mmol), PdCl₂-dppf-CH₂Cl₂ (5 mol %) and bis(pinacolato)diborane (13.9 g, 54.75 mmol). The reaction mixture was refluxed and then filtered through a pad of celite. The filtrate was concentrated in vacuo resulting in crude product. The crude product was purified by column chromatography over silica gel eluting with 1-5% ethyl acetate in hexane. Yield: 80%. Molecular Weight: 366.26. MS (ES+): m/z=367.20 [MH⁺].

### Step-4

A stirred solution of Step-3 product (6.0 g, 16.3 mmol) in carbon tetrachloride (20 vol.) was charged with dibenzoyl peroxide (0.75 g, 3.2 mmol) and N-bromo succinimide (1.2 eq.) and heated to 75° C. for 5 hours. The reaction mixture was partitioned between water and dichloromethane and separated. The organic phase was washed with water, brine, dried over anhydrous sodium sulfate, filtered, and concentrated in vacuo resulting in crude product. The crude product was purified by column chromatography over silica gel eluting with 1-5% ethyl acetate in hexanes. Yield: 80% Molecular Weight: 445.15. MS (ES+): m/z=446.20 [MH⁺].

### Step-5

A stirred solution of Step-4 product (5.8 g, 13 mmol) in acetonitrile (30 vol.) was charged with trifluoro acetic acid (10 vol.) and water (5 vol.). The reaction mixture was heated to 91° C. and monitored by LCMS. The reaction mixture was concentrated in vacuo and the residue was partitioned between water and ethyl acetate and separated. The organic layer was dried over sodium sulfate, filtered and concentrated in vacuo. The crude product was purified by column chromatography over silica gel eluting with 10-35% ethyl acetate in hexanes. Yield: 60%. Molecular Weight: 282.10. MS (ES+): m/z=283.25 [MH⁺].

### Step-6

A mixture of Step-5 product (2 g, 7.08 mmol) in THF (10 vol.) and water (20 vol.) was charged with lithium hydroxide (1.7 g, 70.8 mmol) and heated to 60° C. The reaction mixture was concentrated in vacuo. The reaction mixture was diluted with water and was adjusted to pH=2 using concentrated HCl, upon which a precipitate formed. The precipitate was filtered, washed with water and dried in vacuum oven. Yield: 60%. Molecular Weight: 254.05. MS (ES+): m/z=255.10 [MH⁺].

### Step-7

A mixture of Step-6 product (250 mg, 0.98 mmol), tert-butyl((2H-spiro[benzofuran-3,4′-piperidin]-5-yl)methyl)carbamate (404 mg, 1.27 mmol), EDCI (280 mg, 1.47 mmol), DMAP (240 mg, 1.96 mmol) in dichloromethane (20 vol.) was stirred at room temperature and was monitored by LCMS. The reaction mixture was concentrated in vacuo and diluted with water. The pH of the reaction mixture was adjusted to 4 using dilute HCl, upon which a precipitate formed. The precipitate was filtered and washed with water and dried in vacuum oven. Yield: 60%. Molecular Weight: −554.44. MS (ES+): m/z=555.10 [MH⁺].

### Step-8:

Product of Step-7 (370 mg, 0.66 mmol) was dissolved in dichloromethane (20 vol.) and TFA (20 vol.), and stirred at room temperature until completion of the reaction. The reaction mixture was concentrated in vacuo and the crude residue was purified by preparative HPLC to give Target 112. Yield: 33%. Molecular Weight: 454.33. MS (ES+): m/z=455.20 [MH⁺]. HPLC purity: 96%. ¹H NMR (400 MHz, DMSO-d₆): δ 8.29 (s, 2H), 7.81 (d, J=6.9 Hz, 1H), 7.64-7.41 (m, 7H), 7.26 (d, J=8.2 Hz, 1H), 6.82 (d, J=8.2 Hz, 1H), 5.13 (s, 2H), 4.44 (d, J=46.7 Hz, 4H), 4.13-3.88 (m, 4H), 3.69 (d, J=16.3 Hz, 1H), 3.14 (s, 2H), 1.74 (d, J=42.6 Hz, 4H).

### Synthesis of Target-117 Spiro

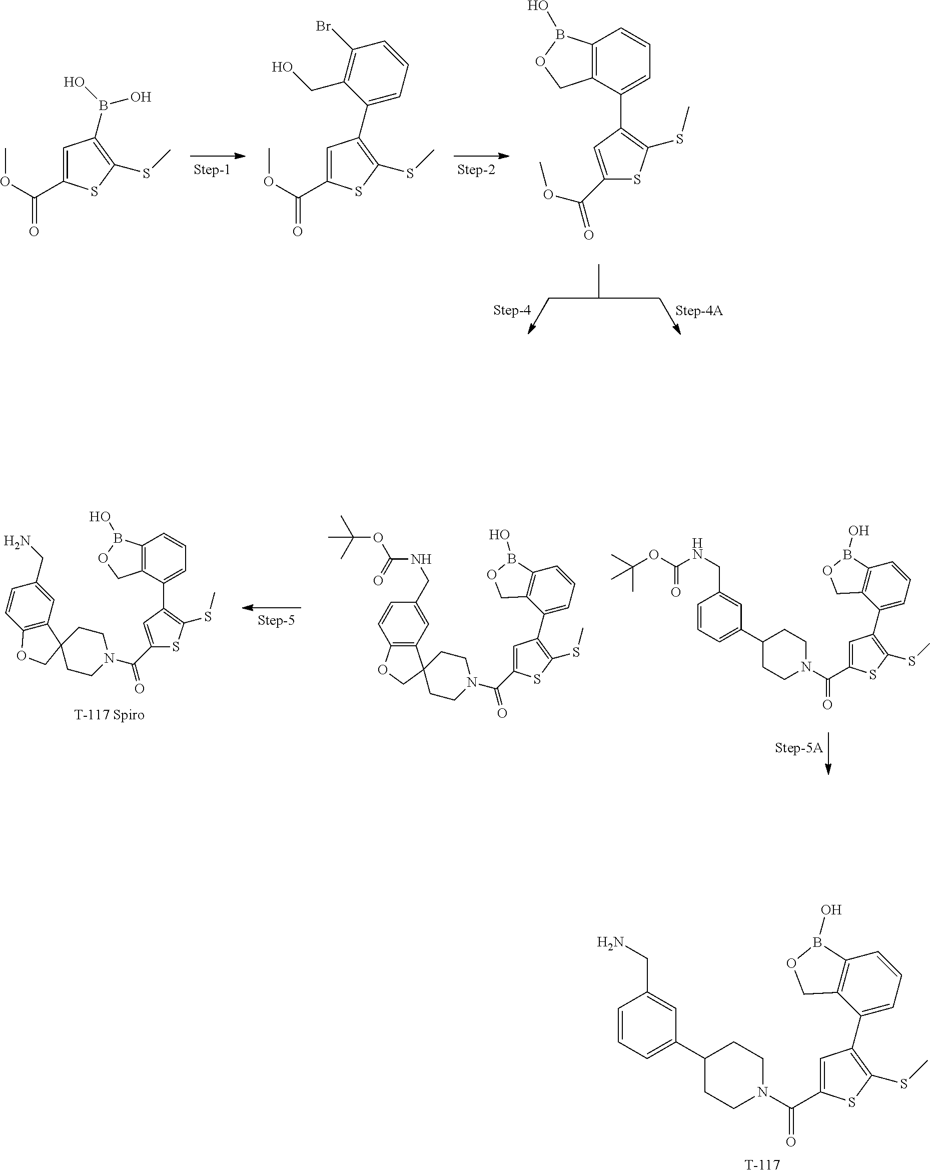

### Step-1:

A solution of (5-(methoxycarbonyl)-2-(methylthio)thiophen-3-yl)boronic acid (8 g, 34.48 mmol), 2,6-dibromobenzyl alcohol (11 g, 41.37 mmol), palladium (0) tetrakis(triphenyl phosphine) (10 mol %), and sodium carbonate (7.3 g, 68.96 mmol) in dioxane (20 vol) was degassed and heated until completion of the reaction. The reaction mixture was filtered through a pad of celite and the filtrate was concentrated in vacuo. The residue was partitioned between water and ethyl acetate and separated. The organic layer was dried over sodium sulfate, filtered, and concentrated in vacuo resulting in crude product. The crude product was purified by column chromatography over silica gel eluting with 5-10% ethyl acetate in hexanes. Yield: 20%. Molecular Weight: 373.29. MS (ES+): m/z=375.10 [MH⁺⁺] (Bromo pattern).

### Step-2:

A stirred suspension of Step-1 product (1.9 g, 5.09 mmol) in toluene (30 vol.) was degassed with argon and charged with potassium acetate (1.5 g, 15.27 mmol), PdCl₂-dppf-CH₂Cl₂ (5 mol %), dppf (3 mol %) and bis(pinacolato)diborane (3.21 g, 12.72 mmol). The reaction mixture was degassed again, heated to reflux and monitored by LCMS until most of the starting material was consumed. The mixture was filtered through a pad of celite and the filtrate was concentrated in vacuo resulting in crude product. The crude product was purified by column chromatography over silica gel eluting with 1-5% ethyl acetate in hexanes. Yield: 40%. Molecular Weight: 320.19. MS (ES+): m/z=321.10 [MH⁺].

### Step-3:

A mixture of Step-2 product (650 mg, 2.03 mmol, potassium hydroxide (570 mg, 10.15 mmol) in THF (10 vol) and water (20 vol.) was heated to 60° C. Reaction was monitored by LCMS until most of the starting material was consumed. The reaction mixture was concentrated in vacuo and the residue was diluted with water and the pH was adjusted to 2 using concentrated HCl upon which a precipitate formed. The precipitate was filtered and washed with water and dried in vacuum oven. Yield: 35%. Molecular Weight: 306.17. MS (ES+): m/z=307.20 [MH⁺].

### Step-4:

A mixture of Step-3 product (150 mg, 0.490 mmol), tert-butyl((2H-spiro[benzofuran-3,4′-piperidin]-5-yl)methyl)carbamate (202 mg, 0.63 mmol), EDCI (142 mg, 0.735 mmol), DMAP (120 mg, 0.98 mmol) in dichloromethane (20 vol.) was stirred at room temperature and monitored by LCMS until most of the starting material was consumed. The reaction mixture was concentrated in vacuo and diluted with water. The pH of the reaction mixture was adjusted to about 4 using dilute HCl, upon which a precipitate formed. The precipitate was filtered and washed with water and dried in vacuum oven. Yield: 55%. Molecular Weight: 606.17. MS (ES+): m/z=607.20 [MH⁺].

### Step-4A:

Same as Step-4, except that tert-butyl 3-(piperidin-4-yl)benzyl carbamate was used instead of ((2H-spiro[benzofuran-3,4′-piperidin]-5-yl)methyl)carbamate. Yield: 51%. Molecular Weight: 578.55. MS (ES+): m/z=579.3 [MH⁺].

### Step-5:

Product of Step-4 (160 mg, 0.263 mmol) was dissolved in dichloromethane (20 vol.)-TFA (20 eq.) and stirred at room temperature. After completion of the reaction, the reaction mixture was concentrated in vacuo and purified by preparative HPLC to afford Target-117 Spiro. Yield: 30%. Molecular Weight: 506.44. MS (ES+): m/z=507.15 [MH⁺]. HPLC purity: 99.2%. ¹H NMR (400 MHz, DMSO-d₆): δ 9.38 (s, 1H), 8.19-8.06 (m, 2H), 7.87 (d, J=7.3 Hz, 1H), 7.52 (t, J=7.4 Hz, 1H), 7.37 (d, J=7.5 Hz, 1H), 7.30-7.29 (m, 1H), 7.20 (d, J=8.2 Hz, 1H), 6.90 (s, 1H), 6.78 (d, J=8.2 Hz, 1H), 5.00 (d, J=26.4 Hz, 2H), 4.26 (s, 2H), 3.96 (p, J=5.6 Hz, 2H), 2.89-2.75 (m, 4H), 2.50 (s, 3H), 1.25 (s, 4H).

### Step-5A:

Same as Step-5, except that tert-butyl 3-(piperidin-4-yl)benzyl carbamate was used instead of ((2H-spiro[benzofuran-3,4′-piperidin]-5-yl)methyl)carbamate. Yield: 20%. Molecular Weight: 478.43. MS (ES+): m/z=479.15 [MH⁺]. HPLC data: 96.79%. ¹H NMR (400 MHz, CDCl₃): δ 8.16 (bs, 1H), 8.03 (m, 1H), 7.79 (d, J=6.8 Hz, 1H), 7.51-7.42 (m, 3H), 7.03-6.97 (m, 3H), 6.62 (s, 1H), 5.34 (m, 1H), 4.16 (s, 2H) 3.77 (m, 2H), 3.63-3.48 (m, 4H), 2.72 (bs, 1H), 2.57 (s, 3H), 2.2-2.0 (m, 4H).

The details of the Final Targets synthesized are described as below.

| Target | Structure | Analytical Data |
|---|---|---|
| 117-Spiro | 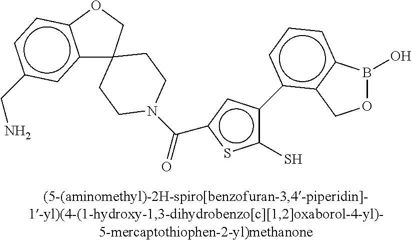 | Mol. Wt: 506.44; MS(ES+): m/z = 507.15 [MH⁺]; HPLC: 99.2%; ¹H NMR (400 MHz, DMSO- d₆): δ 9.38 (s, 1H), 8.19- 8.06 (m, 2H), 7.87 (d, J = 7.3 Hz, 1H), 7.52 (t, J = 7.4 Hz, 1H), 7.37 (d, J = 7.5 Hz, 1H), 7.30-7.29 (m, 1H), 7.20 (d, J = 8.2 Hz, 1H), 6.90 (s, 1H), 6.78 (d, J = 8.2 Hz, 1H), 5.00 (d, J = 26.4 Hz, 2H), 4.26 (s, 2H), 3.96 (d, J = 5.6 Hz, 2H), 2.89-2.75 (m, 4H), 2.50 (s, 3H), 1.25 (s, 4H). |
| 117 | 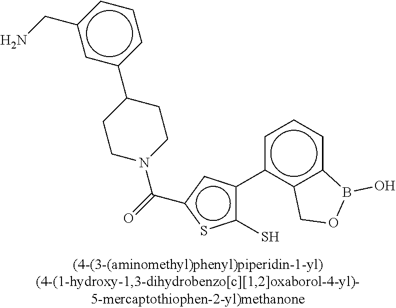 | Mol. Wt: 478.43; MS (ES+): m/z = 479.15 [MH⁺]; HPLC: 96.79%; ¹H NMR (400 MHz, CDCl₃): δ 8.16 (bs, 1H), 8.03 (m, 1H), 7.79 (d, J = 6.8 Hz, 1H), 7.51-7.42 (m, 3H), 7.03-6.97 (m, 3H), 6.62 (s, 1H), 5.34 (m, 1H), 4.16 (s, 2H) 3.77 (m, 2H), 3.63-3.48 (m, 4H), 2.72 (bs, 1H), 2.57 (s, 3H), 2.2-2.0 (m, 4H). |

### General Procedures for Examples 14-17

### A. General Procedure for Coupling Conditions and Work-Up

To a stirred solution of carboxylic acid intermediates in DCM or DMF described in Examples 14-17, was added EDCI, HOBt (in some cases) and DMAP or DIPEA. The reaction mixture was stirred for 15 minutes at 0° C. followed by addition of protected core. Stirring was continued at room temperature and reaction was monitored by LCMS until most of the starting materials were consumed. Reaction mixture was then quenched with water. The aqueous layer was extracted with dichloromethane. Combined organic layers were dried over sodium sulfate, filtered, and concentrated in vacuo to afford the crude product which was either used for next step without purification or purified by chromatographic techniques.

### B. General Procedures for Hydrolysis

Desired ester was dissolved in mixture of water and solvents, such as THF/methanol/acetone that are miscible in water, then charged with lithium/sodium hydroxide. The reaction mixture was stirred at room temperature and monitored by TLC and LCMS until most of the starting material was consumed. Solvent was concentrated in vacuo and partitioned between ethyl acetate and water and separated. The aqueous layer was washed with ethyl acetate (1×), acidified with 2N HCl and extracted with ethyl acetate again. The acidic ethyl acetate extract was dried over sodium sulfate, filtered, and concentrated in vacuo to obtain crude product. In most of the cases, products were sufficiently pure to be used for the next step.

### C. General Procedures for Boc Deprotection

Desired compound was stirred with aqueous hydrochloric acid or trifluoracetic acid (TFA) in a co-solvent, such as acetonitrile, methanol, THF, or DCM. Reaction was monitored by LCMS until most of the starting materials were consumed. The reaction mixture was concentrated in vacuo to remove the solvents and residue obtained was purified by reverse phase preparative HPLC. In some cases, products were purified by column chromatography over silica gel.

The pure fraction of mobile phase was lyophilized to obtain the products as TFA salts. TFA salts were converted to hydrochloride salts by stirring with 2N HCl for 30 minutes under nitrogen atmosphere followed by lyophilization. Sometimes, Only Boc deprotection observed to be taking place with boronate ester functionality intact. In such cases, further hydrolysis of isolated Boc de-protected boronate esters were carried out followed by purification using preparative HPLC.

### Example 18

### Demonstration of Enahancement of Fluorescence for a Cofluoron Pair

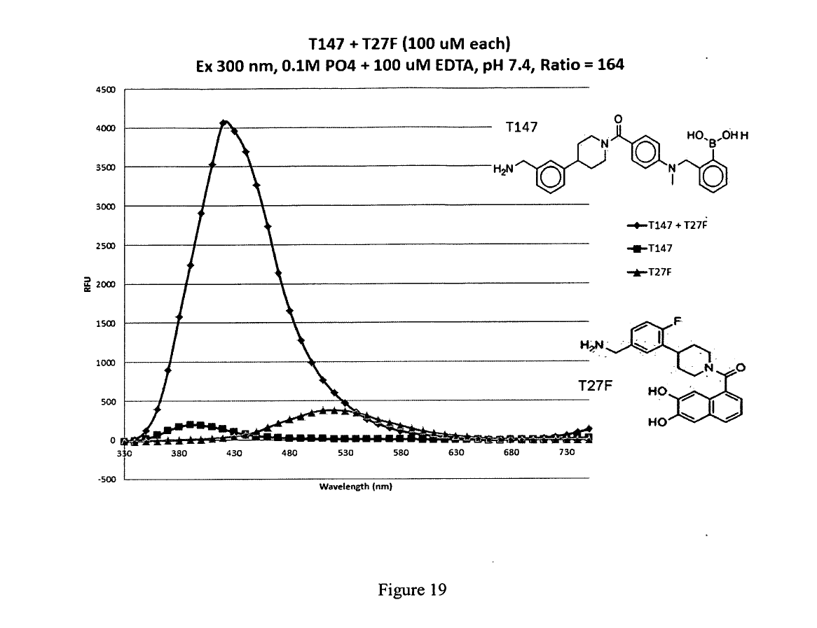

Fluorescence emission spectra were recorded for cofluoron monomers T147 and T27F individually at a concentration of 100 μM as well as in combination where each cofluoron monomer was at a concentration of 100 μM, in a 0.1M phosphate buffer at pH 7.4 containing 100 μM EDTA. The samples were excited at 300 nM and fluorescence emissions were measured between 300-750 nm on a Spectramax M5 spectrofluorometer. Cofluoron monomer T147 alone had a maximum emission of 194 relative fluorescence units (RFU) at 390 nm. Cofluoron monomer T27F alone had a maximum emission of 380 RFU at 520 nm. The fluorescence emission was enhanced to 4062 RFU and the emission wavelength shifted to 420 nm when the two cofluoron monomers were combined (See FIG. 19).

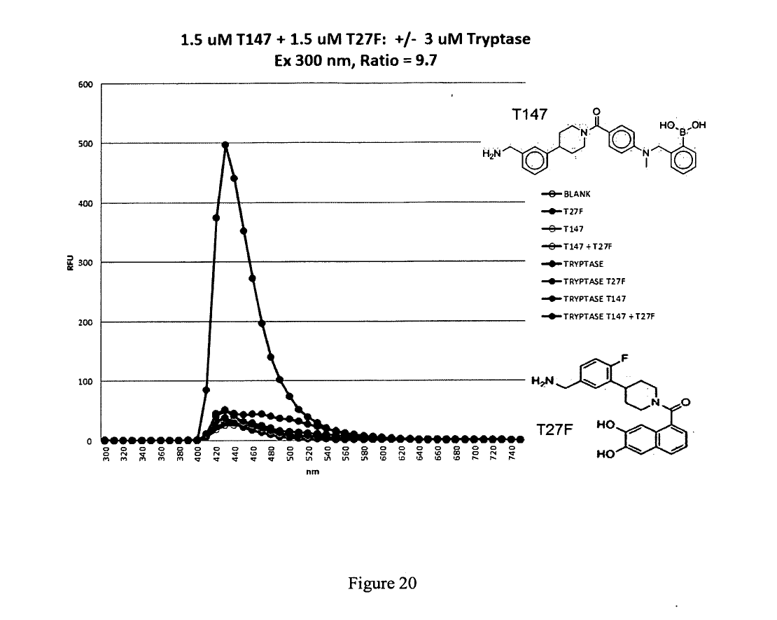

Fluorescence emission spectra were recorded for cofluoron monomers T147 and T27F individually at a concentration of 1.5 μM as well as in combination where each cofluoron monomer was at a concentration of 1.5 μM, either in the absence of recombinant human tryptase or in the presence of 3 μM recombinant human tryptase. The recombinant human tryptase was obtained from Promega (Catalog #G5631). Samples were prepared in a 0.1M phosphate buffer at pH 7.5. An equivalent volume of tryptase buffer (10 mM MES, pH 6.1 2M NaCl) was added to samples not containing tryptase. The samples were excited at 300 nM and fluorescence emissions were measured between 300-750 nm on a Spectramax M5 spectrofluorometer. The fluorescence emission intensity at 430 nm for the two cofluoron monomers combined increased from 25 RFU in the absence of tryptase to 496 RFU in the presence of tryptase (See FIG. 20).

### Example 19

### Demonstration of Enahancement of Fluorescence for a Cofluoron Pair

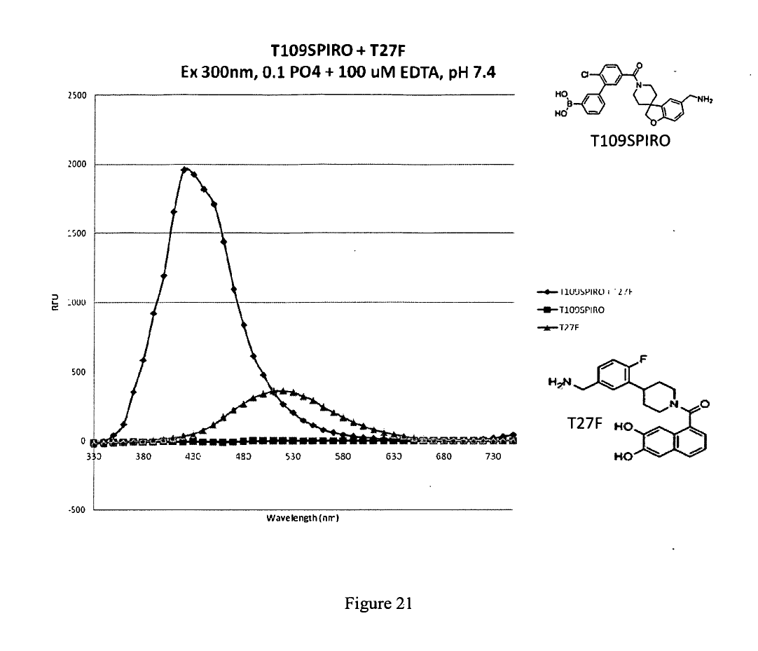

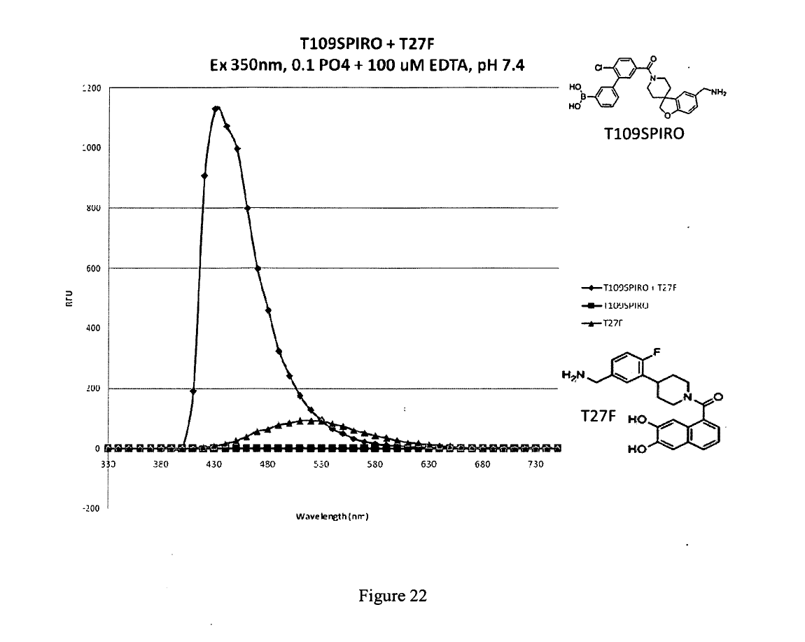

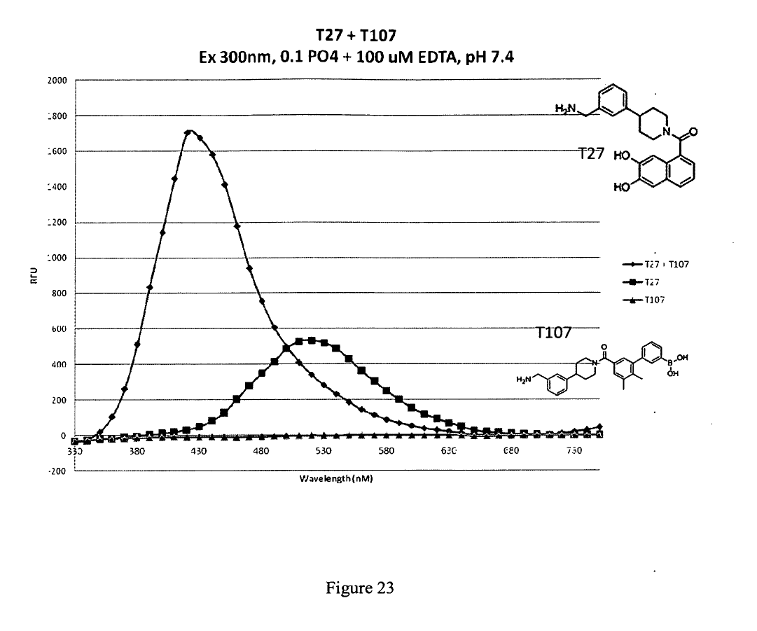

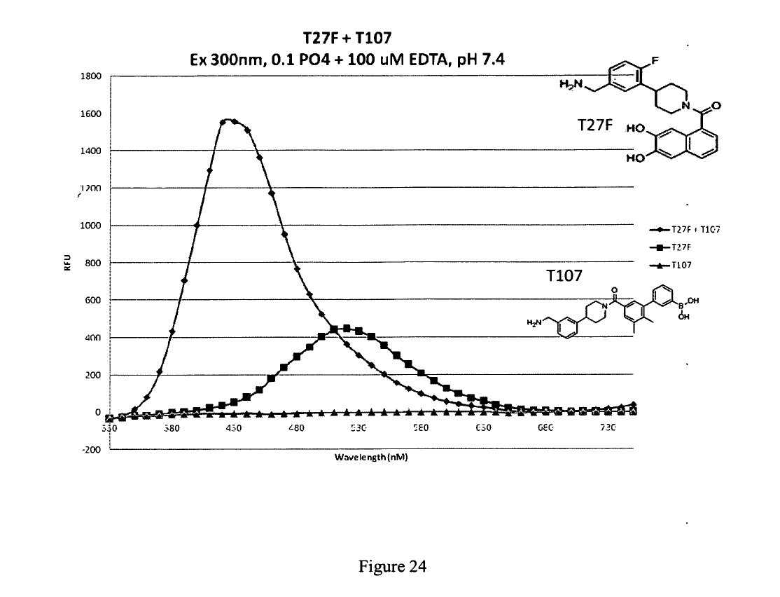

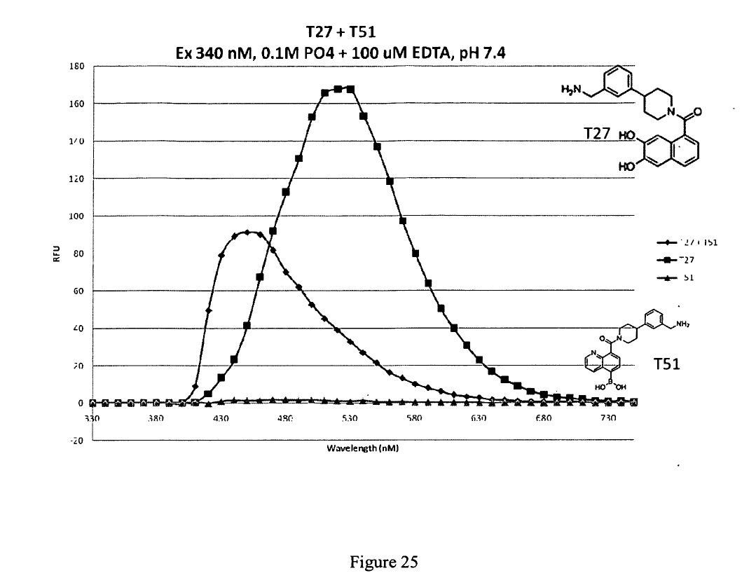

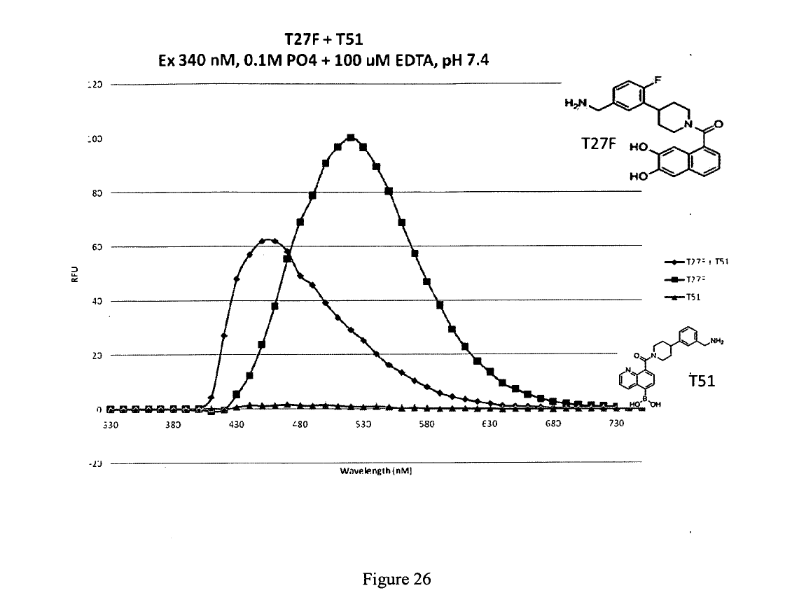

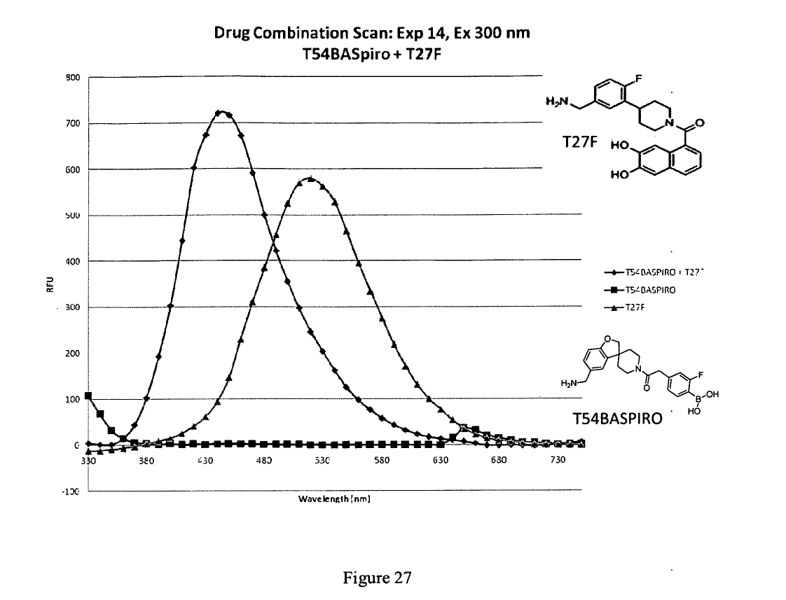

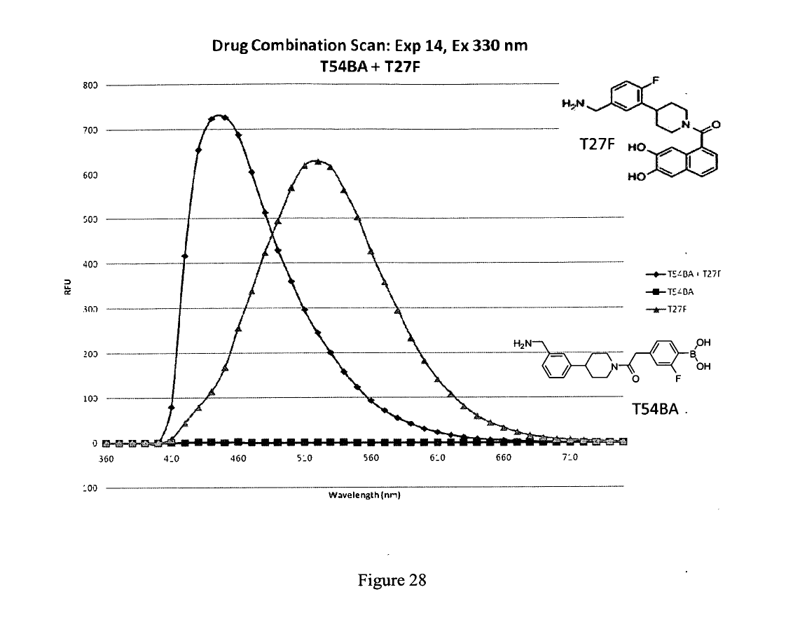

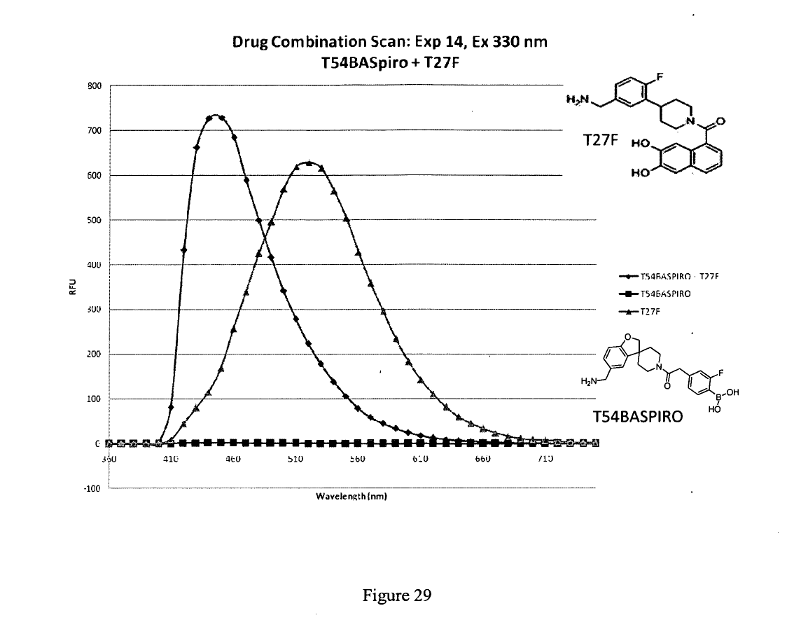

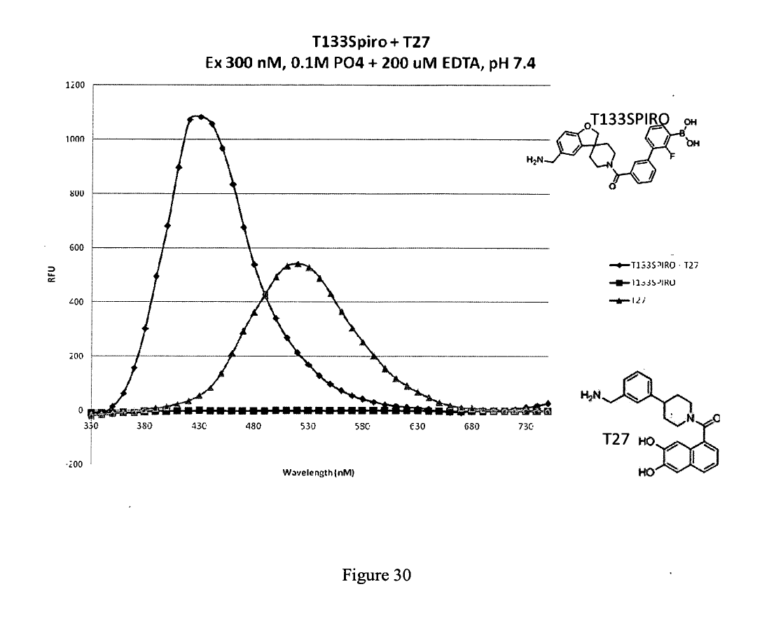

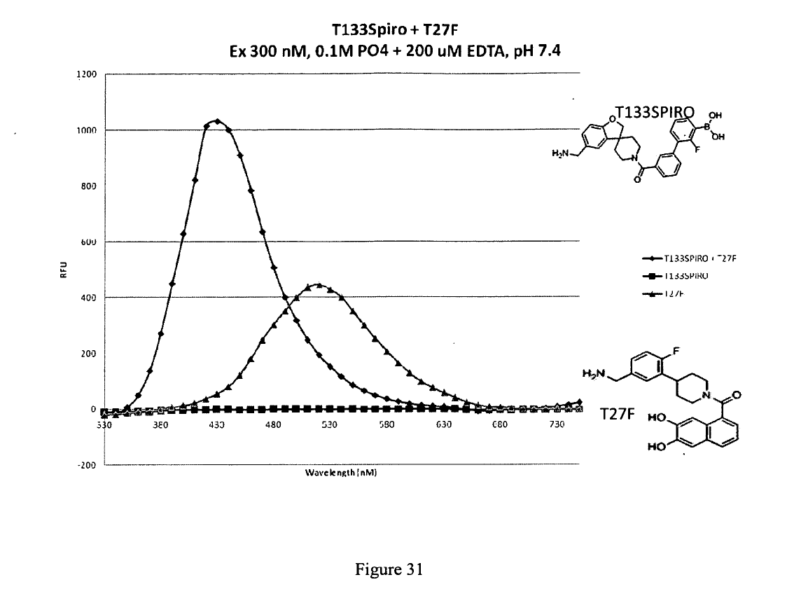

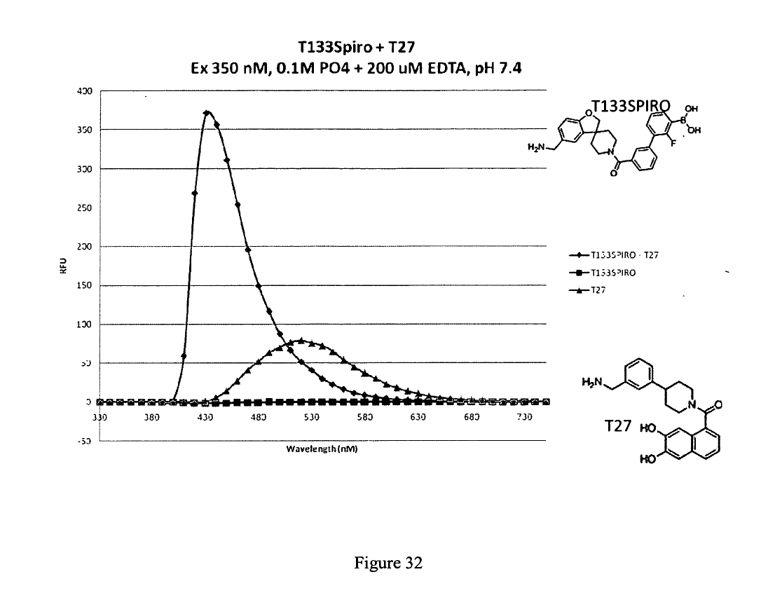

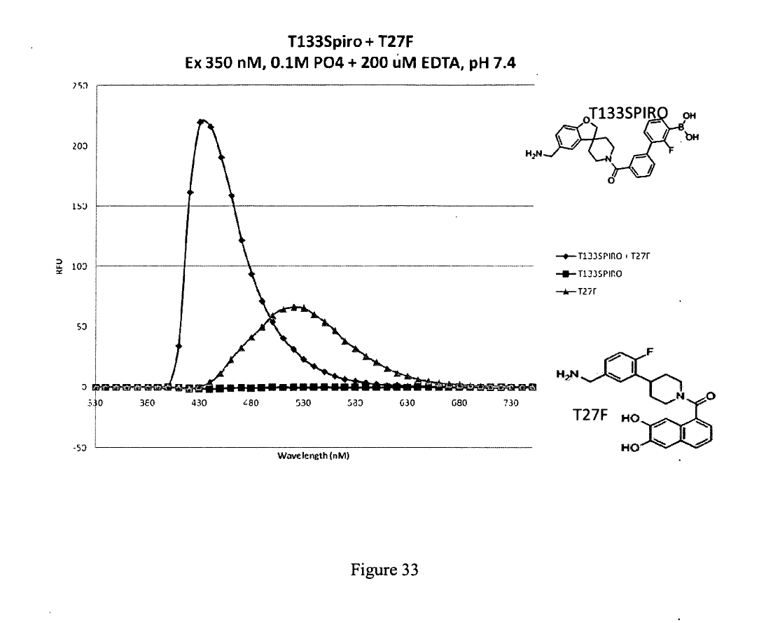

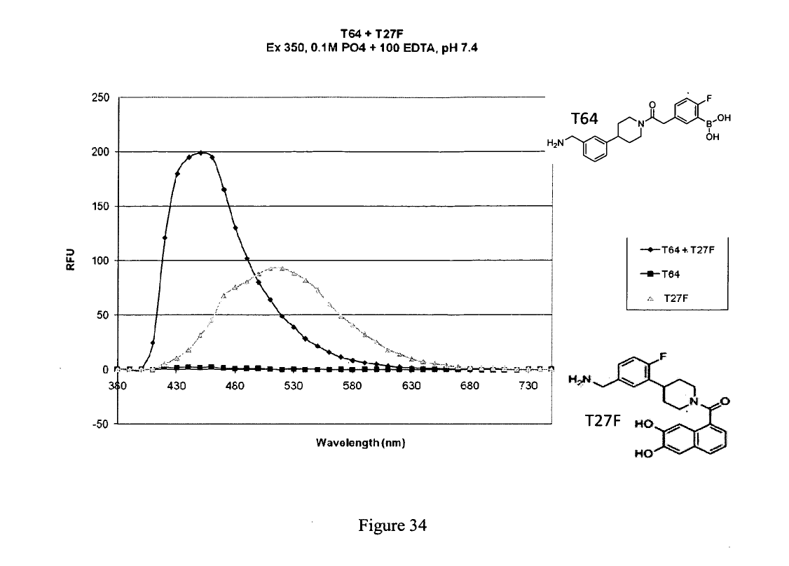

FIGS. 21 through 34 show the results of fluorescent measurements on the monomers T27 or T27F containing a dihydroxy moiety and multimers formed by mixing the dihydroxy compound with various monomer binding partners containing a boronic acid. These multimers have increased affinity for human mast cell β2-tryptase as compared to the monomers. The fluorescence properties of cofluorons were measured 0.1M phosphate buffer at pH 7.4 containing either 100 or 200 μM EDTA, in 96- or 384-well black plates using a Molecular Dynamics SpectraMax M5 plate reader. Cofluoron samples were excited at discrete wavelengths and the fluorescence emission was scanned across a range of wavelengths (between 400 and 750 nm). In each instance, the fluorescent signal of the multimer displayed increased intensity or a shift in the emission wavelength or both.

### Discussions of Examples 18-19

Examples 18-19 are examples of cofluoron monomers that bind to human β-tryptase with some affinity and in a 1:1 combination with T27 or T27F form dimers with higher affinity to human β-tryptase than either monomer. While the dimer may not be detectable in solution, the target macromolecule, human β-tryptase, is primarily occupied by the dimeric species in the 1:1 combination: T147; T109-Spiro; T107; T51; T54BASpiro; T54BA; T133-Spiro and T64. It will be apparent to those skilled in the art that the intensity and wavelength of the fluorescence emission can be affected by adding substituents and modifying the monomers. These modifications and changes include but are not limited to the addition of electron donating substituents, increasing the rigidity of the structure though cyclizing rings and by adding substitutions that extend the aromaticity and conjugation of the parent molecule.

### Example 20

### Demonstration of Enhancement of Fluorescence for a Cofluoron Pair

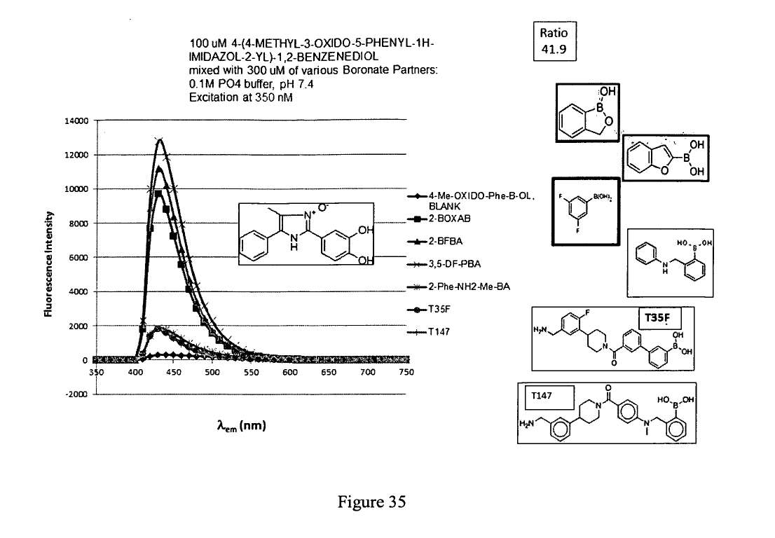

FIG. 35 shows the results of fluorescent measurements on the monomer 4-(4-methyl-3-oxido-5-phenyl-1H-imidazol-2-yl)-1,2-benzene diol and the multimers formed by mixing 4-(4-methyl-3-oxido-5-phenyl-1H-imidazol-2-yl)-1,2-benzene diol with various boronic acid binding partners. The multimers were formed by mixing 100 μM 4-(4-methyl-3-oxido-5-phenyl-1H-imidazol-2-yl)-1,2-benzene diol with 300 μM of various boronic acid binding partners as follows: 2-(hydroxymethyl)phenylboronic acid, benzofuran-2-boronic acid, 3,5-difluorophenylboronic acid, (2-((phenylamino)methyl)phenyl)boronic acid, T35F and T147, respectively. Fluorescent signals were measured on samples in 0.1M phosphate buffer at pH 7.4 (in 50% DMSO), when excited at 350 nm. The multimers formed between 4-(4-methyl-3-oxido-5-phenyl-1H-imidazol-2-yl)-1,2-benzene diol and 2-(hydroxymethyl)phenylboronic acid, benzofuran-2-boronic acid and 3,5-difluorophenylboronic acid showed >30-fold increase in fluorescence intensity.

Although preferred embodiments have been depicted and described in detail herein, it will be apparent to those skilled in the relevant art that various modifications, additions, substitutions and the like can be made without departing from the spirit of the invention and these are therefore considered to be within the scope of the invention as defined in the claims which follow.

1. A method of detecting the presence or absence of a target molecule in a sample, said method comprising:
providing a sample potentially containing one or more target molecules;
providing a set of one to six monomers, each monomer comprising:
one or more ligand elements which are useful for binding to a target molecule with a dissociation constant less than 300 μM and
a linker element being connected directly or indirectly through a connector to said one or more ligand elements, said linker element being capable of forming a bond with one or more linker elements of either the same or a different monomer of said set, wherein association of said linker elements with their ligand elements bound to the target molecule to form a multimer will generate a unique fluorescent signature different from that produced by those monomers either alone or in association with each other in the absence of target, when subjected to electromagnetic excitation;
contacting the sample with the set of monomers under conditions effective to allow the ligand elements to bind to the target molecules, if present in the sample;
subjecting the monomers to reaction conditions effective for the linker elements of either the same or different monomers to undergo bond forming to form multimers if the target molecule is present in the sample; and
detecting the presence or absence of target molecule in the sample based on the fluorescent signature of the sample subjected to said contacting and said subjecting.

2. (canceled)

3. The method of claim 1, wherein said linker element has a molecular weight of less than 2000 daltons.

4. The method of claim 1, wherein said linker element is non-peptidyl.

5-8. (canceled)

9. The method of claim 1, wherein said bond forms under physiological conditions.

10. The method of claim 1, wherein said linker element reversibly associates with one or more linker elements of either the same or a different monomer of said set with a dissociation constant of less than 300 μM.

11. The method of claim 1, said set of monomers comprises:
a first monomer having a first linker, Z₁, selected from the group consisting of: 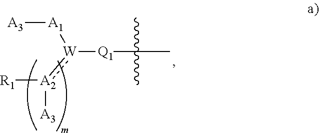
wherein
A₁ is (a) absent; or (b) selected from the group consisting of acyl, substituted or unsubstituted aliphatic, and substituted or unsubstituted heteroaliphatic;
A₂, independently for each occurrence, is (a) absent; or (b) selected from the group consisting of —N—, acyl, substituted or unsubstituted aliphatic, and substituted or unsubstituted heteroaliphatic, provided that at least one of A₁ and A₂ is present; or
A₁ and A₂, together with the atoms to which they are attached, form a substituted or unsubstituted 4-8 membered cycloalkyl or heterocyclic ring;
A₃ is selected from the group consisting of —NHR′, —SH, and —OH;
W is CR′ or N;
R′ is selected from the group consisting of hydrogen, halogen, substituted or unsubstituted aliphatic, substituted or unsubstituted heteroaliphatic, substituted or unsubstituted aryl, substituted or unsubstituted heteroaryl, —NH₂, —NO₂, —SH, and —OH;
m is 1-6; [symbol]
represents a single or double bond; and
R₁ is (a) absent; or (b) selected from the group consisting of hydrogen, halogen, substituted or unsubstituted aliphatic, substituted or unsubstituted heteroaliphatic, substituted or unsubstituted aryl, substituted or unsubstituted heteroaryl, —NH₂, —NO₂, —SH, and —OH;
Q₁ is (a) absent; or (b) selected from the group consisting of substituted or unsubstituted aliphatic and substituted or unsubstituted heteroaliphatic; or
R₁ and Q₁ together with the atoms to which they are attached form a substituted or unsubstituted 4-8 membered cycloalkyl or heterocyclic ring; 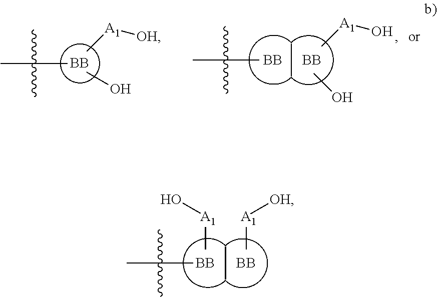
wherein
BB, independently for each occurrence, is a 4-8 membered cycloalkyl, heterocyclic, aryl, or heteroaryl moiety, wherein the cycloalkyl, heterocyclic, aryl, or heteroaryl moiety is optionally substituted with one or more groups represented by R₂, wherein the two substituents comprising —OH have a 1,2 or 1,3 configuration;
each R₂ is independently selected from the group consisting of hydrogen, halogen, oxo, sulfonate, —NO₂, —CN, —OH, —NH₂, —SH, —COON, —CONHR′, substituted or unsubstituted aliphatic, and substituted or unsubstituted heteroaliphatic, or two R₂ together with the atoms to which they are attached form a fused substituted or unsubstituted 4-6 membered cycloalkyl or heterocyclic bicyclic ring system;
A₁, independently for each occurrence, is (a) absent; or (b) selected from the group consisting of acyl, substituted or unsubstituted aliphatic, and substituted or unsubstituted heteroaliphatic;
R′ is selected from the group consisting of hydrogen, halogen, substituted or unsubstituted aliphatic, substituted or unsubstituted heteroaliphatic, substituted or unsubstituted aryl, substituted or unsubstituted heteroaryl, —NH₂, —NO₂, —SH, and —OH; 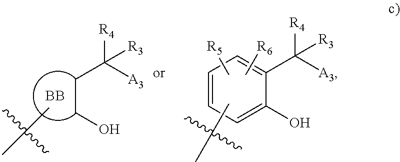
wherein
BB is a substituted or unsubstituted 5- or 6-membered cycloalkyl, heterocyclic, aryl, or heteroaryl moiety;
A₃, independently for each occurrence, is selected from the group consisting of —NHR′ and —OH;
R₃ and R₄ are independently selected from the group consisting of H, C₁₋₄ alkyl, and phenyl, or R₃ and R₄ taken together from a 3-6 membered ring;
R₅ and R₆ are independently selected from the group consisting of H; C₁₋₄ alkyl optionally substituted by hydroxyl, amino, halogen, or thio; C₁₋₄ alkoxy; halogen; —OH; —CN; —COOH; and —CONHR′; or R₅ and R₆ taken together form phenyl or a 4-6 membered heterocycle; and
R′ is selected from the group consisting of hydrogen, halogen, substituted or unsubstituted aliphatic, substituted or unsubstituted heteroaliphatic, substituted or unsubstituted aryl, substituted or unsubstituted heteroaryl, —NH₂, —NO₂, —SH, and —OH; 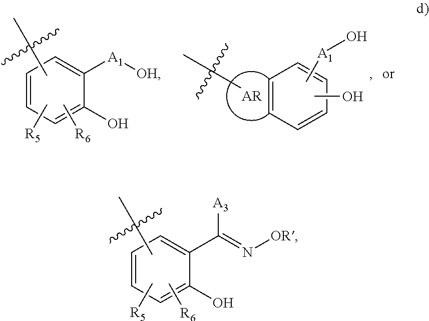
wherein
A₁ is (a) absent; or (b) selected from the group consisting of acyl, substituted or unsubstituted aliphatic, and substituted or unsubstituted heteroaliphatic;
A₃, independently for each occurrence, is selected from the group consisting of —NHR′ and —OH;
AR is a fused phenyl or 4-7 membered aromatic or partially aromatic heterocyclic ring, wherein AR is optionally substituted by oxo; C₁₋₄ alkyl optionally substituted by hydroxyl, amino, halo, or thio; C₁₋₄alkoxy; —S—C₁₋₄ alkyl; halogen; —OH; —CN; —COOH; or —CONHR′; wherein the two substituents comprising —OH are ortho to each other;
R₅ and R₆ are independently selected from the group consisting of H; C₁₋₄ alkyl optionally substituted by hydroxyl, amino, halo, or thio; C₁₋₄ alkoxy; halogen; —OH; —CN; —COOH; and CONHR′; and
R′ is selected from the group consisting of hydrogen, halogen, substituted or unsubstituted aliphatic, substituted or unsubstituted heteroaliphatic, substituted or unsubstituted aryl, substituted or unsubstituted heteroaryl, —NH₂, —NO₂, —SH, and —OH; 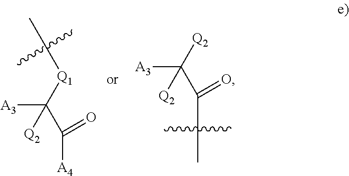
wherein
Q₁ is selected from the group consisting of C₁₋₄ alkyl; alkylene; a bond; C₁₋₆ cycloalkyl; a 5-6 membered heterocyclic ring; and phenyl;
Q₂, independently for each occurrence, is selected from the group consisting of H, C₁₋₄ alkyl, alkylene, a bond, C₁₋₆ cycloalkyl, a 5-6 membered heterocyclic ring, phenyl, substituted or unsubstituted aliphatic, substituted or unsubstituted heteroaliphatic, substituted or unsubstituted aryl, and substituted or unsubstituted heteroaryl;
A₃, independently for each occurrence, is selected from the group consisting of —NH₂ and —OH;
A₄, independently for each occurrence, is selected from the group consisting of —NH—NH₂, —NHOH, —NH—OR″, and —OH;
R″ is selected from the group consisting of H and C₁₋₄ alkyl; and 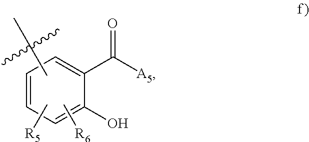
wherein
A₅ is selected from the group consisting of —OH, —NH₂, —SH, and —NHR′″; R′″ is selected from the group consisting of —NH₂, —OH, and C₁₋₄ alkoxy;
R₅ and R₆ are independently selected from the group consisting of H; C₁₋₄ alkyl optionally substituted by hydroxyl, amino, halo, or thio; C₁₋₄ alkoxy; halogen; —OH; —CN; —COOH; and —CONHR′; or R₅ and R₆ taken together may form a 5-6 membered ring; 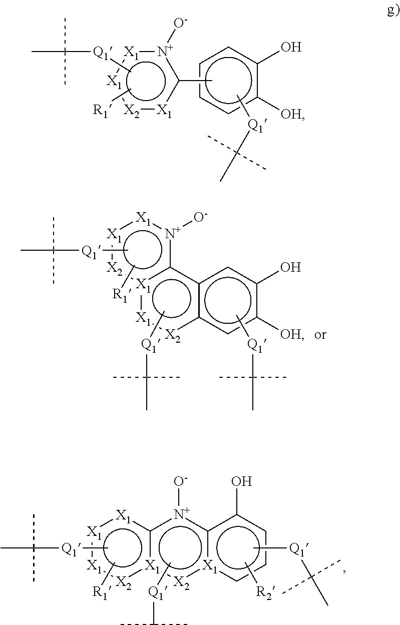
wherein:
------ represents optional connection points where Z₁ is connected to one or more ligand elements, directly or through a connector;
each X₁ is independently C, N, O or S;
each X₂ is independently absent, C, N, O or S;
each R₁′ and R₂′ are independently be H, substituted or unsubstituted aliphatic, substituted or unsubstituted heteroaliphatic, substituted or unsubstituted aryl, substituted or unsubstituted heteroaryl;
each Q₁′ is independently absent, substituted or unsubstituted aliphatic, substituted or unsubstituted heteroaliphatic, substituted or unsubstituted aryl, substituted or unsubstituted heteroaryl, provided that at least one Q₁′ is present, providing at least one connection point of the formula to the one or more ligand element;
or Q₁′ and R₁′ together with the atoms they attach to form a fused 5- or 6-membered aromatic or heteroaromatic ring when Q₁′ and R₁′ are adjacent;
or Q₁′ and R₂′ together with the atoms they attach to form a fused 5- or 6-membered aromatic or heteroaromatic ring when Q₁ and R₂ are adjacent; and
a second monomer having a second linker, Z₂, being a boronic acid or oxaborole moiety capable of binding with the Z₁ moiety of the first monomer to form the multimer.

12-13. (canceled)

14. The method of claim 1, wherein the target molecule is selected from the group consisting of protein, nucleic acid, carbohydrate, and lipid.

15. The method of claim 1, wherein the target molecule is selected from the group consisting of intracellular proteins, surface proteins, viral proteins, viral structural macromolecules, bacterial proteins, or bacterial macromolecules.

16. The method of claim 1, wherein the target molecule is selected from the group consisting of: (1) G-protein coupled receptors; (2) nuclear receptors; (3) voltage gated ion channels; (4) ligand gated ion channels; (5) receptor tyrosine kinases; (6) growth factors; (7) proteases; (8) sequence specific proteases; (9) phosphatases; (10) protein kinases; (11) bioactive lipids; (12) cytokines; (13) chemokines; (14) ubiquitin ligases; (15) viral regulators; (16) cell division proteins; (17) scaffold proteins; (18) DNA repair proteins; (19) bacterial ribosomes; (20) histone deacetylases; (21) apoptosis regulators; (22) chaperone proteins; (23) serine/threonine protein kinases; (24) cyclin dependent kinases; (25) growth factor receptors; (26) proteasome; (27) signaling protein complexes; (28) protein/nucleic acid transporters; (29) viral capsids; and (30) bacterial surface proteins.

17-27. (canceled)

28. The method of claim 1 further comprising:
imaging said sample using said formed multimer as a result of said contacting and said subjecting.

29. The method of claim 28, wherein said imaging is confocal imaging.

30. The method of claim 28, wherein said imaging is carried out in vivo.

31. The method of claim 1, wherein the sample is a biological sample, said method further comprising:
imaging and localizing the target molecule in the biological sample based on its fluorescent signature resulting from said contacting and said subjecting.

32. The method of claim 31, wherein the target molecule is localized to specific cells in the biological sample.

33. The method of claim 32, wherein the target molecule is a marker for cancer cells in the biological sample.

34. The method of claim 31, wherein the target molecule is localized to specific subcellular compartments in the biological sample.

35. The method of claim 34, wherein the target molecule localized is a marker for disease in the biological sample.

36. The method of claim 34, wherein the target molecule localized identifies specific subcellular compartments or the metabolic state of such compartments.

37. The method of claim 1, wherein the amount of target molecule present in the sample is determined, said method comprising:
measuring the fluorescence generated in the sample with an unknown amount of the target molecule;
comparing the measured fluorescence to that produced with a known amount of the target molecule; and
determining the amount of target molecule present in the sample based on said comparing.

38. A method of detecting the presence or absence of a virus, bacterium or fungus in a sample, said method comprising:
providing a sample potentially containing one or more virus, bacterium or fungus;
providing a set of one to six monomers, each monomer comprising:
one or more ligand elements which are useful for binding to one or more target molecules on the surface of, or internally within the virus, bacterium or fungus, with a dissociation constant less than 300 μM and
a linker element being connected directly or indirectly through a connector to said one or more ligand elements, said linker element being capable of forming a bond with one or more linker elements of either the same or a different monomer of said set, wherein association of said linker elements, with their ligand elements bound to the one or more target molecules on the surface of, or internally within the virus, bacterium or fungus to form a multimer, will generate a unique fluorescent signature different from that produced by those monomers either alone or in association with each other in the absence of the virus, bacterium or fungus target, when subjected to electromagnetic excitation;
contacting the sample with the set of monomers under conditions effective to allow the ligand elements to bind to the one or more target molecules on the surface of, or internally within the virus, bacterium or fungus, if present in the sample;
subjecting the monomers to reaction conditions effective for the linker elements of either the same or different monomers to undergo bond forming to form multimers if the virus, bacterium or fungus is present in the sample; and
detecting the presence or absence of the virus, bacterium, or fungus in the sample based on the fluorescent signature of the sample subjected to said contacting and said subjecting.

39-48. (canceled)

49. A method of detecting the macromolecular association of one or more target molecules in a sample, said method comprising:
providing a sample potentially containing one or more target molecules capable of undergoing a molecular association;
providing a set of one to six monomers, each monomer comprising:
one or more ligand elements which are useful for binding to the one or more target molecules capable of undergoing a molecular association, with a dissociation constant between the ligand elements and the target molecules of less than 300 μM and
a linker element being connected directly or indirectly through a connector to said one or more ligand elements, said linker element being capable of forming a bond with one or more linker elements of either the same or a different monomer of said set, wherein association of said linker elements, with their ligand elements bound to the one or more target molecules capable of undergoing a molecular association to form a multimer, will generate a unique fluorescent signature different from that produced by those monomers either alone or in association with each other in the absence of the one or more target molecules capable of undergoing a molecular association, when subjected to electromagnetic excitation;
contacting the sample with the set of monomers under conditions effective to allow the ligand elements to bind to the one or more target molecules capable of undergoing a molecular association, if present in the sample;
subjecting the monomers to reaction conditions effective for the linker elements of either the same or different monomers to undergo bond forming to form multimers if the one or more target molecules capable of undergoing a molecular association is present in the sample; and
detecting the presence or absence of one or more target molecules capable of undergoing a molecular association in the sample based on the fluorescent signature of the sample subjected to said contacting and said subjecting.

50-60. (canceled)

61. A method of screening for combinations of monomers useful as fluorescent reporters, said method comprising:
providing a collection of monomers, each monomer comprising:
one or more ligand elements which are useful for binding to a target molecule with a dissociation constant less than 300 μM and
a linker element being connected directly or indirectly through a connector to said one or more ligand elements, said linker element being capable of forming a bond with one or more linker elements of either the same or a different monomer of said collection, wherein association of said linker elements, with their ligand elements bound to the target molecule to form a multimer, will generate a unique fluorescent signature different from that produced by those monomers either alone or in association with each other in the absence of target, when subjected to electromagnetic excitement;
contacting combinations of the collection of monomers with the target molecule under conditions effective to allow the ligand elements to bind to the target molecule;
subjecting monomers to reaction conditions effective for the linker elements of either the same or different monomers to undergo bond forming to form multimers, wherein said subjecting can be carried out before, after, or during said contacting; and
identifying the combinations of monomers that, as a result of said contacting and said subjecting, form multimers and generate a fluorescent signature different from that produced by those monomers either alone or in association with each other in the absence of target.

62-74. (canceled)

75. A method of screening for ligands, said method comprising:
providing a collection of monomers, each of said monomers comprising:
one or more ligands elements having a potential to bind to a target molecule and
a linker element being connected directly or indirectly through a connector to said one or more ligand elements, said linker element being capable of forming a bond with one or more linker elements of either the same or a different monomer of said collection, wherein association of said linker elements of different combinations of monomers, with their ligand elements bound to the target molecule to form a multimer, will generate a unique fluorescent signature different from that produced by those monomers either alone or in association with each other in the absence of target, when subjected to electromagnetic excitement;
contacting combinations of the collection of monomers with the target molecule under conditions effective to allow the ligand elements to bind to the target molecule;
subjecting monomers to reaction conditions effective for the linker elements of either the same or different monomers to undergo bond forming to form multimers, wherein said subjecting can be carried out before, after, or during said contacting; and
identifying the combinations of monomers that, as a result of said contacting and said subjecting, form multimers by binding of their ligands to the target molecule and binding of their linker elements, generate a fluorescent signature different from that produced by those monomers either alone or in association with each other in the absence of the target molecule.

76-92. (canceled)

93. A collection of monomers capable of forming a multimer useful as a fluorescence reporter, each monomer comprising:
one or more ligand elements which are useful for binding to a target molecule with a dissociation constant less than 300 μM and
a linker element being connected directly or indirectly through a connector to said one or more ligand elements, said linker element being capable of forming a bond with one or more linker elements of either the same or a different monomer of said collection, wherein association of said linker elements with their ligand elements bound to the target molecule to form a multimer, will generate a unique fluorescent signature different from that produced by those monomers either alone or in association with each other in the absence of target, when subjected to electromagnetic excitation.

94-141. (canceled)

142. A multimer useful as a fluorescent reporter comprising:
a plurality of covalently or non-covalently linked monomers, each monomer comprising:
one or more ligand elements which are useful for binding to a target molecule with a dissociation constant less than 300 μM and
a linker element being connected directly or indirectly through a connector to said one or more ligand elements, said linker element being capable of forming a bond with one or more linker elements of either the same or a different monomer of said plurality of monomers, wherein association of said linker elements with their ligand elements bound to the target molecule to form a multimer will generate a unique fluorescent signature different from that produced by those monomers either alone or in association with each other in the absence of target, when subjected to electromagnetic excitement.

143-155. (canceled)
